(19) United States
(12) Patent Application Publication
(10) Pub. No.: US 2024/0342318 A1
(43) Pub. Date: Oct. 17, 2024

(54) CONJUGATES COMPRISING COVALENT BINDERS FOR TARGETING INTRACELLULAR PROTEINS

(71) Applicant: RayzeBio, Inc., San Diego, CA (US)

(72) Inventors: Nicholas D. SMITH, San Diego, CA (US); Joe TRAN, San Diego, CA (US)

(21) Appl. No.: 18/434,621

(22) Filed: Feb. 6, 2024

Related U.S. Application Data
(63) Continuation of application No. PCT/US2022/039601, filed on Aug. 5, 2022.
(60) Provisional application No. 63/230,439, filed on Aug. 6, 2021.

(51) Int. Cl. A61K 51/04; A61K 47/54

(52) U.S. Cl. CPC A61K 51/0459; A61K 47/547; A61K 51/0497

(57) ABSTRACT

Provided herein are radiopharmaceutical conjugate compositions and uses thereof. In one aspect, provided herein are conjugates that comprises a targeting ligand that covalently binds to a mutated or an overexpressed protein, optionally a linker, and a metal chelator. The conjugate can be configured to bind with a mutated or overexpressed protein inside a cell. Further provided herein are methods of treating cancer by administering the described conjugates and compositions.

### CROSS REFERENCE TO RELATED APPLICATIONS

This application is the U.S. By-pass Continuation of International Application No. PCT/US2022/039601, filed Aug. 5, 2022, which claims the benefit of U.S. Provisional Application No. 63/230,439, filed on Aug. 6, 2021, each of which is incorporated herein by reference in its entirety.

### SEQUENCE LISTING

The instant application contains a Sequence Listing which has been submitted electronically in XML format and is hereby incorporated by reference in its entirety. Said XML copy, created on Feb. 1, 2024, is named 59541-720_301_SL.xml and is 29,675 bytes in size.

### BACKGROUND

In the United States, cancer is the leading cause of death for those under 65 years of age, and it accounted for about 21% of all death in 2018. Traditional radiotherapies such as external beam radiation therapy have been used for decades as a standard-of-care treatment for diagnosed cancer patients. While some patients respond to external beam radiation therapy, many others do not. Further, metastasis and circulating tumor cells can spread and remain in the bloodstream or bodily fluids after standard-of-care treatment and lead to resistance to therapy. The presence of cancer cells in various parts of the body reduces the therapeutic efficacy of traditional radiotherapies. Accordingly, strategies for targeted radiotherapies are being developed, and there remains a need for targeted radiotherapies that have the desired affinity, stability, and exertion profile.

Targeted radiotherapy (TRT) has been shown to be of great benefit for treating disease, particularly in the area of oncology (for example, Lutathera or 177Lu-PSMA-617). In the case of oncology, the TRT construct typically consists of a high affinity ligand that binds to the tumor-expressed antigen. The high affinity ligand may be an anti-body, anti-body fragment or peptide (such as a cyclic or linear peptide), and typically has been designed to have negligible passive diffusion through cell membranes. Rather the construct engages and binds to a tumor antigen or target on the surface of the cell and may undergo internalization into the cell following this extra-cellular engagement.

### SUMMARY

In one aspect, described herein are targeted radiotherapy (TRT) conjugates that engage and bind to intracellular oncology targets. In some embodiments, the conjugates described herein form a covalent bond with intracellular mutated proteins of interest, for example, at cysteine, lysine or serine residues. The intracellular protein can be an intracellular mutated protein. The intracellular protein can be an overexpressed protein. In some embodiments, conjugates described herein are useful as therapeutic agents (such as therapeutics for treating cancer). In some embodiments, conjugates described herein are useful as theranostic agents. In some embodiments, conjugates described herein are used to confirm the expression of an intracellular oncology target protein in a subject.

In one aspect, described herein is a conjugate comprising (a) a targeting ligand, wherein the targeting ligand covalently binds to an intracellular mutated protein; (b) a linker; and (c) a metal chelator. In some embodiments, the conjugate further comprises a radionuclide, wherein the radionuclide is bound to the metal chelator. In one aspect, described herein is a conjugate comprising (a) a targeting ligand, wherein the targeting ligand covalently binds to an intracellular protein, wherein the intracellular protein is mutated; (b) a linker; and (c) a metal chelator. In some embodiments, the conjugate further comprises a radionuclide, wherein the radionuclide is bound to the metal chelator. In some embodiments, the targeting ligand irreversibly binds to the intracellular mutated protein. In some embodiments, the intracellular mutated protein comprises one or more mutant-specific cysteine residues that are absent in a corresponding wild-type sequence of the intracellular mutated protein. In some embodiments, the targeting ligand binds to at least one of the mutant-specific cysteine residues. In some embodiments, the intracellular mutated protein comprises one or more endogenous cysteine residues that are present in a corresponding wild-type sequence of the mutated protein. In some embodiments, the intracellular mutated protein comprises a mutant-specific steric environment in the region around at least one of the endogenous cysteine residues. In some embodiments, the targeting ligand selectively binds to at least one of the endogenous cysteine residues in the intracellular mutated protein over a same cysteine residue in a corresponding wild-type protein. In one aspect, described herein is a conjugate comprising: (a) a targeting ligand, wherein the targeting ligand covalently binds to an intracellular protein, wherein the intracellular protein is overexpressed compared to a corresponding wild-type protein, and wherein the intracellular protein comprises one or more endogenous cysteine residues; and (c) a metal chelator. In some embodiments, the targeting ligand comprises an electrophilic functional group. In some embodiments, the electrophilic functional group covalently binds to the intracellular mutated protein at a cysteine residue. In some embodiments, the electrophilic functional group comprises an ester, acrylamide, chloroacetamide, acyl azide, acyl nitrile, aldehyde, ketone, alkyl halide, alkyl sulfonate, anhydride, aryl halides, boronic acid, boronate, carboxylic acid, hydrazide, carbamate, carbodiimide, diazoalkane, epoxide, haloacetamide, halotriazine, imido ester, isocyanate, isothiocyanate, maleimide, phosphoramidite, silyl halide, sulfonate ester, sulfonyl halide, α,β-unsaturated thione, α,β-unsaturated carbonyl, α-ketoamide, vinyl sulfone, vinyl amide, vinyl arylene, sulfonamide, propargyl amide group, or propargyl ketone group, each of the functional group is optionally substituted. In some embodiments, the electrophilic functional group is a substituted or unsubstituted acrylamide. In some embodiments, the substituted acrylamide is halo-acrylamide. In some embodiments, the substituted acrylamide is 2-fluoroacrylamide, 2-chloroacrylamide, or a derivative thereof. In some embodiments, the electrophilic functional group is an α,β-unsaturated carbonyl. In some embodiments, the α,β-unsaturated carbonyl is an α,β-unsaturated ketone, α,β-unsaturated aldehyde, α,β-unsaturated amide, α,β-unsaturated acid, or α,β-unsaturated ester, each of which is optionally substituted.

In some embodiments, the electrophilic functional group comprises a structure of Formula (Ia):

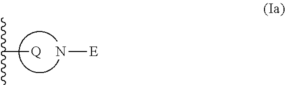

wherein,
ring Q is an optionally substituted 3 to 10 membered heterocycloalkylene; and
E comprises a structure of Formula (Ic),

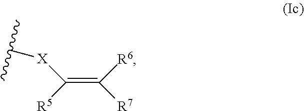

wherein,
X is C(═O), OC(═O), P(═O)OR², C(═S), S(═O)ₙ, or OS(O)ₙ;
n is 1 or 2;
R² is H or substituted or unsubstituted C₁-C₃ alkyl;
R⁵ and R⁷ are each independently selected from H, —CN, halogen, substituted or unsubstituted C₁-C₆ alkyl, substituted or unsubstituted C₁-C₆ heteroalkyl, substituted or unsubstituted C₃-C₇ cycloalkyl, or substituted or unsubstituted C₂-C₇ heterocycloalkyl; or R⁵ and R⁷ taken together form a bond; and
R⁶ is H, halogen, —CN, C₁-C₆ alkyl, C₁-C₆ heteroalkyl, heteroaryl, aryl, alkylaminylalkyl, dialkylaminylalkyl, cycloalkyl or heterocycloalkyl, each of which is optionally substituted.

In some embodiments, the electrophilic functional group comprises a structure of Formula (Ib):

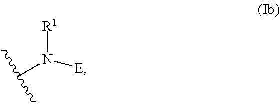

wherein,
R¹ is H, substituted or unsubstituted C₁-C₆ alkyl, substituted or unsubstituted C₁-C₆ heteroalkyl, substituted or unsubstituted C₃-C₆ cycloalkyl, or substituted or unsubstituted C₂-C₅ heterocycloalkyl; and
E comprises a structure of Formula (Ic),

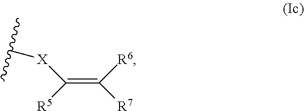

wherein,
X is C(═O), OC(═O), P(═O)OR², C(═S), S(═O)ₙ, or OS(O)ₙ;
n is 1 or 2;
R² is H or substituted or unsubstituted C₁-C₃ alkyl;
R⁵ and R⁷ are each independently selected from H, —CN, halogen, substituted or unsubstituted C₁-C₆ alkyl, substituted or unsubstituted C₁-C₆ heteroalkyl, substituted or unsubstituted C₃-C₆ cycloalkyl, or substituted or unsubstituted C₂-C₅ heterocycloalkyl; or R⁵ and R⁷ taken together form a bond;
R⁶ is H, halogen, —CN, C₁-C₆ alkyl, C₁-C₆ heteroalkyl, heteroaryl, aryl, alkylaminylalkyl, dialkylaminylalkyl, cycloalkyl or heterocycloalkyl, each of which is optionally substituted.

In some embodiments, the electrophilic functional group comprises a structure of Formula (Id),

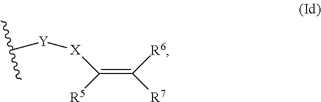

wherein,
X is C(═O), OC(═O), NR²C(═O), N(═NR²), NR²P(═O)OR², C(═S), S(═O)ₙ, OS(O)ₙ, NR²S(═O)ₙ, wherein n is 1 or 2;
Y is a bond, alkylene, heteroalkylene, cycloalkylene, heterocycloalkylene, arylene or heteroarylene, each of which is optionally substituted;
each R² is independently H or substituted or unsubstituted C₁-C₃ alkyl;
R⁵ and R⁷ are each independently selected from H, —CN, halogen, substituted or unsubstituted C₁-C₆ alkyl, substituted or unsubstituted C₁-C₆ heteroalkyl, substituted or unsubstituted C₃-C₆ cycloalkyl, or substituted or unsubstituted C₂-C₅ heterocycloalkyl; or R⁵ and R⁷ taken together form a bond;
R⁶ is H, halogen, —CN, C₁-C₆ alkyl, C₁-C₆ heteroalkyl, heteroaryl, aryl, alkylaminylalkyl, dialkylaminylalkyl, cycloalkyl or heterocycloalkyl, each of which is optionally substituted.

In some embodiments, E is

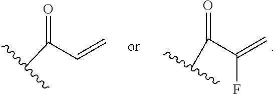

In some embodiments, the intracellular protein (e.g., intracellular mutated protein) is a tumor associated protein. In some embodiments, the intracellular protein (e.g., intracellular mutated protein) is encoded by a gene selected from KRAS, FGFR1, FGFR2, FGFR3, FGFR4, FGFRL1, TP53, IDH1, GNAS, FBXW7, CTNNB1, DNMT3A, EGFR, BTK, ERBB2, ERBB3, and JAK3. In some embodiments, the intracellular protein (e.g., intracellular mutated protein) is a GTPase KRas (KRAS protein), Tumor Protein P53 (TP53 protein), Isocitrate Dehydrogenase (NADP(+)) 1 (IDH1 protein), Guanine Nucleotide binding protein (GNAS protein), F-Box And WD Repeat Domain Containing 7 (FBXW7 protein), Catenin beta-1 (CTNNB1 protein), DNA (cytosine-5)-methyltransferase 3A (DNMT3A protein), Epidermal growth factor receptor (EGFR protein), Bruton's tyrosine kinase (BTK protein), Janus kinase 3 (JAK3 protein), Fibroblast growth factor receptor (FGFR protein), Human epidermal growth factor receptor 2 (HER2), or Human epidermal growth factor receptor 3 (HER3). In some embodiments, the targeting ligand forms a covalent bond with a KRAS protein at position 13 according to SEQ ID NO:1, wherein the KRAS protein comprises a G to C amino acid substitution at position 13. In some embodiments, the targeting ligand covalently binds to a Fibroblast growth factor receptor 3 (FGFR3 protein). In some embodiments, the targeting ligand forms a covalent bond with the FGFR3 protein at position 249 according to SEQ ID NO:2, wherein the FGFR3 protein comprises an S to C amino acid substitution at position 249. In some embodiments, the targeting ligand forms a covalent bond with the FGFR3 protein at position 373 according to SEQ ID NO:2, wherein the FGFR3 protein comprises a Y to C amino acid substitution at position 373. In some embodiments, the targeting ligand forms a covalent bond with the FGFR3 protein at position 248 according to SEQ ID NO:2, wherein the FGFR3 protein comprises an R to C amino acid substitution at position 248. In some embodiments, the targeting ligand forms a covalent bond with the FGFR3 protein at position 370 according to SEQ ID NO:2, wherein the FGFR3 protein comprises a G to C amino acid substitution at position 370. In some embodiments, the targeting ligand forms a covalent bond with a TP53 protein at position 273 according to SEQ ID NO:3, wherein the TP53 protein comprises an R to C amino acid substitution at position 273. In some embodiments, the targeting ligand forms a covalent bond with a TP53 protein at position 220 according to SEQ ID NO:3, wherein the TP53 protein comprises a Y to C amino acid substitution at position 220. In some embodiments, the targeting ligand forms a covalent bond with a TP53 protein at position 163 according to SEQ ID NO:3, wherein the TP53 protein comprises a Y to C amino acid substitution at position 163. In some embodiments, the targeting ligand forms a covalent bond with an IDH1 protein at position 132 according to SEQ ID NO:4, wherein the IDH1 protein comprises an R to C amino acid substitution at position 132. In some embodiments, the targeting ligand forms a covalent bond with a GNAS protein at position 201 according to SEQ ID NO:5, wherein the GNAS protein comprises a mutation at position 201. In some embodiments, the mutation at position 201 comprises an amino acid substitution, wherein the amino acid substitution is R to C. In some embodiments, the targeting ligand forms a covalent bond with an FBXW7 protein at position 465 according to SEQ ID NO:6, wherein the FBXW7 protein comprises a mutation at position 465. In some embodiments, the mutation at position 465 comprises an amino acid substitution, wherein the amino acid substitution is R to C. In some embodiments, the targeting ligand forms a covalent bond with a CTNNB1 protein at position 33 according to SEQ ID NO:7, wherein the CTNNB1 protein comprises a mutation at position 33. In some embodiments, the mutation at position 33 comprises an amino acid substitution, wherein the amino acid substitution is S to C. In some embodiments, the targeting ligand forms a covalent bond with a CTNNB1 protein at position 37 according to SEQ ID NO:7, wherein the CTNNB1 protein comprises a mutation at position 37. In some embodiments, the mutation at position 37 comprises an amino acid substitution, wherein the amino acid substitution is S to C. In some embodiments, the targeting ligand forms a covalent bond with a DNMT3A protein at position 882 according to SEQ ID NO:8, wherein the DNMT3A protein comprises a mutation at position 882. In some embodiments, the mutation at position 37 comprises an amino acid substitution, wherein the amino acid substitution is R to C. In some embodiments, the intracellular protein (e.g., intracellular mutated protein) is an EGFR protein. In some embodiments, the EGFR protein has one or more of the following mutations: G719A, G719S, G719C, deletions of exon 19, insertions in exon 20, T790M, S768I, L858R, and L861Q, wherein the protein sequence numbering is based on SEQ ID NO: 9. In some embodiments, the EGFR protein comprises a T to M amino acid substitution at position 790. In some embodiments, the targeting ligand form a covalent bond with a serine residue of the intracellular protein. In some embodiments, the targeting ligand comprises an α-ketoamide, acrylamide, carbonyl, sulfonyl, beta-lactam, nitrile, carbamate, or oxaborolane group, each of the functional group is optionally substituted. In some embodiments, the targeting ligand comprises a structure selected from

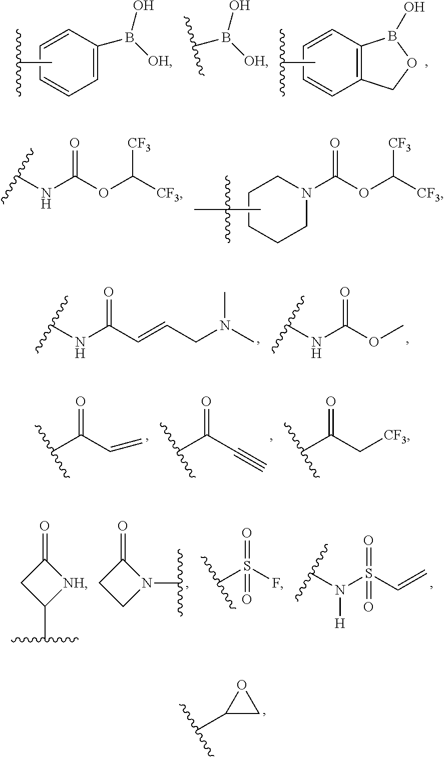

and —CN. In some embodiments, the targeting ligand form a covalent bond with a lysine residue of the intracellular mutated protein. In some embodiments, the targeting ligand comprises an succinimide, thioester, salicylic aldehydes, o-carbonylboronic derivatives, aryl sulfonyl, or saccharine (benzo[d]isothiazole 1,1-dioxide), each of the functional group is optionally substituted. In some embodiments, the targeting ligand comprises a structure selected from

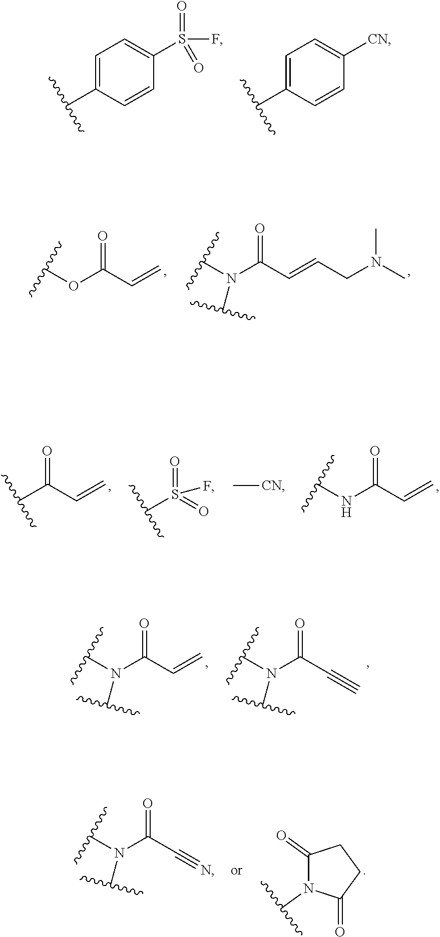

In some embodiments, the targeting ligand acts as an agonist or an antagonist of the intracellular protein (e.g., intracellular mutated protein). In some embodiments, the targeting ligand forms a covalent bond with the intracellular protein (e.g., intracellular mutated protein). In some embodiments, the targeting ligand is covalently linked to the metal chelator. In some embodiments, the metal chelator is selected from AAZTA, BAT, BAT-TM, Crown, Cyclen, DO2A, CB-DO2A, DO3A, H3HP-DO3A, Oxo-DO3A, p-NH₂-Bn-Oxo-DO3A, DOTA, DOTA-3py, DOTA-PA, DOTA-GA, DOTA-4AMP, DOTA-2py, DOTA-1py, p-SCN-Bn-DOTA, CHX-A″-EDTA, MeO-DOTA-NCS EDTA, DOTAMAP, DOTAGA, DOTAGA-anhydride, DOTMA, DOTASA, DOTAM, DOTP, CB-Cyclam, TE2A, CB-TE2A, CB-TE2P, DM-TE2A, MM-TE2A, NOTA, NOTP, HEHA, HEHA-NCS, p-SCN-Bn-HEHA, DTPA, CHX-A″-DTPA, p-NH₂-Bn-CHX-A″-DTPA, p-SCN-DTPA, p-SCN-Bz-Mx-DTPA, 1B4M-DTPA, p-SCN-Bn1B-DTPA, p-SCN-Bn-1B4M-DTPA, p-SCN-Bn-CHX-A″-DTPA, PEPA, p-SCN-Bn-PEPA, TETPA, DOTPA, DOTMP, DOTPM, t-Bu-calix[4]arene-tetracarboxylic acid, macropa, macropa-NCS, macropid, H₃L¹, H₃L⁴, H₂azapa, H₅decapa, bispa², H₄pypa, H₄octapa, H₄CHXoctapa, p-SCN-Bn-H₄octapa, p-SCN-Bn-H₄octapa, TTHA, p-NO₂-Bn-neunpa, H₄octox, H₂macropa, H₂bispa², H₄phospa, H₆phospa, p-SCN-Bn-H₆phospa, TETA, p-NO₂-Bn-TETA, TRAP, TPA, HBED, SHBED, HBED-CC, (HBED-CC)TFP, DMSA, DMPS, DHLA, lipoic acid, TGA, BAL, Bis-thioseminarabazones, p-SCN-NOTA, nNOTA, NODAGA, CB-TE1A1P, 3P-C-NETA-NCS, 3p-C-DEPA, 3P-C-DEPA-NCS, TCMC, PCTA, NODIA-Me, TACN, pycup1A1B, pycup2A, THP, DEDPA, H₂DEDPA, p-SCN-Bn-H₂DEDPA, p-SCN-Bn-TCMC, motexafin, NTA, NOC, 3p-C-NETA, p-NH₂-Bn-TE3A, SarAr, DiAmSar, SarAr-NCS, AmBaSar, BaBaSar, TACN-TM, CP256, C-NE3TA, C-NE3TA-NCS, NODASA, NETA-monoamide, C-NETA, NOPO, BPCA, p-SCN-Bn-DFO, DFO-ChX-Mal, DFO, DFO-IAC, DFO-BAC, DiP-LICAM, EC, SBAD, BAPEN, TACHPYR, NEC-SP, LP^{y}, L1, L2, L3, and EuK-106. In some embodiments, the metal chelator is 2,2′,2″,2′″-((2S,5S,8S,11S)-2,5,8,11-tetramethyl-1,4,7,10-tetraazacyclododecane-1,4,7,10-tetrayl)tetraacetic acid. In some embodiments, the metal chelator is 2,2′,2″,2′″-((2S,5S,8S,11S)-2,5,8,11-tetraethyl-1,4,7,10-tetraazacyclododecane-1,4,7,10-tetrayl)tetraacetic acid. In some embodiments, the metal chelator is a metal chelator in FIG. 1 to FIG. 15. In some embodiments, the metal chelator is DOTA. In some embodiments, the linker is configured to covalently attach the targeting ligand to the metal chelator. In some embodiments, the linker comprises at least one group selected from the group consisting of alkylene, alkenylene, alkynylene, cycloalkylene, arylene, heteroalkylene, heterocycloalkylene and heteroarylene, wherein each of the alkylene, alkenylene, alkynylene, cycloalkylene, arylene, heteroalkylene, heterocycloalkylene or heteroarylene is optionally substituted. In some embodiments, the linker comprises one or more groups selected from the group consisting of —O—, —S—, —NH—, —NH—(CH₂)ₚ—NH, —NH—(CH₂)ₚ—O, —O—(CH₂)ₚ—O, —(C═O)—, —(C═O)—O—, —O(C═O)—, —O(C═O)—O—, —OC(═O)—NH—, —C(═O)NH—, —NHC(═O)—, —NHC(═O)—O—, or —NHC(═O)—NH—, —(C═O)—(CH₂CH₂)_{q}—(C═O)—, —(C═O)—(CH═CH)_{q}—(C═O), —(C═O)—(OCH₂CH₂O)_{q}—(C═O)—, —(CH₂CH₂O)_{q}—, —(C═O)—(CH₂CH₂O)_{q}—, and —(CH(CH₃)C(═O)O)_{q}—, wherein q is 1-20 and p is 1-20. In some embodiments, the linker is or comprises at least one amino acid. In some embodiments, the linker is or comprises two amino acids. In some embodiments, the linker is or comprises three amino acids. In some embodiments, the linker comprises substituted or unsubstituted C₁-C₆ alkylene or substituted or unsubstituted C₁-C₆ heteroalkylene. In some embodiments, the radionuclide is astatine-211, astatine-217, actinium-225, americium-243, radium-223, lead-212, lead-203, copper-64, copper-67, copper-60, copper-61, copper-62, bismuth-212, bismuth-213, gallium-68, gallium-67, dysprosium-154, gadolinium-148, gadolinium-153, samarium-146, samarium-147, samarium-153, terbium-149, thorium-227, thorium-229, iron-59, yttrium-86, indium-111, holmium-166, technetium-94, technetium-99^{m}, yttrium-90, lutetium-177, terbium-161, rhenium-186, rhenium-188, cobalt-55, scandium-43, scandium-44, scandium-47, dysprosium-166, fluorine-18, or iodine-131. In some embodiments, the radionuclide is lutetium-177, actinium-225, yttrium-90, bismuth-213, gallium-68, copper-64 or indium-111. In some embodiments, the radionuclide is an alpha particle-emitting radionuclide. In some embodiments, the alpha particle-emitting radionuclide is actinium-225, astatine-211, thorium-227, terbium-149, or radium-223. In some embodiments, the alpha particle-emitting radionuclide is actinium-225. In some embodiments, the radionuclide is a beta particle-emitting radionuclide. In some embodiments, the beta particle-emitting radionuclide is zirconium-89, yttrium-90, iodine-131, samarium-153, lutetium-177, or lead-212. In some embodiments, the radionuclide is a gamma particle-emitting radionuclide. In some embodiments, the gamma particle-emitting radionuclide is indium-111. In some embodiments, the conjugate has an elimination half-life in a subject of about 0.1 to about 120 hours. In some embodiments, the conjugate has an elimination half-life in a subject of about 0.1 to about 6 hours. In some embodiments, the conjugate has an elimination half-life in a subject of about 0.25 to about 5 hours. In some embodiments, the conjugate has an elimination half-life in a subject of about 0.5 to about 4 hours. In some embodiments, the conjugate has an elimination half-life in a subject of about 1 to about 3 hours. In some embodiments, the conjugate has a residence time of at least about 12 hours in a tumor when administered to a subject having the tumor. In some embodiments, the conjugate has a residence time of about 12 hours to 10 days in a tumor when administered to a subject having the tumor.

In one aspect, provided herein is a pharmaceutical composition comprising a conjugate of the present disclosure and a pharmaceutically acceptable excipient or carrier. In some embodiments, the pharmaceutical composition is formulated for intravenous administration.

In one aspect, provided herein is a method of treating cancer in a subject in need thereof, comprising administering to the subject a conjugate of the present disclosure or a pharmaceutical composition comprising the conjugate. In some embodiments, the cancer is a breast cancer, head and neck squamous cell carcinoma, non-small cell lung cancer, hepatocellular cancer, colorectal cancer, gastric adenocarcinoma, melanoma, or advanced cancer. In some embodiments, a cancer to be treated by the methods of treatment of the present disclosure is selected from the group consisting of carcinoma, squamous carcinoma, adenocarcinoma, sarcomata, endometrial cancer, breast cancer, ovarian cancer, cervical cancer, fallopian tube cancer, primary peritoneal cancer, colon cancer, colorectal cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, glioblastoma, glioma, squamous cell carcinoma of the head and neck, prostate cancer, pancreatic cancer, mesothelioma, sarcoma, hematological cancer, leukemia, lymphoma, neuroma, or any combinations thereof.

In one aspect, provided herein is a method of delivering a radionuclide to a cell comprising administering a conjugate of the present disclosure or a pharmaceutical composition comprising the conjugate. In some embodiments, the conjugate irreversibly binds to an intracellular protein of the cell. In one aspect, provided herein is a method of killing a cell harboring a mutated protein comprising contacting the cell with a conjugate of the present disclosure or a pharmaceutical composition comprising the conjugate, thereby delivering a dose of radiation to the cell.

In one aspect, provided herein is a covalently modified protein, comprising: (a) an intracellular mutated protein comprising a cysteine residue; and (b) a radiopharmaceutical conjugate described herein. In some embodiments, the radiopharmaceutical conjugate comprises an electrophilic functional group, such as a structure of Formula (Ia), (Ib), (Ic) or (Id). In some embodiments, the intracellular mutated protein is selected from KRAS, FGFR1, FGFR2, FGFR3, FGFR4, FGFRL1, TP53, IDH1, GNAS, FBXW7, CTNNB1, DNMT3A, EGFR, BTK, ERBB2, ERBB3, and JAK3.

In one aspect, provided herein is a method of diagnosing cancer patients harboring a mutated or overexpressed protein comprising administering to a patient a conjugate of the present disclosure or a pharmaceutical composition comprising the conjugate. In one aspect, provided herein is a method of imaging a cancer harboring a mutated or overexpressed protein comprising administering to a patient a conjugate of the present disclosure or a pharmaceutical composition comprising the conjugate. In some embodiments, the method further comprises measuring the concentration of the conjugate accumulated in the patient. In some embodiments, the method further comprises measuring the amount of radiation emitted from the radionuclide. In some embodiments, the method further comprises analyzing the elimination profile of the conjugate in the patient. In some embodiments, the method further comprises measuring an elimination half-life of the conjugate in the patient.

### INCORPORATION BY REFERENCE

All publications, patents, and patent applications mentioned in this specification are herein incorporated by reference for the specific purposes identified herein.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawing (also “figure” and “FIG.” herein), of which:

FIG. 1-FIG. 15 illustrate the structures of representative metal chelators.

FIG. 16A and FIG. 16B illustrate exemplary radiopharmaceutical conjugates of the present disclosure forming a covalent bond with an intracellular protein. FIG. 16A illustrates a radiopharmaceutical conjugate comprising a radioisotope R* (metal chelator not shown), where R* falls outside of the protein binding pocket. FIG. 16B illustrates a radiopharmaceutical conjugate comprising a radioisotope R* (metal chelator not shown), where R* is within the protein binding pocket.

### DETAILED DESCRIPTION

The following description and examples illustrate embodiments of the present disclosure in detail. It is to be understood that this present disclosure is not limited to the particular embodiments described herein and as such can vary. Those of skill in the art will recognize that there are numerous variations and modifications of this present disclosure, which are encompassed within its scope.

Although various features of the present disclosure may be described in the context of a single embodiment, the features may also be provided separately or in any suitable combination. Conversely, although the present disclosure may be described herein in the context of separate embodiments for clarity, the present disclosure may also be implemented in a single embodiment.

The section headings used herein are for organizational purposes only and are not to be construed as limiting the subject matter described.

All terms are intended to be understood as they would be understood by a person skilled in the art. Unless defined otherwise, all technical and scientific terms used herein have the same meaning as commonly understood by one of ordinary skill in the art to which the disclosure pertains.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only and is not intended to be limiting.

### I. Definitions

As used in the specification and appended claims, unless specified to the contrary, the following terms have the meaning indicated below.

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Alternatively, particularly with respect to biological systems or processes, the term can mean within an order of magnitude, within 5-fold, or within 2-fold, of a value.

The term “comprising” (and related terms such as “comprise” or “comprises” or “having” or “including”) are to be construed in an open, inclusive sense, that is, as “including, but not limited to.” The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

“Amino” refers to the —NH₂radical.

“Cyano” refers to the —CN radical.

“Nitro” refers to the —NO₂ radical.

“Oxo” refers to the ═O radical.

“Hydroxy” or “hydroxyl” refers to the —OH radical.

“Hydroxyalkyl” refers to an alkyl as defined below substituted with one or more hydroxy radicals. In some embodiments, the alkyl is substituted with 1, 2, 3, or 4 hydroxyl radicals. In some embodiments, the alkyl is substituted with 4 hydroxyl radicals. In some embodiments, the alkyl is substituted with 3 hydroxyl radicals, in some embodiments, the alkyl is substituted with 2 hydroxyl radicals. In some embodiments, the alkyl is substituted with 1 hydroxyl radical.

“Acyl” refers to a substituted or unsubstituted alkylcarbonyl, substituted or unsubstituted alkenylcarbonyl, substituted or unsubstituted alkynylcarbonyl, substituted or unsubstituted cycloalkylcarbonyl, substituted or unsubstituted heterocycloalkylcarbonyl, substituted or unsubstituted arylcarbonyl, substituted or unsubstituted heteroarylcarbonyl, amide, or ester, wherein the carbonyl atom of the carbonyl group is the point of attachment. Unless stated otherwise specifically in the specification, an alkylcarbonyl group, alkenylcarbonyl group, alkynylcarbonyl group, cycloalkylcarbonyl group, amide group, or ester group is optionally substituted, for example, with oxo, halogen, amino, nitrile, nitro, hydroxyl, haloalkyl, alkoxy, aryl, cycloalkyl, heterocycloalkyl, heteroaryl, and the like.

“Alkyl” refers to an optionally substituted straight-chain, or optionally substituted branched-chain saturated hydrocarbon monoradical. An alkyl group can have from one to about twenty carbon atoms, from one to about ten carbon atoms, or from one to six carbon atoms. Examples include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl, and hexyl, and longer alkyl groups, such as heptyl, octyl, and the like. Whenever it appears herein, a numerical range such as “C₁-C₆ alkyl” means that the alkyl group consists of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁-C₁₀ alkyl, a C₁-C₉ alkyl, a C₁-C₈ alkyl, a C₁-C₇ alkyl, a C₁-C₆ alkyl, a C₁-C₅ alkyl, a C₁-C₄ alkyl, a C₁-C₃ alkyl, a C₁-C₂ alkyl, or a C₁ alkyl. Unless stated otherwise specifically in the specification, an alkyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, the alkyl is optionally substituted with oxo, halogen, —CN, —CF₃, —OH, —OMe, —NH₂, —NO₂, or —C═CH. In some embodiments, the alkyl is optionally substituted with oxo, halogen, —CN, —CF₃, —OH, or —OMe. In some embodiments, the alkyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkylene is optionally substituted with oxo, halogen, —CN, —CF₃, —OH, —OMe, —NH₂, or —NO₂. In some embodiments, an alkylene is optionally substituted with oxo, halogen, —CN, —CF₃, —OH, or —OMe. In some embodiments, the alkylene is optionally substituted with halogen. In some embodiments, the alkylene is —CH₂—, —CH₂CH₂—, —CH₂CH₂CH₂—, or —CH₂CH(CH₃)CH₂—. In some embodiments, the alkylene is —CH₂—. In some embodiments, the alkylene is —CH₂CH₂—. In some embodiments, the alkylene is —CH₂CH₂CH₂—.

“Alkenyl” refers to an optionally substituted straight-chain, or optionally substituted branched-chain hydrocarbon monoradical having one or more carbon-carbon double-bonds. In some embodiments, an alkenyl group has from two to about ten carbon atoms, or two to about six carbon atoms. The group may be in either the cis or trans configuration about the double bond(s), and should be understood to include both isomers. Examples include, but are not limited to, ethenyl (—CH═CH₂), 1-propenyl (—CH₂CH═CH₂), isopropenyl [—C(CH₃)═CH₂], butenyl, 1,3-butadienyl, and the like. Whenever it appears herein, a numerical range such as “C₂-C₆ alkenyl” means that the alkenyl group may consist of 2 carbon atoms, 3 carbon atoms, 4 carbon atoms, 5 carbon atoms, or 6 carbon atoms, although the present definition also covers the occurrence of the term “alkenyl” where no numerical range is designated. In some embodiments, the alkenyl is a C₂-C₁₀ alkenyl, a C₂-C₉ alkenyl, a C₂-C₈ alkenyl, a C₂-C₇ alkenyl, a C₂-C₆ alkenyl, a C₂-C₅ alkenyl, a C₂-C₄ alkenyl, a C₂-C₃ alkenyl, or a C₂ alkenyl. Unless stated otherwise specifically in the specification, an alkenyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkenyl is optionally substituted with oxo, halogen, —CN, —CF₃, —OH, —OMe, —NH₂, or —NO₂. In some embodiments, an alkenyl is optionally substituted with oxo, halogen, —CN, —CF₃, —OH, or —OMe. In some embodiments, the alkenyl is optionally substituted with halogen.

The term “alkenylene” or “alkenylene chain” refers to an optionally substituted straight or branched divalent hydrocarbon chain in which at least one carbon-carbon double bond is present linking the rest of the molecule to a radical group. In some embodiments, the alkenylene is —CH═CH—, —CH₂CH═CH—, or —CH═CHCH₂—. In some embodiments, the alkenylene is —CH═CH—. In some embodiments, the alkenylene is —CH₂CH═CH—. In some embodiments, the alkenylene is —CH═CHCH₂—.

“Alkynyl” refers to an optionally substituted straight-chain or optionally substituted branched-chain hydrocarbon monoradical having one or more carbon-carbon triple-bonds. In some embodiments, an alkynyl group has from two to about ten carbon atoms, more preferably from two to about six carbon atoms. Examples include, but are not limited to, ethynyl, 2-propynyl, 2-butynyl, 1,3-butadiynyl, and the like. Whenever it appears herein, a numerical range such as “C₂-C₆ alkynyl” means that the alkynyl group may consist of 2 carbon atoms, 3 carbon atoms, 4 carbon atoms, 5 carbon atoms, or 6 carbon atoms, although the present definition also covers the occurrence of the term “alkynyl” where no numerical range is designated. In some embodiments, the alkynyl is a C₂-C₁₀ alkynyl, a C₂-C₉ alkynyl, a C₂-C₈ alkynyl, a C₂-C₇ alkynyl, a C₂-C₆ alkynyl, a C₂-C₅ alkynyl, a C₂-C₄ alkynyl, a C₂-C₃ alkynyl, or a C₂ alkynyl. Unless stated otherwise specifically in the specification, an alkynyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkynyl is optionally substituted with oxo, halogen, —CN, —CF₃, —OH, —OMe, —NH₂, or —NO₂. In some embodiments, an alkynyl is optionally substituted with oxo, halogen, —CN, —CF₃, —OH, or —OMe. In some embodiments, the alkynyl is optionally substituted with halogen. The term “alkynylene” refers to an optionally substituted straight-chain or optionally substituted branched-chain divalent hydrocarbon having one or more carbon-carbon triple-bonds.

“Alkylamino” refers to a radical of the formula —N(Rₐ)₂ where Rₐ is an alkyl radical as defined, or two Rₐ, taken together with the nitrogen atom, can form a substituted or unsubstituted C₂-C₇ heterocyloalkyl ring. Unless stated otherwise specifically in the specification, an alkylamino group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkylamino is optionally substituted with oxo, halogen, —CN, —CF₃, —OH, —OMe, —NH₂, or —NO₂. In some embodiments, an alkylamino is optionally substituted with oxo, halogen, —CN, —CF₃, —OH, or —OMe. In some embodiments, the alkylamino is optionally substituted with halogen. Alkyl groups, as defined above, may be optionally substituted with an alkylamino group (e.g., an alkylaminylalkyl or dialkylaminylalkyl).

“Alkoxy” or “alkoxyl” refers to a radical of the formula —ORₐ where Rₐ is an alkyl radical as defined. Unless stated otherwise specifically in the specification, an alkoxy group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkoxy is optionally substituted with oxo, halogen, —CN, —CF₃, —OH, —OMe, —NH₂, or —NO₂. In some embodiments, an alkoxy is optionally substituted with oxo, halogen, —CN, —CF₃, —OH, or —OMe. In some embodiments, the alkoxy is optionally substituted with halogen.

An alkoxy substituted with one or more halogen is referred to herein as “haloalkoxy.” In some embodiments, the alkoxy is substituted with one, two, or three halogens. In some embodiments, the alkoxy is substituted with one, two, three, four, five, or six halogens. Haloalkoxy can include, for example, iodoalkoxy, bromoalkoxy chloroalkoxy, and fluoroalkoxy. For example, “fluoroalkoxy” refers to an alkoxy radical, as defined above, that is substituted by one or more fluoro radicals.

“Alkylthio”, “alkylsulfoxide”, and “alkylsulfone” refer to a radical of the formula —SRₐ, —S(O)Rₐ, or —S(O)₂Rₐ, respectively, where Rₐ is an alkyl radical as defined. Unless stated otherwise specifically in the specification, an alkylthio, alkylsulfoxide, or alkylsulfone group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkylthio, alkylsulfoxide, or alkylsulfone is optionally substituted with oxo, halogen, —CN, —CF₃, —OH, —OMe, —NH₂, or —NO₂. In some embodiments, an alkylthio, alkylsulfoxide, or alkylsulfone is optionally substituted with oxo, halogen, —CN, —CF₃, —OH, or —OMe. In some embodiments, the alkylthio, alkylsulfoxide, or alkylsulfone is optionally substituted with halogen.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

The term “aryl” refers to a radical comprising at least one aromatic ring wherein each of the atoms forming the ring is a carbon atom. Aryl groups can be optionally substituted. Examples of aryl groups include, but are not limited to phenyl, and naphthyl. In some embodiments, the aryl is phenyl. Depending on the structure, an aryl group can be monovalent or divalent (i.e., an arylene group). Unless stated otherwise specifically in the specification, the term “aryl” or the prefix “ar-” (such as in “aralkyl”) is meant to include aryl radicals that are optionally substituted. In some embodiments, an aryl group comprises a partially reduced cycloalkyl group defined herein (e.g., 1,2-dihydronaphthalene). In some embodiments, an aryl group comprises a fully reduced cycloalkyl group defined herein (e.g., 1,2,3,4-tetrahydronaphthalene). When aryl comprises a cycloalkyl group, the aryl is bonded to the rest of the molecule through an aromatic ring carbon atom. An aryl radical can be a monocyclic or polycyclic (e.g., bicyclic, tricyclic, or tetracyclic) ring system, which may include fused, spiro or bridged ring systems. Unless stated otherwise specifically in the specification, an aryl may be optionally substituted, for example, with halogen, amino, alkylamino, aminoalkyl, nitrile, nitro, hydroxyl, alkyl, alkenyl, alkynyl, haloalkyl, heteroalkyl, alkoxy, aryl, cycloalkyl, heterocycloalkyl, heteroaryl, —S(O)₂NH—C₁-C₆alkyl, and the like. In some embodiments, an aryl is optionally substituted with halogen, methyl, ethyl, —CN, —CF₃, —OH, —OMe, —NH₂, —NO₂, —S(O)₂NH₂, —S(O)₂NHCH₃, —S(O)₂NHCH₂CH₃, —S(O)₂NHCH(CH₃)₂, —S(O)₂N(CH₃)₂, or —S(O)₂NHC(CH₃)₃. In some embodiments, an aryl is optionally substituted with halogen, methyl, ethyl, —CN, —CF₃, —OH, or —OMe. In some embodiments, the aryl is optionally substituted with halogen. In some embodiments, the aryl is substituted with alkyl, alkenyl, alkynyl, haloalkyl, or heteroalkyl, wherein each alkyl, alkenyl, alkynyl, haloalkyl, heteroalkyl is independently unsubstituted, or substituted with halogen, methyl, ethyl, —CN, —CF₃, —OH, —OMe, —NH₂, or —NO₂.

“Aryloxy” refers to an aryl group as defined above connected to the rest of the molecule through —O—.

“Arylthio” refers to an aryl group as defined above connected to the rest of the molecule through —S—.

“Arylsulfoxide” refers to an aryl group as defined above connected to the rest of the molecule through —S(O)—.

“Arylsulfone” refers to an aryl group as defined above connected to the rest of the molecule through —S(O)₂—.

The term “cycloalkyl” refers to a monocyclic or polycyclic non-aromatic radical, wherein each of the atoms forming the ring (i.e. skeletal atoms) is a carbon atom. In some embodiments, cycloalkyls are saturated or partially unsaturated. In some embodiments, cycloalkyls are spirocyclic or bridged compounds. In some embodiments, cycloalkyls are fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Depending on the structure, a cycloalkyl group can be monovalent or divalent (i.e., a cycloalkylene group). Monocyclic cycloalkyl radicals include, for example, cyclopropyl, cyclobutyl, cyclopentyl, cyclohexyl, cycloheptyl, and cyclooctyl. In some embodiments, the monocyclic cycloalkyl is cyclopentyl. In some embodiments, the monocyclic cycloalkyl is cyclopentenyl or cyclohexenyl. In some embodiments, the monocyclic cycloalkyl is cyclopentenyl. Polycyclic radicals include, for example, adamantyl, 1,2-dihydronaphthalenyl, 1,4-dihydronaphthalenyl, tetrainyl, decalinyl, 3,4-dihydronaphthalenyl-1(2H)-one, spiro[2.2]pentyl, norbornyl and bicycle[1.1.1]pentyl. Unless otherwise stated specifically in the specification, a cycloalkyl group may be optionally substituted. Representative cycloalkyls include, but are not limited to, cycloalkyls having from three to fifteen carbon atoms (C₃-C₁₅ cycloalkyl), from three to ten carbon atoms (C₃-C₁₀ cycloalkyl), from three to eight carbon atoms (C₃-C₈ cycloalkyl), from three to six carbon atoms (C₃-C₆ cycloalkyl), from three to five carbon atoms (C₃-C₅ cycloalkyl), or three to four carbon atoms (C₃-C₄ cycloalkyl). A cycloalkyl can comprise a fused, spiro or bridged ring system. In some embodiments, the cycloalkyl comprises a fused ring system. In some embodiments, the cycloalkyl comprises a spiro ring system. In some embodiments, the cycloalkyl comprises a bridged ring system. In some embodiments, the cycloalkyl comprises an alkene (e.g., a cycloalkenyl). In some embodiments, the cycloalkyl comprises an alkyne (e.g., a cycloalkynyl). In some embodiments, the cycloalkyl is a 3- to 6-membered cycloalkyl. In some embodiments, the cycloalkyl is a 5- to 6-membered cycloalkyl. Monocyclic cycloalkyls include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example, cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a cycloalkyl is optionally substituted with oxo, halogen, methyl, ethyl, —CN, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Halo” or “halogen” refers to bromo, chloro, fluoro, or iodo. In some embodiments, halogen is fluoro or chloro. In some embodiments, halogen is fluoro.

“Haloalkyl” refers to an alkyl radical, as defined above, that is substituted by one or more halogens. In some embodiments, the alkyl is substituted with one, two, or three halogens. In some embodiments, the alkyl is substituted with one, two, three, four, five, or six halogens. Haloalkyl can include, for example, iodoalkyl, bromoalkyl, chloroalkyl, and fluoroalkyl. For example, “fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, or combinations thereof. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, or combinations thereof wherein the heteroalkyl is attached to the rest of the molecule at a carbon atom of the heteroalkyl. Examples of such heteroalkyl are, for example, —CH₂—O—CH₃, —CH₂—N(alkyl)-CH₃, —CH₂—N(aryl)-CH₃, —OCH₂CH₂OH, —OCH₂CH₂OCH₂CH₂OH, or —OCH₂CH₂OCH₂CH₂OCH₂CH₂OH. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen. As used herein, a “heteroalkylene” refers to a divalent heteroalkyl group. Exemplary heteroalkylene includes —CH₂OCH₂—, —CH₂NHCH₂—, —NHCH₂NHCH₂—, —NHCH₂CH₂—, etc.

The term “heterocycloalkyl” refers to a cycloalkyl group that includes at least one heteroatom selected from nitrogen, oxygen, and sulfur. Unless stated otherwise specifically in the specification, the heterocycloalkyl radical may be a monocyclic, or bicyclic ring system, which may include fused (when fused with an aryl or a heteroaryl ring, the heterocycloalkyl is bonded through a non-aromatic ring atom) or bridged ring systems. The nitrogen, carbon or sulfur atoms in the heterocyclyl radical may be optionally oxidized. The nitrogen atom may be optionally quaternized. The heterocycloalkyl radical is partially or fully saturated. Examples of heterocycloalkyl radicals include, but are not limited to, dioxolanyl, thienyl[1,3]dithianyl, tetrahydroquinolyl, tetrahydroisoquinolyl, decahydroquinolyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides and oligosaccharides. A heterocycloalkyl can comprise a fused, spiro or bridged ring system. In some embodiments, the heterocycloalkyl comprises a fused ring system. In some embodiments, the heterocycloalkyl comprises a spiro ring system. In some embodiments, the heterocycloalkyl comprises a bridged ring system. Depending on the structure, a heterocycloalkyl group can be monovalent or divalent (i.e., a heterocycloalkylene group). Unless otherwise noted, heterocycloalkyls have from 2 to 12 carbons in the ring. In some embodiments, heterocycloalkyls have from 2 to 10 carbons in the ring. In some embodiments, heterocycloalkyls have from 2 to 10 carbons in the ring and 1 or 2 N atoms. In some embodiments, heterocycloalkyls have from 2 to 10 carbons in the ring and 3 or 4 N atoms. In some embodiments, heterocycloalkyls have from 2 to 12 carbons, 0-2 N atoms, 0-2 O atoms, 0-2 P atoms, and 0-1 S atoms in the ring. In some embodiments, heterocycloalkyls have from 2 to 12 carbons, 1-3 N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heterocycloalkyl is optionally substituted with oxo, halogen, methyl, ethyl, —CN, —CF₃, —OH, —OMe, —NH₂, or —NO₂. In some embodiments, a heterocycloalkyl is optionally substituted with oxo, halogen, methyl, ethyl, —CN, —CF₃, —OH, or —OMe. In some embodiments, the heterocycloalkyl is optionally substituted with halogen.

“Heteroaryl” refers to a ring system radical comprising carbon atom(s) and one or more ring heteroatoms that selected from the group consisting of nitrogen, oxygen, phosphorous, and sulfur, and at least one aromatic ring. In some embodiments, heteroaryl is monocyclic, bicyclic or polycyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-6 N atoms in the ring. In some embodiments, a heteroaryl contains 1-4 N atoms in the ring. In some embodiments, a heteroaryl contains 4-6 N atoms in the ring. In some embodiments, a heteroaryl contains 0-4 N atoms, 0-1 O atoms, 0-1 P atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉ heteroaryl. In some embodiments, monocyclic heteroaryl is a C₁-C₅ heteroaryl. In some embodiments, monocyclic heteroaryl is a 5-membered or 6-membered heteroaryl. In some embodiments, a bicyclic heteroaryl is a C₆-C₉ heteroaryl. In some embodiments, a heteroaryl group comprises a partially reduced cycloalkyl or heterocycloalkyl group defined herein (e.g., 7,8-dihydroquinoline). In some embodiments, a heteroaryl group comprises a fully reduced cycloalkyl or heterocycloalkyl group defined herein (e.g., 5,6,7,8-tetrahydroquinoline). When heteroaryl comprises a cycloalkyl or heterocycloalkyl group, the heteroaryl is bonded to the rest of the molecule through a heteroaromatic ring carbon or hetero atom. A heteroaryl radical can be a monocyclic or polycyclic (e.g., bicyclic, tricyclic, or tetracyclic) ring system, which may include fused, spiro or bridged ring systems. Depending on the structure, a heteroaryl group may be monovalent or divalent (e.g., a heteroarylene group). Unless stated otherwise specifically in the specification, a heteroaryl is optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroaryl is optionally substituted with halogen, methyl, ethyl, —CN, —CF₃, —OH, —OMe, —NH₂, or —NO₂. In some embodiments, a heteroaryl is optionally substituted with halogen, methyl, ethyl, —CN, —CF₃, —OH, or —OMe. In some embodiments, the heteroaryl is optionally substituted with halogen.

The term “moiety” refers to a specific segment or functional group of a molecule. Chemical moieties are often recognized chemical entities embedded in or appended to a molecule.

The terms “treat,” “prevent,” “ameliorate,” and “inhibit,” as well as words stemming therefrom, as used herein, do not necessarily imply 100% or complete treatment, prevention, amelioration, or inhibition. Rather, there are varying degrees of treatment, prevention, amelioration, and inhibition of which one of ordinary skill in the art recognizes as having a potential benefit or therapeutic effect. In this respect, the disclosed methods can provide any amount of any level of treatment, prevention, amelioration, or inhibition of the disorder in a mammal. For example, a disorder, including symptoms or conditions thereof, may be reduced by, for example, about 100%, about 90%, about 80%, about 70%, about 60%, about 50%, about 40%, about 30%, about 20%, or about 10%. Furthermore, the treatment, prevention, amelioration, or inhibition provided by the methods disclosed herein can include treatment, prevention, amelioration, or inhibition of one or more conditions or symptoms of the disorder, e.g., cancer or an inflammatory disease. Also, for purposes herein, “treatment,” “prevention,” “amelioration,” or “inhibition” encompass delaying the onset of the disorder, or a symptom or condition thereof. As used herein, “treating” includes the concepts of “alleviating”, which refers to lessening the frequency of occurrence or recurrence, or the severity, of any symptoms or other ill effects related to a disorder and/or the associated side effects. The term “treating” also encompasses the concept of “managing” which refers to reducing the severity of a particular disease or disorder in a patient or delaying its recurrence, e.g., lengthening the period of remission in a patient who had suffered from the disease.

The term “therapeutically effective amount” as used herein to refer to an amount effective at the dosage and duration necessary to achieve the desired therapeutic result. A therapeutically effective amount of the composition may vary depending on factors such as the individual's condition, age, sex, and weight, and the ability of the protein to elicit the desired response of the individual. A therapeutically effective amount can also be an amount that exceeds any toxic or deleterious effect of the composition that would have a beneficial effect on the treatment.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.).

As used herein, the term “substituent” means positional variables on the atoms of a core molecule that are substituted at a designated atom position, replacing one or more hydrogens on the designated atom, provided that the designated atom's normal valency is not exceeded, and that the substitution results in a stable compound. Combinations of substituents and/or variables are permissible only if such combinations result in stable compounds. A person of ordinary skill in the art should note that any carbon as well as heteroatom with valences that appear to be unsatisfied as described or shown herein is assumed to have a sufficient number of hydrogen atom(s) to satisfy the valences described or shown. In certain instances one or more substituents having a double bond (e.g., “oxo” or “═O”) as the point of attachment may be described, shown or listed herein within a substituent group, wherein the structure may only show a single bond as the point of attachment to the core structure. A person of ordinary skill in the art would understand that, while only a single bond is shown, a double bond is intended for those substituents.

The term “optionally substituted” or “substituted” means that the referenced group is optionally substituted with one or more additional group(s) individually and independently selected from D, oxo, halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from D, halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, optional substituents are independently selected from D, halogen, oxo, —CN, —NH₂, —OH, —NH(CH₃), —N(CH₃)₂, —NH(cyclopropyl), —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic) includes oxo (═O). When indicating the number of substituents, the term “one or more” means from one substituent to the highest possible number of substitution, i.e. replacement of one hydrogen up to replacement of all hydrogens by substituents.

The term “unsubstituted” means that the specified group bears no substituents.

Certain compounds described herein may exist in tautomeric forms, and all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

The term “protein” as used herein refers to a polypeptide (i.e., a string of at least 3 amino acids linked to one another by peptide bonds). Proteins can include moieties other than amino acids (e.g., may be glycoproteins, proteoglycans, etc.) and/or can be otherwise processed or modified. A protein can be a complete polypeptide as produced by and/or active in a cell (with or without a signal sequence). In some embodiments, a protein is or comprises a characteristic portion such as a polypeptide as produced by and/or active in a cell. A protein can include more than one polypeptide chain.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein, C₁-Cₓ (or C₁₋ₓ) includes C₁-C₂, C₁-C₃ . . . C₁-Cₓ. By way of example only, a group designated as “C₁-C₄” indicates that there are one to four carbon atoms in the moiety, i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl. Also, by way of example, C₀-C₂ alkylene includes a direct bond, —CH₂—, and —CH₂CH₂— linkages.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a companion animal such as a dog or a cat. In one aspect, the mammal is a human.

### II. Radiopharmaceutical Conjugates

Provided herein are radiopharmaceutical conjugates and pharmaceutical compositions comprising the conjugates. The conjugates and compositions can be useful for treating cancer. The conjugates and compositions can also be useful in imaging and disease diagnosis.

In some embodiments, described herein is a conjugate comprising: (a) a targeting moiety that covalently binds to an intracellular protein (e.g., intracellular mutated protein), (b) optionally a linker, and (c) a metal chelator. In some embodiments, the linker is configured to covalently bind the targeting ligand to the chelator. In one aspect, the conjugate further comprises a radionuclide. In one aspect, the radionuclide is bound to the metal chelator. In some embodiments, the conjugate is cell permeable. The conjugate can engage intracellular oncology targets.

In some embodiments, a conjugate described herein is designed to have a prescribed elimination profile. The elimination profile can be designed by adjusting the binding affinity of the targeting ligand, the property of the linker, the type of radionuclide, etc. In some embodiments, the conjugate has an elimination half-life in a subject of about 0.1 to about 120 hours. In some embodiments, the conjugate has an elimination half-life in a subject of about 0.1 to about 12 hours. In some embodiments, the conjugate has an elimination half-life in a subject of about 0.1 to about 6 hours. In some embodiments, the conjugate has an elimination half-life in a subject of about 0.25 to about 5 hours. In some embodiments, the conjugate has an elimination half-life in a subject of about 0.5 to about 4 hours. In some embodiments, the conjugate has an elimination half-life in a subject of about 1 to about 3 hours. In some embodiments, the conjugate has an elimination half-life in a subject of about 1 hour to 2 hours. In some embodiments, the conjugate has an elimination half-life in a subject of about 2 hours to 3 hours. In some embodiments, the conjugate has an elimination half-life in a subject of about 3 hours to 4 hours. In some embodiments, the conjugate has an elimination half-life in a subject of about 4 hours to 5 hours. In some embodiments, the conjugate has an elimination half-life in a subject of about 5 hours to 6 hours. In some embodiments, the conjugate has an elimination half-life in a subject of about at least 1 to about at most 120 hours. In some embodiments, the conjugate has an elimination half-life in a subject of at least about 0.1 hour, 0.25 hour, 0.5 hour 1 hour, 2 hours, 3 hours, 4 hours, 5 hours, 7 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours. In some embodiments, the conjugate has an elimination half-life in a subject of at most about 120 hour, 80 hours, 70 hours, 60 hours, 50 hours, 40 hours, 30 hours, 24 hours, 12 hours, 10 hours, 9 hours, 8 hours, 7 hours, 6 hours, 5 hours, 4 hours, 3 hours, 2 hours, or 1 hour. In some embodiments, the conjugate has an elimination half-life in a subject of about 2 to 24 hours. In some embodiments, the conjugate has an elimination half-life in a subject of about 3 to 9 hours. In some embodiments, the conjugate has an elimination half-life in a subject of about 2 to 12 hours. In some embodiments, the conjugate has an elimination half-life in a subject of about 2 to 8 hours. In some embodiments, the conjugate has an elimination half-life in a subject of about at least 0.1 to about at least 6 hours, least about 0.5 to at least about 4 hours, or least about 1 to at least about 3 hours. In some embodiments, the elimination half-life is determined in mice. In some embodiments, the subject is a human. In some embodiments, the subject is a non-human mammal, such as a rat.

In some embodiments, a conjugate described herein can have an elimination half-life in a tumor and non-tumor tissue of the subject. The elimination half-life in a tumor can be the same as or different from (either longer or shorter than) the elimination half-life in a non-tumor issue. In some embodiments, the elimination half-life of the conjugate in a tumor is at least about 0.1, 0.5, 1, 3, 6, 12, 24, 48, 72, 96 or more than 96 hours. In some embodiments, the elimination half-life of the conjugate in a tumor tissue is at least 1.1, 1.2, 1.3, 1.4, 1.5, 2.0, 2.5, 3.0, 4.0, 5.0, 10, 25, 50, or 100 fold greater than the elimination half-life of the conjugate in a non-tumor tissue of the subject.

As used herein, the “elimination half-life” can refer to the time it takes from the maximum concentration after administration to half maximum concentration. In some embodiments, the elimination half-life is determined after intravenous administration. In some embodiments, the elimination half-life is measured as biological half-life, which is the half-life of the cold pharmaceutical in the living system. In some embodiments, the elimination half-life is measured as effective half-life, which is the half-life of a radiopharmaceutical in a living system taking into account the half-life of the radionuclide.

A herein described conjugate can have a residence time of at least about 12 hours in a tumor when administered to a subject having the tumor. In some embodiments, the residence time is at least about 0.5, 1, 1.5, 2, 2.5, 3, 3.5, 4, 4.5, 5, 5.5, 6, 6.5, 7, 7.5, 8, 8.5, 9, 9.5, or 10 days. In some embodiments, the residence time is about 12 hours to 10 days in a tumor when administered to a subject having the tumor.

A conjugate described herein can have a described time-integrated activity coefficient (i.e., a) in a tumor or non-tumor tissues of a subject. As used herein, a represents the cumulative number of nuclear transformations occurring in a source tissue over a dose-integration period per unit administered activity. The ã value of a conjugate can be tuned by modifications of the targeting ligand in the conjugate. The ã value can be determined using a method known in the art. In some embodiments, the ã value of the conjugate in a tumor is from about 6 hours to 14 days. In some embodiments, the ã value in a tumor is about 2 to 10 days. In some embodiments, the ã value in a tumor is about 4 to 7 days. In some embodiments, the ã value in a tumor is about 7 to 10 days. In some embodiments, the ã value in a tumor is from about 1 day, 2 days, 3 days, or 4 days to about 5 days, 6 days, 7 days, 8 days, 9 days, 10 days, 11 days, or 12 days. In some embodiments, the ã value in a tumor is about 3 days, 4 days, 5 days, 6 days, 7 days, 8 days, 9 days, 10 days, 11 days, or 12 days. In some embodiments, the ã value of the conjugate in a non-tumor tissue is from about 6 hours to 14 days. In some embodiments, the ã value in a non-tumor tissue is about 2 to 10 days. In some embodiments, the ã value in a non-tumor tissue is about 4 to 7 days. In some embodiments, the ã value in a non-tumor tissue is about 7 to 10 days. In some embodiments, the ã value in a non-tumor tissue is from about 1 day, 2 days, 3 days, or 4 days to about 5 days, 6 days, 7 days, 8 days, 9 days, 10 days, 11 days, or 12 days. In some embodiments, the ã value in a non-tumor tissue is about 3 days, 4 days, 5 days, 6 days, 7 days, 8 days, 9 days, 10 days, 11 days, or 12 days. The ã value of the conjugate in a tumor can be the same as the ã value of the conjugate in a non-tumor tissue of the subject. The ã value of the conjugate in a tumor can be longer or shorter than the ã value of the conjugate in a non-tumor tissue of the subject. In some embodiments, the ã value of the conjugate in a tumor is at least 1.1, 1.2, 1.3, 1.4, 1.5, 2.0, 2.5, 3.0, 4.0, or 5.0 fold of the ã value of the conjugate in a non-tumor tissue of the subject.

A conjugate described herein can have an ã value in an organ of a subject. In some embodiments, the conjugate has an ã value in a kidney of the subject of at most 24 hours. In some embodiments, the ã value of the conjugate in a kidney of the subject is at most 18 hours, 15 hours, 12 hours, 10 hours, 8 hours, 6 hours, or 5 hours. In some embodiments, the ã value of the conjugate in a kidney of the subject is about 30 minutes to about 24 hours. In some embodiments, the ã value of the conjugate in a kidney of the subject is about 2 to 24 hours. In some embodiments, the ã value of the conjugate in a kidney of the subject is more than 24 hours. In some embodiments, the ã value of the conjugate in a liver of the subject is at most 24 hours. In some embodiments, the ã value of the conjugate in a liver of the subject is at most 18 hours, 15 hours, 12 hours, 10 hours, 8 hours, 6 hours, or 5 hours. In some embodiments, the ã value of the conjugate in a liver of the subject is about 30 minutes to about 24 hours. In some embodiments, the ã value of the conjugate in a liver of the subject is about 2 to 24 hours. In some embodiments, the ã value of the conjugate in a liver of the subject is more than 24 hours.

In some cases, the elimination profile of the conjugate can be adjusted by a reversible binding between the conjugate and a plasma protein such as albumin. A suitable affinity between the conjugate and the plasma protein can utilize the plasma protein as a reservoir for the conjugates, attaching and preserving the conjugates at high concentration and releasing the conjugates at a lower concentration, thereby improving elimination profile. In some embodiments, a dissociation constant (Kd) between the conjugate and human serum albumin is at most 500 μM, as determined at room temperature in human serum condition. In some embodiments, the Kd is from about 0.1 nM to about 1000 μM. In some embodiments, the Kd is at most 100 μM. In some embodiments, the Kd is at most 15 μM. In some embodiments, the Kd is from about 1 nM to about 10 μM. In some embodiments, the Kd is from about 10 nM to about 10 μM. In some embodiments, the Kd is from about 50 nM to about 1 μM. In some embodiments, the Kd is from about 100 nM to about 10 μM.

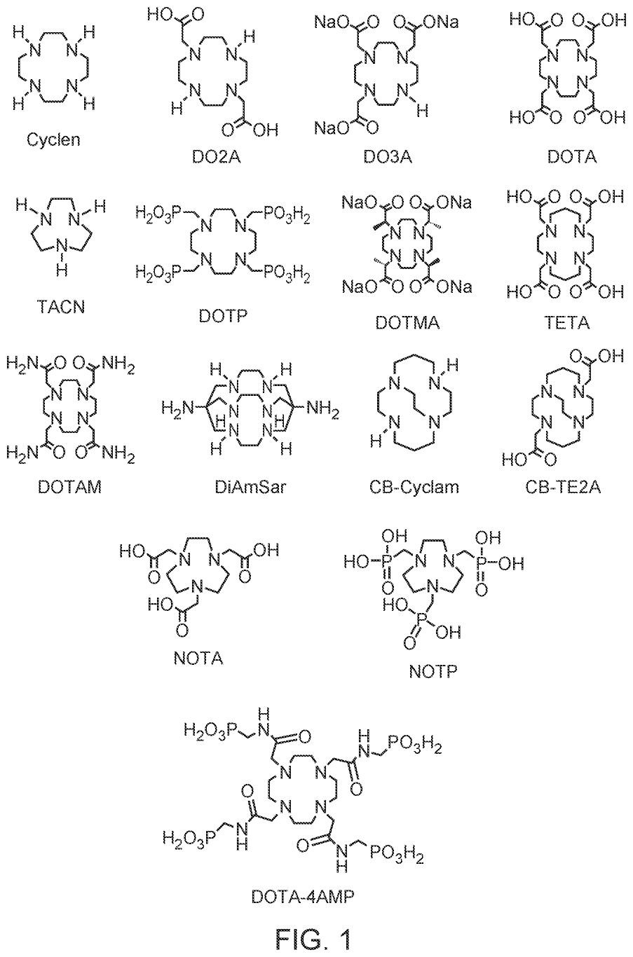

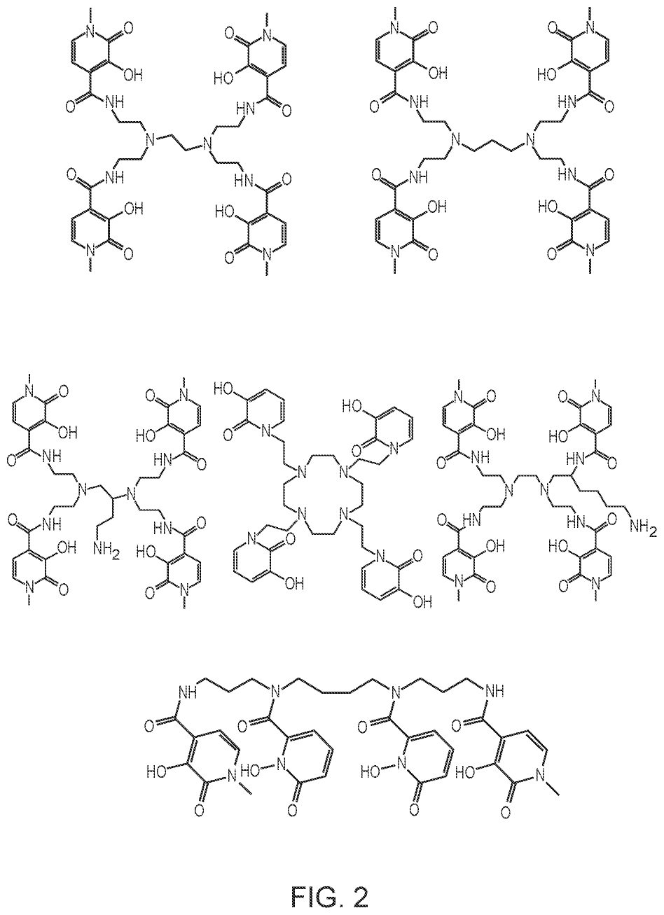

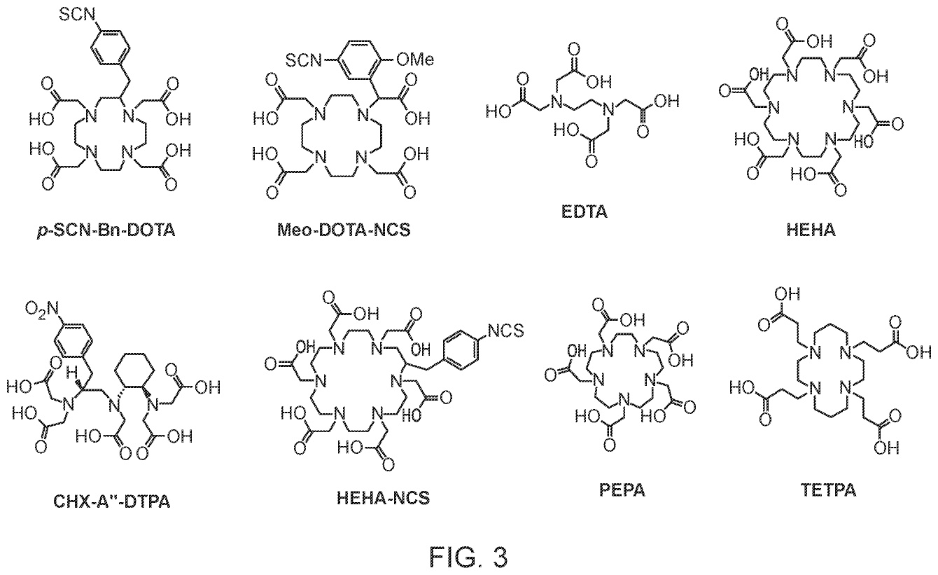

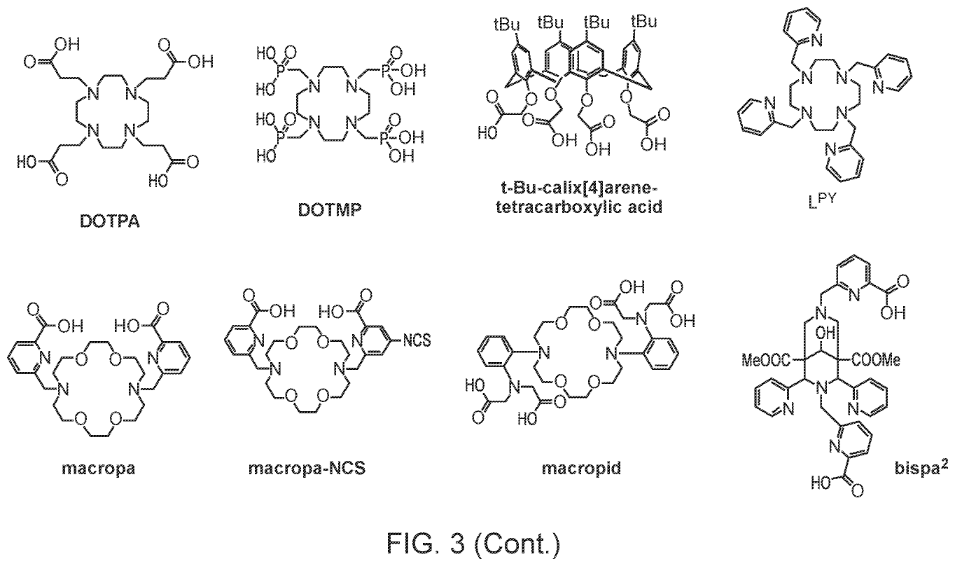

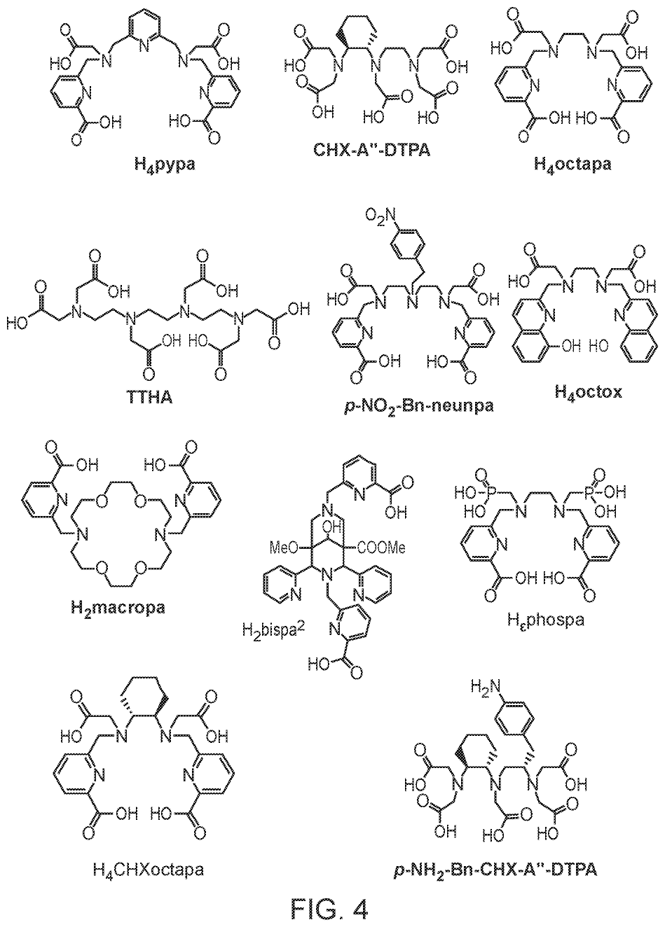

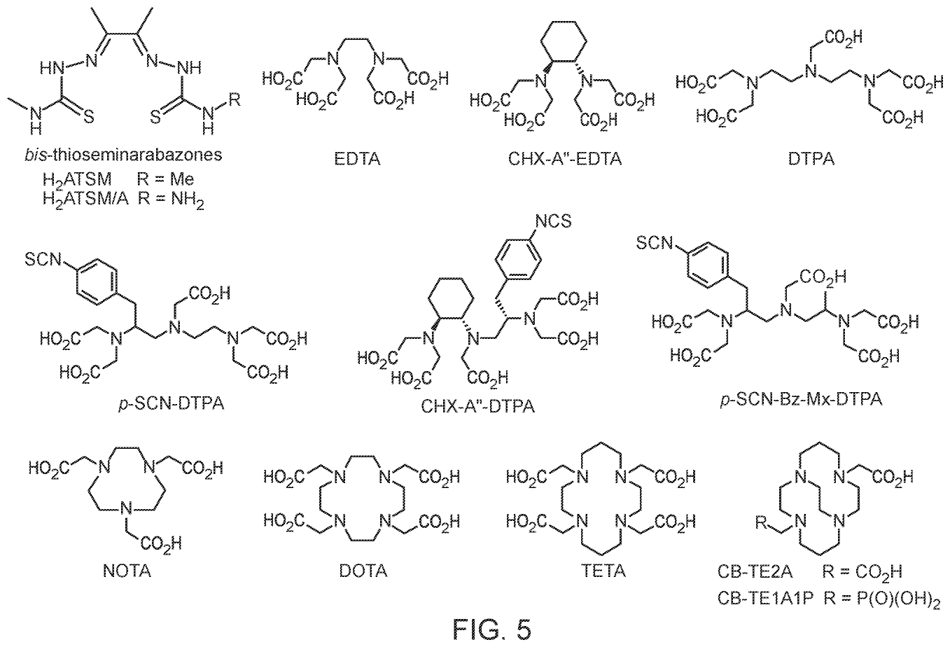

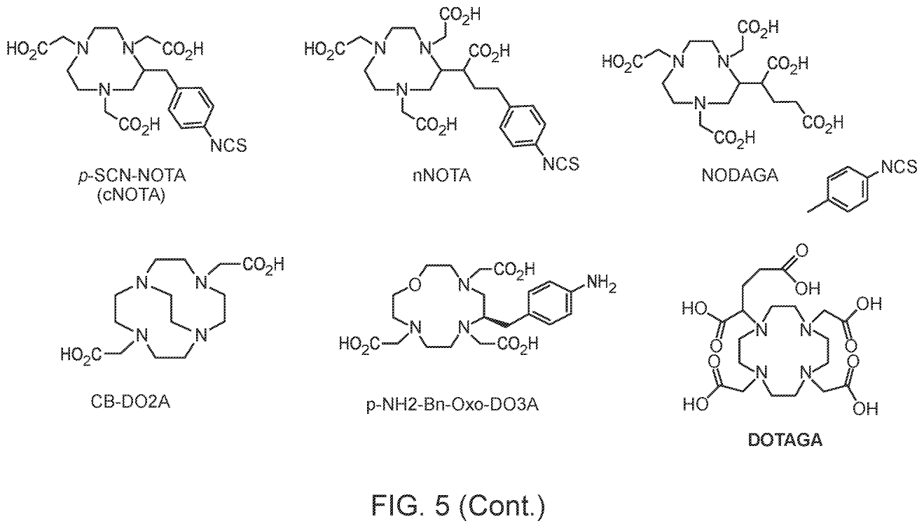

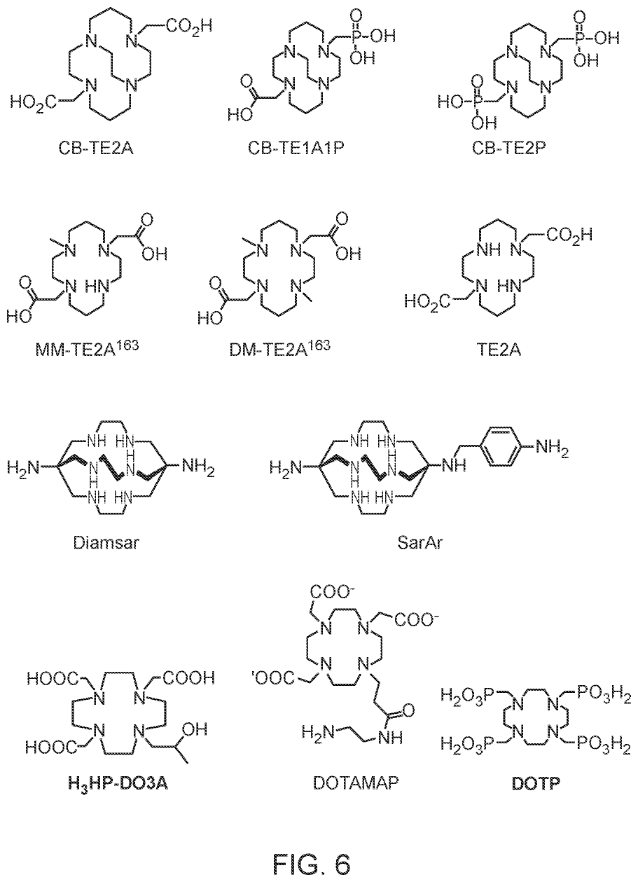

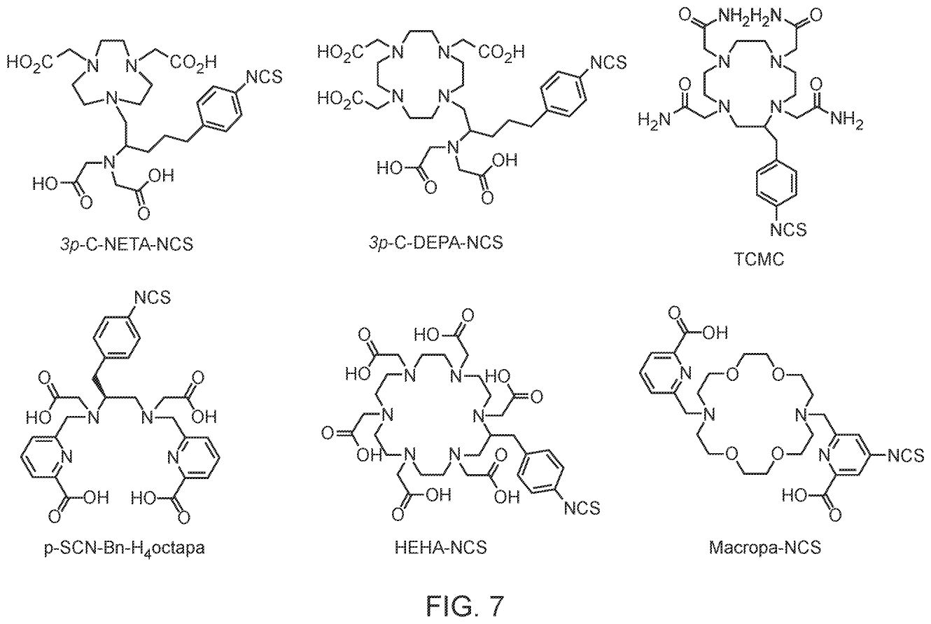

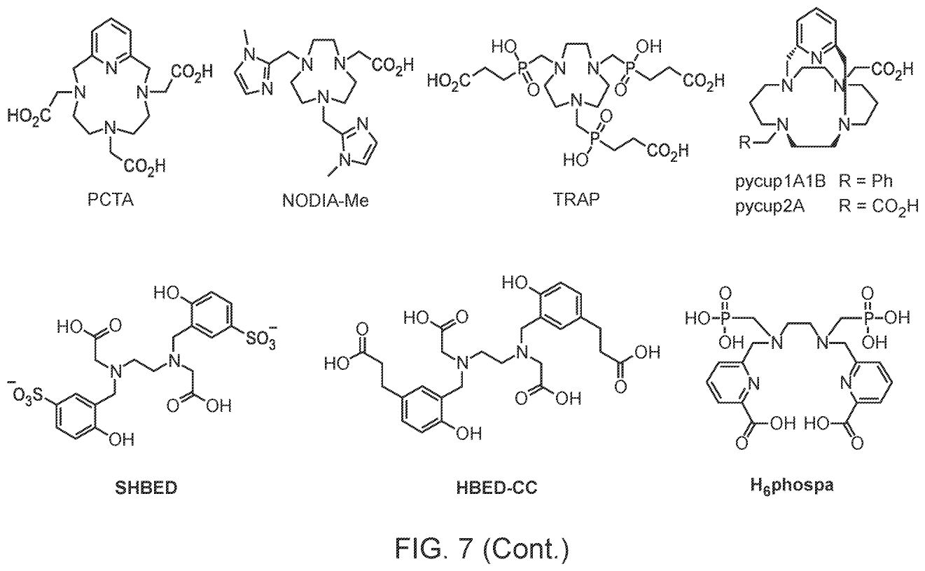

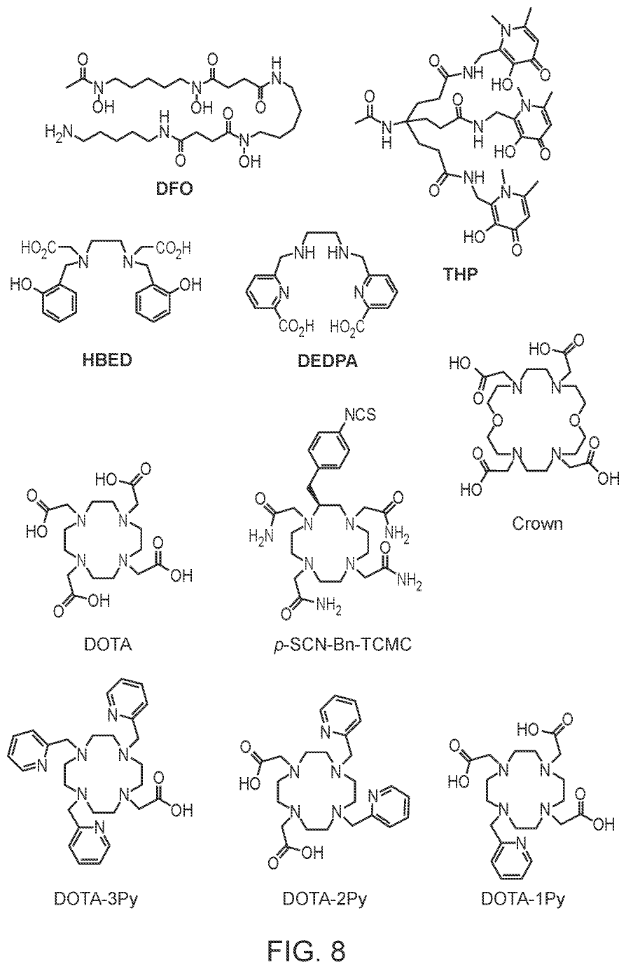

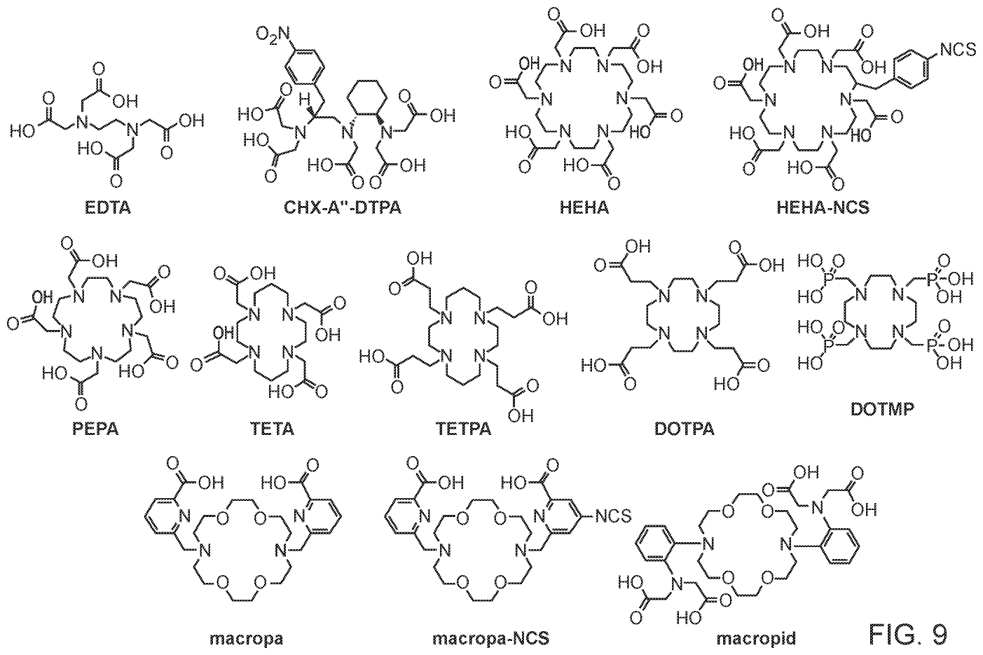

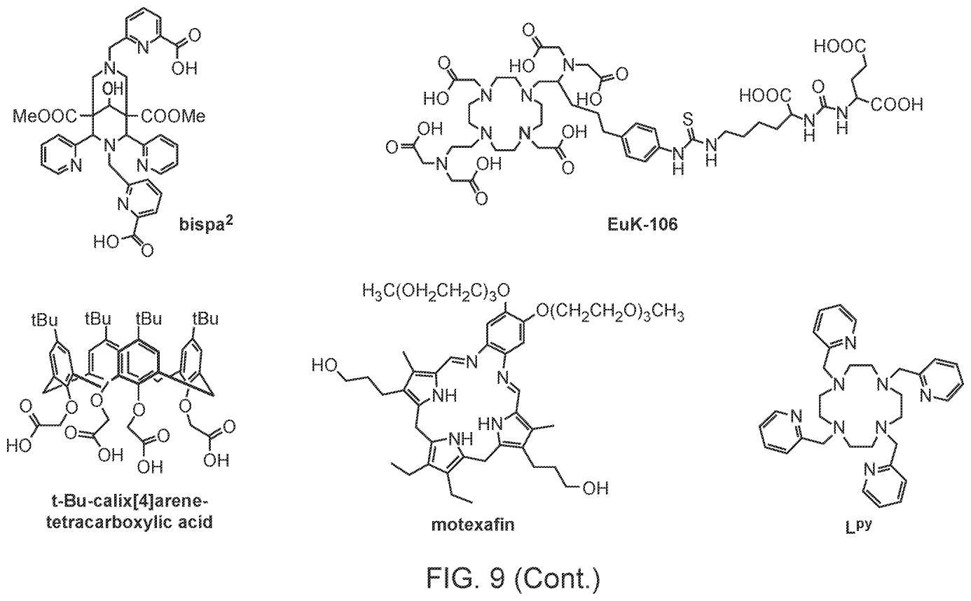

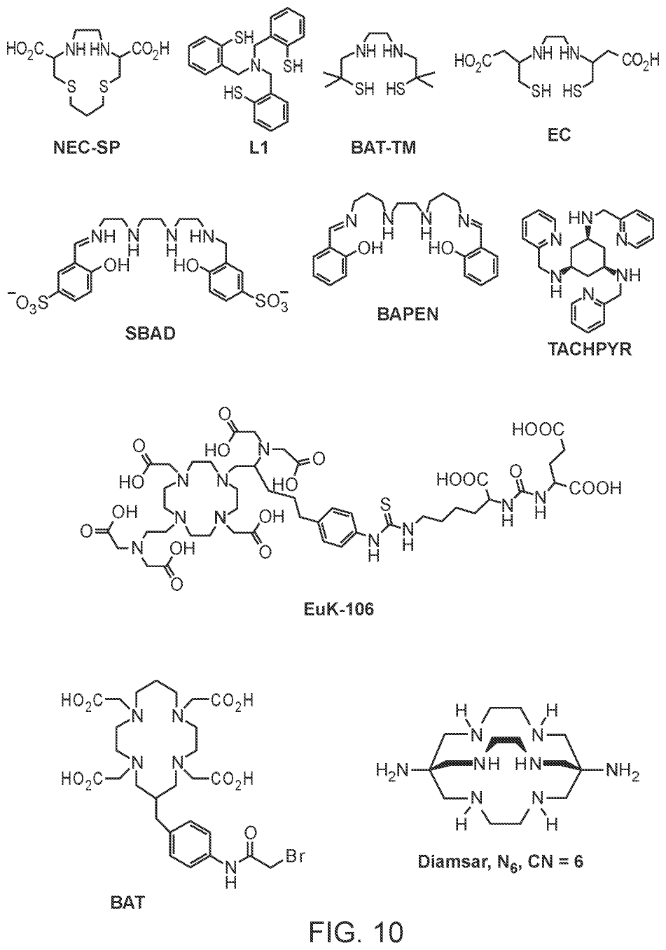

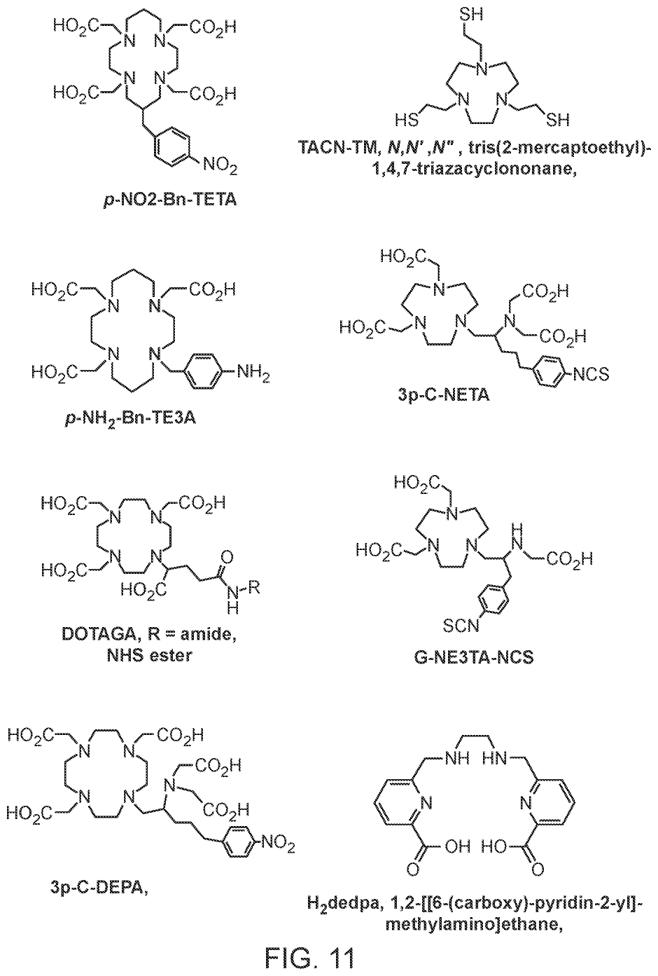

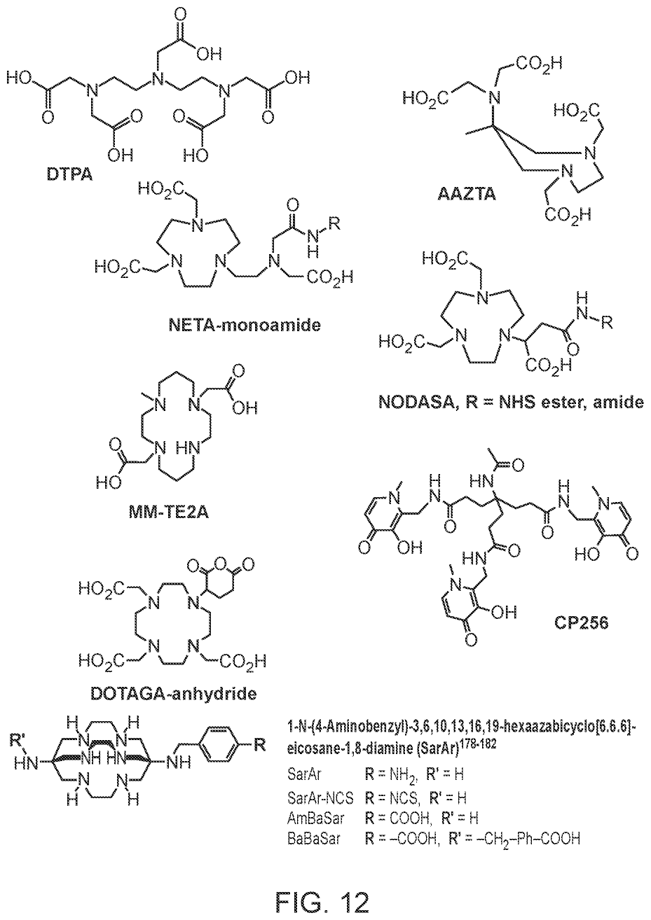

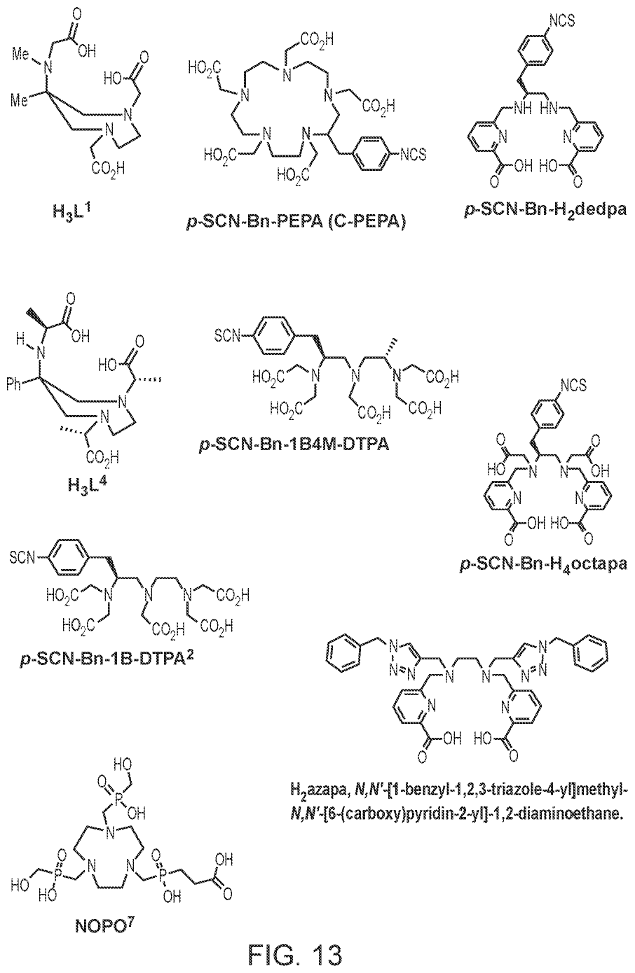

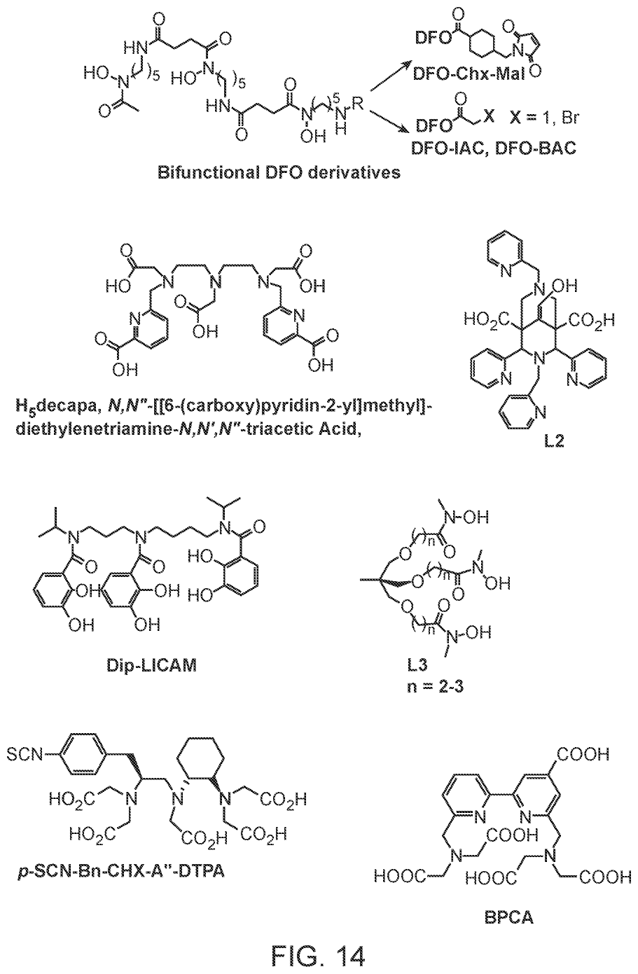

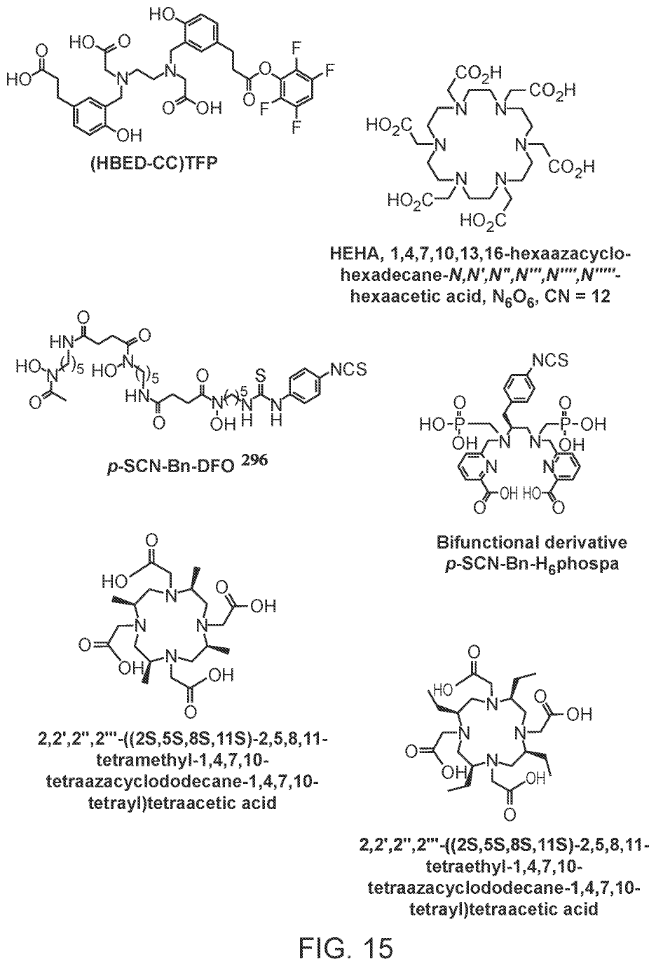

In some embodiments, a herein-described conjugate comprises a targeting ligand selected from Table 1, a radionuclide selected from Tables 4A and 4B, a metal chelator selected from FIGS. 1-15, and optionally a linker that connects the metal chelator with the targeting ligand. In some embodiments, a herein-described conjugate comprises a targeting ligand selected from Table 1 and a metal chelator selected from FIGS. 1-15. In some embodiments, a herein-described conjugate is selected from Tables 2A, 2B and 2C. In some embodiments, the conjugate has a structure of Compound 001. In some embodiments, the conjugate is 001-Ac-225

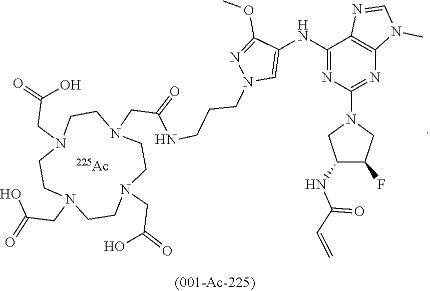

In some embodiments, the conjugate is 001-Lu-177

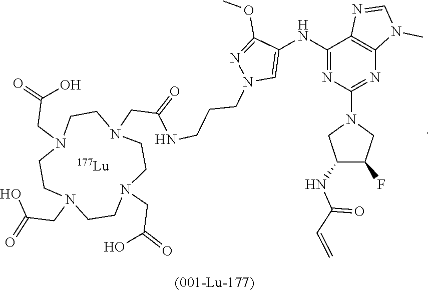

In some embodiments, a conjugate describe herein contains a radioactive isotope, e.g., conjugate 001-Ac-225 or 001-Lu-177. In some embodiments, a conjugate describe herein does not contain a radioactive isotope, e.g., conjugates 001-Lu.

### Targeting Ligand

In one aspect, provided herein are targeting ligands configured to covalently bind to an intracellular protein (e.g., intracellular mutated protein). The targeting ligand can form an irreversible, covalent bond with the mutated protein. In further aspect, provided herein are TRT conjugates that comprise the targeting ligand, a metal chelator, and optionally a linker. The TRT conjugate can further comprise a radionuclide bound to the metal chelator.

The targeting ligand can be an agonist of the mutated protein. The targeting ligand can be an antagonist of the mutated protein. In some embodiments, the targeting ligand does not have any agonism or antagonism effect on the mutated protein.

In some embodiments, the targeting ligand is covalently linked to the metal chelator through a linker. In some embodiments, the targeting ligand is directly linked to the metal chelator.

The targeting ligand can bind to the mutated protein at a mutated amino acid residue. The targeting ligand can also bind to an endogenous amino acid residue within a mutant-specific steric environment. The targeting ligand can form a covalent bond with the mutated protein, e.g., at a cysteine, lysine or serine residue.

In some embodiments, the intracellular mutated protein comprises one or more mutant-specific cysteine residues that are absent in a corresponding wild-type sequence of the intracellular mutated protein. In some embodiments, the targeting ligand binds to (e.g., forming an irreversible, covalent bond with) at least one of the mutant-specific cysteine residues. In some embodiments, the targeting ligand forms a covalent bond with a mutant-specific cysteine residue.

In some embodiments, the intracellular protein is not a mutated protein. For example, in some embodiments, the intracellular protein is overexpressed compared to a corresponding wild-type protein. The intracellular protein can be overexpressed in a cancer tissue.

In some embodiments, the intracellular mutated protein comprises one or more endogenous cysteine residues that are present in a corresponding wild-type sequence of the mutated protein. The intracellular mutated protein can comprise a mutant-specific steric environment in the region around at least one of the endogenous cysteine residues. In some embodiments, the intracellular mutated protein comprises a mutant-specific steric environment in the region around an endogenous cysteine residue. For example, the mutated protein can comprise a mutation in the binding pocket of the endogenous cysteine, thereby allowing a covalent binding between the endogenous cysteine and the targeting ligand. The targeting ligand can selectively bind to at least one of the endogenous cysteine residues in the intracellular mutated protein over a same cysteine residue in a corresponding wild-type protein. In some embodiments, the targeting ligand selectively binds to at least one of the endogenous cysteine residue in the intracellular mutated protein over a same cysteine residue in a corresponding wild-type protein. In some embodiments, the binding affinity of the targeting ligand to the endogenous cysteine residue in the intracellular mutated protein is at least 5 fold, 10 fold, 100 fold, 1,000 fold, or 10,000 fold stronger than its binding affinity to the same cysteine residue in a corresponding wild-type protein. In some embodiments, the rate of formation of the covalent bond between the targeting ligand and the endogenous cysteine residue in the intracellular mutated protein is at least 5 fold, 10 fold, 100 fold, 1,000 fold, 10,000, 100,000 fold faster than the rate of formation of the covalent bond to the same cysteine residue in a corresponding wild-type protein.

In some embodiments, the intracellular mutated protein comprises one or more mutant-specific serine residues that are absent in a corresponding wild-type sequence of the intracellular mutated protein. In some embodiments, the targeting ligand binds to (e.g., forming an irreversible, covalent bond with) at least one of the mutant-specific serine residues. In some embodiments, the targeting ligand forms a covalent bond with a mutant-specific serine residue.

In some embodiments, the intracellular mutated protein comprises one or more endogenous serine residues that are present in a corresponding wild-type sequence of the mutated protein. The intracellular mutated protein can comprise a mutant-specific steric environment in the region around at least one of the endogenous serine residues. In some embodiments, the intracellular mutated protein comprises a mutant-specific steric environment in the region around an endogenous serine residue. For example, the mutated protein can comprise a mutation in the binding pocket of the endogenous serine, thereby allowing a covalent binding between the endogenous serine and the targeting ligand. The targeting ligand can selectively bind to at least one of the endogenous serine residues in the intracellular mutated protein over a same serine residue in a corresponding wild-type protein. In some embodiments, the targeting ligand selectively binds to at least one of the endogenous serine residue in the intracellular mutated protein over a same serine residue in a corresponding wild-type protein. In some embodiments, the binding affinity of the targeting ligand to the endogenous serine residue in the intracellular mutated protein is at least 5 fold, 10 fold, 100 fold, 1000 fold, or 10000 fold stronger than its binding affinity to the same serine residue in a corresponding wild-type protein. In some embodiments, the rate of formation of the covalent bond between the targeting ligand and the endogenous serine residue in the intracellular mutated protein is at least 5 fold, 10 fold, 100 fold, 1,000 fold, 10,000, 100,000 fold faster than the rate of formation of the covalent bond to the same seine residue in a corresponding wild-type protein.

In some embodiments, the intracellular mutated protein comprises one or more mutant-specific lysine residues that are absent in a corresponding wild-type sequence of the intracellular mutated protein. In some embodiments, the targeting ligand binds to (e.g., forming an irreversible, covalent bond with) at least one of the mutant-specific lysine residues. In some embodiments, the targeting ligand forms a covalent bond with a mutant-specific lysine residue.

In some embodiments, the intracellular mutated protein comprises one or more endogenous lysine residues that are present in a corresponding wild-type sequence of the mutated protein. The intracellular mutated protein can comprise a mutant-specific steric environment in the region around at least one of the endogenous lysine residues. In some embodiments, the intracellular mutated protein comprises a mutant-specific steric environment in the region around an endogenous lysine residue. For example, the mutated protein can comprise a mutation in the binding pocket of the endogenous lysine, thereby allowing a covalent binding between the endogenous lysine and the targeting ligand. The targeting ligand can selectively bind to at least one of the endogenous lysine residues in the intracellular mutated protein over a same lysine residue in a corresponding wild-type protein. In some embodiments, the targeting ligand selectively binds to at least one of the endogenous lysine residue in the intracellular mutated protein over a same lysine residue in a corresponding wild-type protein. In some embodiments, the binding affinity of the targeting ligand to the endogenous lysine residue in the intracellular mutated protein is at least 5 fold, 10 fold, 100 fold, 1000 fold, or 10000 fold stronger than its binding affinity to the same lysine residue in a corresponding wild-type protein. In some embodiments, the rate of formation of the covalent bond between the targeting ligand and the endogenous lysine residue in the intracellular mutated protein is at least 5 fold, 10 fold, 100 fold, 1,000 fold, 10,000, 100,000 fold faster than the rate of formation of the covalent bond to the same lysine residue in a corresponding wild-type protein.

The targeting ligand can comprise an electrophilic functional group (or electrophile). In some embodiments, the electrophilic functional group covalently binds to the intracellular protein at a cysteine residue. In some embodiments, the electrophilic functional group irreversibly binds to the intracellular protein at a cysteine residue.

In some embodiments, the electrophilic functional group comprises an ester, acrylamide, chloroacetamide, acyl azide, acyl nitrile, aldehyde, ketone, alkyl halide, alkyl sulfonate, anhydride, aryl halides, boronate, carboxylic acid, hydrazide, carbamate, carbodiimide, diazoalkane, epoxide, haloacetamide, halotriazine, imido ester, isocyanate, isothiocyanate, maleimide, phosphoramidite, silyl halide, sulfonate ester, sulfonyl halide, α,β-unsaturated thione, α,β-unsaturated carbonyl, α-ketoamide, vinyl sulfone, vinyl amide, vinyl arylene, sulfonamide, propargyl amide group, propargyl ketone group, each of the functional group is optionally substituted. In some embodiments, an exemplary vinyl arylene can be or

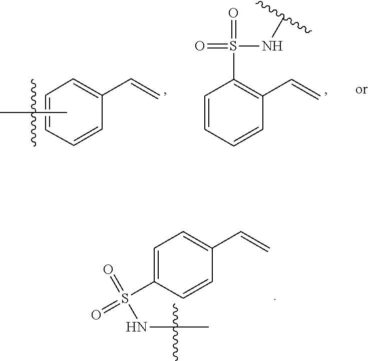

In some embodiments, the electrophilic functional group comprises a substituted or unsubstituted acrylamide group. In some embodiments, the electrophilic functional group comprises a substituted acrylamide. In some embodiments, the electrophilic functional group comprises a unsubstituted acrylamide (or

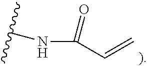 [SMILES: *NC(=O)C=C]

In some embodiments, the electrophilic functional group comprises halo-acrylamide. In some embodiments, the substituted acrylamide is 2-fluoroacrylamide, 2-chloroacrylamide, or a derivative thereof. In some embodiments, the electrophilic functional group comprises 2-fluoroacrylamide. In some embodiments, the electrophilic functional group comprises an α,β-unsaturated carbonyl. In some embodiments, the α,β-unsaturated carbonyl comprises an α,β-unsaturated ketone, α,β-unsaturated aldehyde, α,β-unsaturated amide, α,β-unsaturated acid, or α,β-unsaturated ester, each of which is optionally substituted.

In some embodiments, the electrophilic functional group comprises a substituted or unsubstituted chloroacetamide group. In some embodiments, the electrophilic functional group comprises an unsubstituted chloroacetamide group (or

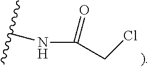 [SMILES: *NC(=O)CCl]

In some embodiments, the electrophilic functional group comprises a substituted or unsubstituted acyl azide group. In some embodiments, the electrophilic functional group comprises a substituted or unsubstituted carbamate group. In some embodiments, the electrophilic functional group comprises a substituted or unsubstituted α,β-unsaturated carbonyl group. In some embodiments, the electrophilic functional group comprises a substituted or unsubstituted α-ketoamide group. In some embodiments, the electrophilic functional group comprises a substituted or unsubstituted propargyl amide group. In some embodiments, the electrophilic functional group comprises a substituted or unsubstituted propargyl ketone group.

In some embodiments, the substituted acrylamide is 2-fluorocryalamide, 2-chloroacrylamide, or a derivative thereof. In some embodiments, the electrophilic group is an α,β-unsaturated carbonyl. In some embodiments, the α,β-unsaturated carbonyl is an α,β-unsaturated ketone, α,β-unsaturated aldehyde, α,β-unsaturated amide, α,β-unsaturated acid, or α,β-unsaturated ester, each of which is optionally substituted.

The targeting ligand can form a covalent bond with a serine residue of the intracellular protein. In some embodiments, the targeting ligand comprises an α-ketoamide, acrylamide, carbonyl, sulfonyl, beta-lactam, nitrile, carbamate, boronic acid or oxaborolane group, each of the functional group is optionally substituted. In some embodiments, the targeting ligand that binds with a serine residue comprises a substituted or unsubstituted α-ketoamide group. In some embodiments, the targeting ligand that binds with a serine residue comprises a substituted or unsubstituted oxaborolane group. In some embodiments, the targeting ligand that binds with a serine residue comprises a carbonyl or sulfonyl group. In some embodiments, the targeting ligand that binds with a serine residue comprises a substituted or unsubstituted beta-lactam group. In some embodiments, the targeting ligand that binds with a serine residue comprises a carbamate group. In some embodiments, an exemplary carbamate can be

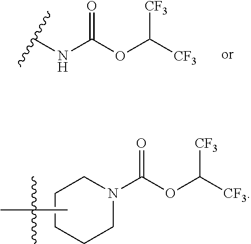

In some embodiments, the targeting ligand comprises a structure selected from

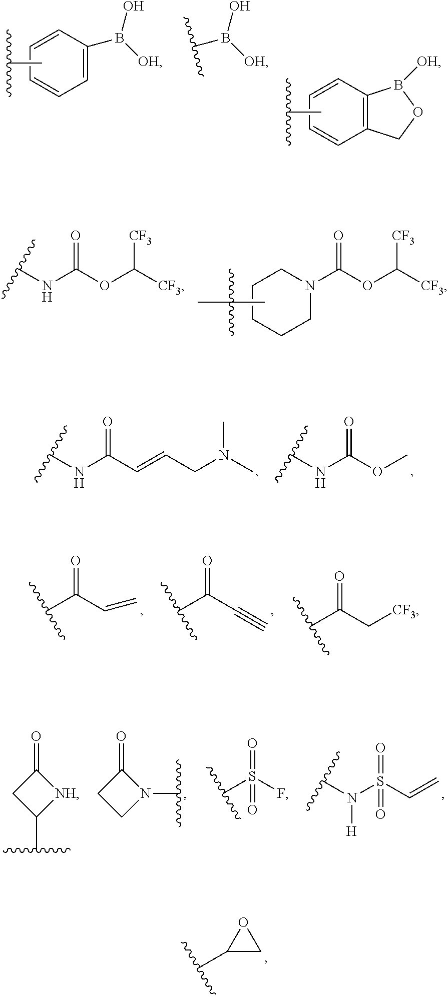

### and —CN.

The targeting ligand can form a covalent bond with a lysine residue of the intracellular protein. In some embodiments, the targeting ligand comprises an electrophilic functional group. In some embodiments, the electrophilic functional group is selected from vinyl sulfone, sulfonamide, acrylamide, β-(dimethylamino)crotylamide and -enones, propargylamide, sulfonyl fluoride, arenesulfonate, haloacetamide, cyanamide, and azine-derived nitrile groups. In some embodiments, the targeting ligand comprises an succinimide, thioester, salicylic aldehydes, o-carbonylboronic derivatives, aryl sulfonyl, or saccharine (benzo[d]isothiazole 1,1-dioxide), each of the functional group is optionally substituted. In some embodiments, the targeting ligand comprises a structure selected from

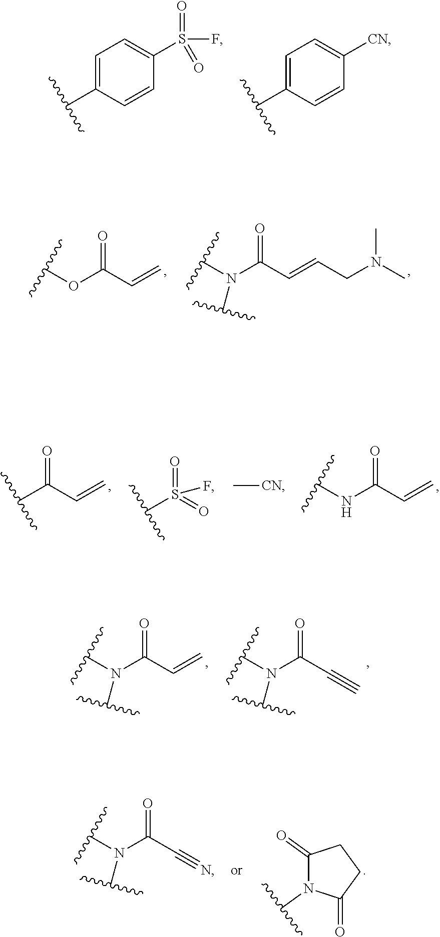

In some embodiments, the electrophilic functional group comprises an acceptor of Michael Addition. In some embodiments, a Michael acceptor comprises a functional group having a structure of

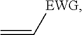

wherein EWG represents an electron withdrawing group. Exemplary Michael acceptors include

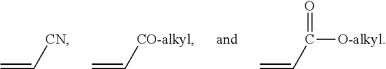

Exemplary Michael acceptors further include

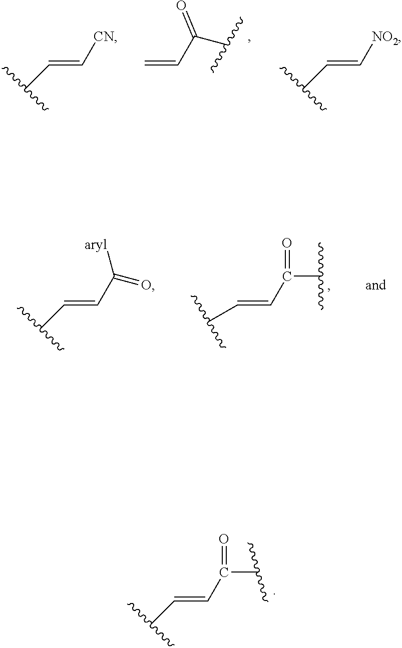

In some embodiments, the electrophilic functional group comprises a structure of Formula (Ia):

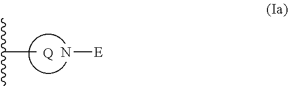

wherein,
ring Q is an optionally substituted 3 to 10 membered heterocycloalkylene; and
E comprises a structure of Formula (Ic),

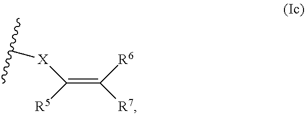

wherein,
   X is C(═O), OC(═O), P(═O)OR², C(═S), S(═O)ₙ, or OS(O)ₙ;
   n is 1 or 2;
   R² is H or substituted or unsubstituted C₁-C₃ alkyl;
   R⁵ and R⁷ are each independently selected from H, —CN, halogen, substituted or unsubstituted C₁-C₆ alkyl, substituted or unsubstituted C₁-C₆ heteroalkyl, substituted or unsubstituted C₃-C₆ cycloalkyl, or substituted or unsubstituted C₂-C₅ heterocycloalkyl; or R⁵ and R⁷ taken together form a bond; and
   R⁶ is H, halogen, —CN, C₁-C₆ alkyl, C₁-C₆ heteroalkyl, heteroaryl, aryl, alkylaminylalkyl, dialkylaminylalkyl, cycloalkyl or heterocycloalkyl, each of which is optionally substituted.

In some embodiments, ring Q of Formula (Ia) is a 3-membered, 4-membered, 5-membered, 6-membered, or 7-membered heterocycloalkylene ring with at least one nitrogen. In some embodiments of Formula (Ia), ring Q is substituted. In some embodiments, ring Q of Formula (Ia) is a diazetidine, azetidine, imidazolidine, pyrrolidine, piperidine, or piperazine ring. In some embodiments, ring Q of Formula (Ia) is a C₂-C₆ optionally substituted monocyclic heterocycloalkylene. In some embodiments, ring Q is 3-6 membered monocyclic heterocycloalkylene. In some embodiments, ring Q comprises 1 or 2 nitrogen. In some embodiments, ring Q of Formula (Ia) is a C₅-C₉ optionally substituted bicyclic heterocycloalkylene In some embodiments, ring Q is a spiro bicyclic heterocycloalkylene. In some embodiments, ring Q is a fused bicyclic heterocycloalkylene. In some embodiments, ring Q is a bridged bicyclic heterocycloalkylene.

In some embodiments, ring Q is optionally substituted with one or more R^{Q} groups, wherein each R^{Q} is independently D, halogen, —CN, —NH₂, —NH(alkyl), —N(alkyl)₂, —OH, oxo, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, or arylsulfone, wherein each of the alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, or arylsulfone is optionally substituted. In some embodiments, ring Q is substituted with 1 R^{Q} group. In some embodiments, ring Q is substituted with 2 R^{Q} groups. In some embodiments, ring Q is substituted with 3 R^{Q} groups. In some embodiments, ring Q is substituted with 4 R^{Q} groups.

In some embodiments, the electrophilic functional group comprises a structure of Formula (Ib):

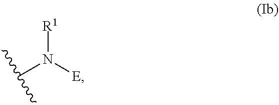

wherein,
R¹ is H, substituted or unsubstituted C₁-C₆ alkyl, substituted or unsubstituted C₁-C₆ heteroalkyl, substituted or unsubstituted C₃-C₆ cycloalkyl, or substituted or unsubstituted C₂-C₅ heterocycloalkyl; and
E comprises a structure of Formula (Ic),

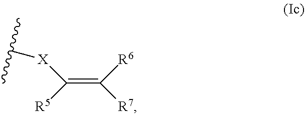

wherein,
   X is C(═O), OC(═O), P(═O)OR², C(═S), S(═O)ₙ, or OS(O)ₙ;
   n is 1 or 2;
   R² is H or substituted or unsubstituted C₁-C₃ alkyl;
   R⁵ and R⁷ are each independently selected from H, —CN, halogen, substituted or unsubstituted C₁-C₆ alkyl, substituted or unsubstituted C₁-C₆ heteroalkyl, substituted or unsubstituted C₃-C₆ cycloalkyl, or substituted or unsubstituted C₂-C₅ heterocycloalkyl; or R⁵ and R⁷ taken together form a bond; and
R⁶ is H, halogen, —CN, C₁-C₆ alkyl, C₁-C₆heteroalkyl, heteroaryl, aryl, alkylaminylalkyl, dialkylaminylalkyl, cycloalkyl or heterocycloalkyl, each of which is optionally substituted.

In some embodiments of Formula (Ia) or Formula (Ib), X is C(═O), P(═O)OR², S(═O), or S(O)₂. In some embodiments of Formula (Ia) or Formula (Ib), X is C(═O). In some embodiments of Formula (Ia) or Formula (Ib), X is S(═O)₂. In some embodiments of Formula (Ia) or Formula (Ib), X is P(═O)OR².

In some embodiments, the electrophilic functional group comprises a structure of Formula (Id):

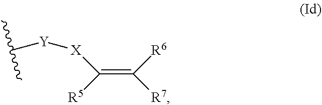

wherein,
X is C(═O), OC(═O), NR²C(═O), N(═NR²), NR²P(═O)OR², C(═S), N(═O), S(═O)ₙ, OS(O)ₙ, NR²S(═O)ₙ, wherein n is 1 or 2;
Y is a bond, alkylene, heteroalkylene, cycloalkylene, heterocycloalkylene, arylene or heteroarylene, each of which is optionally substituted;
each R² is independently H or substituted or unsubstituted C₁-C₃ alkyl;
R⁵ and R⁷ are each independently selected from H, —CN, halogen, substituted or unsubstituted C₁-C₆ alkyl, substituted or unsubstituted C₁-C₆ heteroalkyl, substituted or unsubstituted C₃-C₆ cycloalkyl, or substituted or unsubstituted C₂-C₅ heterocycloalkyl; or R⁵ and R⁷ taken together form a bond; and
R⁶ is H, halogen, —CN, C₁-C₆ alkyl, C₁-C₆ heteroalkyl, heteroaryl, aryl, alkylaminylalkyl, dialkylaminylalkyl, cycloalkyl or heterocycloalkyl, each of which is optionally substituted.

In some embodiments, X of Formula (Id) is C(═O), OC(═O), NR²C(═O), N(═NR²), NR²P(═O)OR², C(═S), S(═O)ₙ, OS(O)ₙ, NR²S(═O)ₙ, wherein n is 1 or 2. In some embodiments of Formula (Id), X is C(═O). In some embodiments, X of Formula (Id) is C(═O). In some embodiments, X of Formula (Id) is NR²C(═O). In some embodiments, X of Formula (Id) is S(═O). In some embodiments, X of Formula (Id) is S(═O)₂. In some embodiments of Formula (Id), X is NR²S(═O)ₙ, wherein n is 1 or 2.

In some embodiments of Formula (Id), Y is a bond, alkylene, heteroalkylene, cycloalkylene, heterocycloalkylene, arylene or heteroarylene, each of which is optionally substituted. In some embodiments of Formula (Id), Y is a bond. In some embodiments of Formula (Id), Y is an alkylene.

In some embodiments, Y of Formula (Id) is substituted or unsubstituted C₁-C₄ alkylene, or substituted or unsubstituted C₁-C₄ heteroalkylene. In some embodiments, Y is an alkylene.

In some embodiments, Y of Formula (Id) is substituted or unsubstituted monocyclic arylene, or substituted or unsubstituted monocyclic heteroarylene. In some embodiments, Y is substituted or unsubstituted phenylene. In some embodiments of Formula (Id), Y is an alkylene or arylene.

In some embodiments, Y of Formula (Id) is substituted or unsubstituted 3 to 10 membered cycloalkylene, or substituted or unsubstituted 3 to 10 membered heterocycloalkylene. In some embodiments, Y of Formula (Id) is substituted or unsubstituted monocyclic or bicyclic cycloalkylene. In some embodiments, Y of Formula (Id) is substituted or unsubstituted monocyclic or bicyclic heterocycloalkylene. In some embodiments, Y is a 3-membered, 4-membered, 5-membered, 6-membered, or 7-membered heterocycloalkylene ring with at least one nitrogen. In some embodiments, Y is substituted. In some embodiments, Y is a diazetidine, azetidine, imidazolidine, pyrrolidine, piperidine, or piperazine ring. In some embodiments, Y is a C₂-C₆ optionally substituted monocyclic heterocycloalkylene. In some embodiments, Y is 3-6 membered monocyclic heterocycloalkylene. In some embodiments, Y comprises 1 or 2 nitrogen. In some embodiments, Y is a C₅-C₉ optionally substituted bicyclic heterocycloalkylene. In some embodiments, Y is a spiro bicyclic heterocycloalkylene. In some embodiments, ring Q is a fused bicyclic heterocycloalkylene. In some embodiments, ring Q is a bridged bicyclic heterocycloalkylene.

In some embodiments, Y of Formula (Id) is optionally substituted with one or more R^{Q} groups, wherein each R^{Q} is independently D, halogen, —CN, —NH₂, —NH(alkyl), —N(alkyl)₂, —OH, oxo, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, or arylsulfone, wherein each of the alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, or arylsulfone is optionally substituted.

In some embodiments, each R^{Q} of Formula (Ia), Formula (Ib) or Formula (Id) is independently oxo, hydroxy, nitro, halogen, C₁₋₆ alkyl, C₁₋₆ alkenyl, C₁₋₆ alkoxy, C₃₋₇ cycloalkyl, C₁₋₆ alkyl-OH, trihalo-C₁₋₆ alkyl, mono-C₁₋₆ alkylamino, di-C₁₋₆ alkylamino, —C(═O)NH₂, —NH₂, —NO₂, hydroxy-C₁₋₆ alkylamino, hydroxy-C₁₋₆ alkyl, 4-7 membered heterocycle-C₁₋₆ alkyl, amino-C₁₋₆ alkyl, mono-C₁₋₆ alkylamino-C₁₋₆ alkyl, and di-C₁₋₆ alkylamino-C₁₋₆ alkyl. In some embodiments, each R^{Q} of Formula (Ia), Formula (Ib) or Formula (Id) is independently D, oxo, halogen, —CN, —NH₂, —NH(CH₃), —N(CH₃)₂, —OH, —CO₂H, —CO₂(C₁-C₄ alkyl), —C(═O)NH₂, —C(═O)NH(C₁-C₄ alkyl), —C(═O)N(C₁-C₄ alkyl)₂, —S(═O)₂NH₂, —S(═O)₂NH(C₁-C₄ alkyl), —S(═O)₂N(C₁-C₄ alkyl)₂, C₁-C₄ alkyl, C₃-C₆ cycloalkyl, C₁-C₄ fluoroalkyl, C₁-C₄heteroalkyl, C₁-C₄ alkoxy, C₁-C₄ fluoroalkoxy, —SC₁-C₄ alkyl, —S(═O)C₁-C₄ alkyl, or —S(═O)₂(C₁-C₄ alkyl). In some embodiments, each R^{Q} of Formula (Ia), Formula (Ib) or Formula (Id) is independently D, oxo, halogen, —CN, —NH₂, —OH, —NH(CH₃), —N(CH₃)₂, —NH(cyclopropyl), —CH₃, —CH₂CH₃, —CF₃, —OCH₃, or —OCF₃. In some embodiments, each R^{Q} is independently substituted or unsubstituted C₁-C₃ alkyl, amino, or —CN. In some embodiments, each R^{Q} is independently methyl, —CH₂CN, or CN.

In some embodiments, R¹ is H, substituted or unsubstituted C₁-C₆ alkyl, substituted or unsubstituted C₁-C₆ heteroalkyl, substituted or unsubstituted C₃-C₆ cycloalkyl, or substituted or unsubstituted C₂-C₅ heterocycloalkyl. In some embodiments, R¹ is H or substituted or unsubstituted C₁-C₃ alkyl. In some embodiments, R¹ is H. In some embodiments, R¹ is substituted or unsubstituted C₃-C₆ cycloalkyl. In some embodiments, R¹ is substituted or unsubstituted C₂-C₅ heterocycloalkyl. In some embodiments, R¹ is substituted or unsubstituted C₁-C₆ heteroalkyl.

In some embodiments of Formula (Ia), Formula (Ib) or Formula (Id), R² is H or substituted or unsubstituted C₁-C₃ alkyl. In some embodiments, R² is H. In some embodiments of Formula (Ia) or Formula (Ib), R² is a substituted C₁-C₃ alkyl. In some embodiments of Formula (Ia) or Formula (Ib), R² is an unsubstituted C₁-C₃ alkyl. In some embodiments, R² is methyl.

In some embodiments of Formula (Ia), Formula (Ib) or Formula (Id), R⁵ and R⁷ are each independently selected from H, —CN, halogen, substituted or unsubstituted C₁-C₄ alkyl, substituted or unsubstituted C₁-C₄ heteroalkyl, substituted or unsubstituted C₃-C₆ cycloalkyl, or substituted or unsubstituted C₂-C₅ heterocycloalkyl. In some embodiments of Formula (Ia), Formula (Ib) or Formula (Ib), R⁵ and R⁷ taken together form a bond.

In some embodiments of Formula (Ia), Formula (Ib) or Formula (Id), R⁵ is a halogen. In some embodiments of Formula (Ia), Formula (Ib) or Formula (Id), R⁵ is hydrogen. In some embodiments of Formula (Ia), Formula (Ib) or Formula (Id), R⁵ is fluorine or chlorine. In some embodiments of Formula (Ia), Formula (Ib) or Formula (Id), R⁵ is —CN.

In some embodiments, R⁵ is H, halogen, methyl, or —OMe.

In some embodiments of Formula (Ia), Formula (Ib) or Formula (Id), R⁷ is substituted or unsubstituted C₂-C₅ heterocycloalkyl. In some embodiments of Formula (Ia), Formula (Ib) or Formula (Id), R⁷ is substituted or unsubstituted C₁-C₄ heteroalkyl. In some embodiments of Formula (Ia), Formula (Ib) or Formula (Id), R⁷ is hydrogen. In some embodiments, R⁷ is substituted or unsubstituted C₁-C₄ alkyl. In some embodiments, R⁷ is —CH₂F, —CH₂OMe, or —CH₂—C₂-C₅ heterocycloalkyl.

In some embodiments of Formula (Ia), Formula (Ib) or Formula (Id), R⁶ is H, halogen, C₁-C₃ alkyl, C₁-C₃ heteroalkyl, C₁₋₃alkylaminyl-C₁₋₃alkyl, di(C₁₋₃)alkylaminyl-C₁₋₃alkyl, C₃-C₆ cycloalkyl or C₂-C₅ heterocycloalkyl, each of which is optionally substituted. In some embodiments of Formula (Ia), Formula (Ib) or Formula (Id), R⁶ is an unsubstituted or substituted C₁-C₃ heteroalkyl, alkylaminylalkyl, or dialkylaminylalkyl. In some embodiments of Formula (Ia), Formula (Ib) or Formula (Id), R⁶ is an unsubstituted or substituted heterocycloalkyl. In some embodiments of Formula (Ia), Formula (Ib) or Formula (Id), R⁶ is hydrogen. In some embodiments of Formula (Ia), Formula (Ib) or Formula (Id), R⁶ is an unsubstituted or substituted heteroaryl. In some embodiments, R⁶ is a substituted 5 or 6-membered heteroaryl. In some embodiments of Formula (Ia), Formula (Ib) or Formula (Id), R⁶ is an unsubstituted or substituted aryl. In some embodiments, R⁶ is a monocyclic ring. In some embodiments, R⁶ is a bicyclic ring.

In some embodiments of Formula (Ia), Formula (Ib), Formula (Ic) or Formula (Id), each of R⁵, R⁶, and R⁷ is hydrogen. In some embodiments of Formula (Ia), Formula (Ib), Formula (Ic) or Formula (Id), R⁵ is fluorine and, R⁶ and R⁷ is hydrogen. In some embodiments of Formula (Ia), Formula (Ib), Formula (Ic) or Formula (Id), R⁵ is —CH₃ and, R⁶ and R⁷ is hydrogen. In some embodiments of Formula (Ia), Formula (Ib), Formula (Ic) or Formula (Id), R⁵ is —OCH₃ and, R⁶ and R⁷ is hydrogen. In some embodiments of Formula (Ia), Formula (Ib), Formula (Ic) or Formula (Id), R⁵ and R⁶ are hydrogen and R⁷ is —CH₂F. In some embodiments of Formula (Ia), Formula (Ib), Formula (Ic) or Formula (Id), R⁵ and R⁶ are hydrogen and R⁷ is —CH₂OMe. In some embodiments of Formula (Ia), Formula (Ib), Formula (Ic) or Formula (Id), R⁵ and R⁶ are hydrogen and R⁷ is —CH₂C₂-C₅heterocycloalkyl. In some embodiments of Formula (Ia), Formula (Ib), Formula (Ic) or Formula (Id), R⁵ and R⁶ are hydrogen and R⁷ is —CH₂-aziridinyl. In some embodiments of Formula (Ia), Formula (Ib), Formula (Ic) or Formula (Id), R⁵ and R⁶ are hydrogen and R⁷ is —CH₂-azetidinyl. In some embodiments of Formula (Ia), Formula (Ib), Formula (Ic) or Formula (Id), R⁵ and R⁶ are hydrogen and R⁷ is —CH₂-pyrrolidinyl. In some embodiments of Formula (Ia), Formula (Ib), Formula (Ic) or Formula (Id), R⁵ and R⁶ are hydrogen and R⁷ is —CH₂— piperidinyl.

In some embodiments, the electrophilic functional group comprises a structure selected from

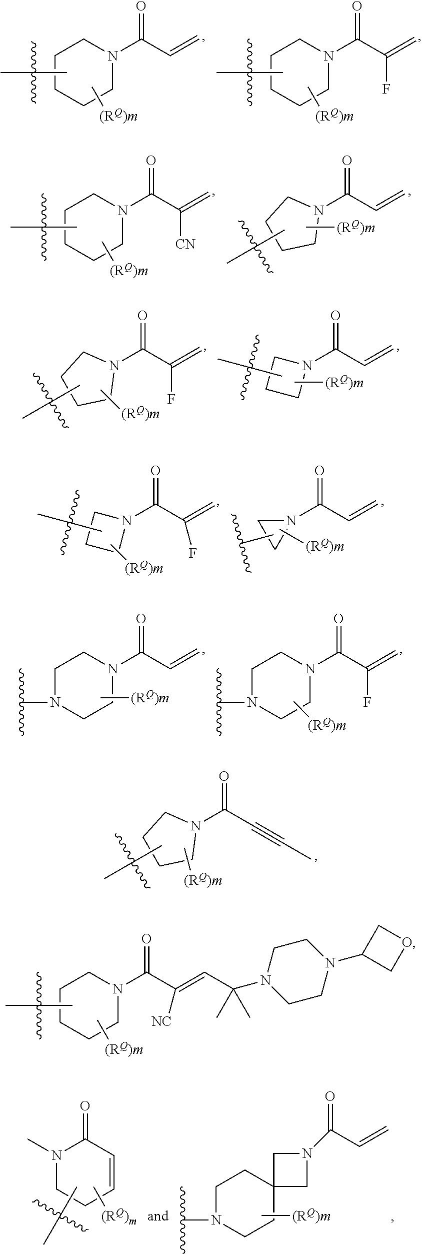

wherein m is 0, 1, 2, 3, 4, or 5. In some embodiments, the electrophilic functional group comprises

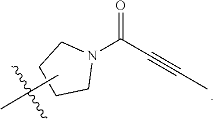

In some embodiments, the electrophilic functional group comprises

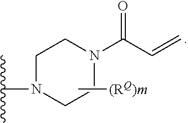

In some embodiments, the electrophilic functional group comprises

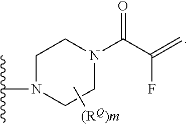

In some embodiments, the electrophilic functional group comprises a structure selected from

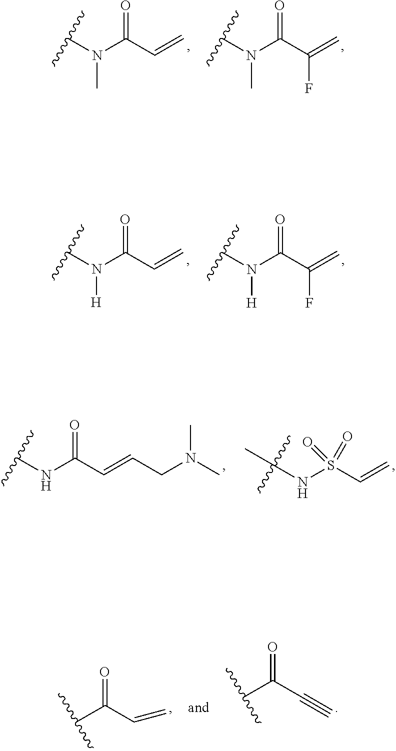

In some embodiments, the electrophilic functional group comprises

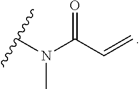 [SMILES: *N(C)C(=O)C=C]

In some embodiments, the electrophilic functional group comprises

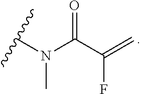 [SMILES: *N(C)C(=O)C(=C)F]

In some embodiments, the electrophilic functional group comprises

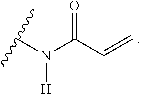 [SMILES: *NC(=O)C=C]

In some embodiments, the electrophilic functional group comprises

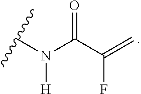 [SMILES: *NC(=O)C(=C)F]

In some embodiments, R⁵ and R⁷ taken together form a bond. In some embodiments, E comprises a structure of Formula (Ic), wherein the structure of Formula (Ic) is

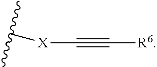

In some embodiments, the structure of Formula (Id) is

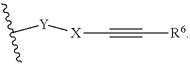

In some embodiments, E is

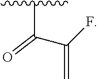 [SMILES: *C(=O)C(=C)F]

In some embodiments, E is

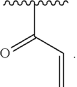 [SMILES: *C(=O)C=C]

In some embodiments, the electrophilic functional group comprises

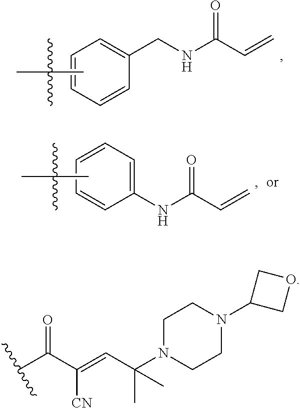

In some embodiments, the targeting ligand comprises the structure

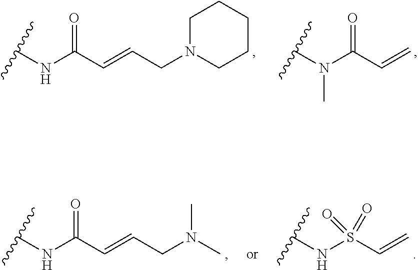

In some embodiments, the electrophilic functional group comprises

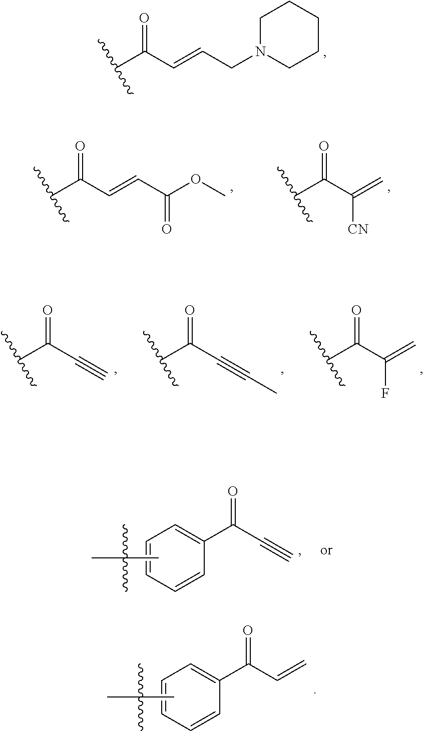

In some embodiments, the targeting ligand comprises a structure selected from Table 1. In some embodiments, the targeting ligand comprises a derivative, or a binding fragment of the structures in Table 1.

TABLE 1
Exemplary Targeting Ligand (attachment point to the conjugate not shown).

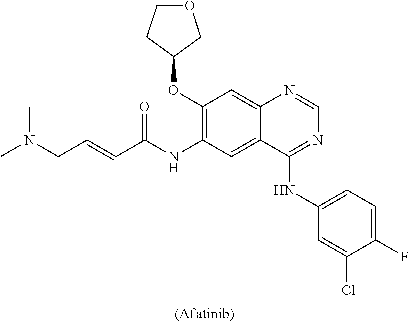 [SMILES: CN(C)C/C=C/C(=O)Nc1cc2c(Nc3ccc(F)c(Cl)c3)ncnc2cc1O[C@H]1CCOC1]

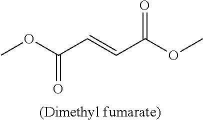

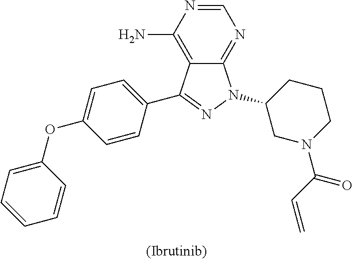 [SMILES: C=CC(=O)N1CCC[C@@H](n2nc(-c3ccc(Oc4ccccc4)cc3)c3c(N)ncnc32)C1]

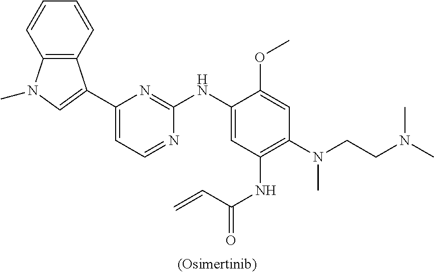

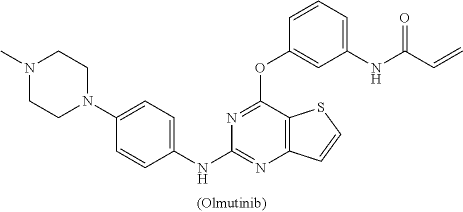 [SMILES: C=CC(=O)Nc1cccc(Oc2nc(Nc3ccc(N4CCN(C)CC4)cc3)nc3ccsc23)c1]

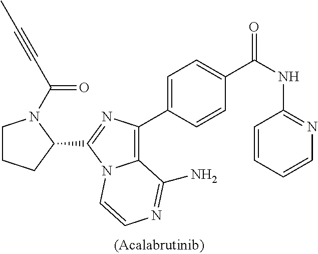 [SMILES: CC#CC(=O)N1CCC[C@H]1c1nc(-c2ccc(C(=O)Nc3ccccn3)cc2)c2c(N)nccn12]

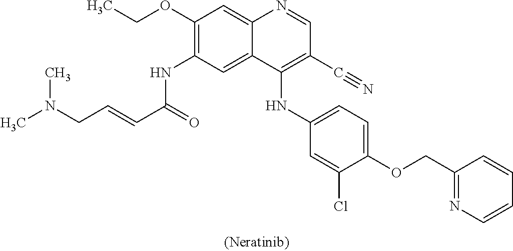 [SMILES: CCOc1cc2ncc(C#N)c(Nc3ccc(OCc4ccccn4)c(Cl)c3)c2cc1NC(=O)/C=C/CN(C)C]

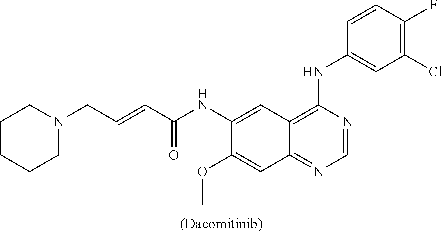 [SMILES: COc1cc2ncnc(Nc3ccc(F)c(Cl)c3)c2cc1NC(=O)/C=C/CN1CCCCC1]

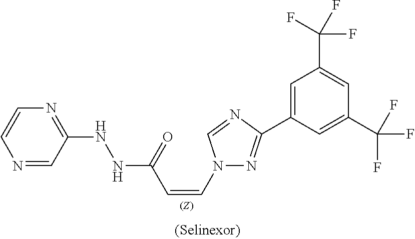 [SMILES: O=C(/C=C\n1cnc(-c2cc(C(F)(F)F)cc(C(F)(F)F)c2)n1)NNc1cnccn1]

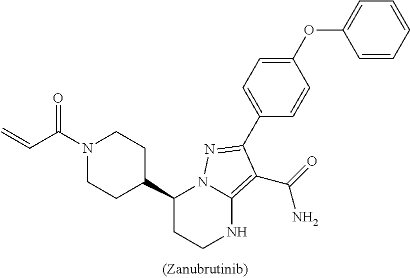 [SMILES: C=CC(=O)N1CCC([C@@H]2CCNc3c(C(N)=O)c(-c4ccc(Oc5ccccc5)cc4)nn32)CC1]

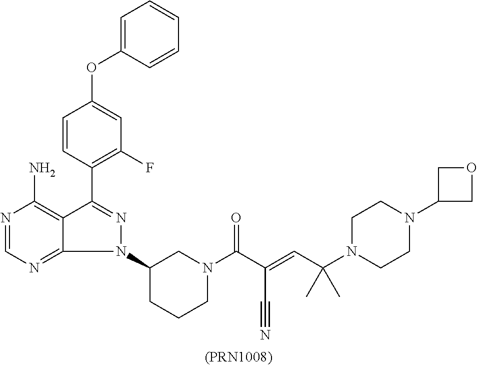 [SMILES: CC(C)(/C=C(\C#N)C(=O)N1CCC[C@@H](n2nc(-c3ccc(Oc4ccccc4)cc3F)c3c(N)ncnc32)C1)N1CCN(C2COC2)CC1]

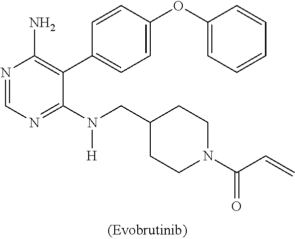 [SMILES: C=CC(=O)N1CCC(CNc2ncnc(N)c2-c2ccc(Oc3ccccc3)cc2)CC1]

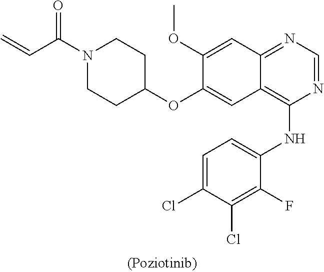 [SMILES: C=CC(=O)N1CCC(Oc2cc3c(Nc4ccc(Cl)c(Cl)c4F)ncnc3cc2OC)CC1]

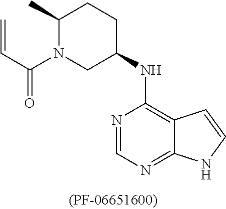 [SMILES: C=CC(=O)N1C[C@H](Nc2ncnc3[nH]ccc23)CC[C@@H]1C]

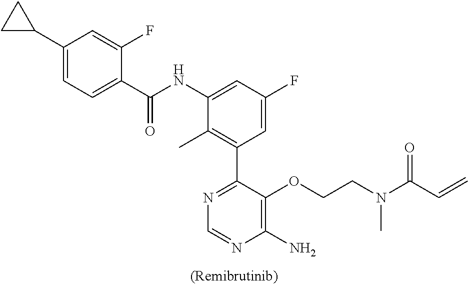 [SMILES: C=CC(=O)N(C)CCOc1c(N)ncnc1-c1cc(F)cc(NC(=O)c2ccc(C3CC3)cc2F)c1C]

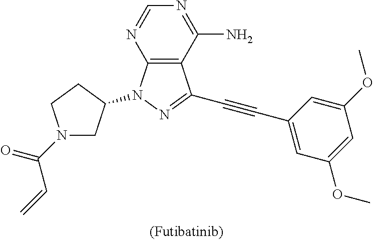 [SMILES: C=CC(=O)N1CC[C@H](n2nc(C#Cc3cc(OC)cc(OC)c3)c3c(N)ncnc32)C1]

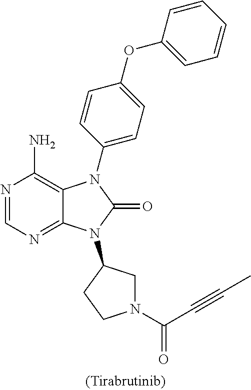 [SMILES: CC#CC(=O)N1CC[C@@H](n2c(=O)n(-c3ccc(Oc4ccccc4)cc3)c3c(N)ncnc32)C1]

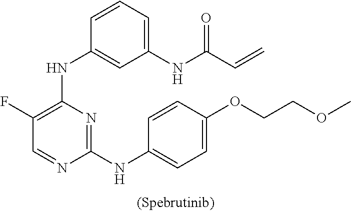 [SMILES: C=CC(=O)Nc1cccc(Nc2nc(Nc3ccc(OCCOC)cc3)ncc2F)c1]

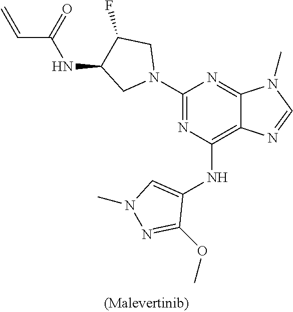 [SMILES: C=CC(=O)N[C@@H]1CN(c2nc(Nc3cn(C)nc3OC)c3ncn(C)c3n2)C[C@H]1F]

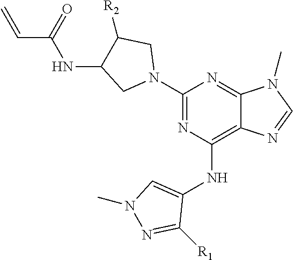

R₂ is H, C₁-C₆
alkyl, C₁-C₆ alkoxyl (e.g., methoxy), or
halogen, and R₁ is H, C₁-C₆ alkyl (e.g.,
methyl, ethyl), or C₁-C₆ alkoxyl (e.g.,
methoxy)
R₂ is H, C₁-C₆ alkyl,
C₁-C₆ alkoxyl (e.g., methoxy), or halogen, and
R₁ is H, C₁-C₆ alkyl (e.g., methyl, ethyl), or C₁-
C₆ alkoxyl (e.g., methoxy)
R₂ is H, C₁-C₆
alkyl, C₁-C₆ alkoxyl (e.g., methoxy), or
halogen, and R₁ is H, C₁-C₆ alkyl (e.g.,
methyl, ethyl), or C₁-C₆ alkoxyl (e.g.,
methoxy)

In some embodiments, the targeting ligand has a structure of

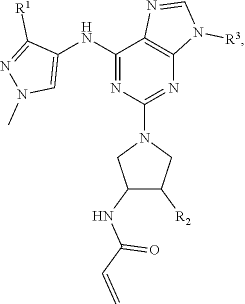

wherein R₃ is H or C₁-C₆ alkyl (e.g., methyl, t-butyl); R₂ is H, C₁-C₆ alkyl, C₁-C₆ alkoxyl, or halogen; and R₁ is H, C₁-C₆ alkyl (e.g., methyl or ethyl), or C₁-C₆ alkoxyl (e.g., methoxy). In some embodiments, the targeting ligand has a structure of

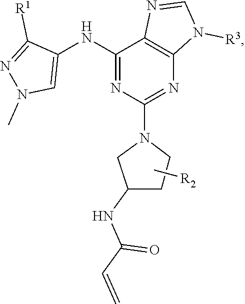

wherein R₃ is H or C₁-C₆ alkyl (e.g., methyl, t-butyl); R₂ is H, C₁-C₆ alkyl, C₁-C₆ alkoxyl, or halogen; and R¹ is H, C₁-C₆ alkyl (e.g., methyl or ethyl), or C₁-C₆ alkoxyl (e.g., methoxy). In some embodiments, the targeting ligand has a structure of

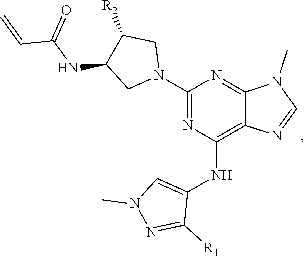

wherein R₂ is H, C₁-C₆ alkyl, C₁-C₆ alkoxyl, or halogen, and R₁ is H, C₁-C₆ alkyl (e.g., methyl or ethyl), or C₁-C₆ alkoxyl (e.g., methoxy). In some embodiments, the targeting ligand has a structure of

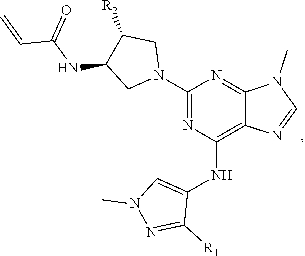

wherein R₂ is methoxy or fluoro, and R¹ is H, methyl, methoxy or ethyl.

In some embodiments, the targeting ligand has a structure of

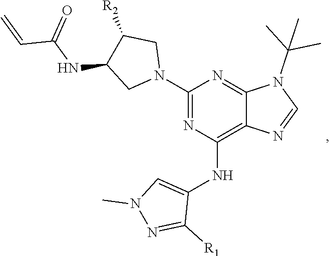

wherein R₂ is H, C₁-C₆ alkyl, C₁-C₆ alkoxyl, or halogen, and R₁ is H, C₁-C₆ alkyl, or C₁-C₆ alkoxyl.
In some embodiments, the targeting ligand has a structure of

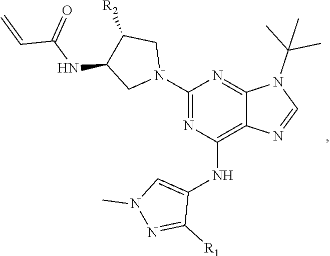

wherein R₂ is methoxy or fluoro, and R₁ is H, methyl, methoxy or ethyl.

In some embodiments, the targeting ligand has a structure of

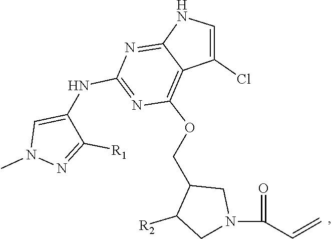

wherein R₂ is H, C₁-C₆ alkyl, C₁-C₆ alkoxyl, or halogen, and R¹ is H, C₁-C₆ alkyl, or C₁-C₆ alkoxyl.
In some embodiments, the targeting ligand has a structure of

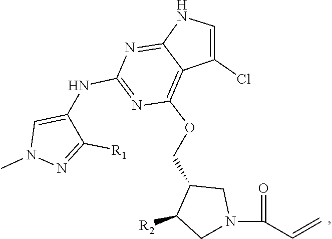

wherein R₂ is H, C₁-C₆ alkyl, C₁-C₆ alkoxyl, or halogen, and R¹ is H, C₁-C₆ alkyl, or C₁-C₆ alkoxyl. In some embodiments, the targeting ligand has a structure of

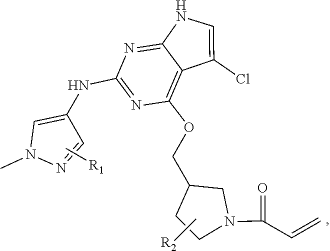

wherein R₂ is H, C₁-C₆ alkyl, C₁-C₆ alkoxyl, or halogen, and R¹ is H, C₁-C₆ alkyl, or C₁-C₆ alkoxyl. In some embodiments, the targeting ligand has a structure of

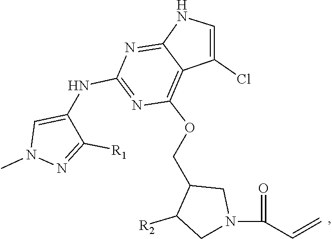

wherein R₂ is methoxy or fluoro, and R₁ is H, methyl, methoxy or ethyl.

In some embodiments, described herein is a radiopharmaceutical conjugate selected from Table 2A, or salt thereof.

TABLE 2A

| Compound ID | Structure |
|---|---|
| Compound 001 | |
| Compound 002 | |
| Compound 003 | 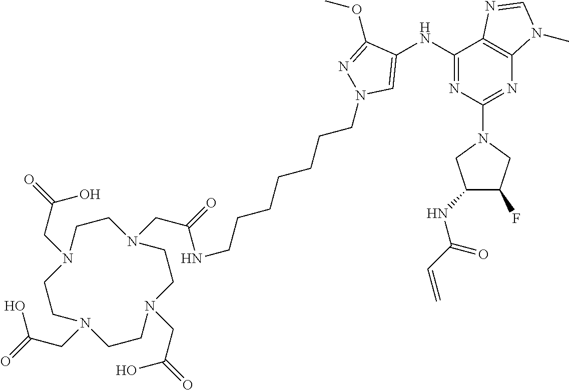 [SMILES: C=CC(=O)N[C@@H]1CN(c2nc(Nc3cn(CCCCCCCNC(=O)CN4CCN(CC(=O)O)CCN(CC(=O)O)CCN(CC(=O)O)CC4)nc3OC)c3ncn(C)c3n2)C[C@H]1F] |
| Compound 004 | 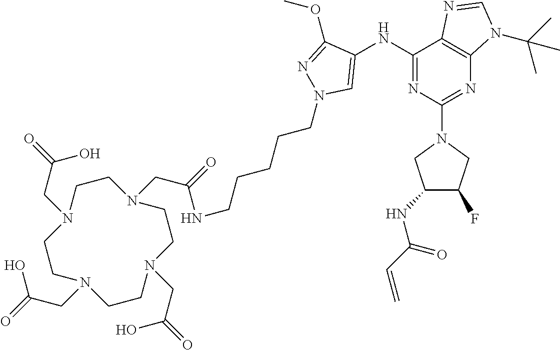 [SMILES: C=CC(=O)N[C@@H]1CN(c2nc(Nc3cn(CCCCCNC(=O)CN4CCN(CC(=O)O)CCN(CC(=O)O)CCN(CC(=O)O)CC4)nc3OC)c3ncn(C(C)(C)C)c3n2)C[C@H]1F] |
| Compound 005 | 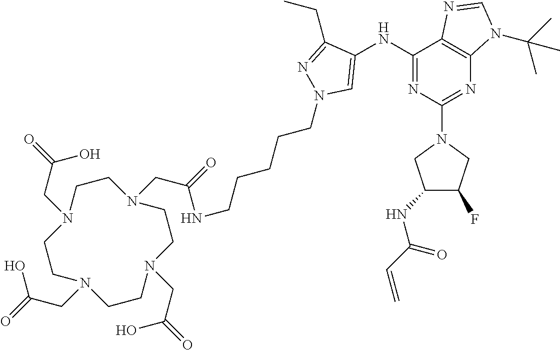 [SMILES: C=CC(=O)N[C@@H]1CN(c2nc(Nc3cn(CCCCCNC(=O)CN4CCN(CC(=O)O)CCN(CC(=O)O)CCN(CC(=O)O)CC4)nc3CC)c3ncn(C(C)(C)C)c3n2)C[C@H]1F] |
| Compound 006 | 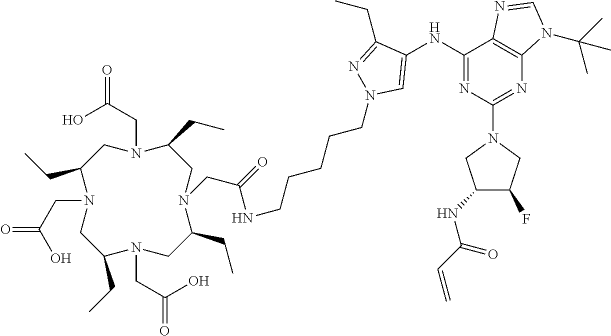 [SMILES: C=CC(=O)N[C@@H]1CN(c2nc(Nc3cn(CCCCCNC(=O)CN4C[C@H](CC)N(CC(=O)O)C[C@H](CC)N(CC(=O)O)C[C@H](CC)N(CC(=O)O)C[C@@H]4CC)nc3CC)c3ncn(C(C)(C)C)c3n2)C[C@H]1F] |
| Compound 007 | |

In some embodiments, described herein is a radiopharmaceutical conjugate selected from Table 2B3, or salt thereof.

TABLE 2B

| Compound ID | Structure |
|---|---|
| Compound 001- | |
| Lu-177 | |
| Compound 002- | |
| Lu-177 | |
| Compound 003- Lu-177 | 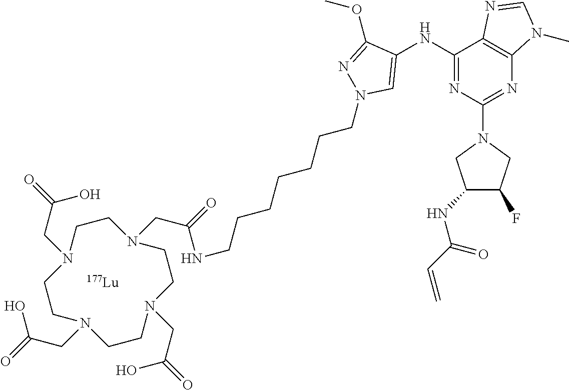 |
| Compound 004- Lu-177 | 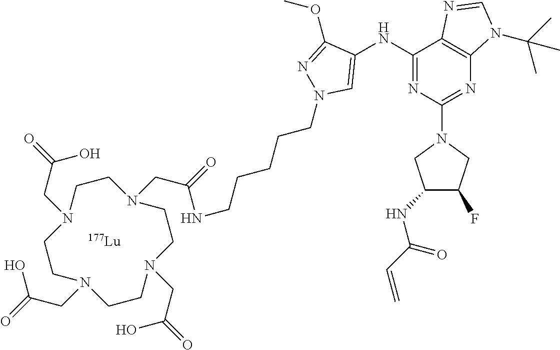 |
| Compound 005- Lu-177 | 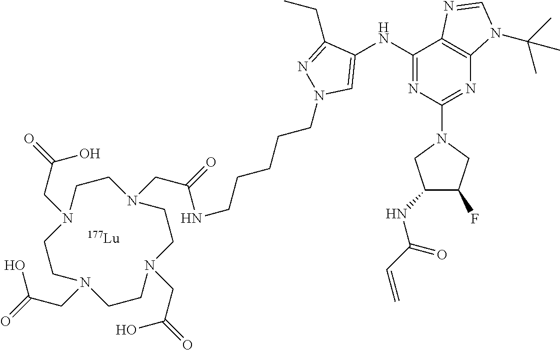 |
| Compound 006- Lu-177 | 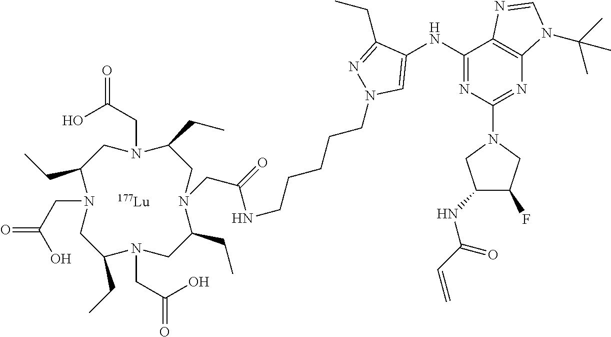 |
| Compound 007- | |
| Lu-177 | |

In some embodiments, described herein is a radiopharmaceutical conjugate selected from Table 2C, or salt thereof.

TABLE 2C

| Compound ID | Structure |
|---|---|
| Compound 001- | |
| Ac-225 | |
| Compound 002- | |
| Ac-225 | |
| Compound 003- Ac-225 | 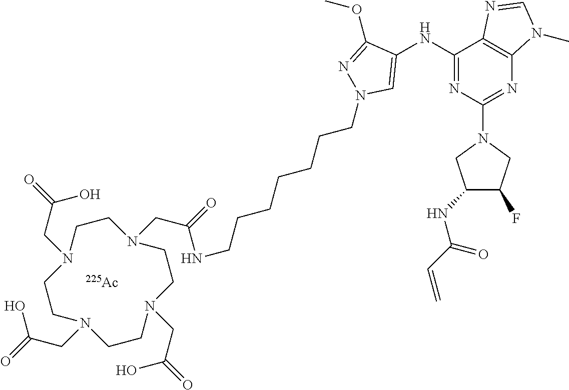 |
| Compound 004- Ac-225 | 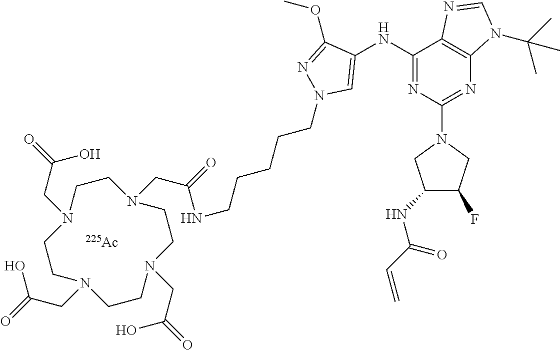 |
| Compound 005- Ac-225 | 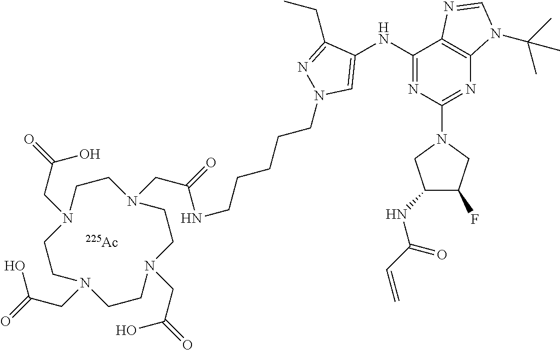 |
| Compound 006- Ac-225 | 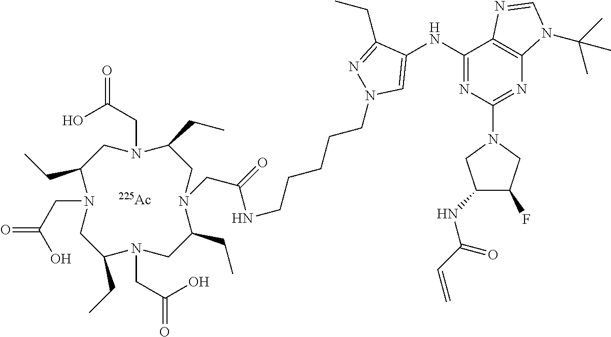 |
| Compound 007- | |
| Ac-225 | |

### Target—Mutated Protein

In one aspect, described herein are conjugates that are designed to covalently and irreversibly bind to an intracellular protein (e.g., intracellular mutated protein). The intracellular protein can be a tumor associated protein.

Some mutations in cancer cells lead to the changing of an amino acid in the normal functioning protein to a cysteine (for example KRAS G13C). This mutant-specific cysteine could be used to selectively form a covalent bond with a suitably designed TRT construct. Alternatively, some normally functioning proteins naturally contain a cysteine, and mutations in the region of that cysteine may create a new steric environment in the region around the endogenous cysteine (for example EGFR mutations). This new steric environment that is specific to the mutant protein could be used to selectively form a covalent bond with a suitably designed TRT construct.

In some embodiments, the intracellular mutated protein is encoded by a gene selected from KRAS, FGFR1, FGFR2, FGFR3, FGFR4, FGFRL1, TP53, IDH1, GNAS, FBXW7, CTNNB1, DNMT3A, EGFR, BTK, ERBB2, ERBB3, and JAK3. In some embodiments, the wherein the intracellular mutated protein is a GTPase KRas (KRAS protein), Tumor Protein P53 (TP53 protein), Isocitrate Dehydrogenase (NADP(+)) 1 (IDH1 protein), Guanine Nucleotide binding protein (GNAS protein), F-Box And WD Repeat Domain Containing 7 (FBXW7 protein), Catenin beta-1 (CTNNB1 protein), DNA (cytosine-5)-methyltransferase 3A (DNMT3A protein), Epidermal growth factor receptor (EGFR protein), Bruton's tyrosine kinase (BTK protein), Janus kinase 3 (JAK3 protein), Fibroblast growth factor receptor (FGFR protein), Human epidermal growth factor receptor 2 (HER2), or Human epidermal growth factor receptor 3 (HER3). In some embodiments, the intracellular mutated protein comprises one or more endogenous cysteine residues that are present in a corresponding wild-type sequence in Table 3 of the mutated protein.

TABLE 3
Exemplary Protein Wild-type Sequences

| SEQ | | | |
|---|---|---|---|
| ID NO | Amino Acid Sequence | Protein | Gene |
| 1 | MTEYKLVVVGAGGVGKSALTIQLIQNHFVDEYDPTIEDSYRKQ | GTPase | KRAS |
| | VVIDGETCLLDILDTAGQEEYSAMRDQYMRTGEGFLCVFAINN | KRas | |
| | TKSFEDIHHYREQIKRVKDSEDVPMVLVGNKCDLPSRTVDTKQ | | |
| | AQDLARSYGIPFIETSAKTRQRVEDAFYTLVREIRQYRLKKIS | | |
| | KEEKTPGCVKIKKCIIM | | |
| 2 | MGAPACALALCVAVAIVAGASSESLGTEQRVVGRAAEVPGPEP | Fibroblast | FGFR3 |
| | GQQEQLVFGSGDAVELSCPPPGGGPMGPTVWVKDGTGLVPSER | growth | |
| | VLVGPQRLQVLNASHEDSGAYSCRQRLTQRVLCHFSVRVTDAP | factor | |
| | SSGDDEDGEDEAEDTGVDTGAPYWTRPERMDKKLLAVPAANTV | receptor | |
| | RFRCPAAGNPTPSISWLKNGREFRGEHRIGGIKLRHQQWSLVM | 3 | |
| | ESVVPSDRGNYTCVVENKFGSIRQTYTLDVLERSPHRPILQAG | | |
| | LPANQTAVLGSDVEFHCKVYSDAQPHIQWLKHVEVNGSKVGPD | | |
| | GTPYVTVLKTAGANTTDKELEVLSLHNVTFEDAGEYTCLAGNS | | |
| | IGFSHHSAWLVVLPAEEELVEADEAGSVYAGILSYGVGFFLFI | | |
| | LVVAAVTLCRLRSPPKKGLGSPTVHKISRFPLKRQVSLESNAS | | |
| | MSSNTPLVRIARLSSGEGPTLANVSELELPADPKWELSRARLT | | |
| | LGKPLGEGCFGQVVMAEAIGIDKDRAAKPVTVAVKMLKDDATD | | |
| | KDLSDLVSEMEMMKMIGKHKNIINLLGACTQGGPLYVLVEYAA | | |
| | KGNLREFLRARRPPGLDYSFDTCKPPEEQLTFKDLVSCAYQVA | | |
| | RGMEYLASQKCIHRDLAARNVLVTEDNVMKIADFGLARDVHNL | | |
| | DYYKKTTNGRLPVKWMAPEALFDRVYTHQSDVWSFGVLLWEIF | | |
| | TLGGSPYPGIPVEELFKLLKEGHRMDKPANCTHDLYMIMRECW | | |
| | HAAPSQRPTFKQLVEDLDRVLTVTSTDEYLDLSAPFEQYSPGG | | |
| | QDTPSSSSSGDDSVFAHDLLPPAPPSSGGSRT | | |
| 3 | MEEPQSDPSVEPPLSQETFSDLWKLLPENNVLSPLPSQAMDDL | Tumor | TP3 |
| | MLSPDDIEQWFTEDPGPDEAPRMPEAAPPVAPAPAAPTPAAPA | protein | |
| | PAPSWPLSSSVPSQKTYQGSYGFRLGFLHSGTAKSVTCTYSPA | P53 | |
| | LNKMFCQLAKTCPVQLWVDSTPPPGTRVRAMAIYKQSQHMTEV | | |
| | VRRCPHHERCSDSDGLAPPQHLIRVEGNLRVEYLDDRNTFRHS | | |
| | VVVPYEPPEVGSDCTTIHYNYMCNSSCMGGMNRRPILTIITLE | | |
| | DSSGNLLGRNSFEVRVCACPGRDRRTEEENLRKKGEPHHELPP | | |
| | GSTKRALPNNTSSSPQPKKKPLDGEYFTLQIRGRERFEMFREL | | |
| | NEALELKDAQAGKEPGGSRAHSSHLKSKKGQSTSRHKKLMFKT | | |
| | EGPDSD | | |
| 4 | MSKKISGGSVVEMQGDEMTRIIWELIKEKLIFPYVELDLHSYD | Isocitrate | IDH1 |
| | LGIENRDATNDQVTKDAAEAIKKHNVGVKCATITPDEKRVEEF | Dehydro- | |
| | KLKQMWKSPNGTIRNILGGTVFREAIICKNIPRLVSGWVKPII | genase | |
| | IGRHAYGDQYRATDFVVPGPGKVEITYTPSDGTQKVTYLVHNF | (NADP(+)) | |
| | EEGGGVAMGMYNQDKSIEDFAHSSFQMALSKGWPLYLSTKNTI | 1 | |
| | LKKYDGRFKDIFQEIYDKQYKSQFEAQKIWYEHRLIDDMVAQA | | |
| | MKSEGGFIWACKNYDGDVQSDSVAQGYGSLGMMTSVLVCPDGK | | |
| | TVEAEAAHGTVTRHYRMYQKGQETSTNPIASIFAWTRGLAHRA | | |
| | KLDNNKELAFFANALEEVSIETIEAGFMTKDLAACIKGLPNVQ | | |
| | RSDYLNTFEFMDKLGENLKIKLAQAKL | | |
| 5 | MGVRNCLYGNNMSGQRDIPPEIGEQPEQPPLEAPGAAAPGAGP | Guanine | GNAS |
| | SPAEEMETEPPHNEPIPVENDGEACGPPEVSRPNFQVLNPAFR | Nucleotide | |
| | EAGAHGSYSPPPEEAMPFEAEQPSLGGFWPTLEQPGFPSGVHA | binding | |
| | GLEAFGPALMEPGAFSGARPGLGGYSPPPEEAMPFEFDQPAQR | protein | |
| | GCSQLLLQVPDLAPGGPGAAGVPGAPPEEPQALRPAKAGSRGG | | |
| | YSPPPEETMPFELDGEGFGDDSPPPGLSRVIAQVDGSSQFAAV | | |
| | AASSAVRLTPAANAPPLWVPGAIGSPSQEAVRPPSNFTGSSPW | | |
| | MEISGPPFEIGSAPAGVDDTPVNMDSPPIALDGPPIKVSGAPD | | |
| | KRERAERPPVEEEAAEMEGAADAAEGGKVPSPGYGSPAAGAAS | | |
| | ADTAARAAPAAPADPDSGATPEDPDSGTAPADPDSGAFAADPD | | |
| | SGAAPAAPADPDSGAAPDAPADPDSGAAPDAPADPDAGAAPEA | | |
| | PAAPAAAETRAAHVAPAAPDAGAPTAPAASATRAAQVRRAASA | | |
| | APASGARRKIHLRPPSPEIQAADPPTPRPTRASAWRGKSESSR | | |
| | GRRVYYDEGVASSDDDSSGDESDDGTSGCLRWFQHRRNRRRRK | | |
| | PQRNLLRNFLVQAFGGCFGRSESPQPKASRSLKVKKVPLAEKR | | |
| | RQMRKEALEKRAQKRAEKKRSKLIDKQLQDEKMGYMCTHRLLL | | |
| | LGAGESGKSTIVKQMRILHVNGFNGEGGEEDPQAARSNSDGEK | | |
| | ATKVQDIKNNLKEAIETIVAAMSNLVPPVELANPENQFRVDYI | | |
| | LSVMNVPDFDFPPEFYEHAKALWEDEGVRACYERSNEYQLIDC | | |
| | AQYFLDKIDVIKQADYVPSDQDLLRCRVLTSGIFETKFQVDKV | | |
| | NFHMFDVGGQRDERRKWIQCFNDVTAIIFVVASSSYNMVIRED | | |
| | NQTNRLQEALNLFKSIWNNRWLRTISVILFLNKQDLLAEKVLA | | |
| | GKSKIEDYFPEFARYTTPEDATPEPGEDPRVTRAKYFIRDEFL | | |
| | RISTASGDGRHYCYPHFTCAVDTENIRRVFNDCRDIIQRMHLR | | |
| | QYELL | | |
| 6 | MNQELLSVGSKRRRTGGSLRGNPSSSQVDEEQMNRVVEEEQQQ | F-Box | FBXW7 |
| | QLRQQEEEHTARNGEVVGVEPRPGGQNDSQQGQLEENNNRFIS | and WD | |
| | VDEDSSGNQEEQEEDEEHAGEQDEEDEEEEEMDQESDDFDQSD | Repeating | |
| | DSSREDEHTHTNSVTNSSSIVDLPVHQLSSPFYTKTTKMKRKL | Domain | |
| | DHGSEVRSFSLGKKPCKVSEYTSTTGLVPCSATPTTFGDLRAA | Containing | |
| | NGQGQQRRRITSVQPPTGLQEWLKMFQSWSGPEKLLALDELID | 7 | |
| | SCEPTQVKHMMQVIEPQFORDFISLLPKELALYVLSFLEPKDL | | |
| | LQAAQTCRYWRILAEDNLLWREKCKEEGIDEPLHIKRRKVIKP | | |
| | GFIHSPWKSAYIRQHRIDTNWRRGELKSPKVLKGHDDHVITCL | | |
| | QFCGNRIVSGSDDNTLKVWSAVTGKCLRTLVGHTGGVWSSQMR | | |
| | DNIIISGSTDRTLKVWNAETGECIHTLYGHTSTVRCMHLHEKR | | |
| | VVSGSRDATLRVWDIETGQCLHVLMGHVAAVRCVQYDGRRVVS | | |
| | GAYDFMVKVWDPETETCLHTLQGHTNRVYSLQFDGIHVVSGSL | | |
| | DTSIRVWDVETGNCIHTLTGHQSLTSGMELKDNILVSGNADST | | |
| | VKIWDIKTGQCLQTLQGPNKHQSAVTCLQFNKNFVITSSDDGT | | |
| | VKLWDLKTGEFIRNLVTLESGGSGGVVWRIRASNTKLVCAVGS | | |
| | RNGTEETKLLVLDFDVDMK | | |
| 7 | MATQADLMELDMAMEPDRKAAVSHWQQQSYLDSGIHSGATTTA | Catenin | CTNNB1 |
| | PSLSGKGNPEEEDVDTSQVLYEWEQGFSQSFTQEQVADIDGQY | beta-1 | |
| | AMTRAQRVRAAMFPETLDEGMQIPSTQFDAAHPTNVQRLAEPS | | |
| | QMLKHAVVNLINYQDDAELATRAIPELTKLLNDEDQVVVNKAA | | |
| | VMVHQLSKKEASRHAIMRSPQMVSAIVRTMQNTNDVETARCTA | | |
| | GTLHNLSHHREGLLAIFKSGGIPALVKMLGSPVDSVLFYAITT | | |
| | LHNLLLHQEGAKMAVRLAGGLQKMVALLNKTNVKFLAITTDCL | | |
| | QILAYGNQESKLIILASGGPQALVNIMRTYTYEKLLWTTSRVL | | |
| | KVLSVCSSNKPAIVEAGGMQALGLHLTDPSQRLVQNCLWTLRN | | |
| | LSDAATKQEGMEGLLGTLVQLLGSDDINVVTCAAGILSNLTCN | | |
| | NYKNKMMVCQVGGIEALVRTVLRAGDREDITEPAICALRHLTS | | |
| | RHQEAEMAQNAVRLHYGLPVVVKLLHPPSHWPLIKATVGLIRN | | |
| | LALCPANHAPLREQGAIPRLVQLLVRAHQDTORRTSMGGTQQQ | | |
| | FVEGVRMEEIVEGCTGALHILARDVHNRIVIRGLNTIPLFVQL | | |
| | LYSPIENIQRVAAGVLCELAQDKEAAEAIEAEGATAPLTELLH | | |
| | SRNEGVATYAAAVLFRMSEDKPQDYKKRLSVELTSSLFRTEPM | | |
| | AWNETADLGLDIGAQGEPLGYRQDDPSYRSFHSGGYGQDALGM | | |
| | DPMMEHEMGGHHPGADYPVDGLPDLGHAQDLMDGLPPGDSNQL | | |
| | AWFDTDL | | |
| 8 | MPAMPSSGPGDTSSSAAEREEDRKDGEEQEEPRGKEERQEPST | DNA | DNMT3A |
| | TARKVGRPGRKRKHPPVESGDTPKDPAVISKSPSMAQDSGASE | (cytosine- | |
| | LLPNGDLEKRSEPQPEEGSPAGGQKGGAPAEGEGAAETLPEAS | 5)-methyl- | |
| | RAVENGCCTPKEGRGAPAEAGKEQKETNIESMKMEGSRGRLRG | trans- | |
| | GLGWESSLRQRPMPRLTFQAGDPYYISKRKRDEWLARWKREAE | ferase | |
| | KKAKVIAGMNAVEENQGPGESQKVEEASPPAVQQPTDPASPTV | 3A | |
| | ATTPEPVGSDAGDKNATKAGDDEPEYEDGRGFGIGELVWGKLR | | |
| | GFSWWPGRIVSWWMTGRSRAAEGTRWVMWFGDGKFSVVCVEKL | | |
| | MPLSSFCSAFHQATYNKQPMYRKAIYEVLQVASSRAGKLFPVC | | |
| | HDSDESDTAKAVEVQNKPMIEWALGGFQPSGPKGLEPPEEEKN | | |
| | PYKEVYTDMWVEPEAAAYAPPPPAKKPRKSTAEKPKVKEIIDE | | |
| | RTRERLVYEVRQKCRNIEDICISCGSLNVTLEHPLFVGGMCQN | | |
| | CKNCFLECAYQYDDDGYQSYCTICCGGREVLMCGNNNCCRCFC | | |
| | VECVDLLVGPGAAQAAIKEDPWNCYMCGHKGTYGLLRRREDWP | | |
| | SRLQMFFANNHDQEFDPPKVYPPVPAEKRKPIRVLSLFDGIAT | | |
| | GLLVLKDLGIQVDRYIASEVCEDSITVGMVRHQGKIMYVGDVR | | |
| | SVTQKHIQEWGPFDLVIGGSPCNDLSIVNPARKGLYEGTGRLF | | |
| | FEFYRLLHDARPKEGDDRPFFWLFENVVAMGVSDKRDISRFLE | | |
| | SNPVMIDAKEVSAAHRARYFWGNLPGMNRPLASTVNDKLELQE | | |
| | CLEHGRIAKFSKVRTITTRSNSIKQGKDQHFPVFMNEKEDILW | | |
| | CTEMERVFGFPVHYTDVSNMSRLARQRLLGRSWSVPVIRHLFA | | |
| | PLKEYFACV | | |
| 9 | MRPSGTAGAALLALLAALCPASRALEEKKVCQGTSNKLTQLGT | Epidermal | EGFR |
| | FEDHFLSLQRMFNNCEVVLGNLEITYVQRNYDLSFLKTIQEVA | growth | |
| | GYVLIALNTVERIPLENLQIIRGNMYYENSYALAVLSNYDANK | factor | |
| | TGLKELPMRNLQEILHGAVRFSNNPALCNVESIQWRDIVSSDF | receptor | |
| | LSNMSMDFQNHLGSCQKCDPSCPNGSCWGAGEENCQKLTKIIC | (EGFR | |
| | AQQCSGRCRGKSPSDCCHNQCAAGCTGPRESDCLVCRKFRDEA | protein) | |
| | TCKDTCPPLMLYNPTTYQMDVNPEGKYSFGATCVKKCPRNYVV | | |
| | TDHGSCVRACGADSYEMEEDGVRKCKKCEGPCRKVCNGIGIGE | | |
| | FKDSLSINATNIKHFKNCTSISGDLHILPVAFRGDSFTHTPPL | | |
| | DPQELDILKTVKEITGFLLIQAWPENRTDLHAFENLEIIRGRT | | |
| | KQHGQFSLAVVSLNITSLGLRSLKEISDGDVIISGNKNLCYAN | | |
| | TINWKKLFGTSGQKTKIISNRGENSCKATGQVCHALCSPEGCW | | |
| | GPEPRDCVSCRNVSRGRECVDKCNLLEGEPREFVENSECIQCH | | |
| | PECLPQAMNITCTGRGPDNCIQCAHYIDGPHCVKTCPAGVMGE | | |
| | NNTLVWKYADAGHVCHLCHPNCTYGCTGPGLEGCPTNGPKIPS | | |
| | IATGMVGALLLLLVVALGIGLFMRRRHIVRKRTLRRLLQEREL | | |
| | VEPLTPSGEAPNQALLRILKETEFKKIKVLGSGAFGTVYKGLW | | |
| | IPEGEKVKIPVAIKELREATSPKANKEILDEAYVMASVDNPHV | | |
| | CRLLGICLTSTVQLITQLMPFGCLLDYVREHKDNIGSQYLLNW | | |
| | CVQIAKGMNYLEDRRLVHRDLAARNVLVKTPQHVKITDFGLAK | | |
| | LLGAEEKEYHAEGGKVPIKWMALESILHRIYTHQSDVWSYGVT | | |
| | VWELMTFGSKPYDGIPASEISSILEKGERLPQPPICTIDVYMI | | |
| | MVKCWMIDADSRPKFRELIIEFSKMARDPQRYLVIQGDERMHL | | |
| | PSPTDSNFYRALMDEEDMDDVVDADEYLIPQQGFFSSPSTSRT | | |
| | PLLSSLSATSNNSTVACIDRNGLQSCPIKEDSFLQRYSSDPTG | | |
| | ALTEDSIDDTFLPVPEYINQSVPKRPAGSVQNPVYHNQPLNPA | | |
| | PSRDPHYQDPHSTAVGNPEYLNTVQPTCVNSTFDSPAHWAQKG | | |
| | SHQISLDNPDYQQDFFPKEAKPNGIFKGSTAENAEYLRVAPQS | | |
| | SEFIGA | | |
| 10 | MAPPSEETPLIPQRSCSLLSTEAGALHVLLPARGPGPPQRLSF | Tyrosine- | JAK3 |
| | SFGDHLAEDLCVQAAKASGILPVYHSLFALATEDLSCWFPPSH | protein | |
| | IFSVEDASTQVLLYRIRFYFPNWFGLEKCHRFGLRKDLASAIL | kinase | |
| | DLPVLEHLFAQHRSDLVSGRLPVGLSLKEQGECLSLAVLDLAR | JAK3 | |
| | MAREQAQRPGELLKTVSYKACLPPSLRDLIQGLSFVTRRRIRR | | |
| | TVRRALRRVAACQADRHSLMAKYIMDLERLDPAGAAETFHVGL | | |
| | PGALGGHDGLGLLRVAGDGGIAWTQGEQEVLQPFCDFPEIVDI | | |
| | SIKQAPRVGPAGEHRLVTVTRTDNQILEAEFPGLPEALSFVAL | | |
| | VDGYFRLTTDSQHFFCKEVAPPRLLEEVAEQCHGPITLDFAIN | | |
| | KLKTGGSRPGSYVLRRSPQDFDSFLLTVCVQNPLGPDYKGCLI | | |
| | RRSPTGTFLLVGLSRPHSSLRELLATCWDGGLHVDGVAVTLTS | | |
| | CCIPRPKEKSNLIVVQRGHSPPTSSLVQPQSQYQLSQMTFHKI | | |
| | PADSLEWHENLGHGSFTKIYRGCRHEVVDGEARKTEVLLKVMD | | |
| | AKHKNCMESFLEAASLMSQVSYRHLVLLHGVCMAGDSTMVQEF | | |
| | VHLGAIDMYLRKRGHLVPASWKLQVVKQLAYALNYLEDKGLPH | | |
| | GNVSARKVLLAREGADGSPPFIKLSDPGVSPAVLSLEMLTDRI | | |
| | PWVAPECLREAQTLSLEADKWGFGATVWEVFSGVTMPISALDP | | |
| | AKKLQFYEDRQQLPAPKWTELALLIQQCMAYEPVQRPSFRAVI | | |
| | RDLNSLISSDYELLSDPTPGALAPRDGLWNGAQLYACQDPTIF | | |
| | EERHLKYISQLGKGNFGSVELCRYDPLGDNTGALVAVKQLQHS | | |
| | GPDQQRDFQREIQILKALHSDFIVKYRGVSYGPGRQSLRLVME | | |
| | YLPSGCLRDFLQRHRARLDASRLLLYSSQICKGMEYLGSRRCV | | |
| | HRDLAARNILVESEAHVKIADFGLAKLLPLDKDYYVVREPGQS | | |
| | PIFWYAPESLSDNIFSRQSDVWSFGVVLYELFTYCDKSCSPSA | | |
| | EFLRMMGCERDVPALCRLLELLEEGQRLPAPPACPAEVHELMK | | |
| | LCWAPSPQDRPSFSALGPQLDMLWSGSRGCETHAFTAHPEGKH | | |
| | HSLSFS | | |
| 11 | MAAVILESIFLKRSQQKKKTSPLNFKKRLFLLTVHKLSYYEYD | Tyrosine- | BTK |
| | FERGRRGSKKGSIDVEKITCVETVVPEKNPPPERQIPRRGEES | protein | |
| | SEMEQISIIERFPYPFQVVYDEGPLYVFSPTEELRKRWIHQLK | kinase | |
| | NVIRYNSDLVQKYHPCFWIDGQYLCCSQTAKNAMGCQILENRN | BTK | |
| | GSLKPGSSHRKTKKPLPPTPEEDQILKKPLPPEPAAAPVSTSE | | |
| | LKKVVALYDYMPMNANDLQLRKGDEYFILEESNLPWWRARDKN | | |
| | GQEGYIPSNYVTEAEDSIEMYEWYSKHMTRSQAEQLLKQEGKE | | |
| | GGFIVRDSSKAGKYTVSVFAKSTGDPQGVIRHYVVCSTPQSQY | | |
| | YLAEKHLFSTIPELINYHQHNSAGLISRLKYPVSQQNKNAPST | | |
| | AGLGYGSWEIDPKDLTFLKELGTGQFGVVKYGKWRGQYDVAIK | | |
| | MIKEGSMSEDEFIEEAKVMMNLSHEKLVQLYGVCTKQRPIFII | | |
| | TEYMANGCLLNYLREMRHRFQTQQLLEMCKDVCEAMEYLESKQ | | |
| | FLHRDLAARNCLVNDQGVVKVSDFGLSRYVLDDEYTSSVGSKF | | |
| | PVRWSPPEVLMYSKFSSKSDIWAFGVLMWEIYSLGKMPYERFT | | |
| | NSETAEHIAQGLRLYRPHLASEKVYTIMYSCWHEKADERPTFK | | |
| | ILLSNILDVMDEES | | |
| 12 | MWSWKCLLFWAVLVTATLCTARPSPTLPEQAQPWGAPVEVESF | Fibroblast | FGFR1 |
| | LVHPGDLLQLRCRLRDDVQSINWLRDGVQLAESNRTRITGEEV | growth | |
| | EVQDSVPADSGLYACVTSSPSGSDTTYFSVNVSDALPSSEDDD | factor | |
| | DDDDSSSEEKETDNTKPNRMPVAPYWTSPEKMEKKLHAVPAAK | receptor | |
| | TVKFKCPSSGTPNPTLRWLKNGKEFKPDHRIGGYKVRYATWSI | 1 | |
| | IMDSVVPSDKGNYTCIVENEYGSINHTYQLDVVERSPHRPILQ | (FGFR1 | |
| | AGLPANKTVALGSNVEFMCKVYSDPQPHIQWLKHIEVNGSKIG | protein) | |
| | PDNLPYVQILKTAGVNTTDKEMEVLHLRNVSFEDAGEYTCLAG | | |
| | NSIGLSHHSAWLTVLEALEERPAVMTSPLYLEIIIYCTGAFLI | | |
| | SCMVGSVIVYKMKSGTKKSDFHSQMAVHKLAKSIPLRRQVTVS | | |
| | ADSSASMNSGVLLVRPSRLSSSGTPMLAGVSEYELPEDPRWEL | | |
| | PRDRLVLGKPLGEGCFGQVVLAEAIGLDKDKPNRVTKVAVKML | | |
| | KSDATEKDLSDLISEMEMMKMIGKHKNIINLLGACTQDGPLYV | | |
| | IVEYASKGNLREYLQARRPPGLEYCYNPSHNPEEQLSSKDLVS | | |
| | CAYQVARGMEYLASKKCIHRDLAARNVLVTEDNVMKIADFGLA | | |
| | RDIHHIDYYKKTTNGRLPVKWMAPEALFDRIYTHQSDVWSFGV | | |
| | LLWEIFTLGGSPYPGVPVEELFKLLKEGHRMDKPSNCTNELYM | | |
| | MMRDCWHAVPSQRPTFKQLVEDLDRIVALTSNQEYLDLSMPLD | | |
| | QYSPSFPDTRSSTCSSGEDSVFSHEPLPEEPCLPRHPAQLANG | | |
| | GLKRR | | |
| 13 | MVSWGRFICLVVVTMATLSLARPSFSLVEDTTLEPEEPPTKYQ | Fibroblast | FGFR2 |
| | ISQPEVYVAAPGESLEVRCLLKDAAVISWTKDGVHLGPNNRTV | growth | |
| | LIGEYLQIKGATPRDSGLYACTASRTVDSETWYFMVNVTDAIS | factor | |
| | SGDDEDDTDGAEDFVSENSNNKRAPYWTNTEKMEKRLHAVPAA | receptor | |
| | NTVKFRCPAGGNPMPTMRWLKNGKEFKQEHRIGGYKVRNQHWS | 2 | |
| | LIMESVVPSDKGNYTCVVENEYGSINHTYHLDVVERSPHRPIL | (FGFR2 | |
| | QAGLPANASTVVGGDVEFVCKVYSDAQPHIQWIKHVEKNGSKY | protein) | |
| | GPDGLPYLKVLKAAGVNTTDKEIEVLYIRNVTFEDAGEYTCLA | | |
| | GNSIGISFHSAWLTVLPAPGREKEITASPDYLEIAIYCIGVFL | | |
| | IACMVVTVILCRMKNTTKKPDFSSQPAVHKLTKRIPLRRQVTV | | |
| | SAESSSSMNSNTPLVRITTRLSSTADTPMLAGVSEYELPEDPK | | |
| | WEFPRDKLTLGKPLGEGCFGQVVMAEAVGIDKDKPKEAVTVAV | | |
| | KMLKDDATEKDLSDLVSEMEMMKMIGKHKNIINLLGACTQDGP | | |
| | LYVIVEYASKGNLREYLRARRPPGMEYSYDINRVPEEQMTFKD | | |
| | LVSCTYQLARGMEYLASQKCIHRDLAARNVLVTENNVMKIADF | | |
| | GLARDINNIDYYKKTTNGRLPVKWMAPEALFDRVYTHQSDVWS | | |
| | FGVLMWEIFTLGGSPYPGIPVEELFKLLKEGHRMDKPANCTNE | | |
| | LYMMMRDCWHAVPSQRPTFKQLVEDLDRILTLTTNEEYLDLSQ | | |
| | PLEQYSPSYPDTRSSCSSGDDSVFSPDPMPYEPCLPQYPHING | | |
| | SVKT | | |
| 14 | MGAPACALALCVAVAIVAGASSESLGTEQRVVGRAAEVPGPEP | Fibroblast | FGFR3 |
| | GQQEQLVFGSGDAVELSCPPPGGGPMGPTVWVKDGTGLVPSER | growth | |
| | VLVGPQRLQVLNASHEDSGAYSCRQRLTQRVLCHFSVRVTDAP | factor | |
| | SSGDDEDGEDEAEDTGVDTGAPYWTRPERMDKKLLAVPAANTV | receptor | |
| | RFRCPAAGNPTPSISWLKNGREFRGEHRIGGIKLRHQQWSLVM | 3 | |
| | ESVVPSDRGNYTCVVENKFGSIRQTYTLDVLERSPHRPILQAG | (FGFR3 | |
| | LPANQTAVLGSDVEFHCKVYSDAQPHIQWLKHVEVNGSKVGPD | protein) | |
| | GTPYVTVLKTAGANTTDKELEVLSLHNVTFEDAGEYTCLAGNS | | |
| | IGFSHHSAWLVVLPAEEELVEADEAGSVYAGILSYGVGFFLFI | | |
| | LVVAAVTLCRLRSPPKKGLGSPTVHKISRFPLKRQVSLESNAS | | |
| | MSSNTPLVRIARLSSGEGPTLANVSELELPADPKWELSRARLT | | |
| | LGKPLGEGCFGQVVMAEAIGIDKDRAAKPVTVAVKMLKDDATD | | |
| | KDLSDLVSEMEMMKMIGKHKNIINLLGACTQGGPLYVLVEYAA | | |
| | KGNLREFLRARRPPGLDYSFDTCKPPEEQLTFKDLVSCAYQVA | | |
| | RGMEYLASQKCIHRDLAARNVLVTEDNVMKIADFGLARDVHNL | | |
| | DYYKKTTNGRLPVKWMAPEALFDRVYTHQSDVWSFGVLLWEIF | | |
| | TLGGSPYPGIPVEELFKLLKEGHRMDKPANCTHDLYMIMRECW | | |
| | HAAPSQRPTFKQLVEDLDRVLTVTSTDEYLDLSAPFEQYSPGG | | |
| | QDTPSSSSSGDDSVFAHDLLPPAPPSSGGSRT | | |
| 15 | MELAALCRWGLLLALLPPGAASTQVCTGTDMKLRLPASPETHL | Receptor | HER2 |
| | DMLRHLYQGCQVVQGNLELTYLPTNASLSFLQDIQEVQGYVLI | tyrosine- | |
| | AHNQVRQVPLQRLRIVRGTQLFEDNYALAVLDNGDPLNNTTPV | protein | |
| | TGASPGGLRELQLRSLTEILKGGVLIQRNPQLCYQDTILWKDI | kinase | |
| | FHKNNQLALTLIDTNRSRACHPCSPMCKGSRCWGESSEDCQSL | erbB-2 | |
| | TRTVCAGGCARCKGPLPTDCCHEQCAAGCTGPKHSDCLACLHF | (ERBB2 | |
| | NHSGICELHCPALVTYNTDTFESMPNPEGRYTFGASCVTACPY | protein) | |
| | NYLSTDVGSCTLVCPLHNQEVTAEDGTQRCEKCSKPCARVCYG | | |
| | LGMEHLREVRAVTSANIQEFAGCKKIFGSLAFLPESFDGDPAS | | |
| | NTAPLQPEQLQVFETLEEITGYLYISAWPDSLPDLSVFQNLQV | | |
| | IRGRILHNGAYSLTLQGLGISWLGLRSLRELGSGLALIHHNTH | | |
| | LCFVHTVPWDQLFRNPHQALLHTANRPEDECVGEGLACHQLCA | | |
| | RGHCWGPGPTQCVNCSQFLRGQECVEECRVLQGLPREYVNARH | | |
| | CLPCHPECQPQNGSVTCFGPEADQCVACAHYKDPPFCVARCPS | | |
| | GVKPDLSYMPIWKFPDEEGACQPCPINCTHSCVDLDDKGCPAE | | |
| | QRASPLTSIISAVVGILLVVVLGVVFGILIKRRQQKIRKYTMR | | |
| | RLLQETELVEPLTPSGAMPNQAQMRILKETELRKVKVLGSGAF | | |
| | GTVYKGIWIPDGENVKIPVAIKVLRENTSPKANKEILDEAYVM | | |
| | AGVGSPYVSRLLGICLTSTVQLVTQLMPYGCLLDHVRENRGRL | | |
| | GSQDLLNWCMQIAKGMSYLEDVRLVHRDLAARNVLVKSPNHVK | | |
| | ITDFGLARLLDIDETEYHADGGKVPIKWMALESILRRRFTHQS | | |
| | DVWSYGVTVWELMTFGAKPYDGIPAREIPDLLEKGERLPQPPI | | |
| | CTIDVYMIMVKCWMIDSECRPRFRELVSEFSRMARDPQRFVVI | | |
| | QNEDLGPASPLDSTFYRSLLEDDDMGDLVDAEEYLVPQQGFFC | | |
| | PDPAPGAGGMVHHRHRSSSTRSGGGDLTLGLEPSEEEAPRSPL | | |
| | APSEGAGSDVFDGDLGMGAAKGLQSLPTHDPSPLQRYSEDPTV | | |
| | PLPSETDGYVAPLTCSPQPEYVNQPDVRPQPPSPREGPLPAAR | | |
| | PAGATLERPKTLSPGKNGVVKDVFAFGGAVENPEYLTPQGGAA | | |
| | PQPHPPPAFSPAFDNLYYWDQDPPERGAPPSTFKGTPTAENPE | | |
| | YLGLDVPV | | |
| 16 | MRANDALQVLGLLFSLARGSEVGNSQAVCPGTLNGLSVTGDAE | Receptor | HER3 |
| | NQYQTLYKLYERCEVVMGNLEIVLTGHNADLSFLQWIREVTGY | tyrosine- | |
| | VLVAMNEFSTLPLPNLRVVRGTQVYDGKFAIFVMLNYNTNSSH | protein | |
| | ALRQLRLTQLTEILSGGVYIEKNDKLCHMDTIDWRDIVRDRDA | kinase | |
| | EIVVKDNGRSCPPCHEVCKGRCWGPGSEDCQTLTKTICAPQCN | erbB-3 | |
| | GHCFGPNPNQCCHDECAGGCSGPQDTDCFACRHFNDSGACVPR | (ERBB3 | |
| | CPQPLVYNKLTFQLEPNPHTKYQYGGVCVASCPHNFVVDQTSC | protein) | |
| | VRACPPDKMEVDKNGLKMCEPCGGLCPKACEGTGSGSRFQTVD | | |
| | SSNIDGFVNCTKILGNLDFLITGLNGDPWHKIPALDPEKLNVF | | |
| | RTVREITGYLNIQSWPPHMHNFSVFSNLTTIGGRSLYNRGFSL | | |
| | LIMKNLNVTSLGFRSLKEISAGRIYISANRQLCYHHSLNWTKV | | |
| | LRGPTEERLDIKHNRPRRDCVAEGKVCDPLCSSGGCWGPGPGQ | | |
| | CLSCRNYSRGGVCVTHCNFLNGEPREFAHEAECFSCHPECQPM | | |
| | EGTATCNGSGSDTCAQCAHFRDGPHCVSSCPHGVLGAKGPIYK | | |
| | YPDVQNECRPCHENCTQGCKGPELQDCLGQTLVLIGKTHLTMA | | |
| | LTVIAGLVVIFMMLGGTFLYWRGRRIQNKRAMRRYLERGESIE | | |
| | PLDPSEKANKVLARIFKETELRKLKVLGSGVFGTVHKGVWIPE | | |
| | GESIKIPVCIKVIEDKSGRQSFQAVTDHMLAIGSLDHAHIVRL | | |
| | LGLCPGSSLQLVTQYLPLGSLLDHVRQHRGALGPQLLLNWGVQ | | |
| | IAKGMYYLEEHGMVHRNLAARNVLLKSPSQVQVADFGVADLLP | | |
| | PDDKQLLYSEAKTPIKWMALESIHFGKYTHQSDVWSYGVTVWE | | |
| | LMTFGAEPYAGLRLAEVPDLLEKGERLAQPQICTIDVYMVMVK | | |
| | CWMIDENIRPTFKELANEFTRMARDPPRYLVIKRESGPGIAPG | | |
| | PEPHGLTNKKLEEVELEPELDLDLDLEAEEDNLATTTLGSALS | | |
| | LPVGTLNRPRGSQSLLSPSSGYMPMNQGNLGESCQESAVSGSS | | |
| | ERCPRPVSLHPMPRGCLASESSEGHVTGSEAELQEKVSMCRSR | | |
| | SRSRSPRPRGDSAYHSQRHSLLTPVTPLSPPGLEEEDVNGYVM | | |
| | PDTHLKGTPSSREGTLSSVGLSSVLGTEEEDEDEEYEYMNRRR | | |
| | RHSPPHPPRPSSLEELGYEYMDVGSDLSASLGSTQSCPLHPVP | | |
| | IMPTAGTTPDEDYEYMNRQRDGGGPGGDYAAMGACPASEQGYE | | |
| | EMRAFQGPGHQAPHVHYARLKTLRSLEATDSAFDNPDYWHSRL | | |
| | FPKANAQRT | | |

The intracellular protein (e.g., mutated or overexpressed) can comprise a cysteine residue. In some embodiments, the intracellular mutated protein comprises a mutant-specific cysteine residue, which is absent in a corresponding wild-type protein. The targeting ligand can form a covalent bond with the mutant-specific cysteine residue. In some embodiments, the intracellular mutated protein comprises a mutant-specific steric environment in the region around at least one of the endogenous cysteine residues. In some embodiments, the targeting ligand selectively binds to at least one of the endogenous cysteine residues in the intracellular mutated protein over a same cysteine residue in a corresponding wild-type protein. In some embodiments, the intracellular protein is overexpressed compared to a corresponding wild-type protein. The intracellular protein can be an overexpressed protein that comprises an endogenous cysteine residue. In some embodiments, the intracellular protein is not mutated. Accordingly, in one aspect, disclosed herein are covalently modified proteins comprising a covalently and irreversibly bound radiopharmaceutical conjugate, and an intracellular protein (e.g., mutated or overexpressed) that comprises a cysteine residue.

In some embodiments, the overexpressed intracellular protein is a cysteine-rich intestinal protein 1 (CRIP1), cysteine-rich secretory protein 3 (CRISP3), or cysteine-rich 61 protein (CYR61). In some embodiments, described herein is a covalently modified protein comprising a covalently bound radiopharmaceutical conjugate, and a CRIP1 protein. In some embodiments, described herein is a covalently modified protein comprising a covalently bound radiopharmaceutical conjugate, and a CRISP3 protein. In some embodiments, described herein is a covalently modified protein comprising a covalently bound radiopharmaceutical conjugate, and a CYR61 protein.

In some embodiments, the electrophilic group of the targeting ligand covalently binds to the intracellular protein (e.g., intracellular mutated protein) at a cysteine residue.

The intracellular mutated protein can be a KRAS protein. In some embodiments, described herein is a covalently modified protein comprising a covalently bound radiopharmaceutical conjugate, and a KRAS protein. In some embodiments, the radiopharmaceutical conjugate comprises a targeting ligand, a linker, a metal chelator and optionally a radionuclide. In some embodiments, the targeting ligand forms a covalent bond with the mutated KRAS protein. In some embodiments, the covalent bond is formed between the targeting ligand and a cysteine, lysine or serine residue of the KRAS protein. In some embodiments, the targeting ligand does not form a covalent bond with a wild-type KRAS protein. In some embodiments, the targeting ligand forms a covalent bond with a KRAS protein at position 13 according to SEQ ID NO:1. In some embodiments, the KRAS protein comprises a G to C amino acid substitution at position 13.

The intracellular mutated protein can be a Fibroblast growth factor receptor 3 (FGFR3 protein). In some embodiments, described herein is a covalently modified protein comprising a covalently bound radiopharmaceutical conjugate, and a FGFR3 protein. In some embodiments, the radiopharmaceutical conjugate comprises a targeting ligand, a linker, a metal chelator and optionally a radionuclide. In some embodiments, the targeting ligand forms a covalent bond with the mutated FGFR3 protein. In some embodiments, the covalent bond is formed between the targeting ligand and a cysteine, lysine or serine residue of the FGFR3 protein. In some embodiments, the targeting ligand does not form a covalent bond with a wild-type FGFR3 protein. In some embodiments, the targeting ligand forms a covalent bond with the FGFR3 protein at position 249 according to SEQ ID NO:2. In some embodiments, the FGFR3 protein comprises an S to C amino acid substitution at position 249. In some embodiments, the targeting ligand forms a covalent bond with the FGFR3 protein at position 373 according to SEQ ID NO:2. In some embodiments, the FGFR3 protein comprises a Y to C amino acid substitution at position 373. In some embodiments, the targeting ligand forms a covalent bond with the FGFR3 protein at position 248 according to SEQ ID NO:2. In some embodiments, the FGFR3 protein comprises an R to C amino acid substitution at position 248. In some embodiments, the targeting ligand forms a covalent bond with the FGFR3 protein at position 370 according to SEQ ID NO:2. In some embodiments, the FGFR3 protein comprises a G to C amino acid substitution at position 370.

The intracellular mutated protein can be a TP53 protein. In some embodiments, described herein is a covalently modified protein comprising a covalently bound radiopharmaceutical conjugate, and a TP53 protein. In some embodiments, the radiopharmaceutical conjugate comprises a targeting ligand, a linker, a metal chelator and optionally a radionuclide. In some embodiments, the targeting ligand forms a covalent bond with the mutated TP53 protein. In some embodiments, the covalent bond is formed between the targeting ligand and a cysteine, lysine or serine residue of the TP53 protein. In some embodiments, the targeting ligand does not form a covalent bond with a wild-type TP53 protein. In some embodiments, the targeting ligand forms a covalent bond with a TP53 protein at position 273 according to SEQ ID NO:3. In some embodiments, the TP53 protein comprises an R to C amino acid substitution at position 273. In some embodiments, the targeting ligand forms a covalent bond with a TP53 protein at position 220 according to SEQ ID NO:3. In some embodiments, the TP53 protein comprises a Y to C amino acid substitution at position 220. In some embodiments, the targeting ligand forms a covalent bond with a TP53 protein at position 163 according to SEQ ID NO:3. In some embodiments, the TP53 protein comprises a Y to C amino acid substitution at position 163.

The intracellular mutated protein can be an IDH1 protein. In some embodiments, described herein is a covalently modified protein comprising a covalently bound radiopharmaceutical conjugate, and an IDH1 protein. In some embodiments, the radiopharmaceutical conjugate comprises a targeting ligand, a linker, a metal chelator and optionally a radionuclide. In some embodiments, the targeting ligand forms a covalent bond with the mutated IDH1 protein. In some embodiments, the covalent bond is formed between the targeting ligand and a cysteine, lysine or serine residue of the IDH1 protein. In some embodiments, the targeting ligand does not form a covalent bond with a wild-type IDH1 protein. In some embodiments, the targeting ligand forms a covalent bond with an IDH1 protein at position 132 according to SEQ ID NO:4. In some embodiments, the IDH1 protein comprises an R to C amino acid substitution at position 132.

The intracellular mutated protein can be a GNAS protein. In some embodiments, described herein is a covalently modified protein comprising a covalently bound radiopharmaceutical conjugate, and a GNAS protein. In some embodiments, the radiopharmaceutical conjugate comprises a targeting ligand, a linker, a metal chelator and optionally a radionuclide. In some embodiments, the targeting ligand forms a covalent bond with the mutated GNAS protein. In some embodiments, the covalent bond is formed between the targeting ligand and a cysteine, lysine or serine residue of the GNAS protein. In some embodiments, the targeting ligand does not form a covalent bond with a wild-type GNAS protein. In some embodiments, the targeting ligand forms a covalent bond with a GNAS protein at position 201 according to SEQ ID NO:5. In some embodiments, the GNAS protein comprises a mutation at position 201. In some embodiments, the mutation at position 201 comprises an amino acid substitution, wherein the amino acid substitution is R to C.

The intracellular mutated protein can be an FBXW7 protein. In some embodiments, described herein is a covalently modified protein comprising a covalently bound radiopharmaceutical conjugate, and a FBXW7 protein. In some embodiments, the radiopharmaceutical conjugate comprises a targeting ligand, a linker, a metal chelator and optionally a radionuclide. In some embodiments, the targeting ligand forms a covalent bond with the mutated FBXW7 protein. In some embodiments, the covalent bond is formed between the targeting ligand and a cysteine, lysine or serine residue of the FBXW7 protein. In some embodiments, the targeting ligand does not form a covalent bond with a wild-type FBXW7 protein. In some embodiments, the targeting ligand forms a covalent bond with an FBXW7 protein at position 465 according to SEQ ID NO:6. In some embodiments, the FBXW7 protein comprises a mutation at position 465. In some embodiments, the mutation at position 465 comprises an amino acid substitution, wherein the amino acid substitution is R to C.

The intracellular mutated protein can be a CTNNB1 protein. In some embodiments, described herein is a covalently modified protein comprising a covalently bound radiopharmaceutical conjugate, and a CTNNB1 protein. In some embodiments, the radiopharmaceutical conjugate comprises a targeting ligand, a linker, a metal chelator and optionally a radionuclide. In some embodiments, the targeting ligand forms a covalent bond with the mutated CTNNB1 protein. In some embodiments, the covalent bond is formed between the targeting ligand and a cysteine, lysine or serine residue of the CTNNB1 protein. In some embodiments, the targeting ligand does not form a covalent bond with a wild-type CTNNB1 protein. In some embodiments, the targeting ligand forms a covalent bond with a CTNNB1 protein at position 33 according to SEQ ID NO:7. In some embodiments, the CTNNB1 protein comprises a mutation at position 33. In some embodiments, the mutation at position 33 comprises an amino acid substitution. In some embodiments, the amino acid substitution is S to C. In some embodiments, the targeting ligand forms a covalent bond with a CTNNB1 protein at position 37 according to SEQ ID NO:7. In some embodiments, the CTNNB1 protein comprises a mutation at position 37. In some embodiments, the mutation at position 37 comprises an amino acid substitution. In some embodiments, the amino acid substitution is S to C.

The intracellular mutated protein can be a DNMT3A protein. In some embodiments, described herein is a covalently modified protein comprising a covalently bound radiopharmaceutical conjugate, and a DNMT3A protein. In some embodiments, the radiopharmaceutical conjugate comprises a targeting ligand, a linker, a metal chelator and optionally a radionuclide. In some embodiments, the targeting ligand forms a covalent bond with the mutated DNMT3A protein. In some embodiments, the covalent bond is formed between the targeting ligand and a cysteine, lysine or serine residue of the DNMT3A protein. In some embodiments, the targeting ligand does not form a covalent bond with a wild-type DNMT3A protein. In some embodiments, the targeting ligand forms a covalent bond with a DNMT3A protein at position 882 according to SEQ ID NO:8. In some embodiments, the DNMT3A protein comprises a mutation at position 882. In some embodiments, the mutation at position 37 comprises an amino acid substitution. In some embodiments, the amino acid substitution is R to C.

The intracellular mutated protein can be an EGFR protein. In some embodiments, described herein is a covalently modified protein comprising a covalently bound radiopharmaceutical conjugate, and a EGFR protein. In some embodiments, the radiopharmaceutical conjugate comprises a targeting ligand, a linker, a metal chelator and optionally a radionuclide. In some embodiments, the targeting ligand forms a covalent bond with the mutated EGFR protein. In some embodiments, the covalent bond is formed between the targeting ligand and a cysteine, lysine or serine residue of the EGFR protein. In some embodiments, the targeting ligand does not form a covalent bond with a wild-type EGFR protein. In some embodiments, the covalent bond is formed between the targeting ligand and an endogenous cysteine residue of the EGFR protein. The EGFR protein can comprise a mutation in the steric environment of the endogenous cysteine residue, thereby allowing the covalent binding. In some embodiments, the mutated EGFR protein has one or more of the following mutations: G719A, G719S, G719C, deletions of exon 19, insertions in exon 20, T790M, S768I, L858R, and L861Q, wherein the protein sequence numbering is based on SEQ ID NO: 9. In some embodiments, the mutated EGFR protein comprises a G719A mutation. In some embodiments, the mutated EGFR protein comprises a G719S mutation. In some embodiments, the mutated EGFR protein comprises a G719C mutation. In some embodiments, the mutated EGFR protein comprises a deletion of exon 19. In some embodiments, the mutated EGFR protein comprises an insertion in exon 20. In some embodiments, the mutated EGFR protein comprises a T790M mutation. In some embodiments, the mutated EGFR protein comprises a S768I mutation. In some embodiments, the mutated EGFR protein comprises an L858R mutation. In some embodiments, the mutated EGFR protein comprises an L861Q. In some embodiments, the mutated EGFR protein comprises a T to M amino acid substitution at position 790.

The intracellular mutated protein can be an JAK3 protein. In some embodiments, described herein is a covalently modified protein comprising a covalently bound radiopharmaceutical conjugate, and a JAK3 protein. In some embodiments, the radiopharmaceutical conjugate comprises a targeting ligand, a linker, a metal chelator and optionally a radionuclide. In some embodiments, the targeting ligand forms a covalent bond with the mutated JAK3 protein. In some embodiments, the covalent bond is formed between the targeting ligand and a cysteine, lysine or serine residue of the JAK3 protein. In some embodiments, the targeting ligand does not form a covalent bond with a wild-type JAK3 protein. In some embodiments, the covalent bond is formed between the targeting ligand and an endogenous cysteine residue of the JAK3 protein. The JAK3 protein can comprise a mutation in the steric environment of the endogenous cysteine residue, thereby allowing the covalent binding. In some embodiments, the mutated JAK3 protein has one or more of the following mutations: M511I, A572V, A572T, A573V, R657Q, V722I, and L875H, wherein the protein sequence numbering is based on SEQ ID NO: 10. In some embodiments, the mutated JAK3 protein comprises an M511I mutation. In some embodiments, the mutated JAK3 protein comprises an A572V mutation. In some embodiments, the mutated JAK3 protein comprises an A572T mutation. In some embodiments, the mutated JAK3 protein comprises an A573V mutation. In some embodiments, the mutated JAK3 protein comprises an R657Q mutation. In some embodiments, the mutated JAK3 protein comprises an V722I mutation. In some embodiments, the mutated JAK3 protein comprises an L875H mutation.

The intracellular mutated protein can be an BTK protein. In some embodiments, described herein is a covalently modified protein comprising a covalently bound radiopharmaceutical conjugate, and a BTK protein. In some embodiments, the radiopharmaceutical conjugate comprises a targeting ligand, a linker, a metal chelator and optionally a radionuclide. In some embodiments, the targeting ligand forms a covalent bond with the mutated BTK protein. In some embodiments, the covalent bond is formed between the targeting ligand and a cysteine, lysine or serine residue of the BTK protein. In some embodiments, the targeting ligand does not form a covalent bond with a wild-type BTK protein. In some embodiments, the covalent bond is formed between the targeting ligand and an endogenous cysteine residue of the BTK protein. The BTK protein can comprise a mutation in the steric environment of the endogenous cysteine residue, thereby allowing the covalent binding. In some embodiments, the mutated BTK protein has one or more of the following mutations: G1442C, C1443T, and C481S, wherein the protein sequence numbering is based on SEQ ID NO: 11. In some embodiments, the mutated BTK protein comprises a G1442C mutation. In some embodiments, the mutated BTK protein comprises a C1443T mutation. In some embodiments, the mutated BTK protein comprises a C481S mutation.

The intracellular mutated protein can be an FGFR1 protein. In some embodiments, described herein is a covalently modified protein comprising a covalently bound radiopharmaceutical conjugate, and a FGFR1 protein. In some embodiments, the radiopharmaceutical conjugate comprises a targeting ligand, a linker, a metal chelator and optionally a radionuclide. In some embodiments, the targeting ligand forms a covalent bond with the mutated FGFR1 protein. In some embodiments, the covalent bond is formed between the targeting ligand and a cysteine, lysine or serine residue of the FGFR1 protein. In some embodiments, the targeting ligand does not form a covalent bond with a wild-type FGFR1 protein. In some embodiments, the covalent bond is formed between the targeting ligand and an endogenous cysteine residue of the FGFR1 protein. The FGFR1 protein can comprise a mutation in the steric environment of the endogenous cysteine residue, thereby allowing the covalent binding. In some embodiments, the mutated FGFR1 protein has one or more of the following mutations: T141R, R445W, K656E, G818R, and N546K, wherein the protein sequence numbering is based on SEQ ID NO: 12. In some embodiments, the mutated FGFR1 protein comprises a T141R mutation. In some embodiments, the mutated FGFR1 protein comprises a R445W mutation. In some embodiments, the mutated FGFR1 protein comprises a K656E mutation. In some embodiments, the mutated FGFR1 protein comprises a G818R mutation. In some embodiments, the mutated FGFR1 protein comprises a N546K mutation.

The intracellular mutated protein can be an FGFR2 protein. In some embodiments, described herein is a covalently modified protein comprising a covalently bound radiopharmaceutical conjugate, and a FGFR2 protein. In some embodiments, the radiopharmaceutical conjugate comprises a targeting ligand, a linker, a metal chelator and optionally a radionuclide. In some embodiments, the targeting ligand forms a covalent bond with the mutated FGFR2 protein. In some embodiments, the covalent bond is formed between the targeting ligand and a cysteine, lysine or serine residue of the FGFR2 protein. In some embodiments, the targeting ligand does not form a covalent bond with a wild-type FGFR2 protein. In some embodiments, the covalent bond is formed between the targeting ligand and an endogenous cysteine residue of the FGFR2 protein. The FGFR2 protein can comprise a mutation in the steric environment of the endogenous cysteine residue, thereby allowing the covalent binding. In some embodiments, the mutated FGFR2 protein has one or more of the following mutations: S252W, P253R, A315T, D336N, Y375C, C382R, V395D, D471N, I547V, N549K, N549Y, and K659E, wherein the protein sequence numbering is based on SEQ ID NO: 13.

The intracellular mutated protein can be an FGFR3 protein. In some embodiments, described herein is a covalently modified protein comprising a covalently bound radiopharmaceutical conjugate, and a FGFR3 protein. In some embodiments, the radiopharmaceutical conjugate comprises a targeting ligand, a linker, a metal chelator and optionally a radionuclide. In some embodiments, the targeting ligand forms a covalent bond with the mutated FGFR3 protein. In some embodiments, the covalent bond is formed between the targeting ligand and a cysteine, lysine or serine residue of the FGFR3 protein. In some embodiments, the targeting ligand does not form a covalent bond with a wild-type FGFR3 protein. In some embodiments, the covalent bond is formed between the targeting ligand and an endogenous cysteine residue of the FGFR3 protein. The FGFR3 protein can comprise a mutation in the steric environment of the endogenous cysteine residue, thereby allowing the covalent binding. In some embodiments, the mutated FGFR3 protein has one or more of the following mutations: S131L, R248C, S249C, G370C, S371C, Y373C, G380R, R399C, E627K, K650E, K650M, V677I, and D785Y, wherein the protein sequence numbering is based on SEQ ID NO: 14.

The intracellular mutated protein can be an ERBB2 protein. In some embodiments, described herein is a covalently modified protein comprising a covalently bound radiopharmaceutical conjugate, and a ERBB2 protein. In some embodiments, the radiopharmaceutical conjugate comprises a targeting ligand, a linker, a metal chelator and optionally a radionuclide. In some embodiments, the targeting ligand forms a covalent bond with the mutated ERBB2 protein. In some embodiments, the covalent bond is formed between the targeting ligand and a cysteine, lysine or serine residue of the ERBB2 protein. In some embodiments, the targeting ligand does not form a covalent bond with a wild-type ERBB2 protein. In some embodiments, the covalent bond is formed between the targeting ligand and an endogenous cysteine residue of the ERBB2 protein. The ERBB2 protein can comprise a mutation in the steric environment of the endogenous cysteine residue, thereby allowing the covalent binding. In some embodiments, the mutated ERBB2 protein has one or more of the following mutations: G776insYVMA, (“YVMA” disclosed as SEQ ID NO: 18), G309E, S310F, V659, G660, H878Y, L755S, P780-Y781insertionGSP, L755P, T798M, T798I, L869R, and V777L, wherein the protein sequence numbering is based on SEQ ID NO: 15.

The intracellular mutated protein can be an ERBB3 protein. In some embodiments, described herein is a covalently modified protein comprising a covalently bound radiopharmaceutical conjugate, and a ERBB3 protein. In some embodiments, the radiopharmaceutical conjugate comprises a targeting ligand, a linker, a metal chelator and optionally a radionuclide. In some embodiments, the targeting ligand forms a covalent bond with the mutated ERBB3 protein. In some embodiments, the covalent bond is formed between the targeting ligand and a cysteine, lysine or serine residue of the ERBB3 protein. In some embodiments, the targeting ligand does not form a covalent bond with a wild-type ERBB3 protein. In some embodiments, the covalent bond is formed between the targeting ligand and an endogenous cysteine residue of the ERBB3 protein. The ERBB3 protein can comprise a mutation in the steric environment of the endogenous cysteine residue, thereby allowing the covalent binding. In some embodiments, the mutated ERBB3 protein has one or more of the following mutations: F94L, G284R, D297Y, D313H, K329T, and T355I, wherein the protein sequence numbering is based on SEQ ID NO: 16.

### Metal Chelator

In one aspect, described herein are conjugates that comprise a metal chelator that is configured to bind with a radionuclide. The metal chelator can refer to a moiety of the conjugate that is configured to bind with a radionuclide. In some embodiments, a conjugate described herein comprises two or more independent metal chelators, e.g., 2, 3, 4, 5, or more metal chelators. In some embodiments, a conjugate described herein comprises two metal chelators, which can be the same or different. The metal chelator can be attached to the linker or the targeting ligand through any suitable group/atom of the chelator.

In some embodiments, the metal chelator is capable of binding a radioactive atom. The binding can be direct, e.g., the metal chelator can make hydrogen bonds or electrostatic interactions with the radioactive atom. The binding can also be indirect, e.g., the metal chelator binds to a molecule that comprises a radioactive atom. In some embodiments, the metal chelator comprises, or is, a macrocycle. In some embodiments, the metal chelator comprises, or is, 2,2′,2″,2′″-(1,4,7,10-Tetraazacyclododecane-1,4,7,10-tetrayl)tetraacetic acid (DOTA) or 1,4,7-triazacyclononane-1,4,7-triacetic acid (NOTA). In some embodiments, the metal chelator comprises a macrocycle, e.g., a macrocycle comprising an O and/or a N, DOTA, NOTA, one or more amines, one or more ethers, one or more carboxylic acids, EDTA, DTPA, TETA, DO3A, PCTA, or desferrioxamine.

In some embodiments, the metal chelator comprises a plurality of amines. In some embodiments, the metal chelator includes 4 or more N, 4 or more carboxylic acid groups, or a combination thereof. In some embodiments, the metal chelator does not comprise S. In some embodiments, the metal chelator comprises a ring. In some embodiments, the ring comprises an O and/or an N. In some embodiments, the metal chelator is a ring that includes 3 or more N, 3 or more carboxylic acid groups, or a combination thereof. In some embodiments, the metal chelator is poly polydentate.

In some embodiments of a conjugate described herein, a metal chelator described herein is selected from: DOTA, DOTA-GA, pBn-DOTA, pBn-SCN-DOTA, NH₂-DOTA, NH₂-DOTA-GA, p-NCS-Bn-DOTA-GA, p-NH₂-Bn-oxo-DO3A, p-SCN-Bn-oxo-DO3A, NOTA, NODA-GA, NH₂-NODA-GA, p-NCS-Bn-NODA-GA, p-NH₂-Bn-NOTA, p-SCN-Bn-NOTA, NCS-MP-NODA, NH₂-MPAA-NODA, PCTA, p-NH₂-Bn-PCTA, p-SCN-Bn-PCTA, p-SCN-Bn-HEHA, H2-MACROPA-NCS, H1-MACROPA, H2-MACROPA-NH₂, H4-OCTAPA, tetra-(S, S, S, S)-Me-DOTA, tetra-(S, S, S, S)-Et-DOTA, tetra-(S, S, S, S)-iBu-DOTA, or maleimide-nBu-DOTA.

In some embodiments of a conjugate described herein, a metal chelator described herein has a structure of

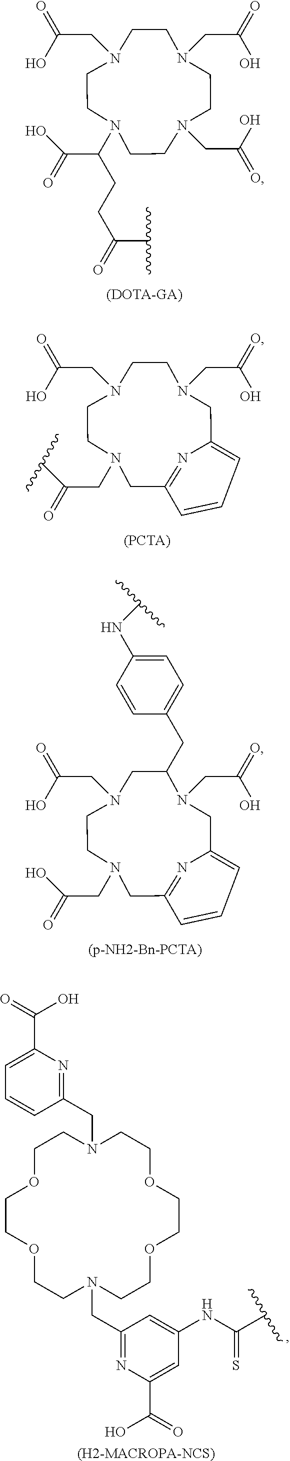

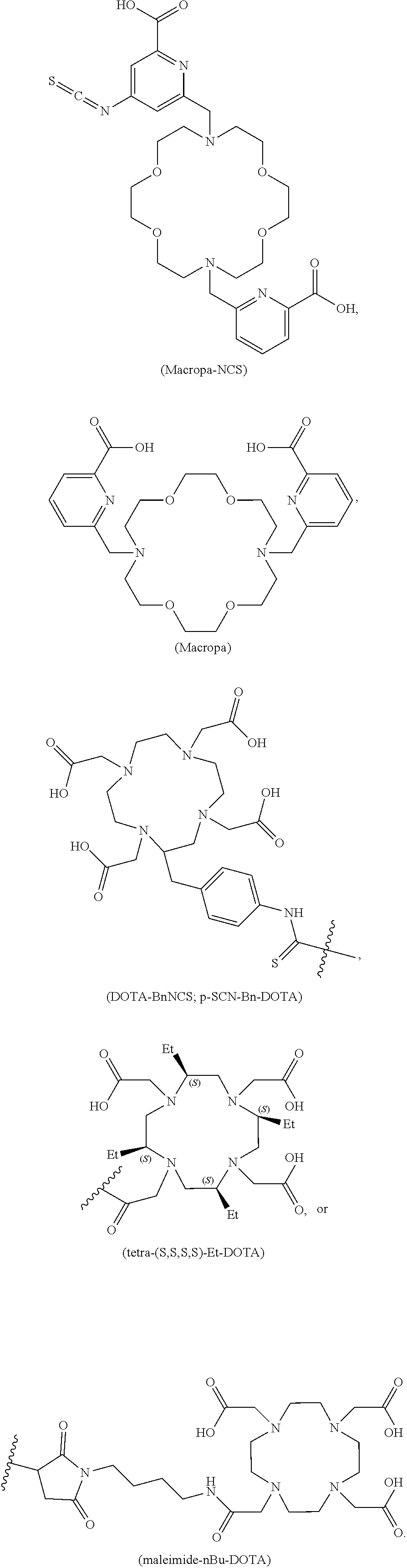

some embodiments, a metal chelator described herein has a structure of

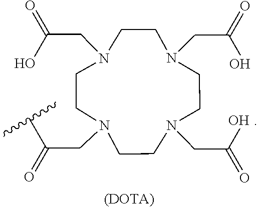

In some embodiments, a metal chelator described herein comprises a cyclic chelating agent. Exemplary cyclic chelating agents include, but are not limited to, AAZTA, BAT, BAT-TM, Crown, Cyclen, DO2A, CB-DO2A, DO3A, H3HP-DO3A, Oxo-DO3A, p-NH₂-Bn-Oxo-DO3A, DOTA, DOTA-3py, DOTA-PA, DOTA-GA, DOTA-4AMP, DOTA-2py, DOTA-1py, p-SCN-Bn-DOTA, CHX-A″-EDTA, MeO-DOTA-NCS EDTA, DOTAMAP, DOTAGA, DOTAGA-anhydride, DOTMA, DOTASA, DOTAM, DOTP, CB-Cyclam, TE2A, CB-TE2A, CB-TE2P, DM-TE2A, MM-TE2A, NOTA, NOTP, HEHA, HEHA-NCS, p-SCN-Bn-HEHA, DTPA, CHX-A″-DTPA, p-NH₂-Bn-CHX-A″-DTPA, p-SCN-DTPA, p-SCN-Bz-Mx-DTPA, 1B4M-DTPA, p-SCN-Bn1B-DTPA, p-SCN-Bn-1B4M-DTPA, p-SCN-Bn-CHX-A″-DTPA, PEPA, p-SCN-Bn-PEPA, TETPA, DOTPA, DOTMP, DOTPM, t-Bu-calix[4]arene-tetracarboxylic acid, macropa, macropa-NCS, macropid, H₃L¹, H₃L⁴, H₂azapa, H₅decapa, bispa², H4pypa, H4octapa, H₄CHXoctapa, p-SCN-Bn-H4octapa, p-SCN-Bn-H₄octapa, TTHA, p-NO₂-Bn-neunpa, H₄octox, H₂macropa, H₂bispa², H₄phospa, H₆phospa, p-SCN-Bn-H₆phospa, TETA, p-NO₂-Bn-TETA, TRAP, TPA, HBED, SHBED, HBED-CC, (HBED-CC)TFP, DMSA, DMPS, DHLA, lipoic acid, TGA, BAL, Bis-thioseminarabazones, p-SCN-NOTA, nNOTA, NODAGA, CB-TE1A1P, 3P-C-NETA-NCS, 3p-C-DEPA, 3P-C-DEPA-NCS, TCMC, PCTA, NODIA-Me, TACN, pycup1A1B, pycup2A, THP, DEDPA, H₂DEDPA, p-SCN-Bn-H₂DEDPA, p-SCN-Bn-TCMC, motexafin, NTA, NOC, 3p-C-NETA, p-NH₂-Bn-TE3A, SarAr, DiAmSar, SarAr-NCS, AmBaSar, BaBaSar, TACN-TM, CP256, C-NE3TA, C-NE3TA-NCS, NODASA, NETA-monoamide, C-NETA, NOPO, BPCA, p-SCN-Bn-DFO, DFO-ChX-Mal, DFO, DFO-IAC, DFO-BAC, DiP-LICAM, EC, SBAD, BAPEN, TACHPYR, NEC-SP, L^{py}, L¹, L², L³, and EuK-106.

In some embodiments, the metal chelator is DO3A. In some embodiments, the metal chelator is PEPA. In some embodiments, the metal chelator is EDTA. In some embodiments, the metal chelator is CHX-A″-DTPA. In some embodiments, the metal chelator is HEHA. In some embodiments, the metal chelator is DOTMP. In some embodiments, the metal chelator is t-Bu-calix[4]arene-tetracarboxylic acid. In some embodiments, the metal chelator is macropa. In some embodiments, the metal chelator is macropa-NCS. In some embodiments, the metal chelator is H⁴py4pa. In some embodiments, the metal chelator is H₄octapa. In some embodiments, the metal chelator is H4CHXoctapa. In some embodiments, the metal chelator is DOTP. In some embodiments, the metal chelator is crown.

In some embodiments, the metal chelator is DOTA. In some embodiments, the metal chelator is DOTA-GA. In some embodiments, the metal chelator is MACROPA. In some embodiments, the metal chelator is a chiral derivative of DOTA. Exemplary chiral DOTA chelators are described in Dai et al., Nature Communications (2018) 9:857. In some embodiments, the metal chelator is 2,2′,2″,2′″-((2S,5S,8S,11S)-2,5,8,11-tetramethyl-1,4,7,10-tetraazacyclododecane-1,4,7,10-tetrayl)tetraacetic acid. In some embodiments, the metal chelator has a structure of

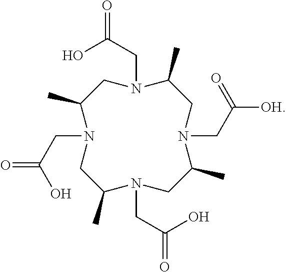 [SMILES: C[C@H]1CN(CC(=O)O)[C@@H](C)CN(CC(=O)O)[C@@H](C)CN(CC(=O)O)[C@@H](C)CN1CC(=O)O]

In some embodiments, the metal chelator is 2,2′,2″,2′″-((2S,5S,8S,11S)-2,5,8,11-tetraethyl-1,4,7,10-tetraazacyclododecane-1,4,7,10-tetrayl)tetraacetic acid. In some embodiments, the metal chelator has a structure of

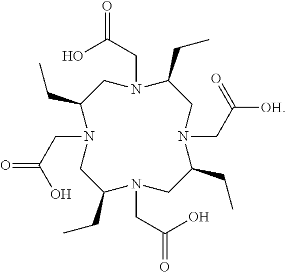 [SMILES: CC[C@H]1CN(CC(=O)O)[C@@H](CC)CN(CC(=O)O)[C@@H](CC)CN(CC(=O)O)[C@@H](CC)CN1CC(=O)O]

In some embodiments of a conjugate described herein, the metal chelator has a structure of

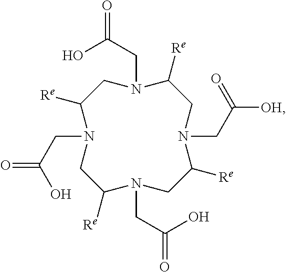

wherein each R^{e} is independently selected from hydrogen, alkyl, haloalkyl, hydroxyalkyl, aminoalkyl, heteroalkyl, alkenyl, alkynyl, cycloalkyl, heterocycloalkyl, aryl, heteroaryl, alkylcycloalkyl, alkylheterocycloalkyl, alkylaryl, alkylheteroaryl, or an amino acid side chain. In some embodiments, the metal chelator has a structure of

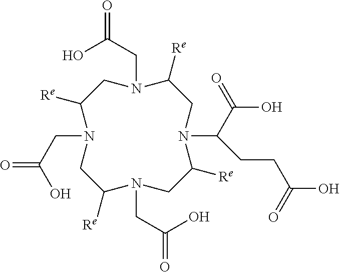

wherein each R^{e} is independently selected from hydrogen, alkyl, haloalkyl, hydroxyalkyl, aminoalkyl, heteroalkyl, alkenyl, alkynyl, cycloalkyl, heterocycloalkyl, aryl, heteroaryl, alkylcycloalkyl, alkylheterocycloalkyl, alkylaryl, alkylheteroaryl, or an amino acid side chain.

In some embodiments, the conjugate comprises DOTA. In some embodiments, the conjugate comprises a DOTA derivative such as p-SCN-Bn-DOTA and MeO-DOTA-NCS. In some embodiments, the conjugate comprises two independent metal chelators, and at least one or both are DOTA. The structures of some exemplary metal chelators are illustrated in FIGS. 1-15 (without showing the attachment points). Exemplary metal chelators are further described in WO2012/174136; US20130183235A1; US20120219495A1; Ramogidaand et al., EJNMMI radiopharm. chem. 4, 21 (2019); Thiele et al., Cancer Biotherapy and Radiopharmaceuticals 2018; Li et al., Bioconjugate Chem. 2019, 30, 5, 1539-1553; and Baranyai et al., Eur. J. Inorg. Chem. 36-56 (2020), each of which is incorporated by reference in its entirety.

A metal chelator such as DOTA can interact with a radionuclide (e.g., ¹⁷⁷Lu or ²²⁵Ac) via one or more functional groups and/or atoms. For example, a metal chelator can interact with a radionuclide via nitrogen and/or oxygen atoms. As another example, a metal chelator can interact with a radionuclide via carbonyl, carboxylic acid, amino, and/or amide groups of the metal chelator. In some embodiments, the interaction of a metal chelator and a radionuclide of the conjugates disclosed herein can be illustrated as

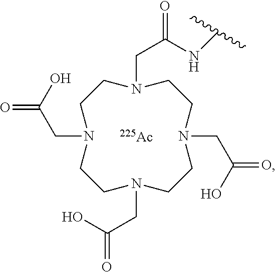

i.e., without specifying any interactions. In some embodiments, the interaction of a metal chelator and a radionuclide of the conjugates disclosed herein can be illustrated as

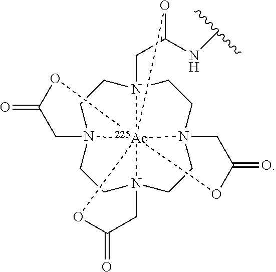

In some embodiments, the interaction of a metal chelator and a radionuclide of the conjugates disclosed herein can be illustrated as

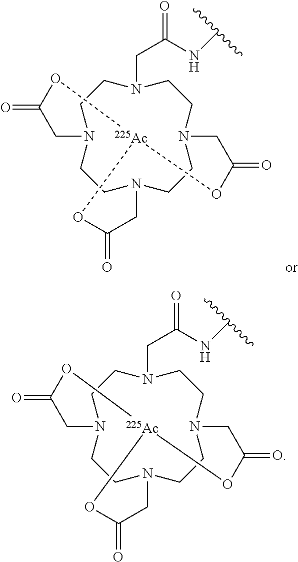

In some embodiments, the interaction of a metal chelator and a radionuclide of the conjugates disclosed herein can be illustrated as

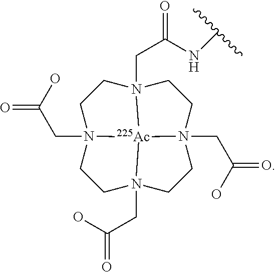

In some embodiments, the interaction of a metal chelator and a radionuclide of the conjugates disclosed herein can be illustrated as

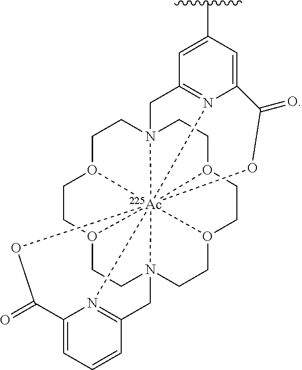

In some embodiments, the radionuclide exists in a positive oxidation state e.g., ²²⁵Ac³⁺, ¹⁷⁷L³⁺. In some embodiments, for example in certain aqueous conditions, the radionuclide exists in a salt form, e.g., as ²²⁵Ac³⁺, ¹⁷⁷Lu³⁺. In some embodiments, for example in certain acidic aqueous conditions, the radionuclide exists in a salt form, e.g., as ²²⁵Ac³⁺, ¹⁷⁷Lu³⁺. In some embodiments, the conjugate is in a salt form. In some embodiments, one or more of the carboxylic acid groups of the conjugate may exist as carboxylate anions. In some embodiments, one or more of the carboxylate anions of the conjugate may coordinate to the radionuclide. A person of ordinary skill would appreciate that the dissociation of an acid can depend on the pH value of the environment and its pK value. Accordingly, in some embodiments, a conjugate described herein can exist in a completely ionized, partially ionized or non-ionized form.

### Radionuclide

In one aspect, described herein are conjugates that comprise a radionuclide. Exemplary radionuclides include, but are not limited to, astatine-211, astatine-217, actinium-225, americium-243, francium-223, radium-223, polonium-213, lead-212, lead-203, copper-64, copper-67, copper-60, copper-61, copper-62, bismuth-212, bismuth-213, Bismuth-209, gallium-68, gallium-67, zirconium-89, cerium-134, terbium-152, dysprosium-154, gadolinium-148, gadolinium-153, samarium-146, samarium-147, samarium-153, terbium-149, thorium-227, thorium-229, iron-59, yttrium-86, rhodium-105, ytterbium-175, thulium-167, promethium-153, indium-111, holmium-166, technetium-94, technetium-99^{m}, yttrium-90, lutetium-177, terbium-161, rhenium-186, rhenium-188, cobalt-55, scandium-43, scandium-44, scandium-47, dysprosium-166, fluorine-18, or iodine-131.

Generally, the type of radionuclide used in a therapeutic radiopharmaceutical can be tailored to the specific type of cancer, the type of targeting moiety, etc. Radionuclides that undergo α-decay produce particles composed of two neutrons and two protons, and radionuclides that undergo β-decay emit energetic electrons from their nuclei. Some radionuclides can also emit Auger. In some embodiments, the conjugate comprises an alpha particle-emitting radionuclide. Alpha radiation can cause direct, irreparable double-strand DNA breaks compared with gamma and beta radiation, which can cause single-stranded breaks via indirect DNA damage. The range of these particles in tissue and the half-life of the radionuclide can also be considered in designing the radiopharmaceutical conjugate. Tables 4A and 4B below illustrate some properties of exemplary radionuclides.

TABLE 4A
Exemplary radionuclides

| | | Half-life |
|---|---|---|
| Radionuclide | Emission | (days) |
| Copper-67 | β | about 2.6 |
| Rhodium-105 | β | about 1.5 |
| Indium-111 | β | about 2.8 |
| Actinium-225 (Ac-225) | α | about 10.0 |
| Lutetium (Lu-177) | β | about 6.7 |
| Radium-223 | α | about 11.4 |
| Radium-224 | α | about 3.63 |
| Astatine-211 | α | about 0.3 |
| Yttrium-90 | β | about 2.7 |
| Gadolinium-148 | α | about 74 |
| Samarium-153 | β | about 1.9 |
| Promethium-153 | β | about 5.25 min. |
| Thulium-167 | β | about 9.3 |
| Ytterbium-175 | β | about 4.2 |
| Lead-212 | β | about 0.4 |
| Bismuth-209 | α | stable |
| Bismuth-212 | α | about 0.04 |
| Bismuth-213 | α | about 0.03 |
| Polonium-213 | α | about 4.2 μ sec. |
| Francium-223 | α | about 0.015 |
| Thorium-227 | α | about 18.7 |
| Thorium-229 | α | about 7.3 |
| Terbium-149 | α | about 0.17 |

TABLE 4B
Exemplary radionuclides

| | |
|---|---|
| Lutetium-177 (Lu-177) | about 6.6 days |
| Indium-111 (In-111) | about 2.8 days |
| Gallium-68 (Ga-68) | about 68 minutes |
| Copper-62 (Cu-62) | about 9.6 minutes |
| Copper-64 (Cu-64) | about 12.7 hours |
| Zirconium-89 (Zr-89) | about 78.4 hours |
| Technetium-99m | about 6 hours |
| Cerium-134 (Ce-134) | about 3.2 days |
| Terbium-152 | about 17.5 hours |

In some embodiments, a conjugate described herein comprises one or more independent radionuclides. In some embodiments, the conjugate comprises two radionuclides. In some embodiments, each of the one or more radionuclides is bound to a metal chelator of the conjugate. In some embodiments, two radionuclides of a conjugate are bound to the same metal chelator. In some embodiments, two radionuclides of a conjugate are bound to two independent metal chelators. In some embodiments, each of the one or more radionuclides is an alpha particle-emitting radionuclide.

In some embodiments, a conjugate described herein comprises an alpha particle-emitting radionuclide. In some embodiments, the alpha particle-emitting radionuclide is actinium-225 (²²⁵Ac), astatine-211 (²¹¹At), radium-223 (²²³Ra), radium-224 (²²⁴Ra), bismuth-213 (²¹³Bi), bismuth-209 (209Bi), terbium-149 (¹⁴⁹Tb), thorium-227 (²²⁷Th), thorium-229 (229Th) francium-223 (223Fr), galodinium-148 (148Gd), or polonium-213 (213Po). In some embodiments, the alpha particle-emitting radionuclide is ²²⁵Ac. In some embodiments, the alpha particle-emitting radionuclide is ²¹³Bi. In some embodiments, the alpha particle-emitting radionuclide is ²¹²Bi. In some embodiments, the alpha particle-emitting radionuclide is ²⁰⁹Bi In some embodiments, the alpha particle-emitting radionuclide is ²¹²Pb. In some embodiments, the alpha particle-emitting radionuclide is ²¹³Pb In some embodiments, the alpha particle-emitting radionuclide is ²¹³Po. In some embodiments, the alpha particle-emitting radionuclide is ²²⁴Ra. In some embodiments, the alpha particle-emitting radionuclide is ²²³Ra. In some embodiments, the alpha particle-emitting radionuclide is ²²³Fr In some embodiments, the alpha particle-emitting radionuclide is ²²⁷Th. In some embodiments, the alpha particle-emitting radionuclide is ²²⁹Th In some embodiments, the alpha particle-emitting radionuclide is ²¹¹At. In some embodiments, the alpha particle-emitting radionuclide is ¹⁴⁹Tb. In some embodiments, the alpha particle-emitting radionuclide is ¹⁴⁸Gd In some embodiments, the radionuclide is Zirconium-89 (⁸⁹Zr). In some embodiments, a conjugate described herein comprises a radionuclide selected from ⁶⁷Cu, ⁶⁴Cu, ⁸⁹Zr, ⁹⁰Y, ¹⁰⁹Pd, ¹¹¹Ag, ¹⁴⁹Pm, ¹⁵³Sm, ¹⁶⁶Ho, ^{99m}Tc, ⁶⁷Ga, ⁶⁸Ga, ¹¹¹In, ⁹⁰Y, ¹⁷⁷Lu, ¹⁸⁶Re, ¹⁸⁸Re, ¹⁹⁷Au, ¹⁹⁸Au, ¹⁹⁹Au, ¹⁰⁵Rh, ¹⁶⁵Ho ¹⁶¹Tb, ¹⁴⁹Pm, ⁴⁴Sc, ⁴⁷Sc, ⁷⁰As, ⁷¹As, ⁷²As, ⁷³As, ⁷⁴As, ⁷⁶As, ⁷⁷As, ²¹²Pb, ²¹²Bi, ²¹³Bi, ²²⁵Ac, ^{117m}Sn, ⁶⁷Ga, ²⁰¹Tl, ¹²³I, ¹³¹I, ¹⁶⁰Gd, ¹⁴⁸Nd, ⁸⁹Sr, and, ²¹¹At. In some embodiments, the radionuclide is, ²²⁵Ac. In some embodiments, the radionuclide is a decay daughter of ²²⁵Ac such as ²²¹Fr, ²¹⁷At, ²¹³Bi, ²¹³Po, ²⁰⁹Tl, ²⁰⁹Pb, or ²⁰⁹Bi. In some embodiments, the conjugate comprises two ²²⁵Ac radionuclides. In some embodiments, the radionuclide is ¹⁷⁷Lu. In some embodiments, the conjugate comprises two ¹⁷⁷Lu radionuclides. In some embodiments, the radionuclide is no-carrier added (i.e., non-carrier-added or n.c.a.) ¹⁷⁷Lu. In some embodiments, the radionuclide is 177Lu free of long-lived radioactive contaminants and byproducts. In some embodiments, the radionuclide is no-carrier added (i.e., non-carrier-added or n.c.a.) ²⁵⁵Ac. In some embodiments, the radionuclide is a non-carrier-added radionuclide.

In some embodiments, the alpha particle-emitting radionuclide is ²²⁵Ac. In some embodiments, the alpha particle-emitting radionuclide is no-carrier added (i.e., non-carrier-added or n.c.a.) 225Ac. In some embodiments, the ²²⁵Ac is free of long-lived radioactive contaminants and by products. In some embodiments, the alpha particle-emitting radionuclide is carrier added ²²⁵Ac. In some embodiments, ²²⁵Ac is generated from a heavier radionuclide. In some embodiments, ²²⁵Ac is generated using a Thorium-229 generator. In some embodiments, ²²⁵Ac is generated via 229Th decay to ²²⁵Ra from which ²²⁵Ra and ²²⁵Ac are then separated via ionic exchange resins. In some embodiments, ²²⁹Th is generated by legacy ²³³U stock provided by government sources. In some embodiments, ²²⁵Ac is generated using Electron Linear Accelerators (LINAC) with radium-226 as a target source. In some embodiments, ²²⁵Ac is be generated by the ²²⁶Ra(γ, n) ²²⁵Ra reaction using an electron linear accelerator, and subsequent separation and purification of ²²⁵Ra and ²²⁵Ac using a series of ionic exchange resins. In some embodiments, ²²⁵Ac is generated using a cyclical proton accelerator using radium-226 as a target source. In some embodiments, ²²⁵Ac is generated by the ²²⁶Ra(p, 2n) ²²⁵Ac reaction using a low energy cyclical proton accelerator (aka cyclotron). In some embodiments, ²²⁵Ac is generated by the ²²⁶Ra(p, 2n) ²²⁵Ac reaction using a low energy cyclical proton accelerator (aka cyclotron) having an energy range between 10-20 MeV. In some embodiments, ²²⁵Ac is generated using electron beam accelerators (rhodotron) using radium-226 as a target source. In some embodiments, ²²⁵Ac is generated by the ²²⁶Ra(γ, n) ²²⁵Ra reaction using an electron beam accelerator, and subsequent separation and purification of ²²⁵Ra and ²²⁵Ac using a series of ionic exchange resins. In some embodiments, ²²⁵Ac is generated using high energy proton spallation using thorium-232 as a target source. In some embodiments, ²²⁵Ac is generated using by the ²³²Th(p, nxp) ²²⁵Ac reaction using a 70-100 MeV+ high energy proton accelerator.

In some embodiments, the conjugate comprises an alpha particle-emitting radionuclide bound to the metal chelator. In some embodiments, the alpha particle-emitting radionuclide is actinium-225, astatine-211, thorium-227, or radium-223. In some embodiments, the alpha particle-emitting radionuclide is actinium-225.

In some embodiments, the conjugate comprises a beta particle-emitting radionuclide bound to the metal chelator. In some embodiments, the beta particle emitting radionuclide is zirconium-89, yttrium-90, iodine-131, samarium-153, lutetium-177, or lead-212.

In some embodiments, the conjugate comprises a gamma particle emitting radionuclide. In some embodiments, the gamma particle emitting radionuclide is indium-111.

In some embodiments, conjugates described herein do not contain any radionuclide, i.e., a cold conjugate. For example, in some cases, a radionuclide can be replaced with a surrogate (e.g., ²²⁵Ac replaced with lanthanum) for testing and experimental purposes.

### Linker

A conjugate described herein can comprise one or more linkers. In some embodiment, the linker or linkers attach the targeting ligand to the metal chelator. In some embodiment, the linker covalently attaches the targeting ligand with the metal chelator. The targeting ligand can be covalently linked to the metal chelator through a linker. The linker can covalently attach the targeting ligand with the metal chelator. The targeting ligand can also attach directly to the metal chelator without a linker.

The linker can have a prescribed length thereby linking the metal chelator (and optionally radionuclide) and the targeting ligand while allowing an appropriate distance therebetween. In some embodiments, the linker has 1 to 10 O atoms, 1 to 6 O atoms, 1 to 3 O atoms, 1 to 15 atoms, 1 to 1 O atoms, 1 to 5, or 2 to 2 O atoms in length. In some embodiments, the linker has 1 to 1 O atoms in length.

The linker can comprise flexible and/or rigid regions. Exemplary flexible linker regions include those comprising Gly and Ser residues (“GS” linker), glycine residues, alkylene chain, PEG chain, etc. Exemplary rigid linker regions include those comprising alpha helix-forming sequences (e.g., EAAAK (SEQ ID NO: 17)), proline-rich sequences, and regions rich in double and/or triple bonds.

The linker can be cleavable, e.g., under physiological conditions, e.g., under intracellular conditions, such that cleavage of the linker releases the chelator and radionuclide in the intracellular environment. The linker can be, e.g., a peptidyl linker that is cleaved by an intracellular peptidase or protease enzyme, including, but not limited to, a lysosomal or endosomal protease. In some embodiments, the peptidyl linker is at least two amino acids long or at least three amino acids long. Cleaving agents can include cathepsins B and D and plasmin. In other embodiments, the linker is not cleavable. In some embodiments, the linker is pH-sensitive, i.e., sensitive to hydrolysis at certain pH values. For example, the pH-sensitive linker can be hydrolyzable under acidic conditions. For example, a linker can be an acid-labile linker that is hydrolyzable in the lysosome (e.g., a hydrazone, semicarbazone, thiosemicarbazone, cis-aconitic amide, orthoester, acetal, ketal, or the like). Such linkers can be relatively stable under neutral pH conditions, such as those in the blood, but are unstable at below pH 5.5 or 5.0, the approximate pH of the lysosome. In some embodiments, the hydrolyzable linker is a thioether linker.

In some embodiments, the linker comprises one or more of substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. In some embodiments, the linker comprises substituted or unsubstituted C₁-C₃₀ alkylene. In some embodiments, the linker comprises polyethylene glycol such as (—CH₂—CH₂—O—)₁₋₁₀. In some embodiments, the linker comprises a structure selected from:

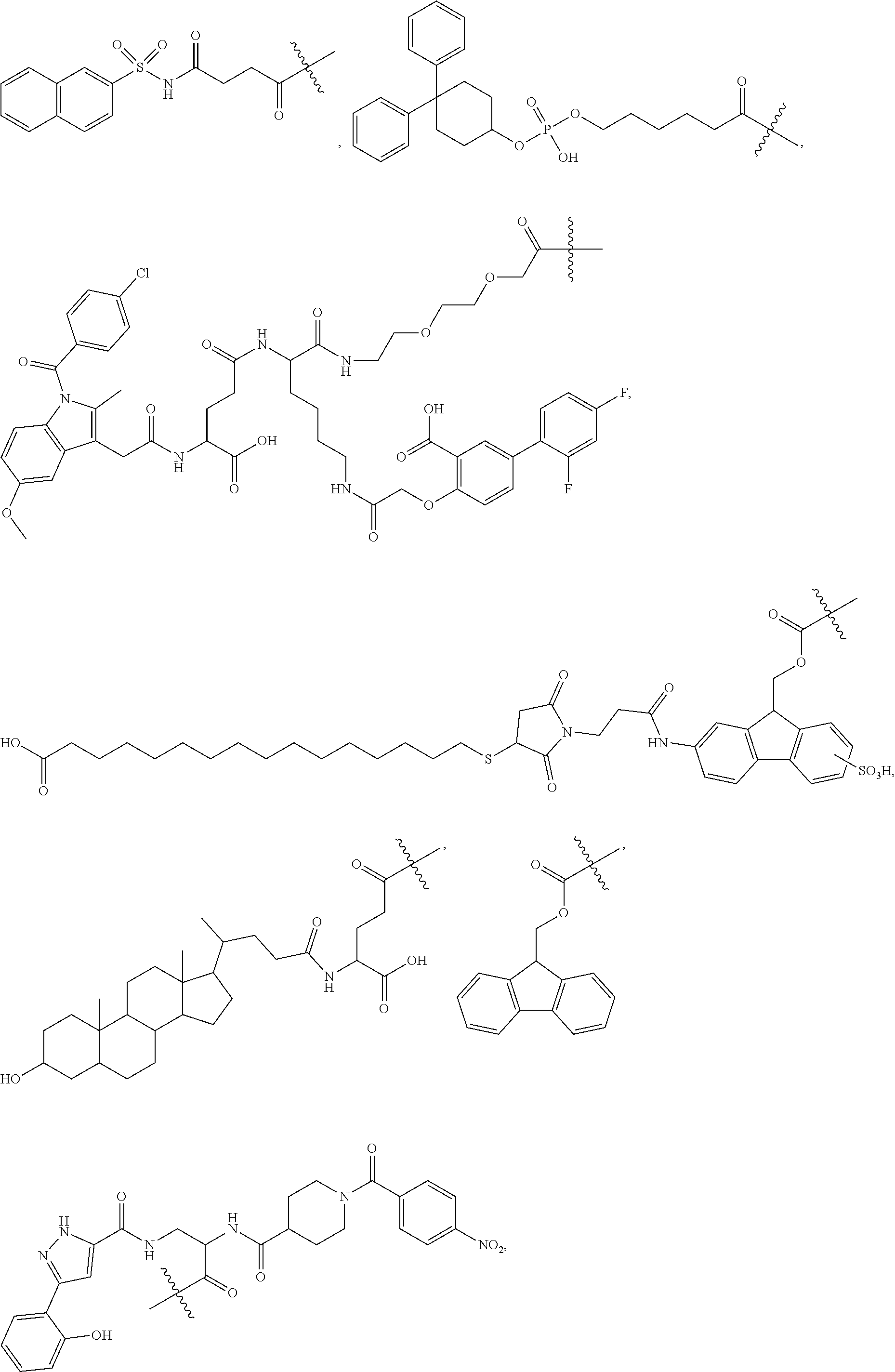

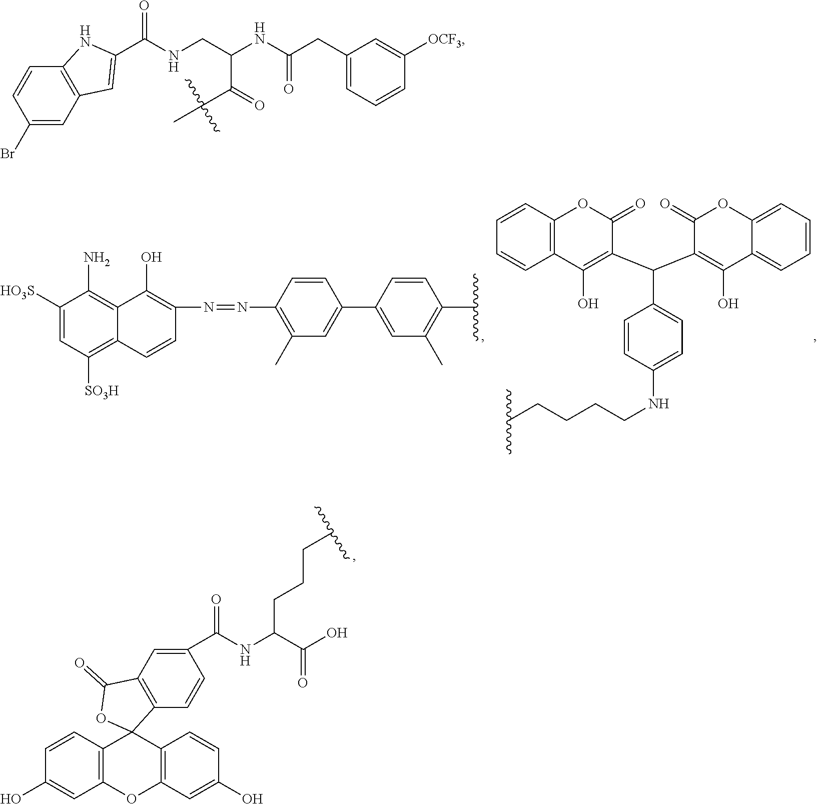 [SMILES: *C(=O)C(CNC(=O)c1cc2cc(Br)ccc2[nH]1)NC(=O)Cc1cccc(OC(F)(F)F)c1 ; *c1ccc(-c2ccc(N=Nc3ccc4c(S(=O)(=O)O)cc(S(=O)(=O)O)c(N)c4c3O)c(C)c2)cc1C ; *CCCCNc1ccc(C(c2c(O)c3ccccc3oc2=O)c2c(O)c3ccccc3oc2=O)cc1 ; *CCCC(NC(=O)c1ccc2c(c1)C(=O)OC21c2ccc(O)cc2Oc2cc(O)ccc21)C(=O)O]

and structures derived from any one thereof.

In some embodiments, the linker comprises a click chemistry residue. In some embodiments, the linker is attached to the targeting ligand, to the metal chelator, or both via click chemistry, thereby forming a click chemistry residue. For example, the targeting ligand can comprise an azide group (at N- or C-terminus or at a non-terminal amino acid) that reacts with an alkyne moiety of the linker. For another example, the targeting ligand can comprise an alkyne group that reacts with an azide of the linker. The metal chelator and the linker can be attached similarly. In some embodiments, the linker comprises an azide moiety, an alkyne moiety, or both. In some embodiments, the linker comprises a triazole. In some embodiments, the click chemistry residue is

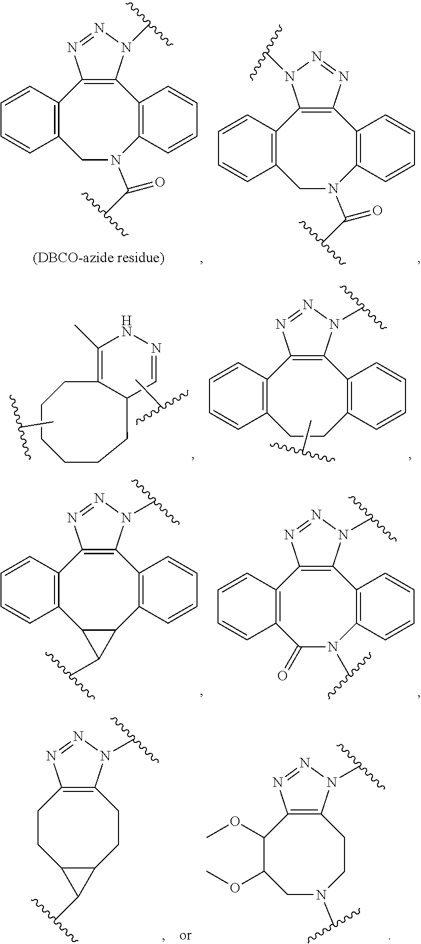

In some embodiments the click chemistry residue is a DIBO-azide residue, BARAC-azide residue, DBCO-azide residue, DIFO-azide residue, COMBO-azide residue, BCN-azide residue, or DIMAC-azide residue. In some embodiments, the linker comprises a residue of nitrone dipole cycloaddition. In some embodiments, the linker comprises a residue of tetrazine ligation. In some embodiments, the linker comprises a residue of quadricyclane ligation. Exemplary groups of click chemistry residue are shown in Hein at al., “Click Chemistry, A Powerful Tool for Pharmaceutical Sciences,” Pharmaceutical Research volume 25, pages 2216-2230 (2008); Thirumurugan et al, “Click Chemistry for Drug Development and Diverse Chemical-Biology Applications,” Chem. Rev. 2013, 113, 7, 4905-4979; US20160107999A1; U.S. Ser. No. 10/266,502B2; and US20190204330A1, each of which is incorporated by reference in its entirety.

In some embodiments, a linker described herein comprises two or more motifs. In some embodiments, one or more of the motifs are connected via click chemistry such that they can be clicked in/out of the linker. Each of the motifs in a linker can have independent functions. For example, a linker can comprise a motif that functions to adjust plasma half-life and/or a motif that functions as a spacer between the targeting ligand and metal chelator.

In some embodiments, the linker comprises at least one group selected from the group consisting of a bond, alkylene, alkenylene, alkynylene, cycloalkylene, arylene, heteroalkylene, heterocycloalkylene and heteroarylene, wherein each of the alkylene, alkenylene, alkynylene, cycloalkylene, arylene, heteroalkylene, heterocycloalkylene or heteroarylene, is optionally substituted. In some embodiments, the alkylene, alkenylene, alkynylene, cycloalkylene, arylene, heteroalkylene, heterocycloalkylene or heteroarylene are each independently substituted with one or more groups, each substituent group being independently selected from the group consisting of —O—, —S—, silicone, amino, optionally substituted alkyl (e.g., alkoxy, haloalkyl) and optionally substituted heterocycloalkylene (e.g., polyTHF). In some embodiments, the linker comprises substituted or unsubstituted C₁-C₆ alkylene or substituted or unsubstituted C₁-C₆ heteroalkylene. In some embodiments, the linker is or comprises propyl ethyl ether.

In some embodiments, the linker is or comprises at least one amino acid. In some embodiments, the linker L is or comprises two amino acids. In some embodiments, the linker L is or comprises three amino acids. The linker can comprise 1 to 3, 1 to 5, 1 to 10, 5 to 10, or 5 to 20 amino acid residues. The linker can comprise 1, 2, 3, 4, 5, 6, 7, 8, 9, or 10 amino acid residues. The linker can comprise 1 to 5 amino acid residues. For example, the linker can comprise one or more lysine (K) residues such as K, KK, or KKK sequences. The linker can comprise a lysine or a derivative thereof. The linker can comprise a lysine. The linker can comprise one or more amino acids that are unnatural amino acids.

In some embodiments, the linker comprises one or more groups selected from —O—, —S—, —NH—, —NH—(CH₂)ₚ—NH, —NH—(CH₂)ₚ—O, —O—(CH₂)ₚ—O, —(C═O)—, —(C═O)—O—, —O(C═O)—, —O(C═O)—O—, —OC(═O)—NH—, —C(═O)NH—, —NHC(═O)—, —NHC(═O)—O—, or —NHC(═O)—NH—, —(C═O)—(CH₂CH₂)_{q}—(C═O)—, —(C═O)—(CH═CH)_{q}—(C═O), —(C═O)—(OCH₂CH₂O)_{q}—(C═O)—, —(CH₂CH₂O)_{q}—, —(OCH₂CH₂)_{q}—, —(C═O)—(CH₂CH₂O)_{q}—, and —(CH(CH₃)C(═O)O)_{q}—, wherein q is 1-20 and p is 1-20. In some embodiments, the linker is or comprises a polyethylene glycol (PEG) or polypropylene glycol (PPG) linker. In some embodiments, the linker is or comprises —(CH₂CH₂O)_{q}— or —(OCH₂CH₂)_{q}—. In some embodiments, the linker comprises —O—. In some embodiments, the linker comprises substituted or unsubstituted C₁-C₆ alkylene. In some embodiments, the linker comprises substituted or unsubstituted C₁-C₆ heteroalkylene. In some embodiments, the linker is —NH—(CH₂)ₚ—. In some embodiments, the linker is —NH—(CH₂)₂—. In some embodiments, the linker is —NH—(CH₂)₃—. In some embodiments, the linker is —NH—(CH₂)₄—. In some embodiments, the linker is —NH—(CH₂)₅—. In some embodiments, the linker is —NH—(CH₂)₆—. In some embodiments, the linker is —NH—(CH₂)₇—. In some embodiments, the linker is —NH—(CH₂)₈—.

In some embodiments, q is 1, 2, 3, 4, 5, 6, 7, 8, 9, or 10. In some embodiments, p is 1, 2, 3, 4, 5, 6, 7, 8, 9, or 10.

In some embodiments, provided herein a conjugate that has a structure of Formula (X),

TL-LK¹-LK²-LK³—CHL Formula (X)
wherein,
   TL represents the targeting ligand;
   CHL represents the metal chelator, optionally bound to a radionuclide; and
   each of LK¹, LK², and LK³ is independently selected from substituted or unsubstituted C₁-C₁₂ alkylene, substituted or unsubstituted C₁-C₁₂ heteroalkylene, substituted or unsubstituted C₂-C₁₂ alkenylene, substituted or unsubstituted C₂-C₁₂ alkynylene, —(CH₂CH₂O)_{q}—, —(OCH₂CH₂)_{q}—, —O—, —S—, —S(═O)—, —S(═O)₂—, —S(═O)(═NR^{LK})—, —C(═O)—, —C(═N—OR^{LK})—, —C(═O)O—, —OC(═O)—, —C(═O)C(═O)—, —C(═O)NR^{LK}—, —NR^{LK}C(═O)—, —OC(═O)NR^{LK}—, —NR^{LK}C(═O)O—, —NR^{LK}C(═O)NR^{LK}—, —C(═O)NR^{LK}C(═O)—, —S(═O)₂NR^{LK}—, —NR^{LK}S(═O)₂—, —NR^{LK}—, —N(OR^{LK})—, and a bond;
   each R^{LK} is independently H or substituted or unsubstituted C₁-C₆ alkyl; and q is an integer selected from 1 to 10.

In some embodiments, LK¹ is substituted or unsubstituted C₁-C₁₂ alkylene. In some embodiments, LK¹ is substituted or unsubstituted C₁-C₃ alkylene. In some embodiments, LK¹ is

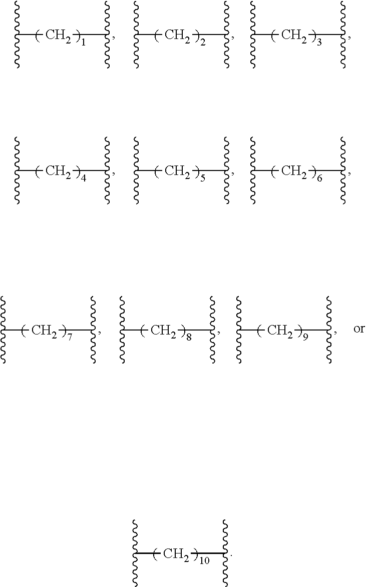

In some embodiments, LK¹ is

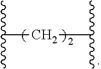

In some embodiments, LK¹ is

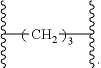

In some embodiments, LK¹ is

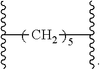

In some embodiments, LK¹ is

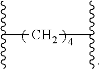

In some embodiments, LK¹ is

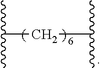

In some embodiments, LK¹ is

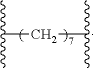

In some embodiments, LK¹ is

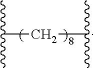

In some embodiments, LK¹ is substituted or unsubstituted C₁-C₁₂ heteroalkylene. In some embodiments, LK¹ is substituted or unsubstituted C₂-C₆ heteroalkylene. In some embodiments, LK¹ is substituted or unsubstituted C₃-C₈ heteroalkylene.

In some embodiments, LK¹ is C₁-C₆ alkylene, C₁-C₆ heteroalkylene, —(CH₂CH₂O)₁₋₆—, —(OCH₂CH₂)₁₋₆—, —O—, or —S—. In some embodiments, LK¹ is —(CH₂CH₂O)₁₋₆— or —(OCH₂CH₂)₁₋₆—. In some embodiments, LK¹ is —(CH₂CH₂O)₅—. In some embodiments, LK¹ is —(CH₂CH₂O)₄—. In some embodiments, LK¹ is —(CH₂CH₂O)₃—. In some embodiments, LK¹ is —(CH₂CH₂O)₂—. In some embodiments, LK¹ is —CH₂CH₂O—. In some embodiments, LK¹ is —(OCH₂CH₂)₅—. In some embodiments, LK¹ is —(OCH₂CH₂)₄—. In some embodiments, LK¹ is —(OCH₂CH₂)₃—. In some embodiments, LK¹ is —(OCH₂CH₂)₂—. In some embodiments, LK¹ is —OCH₂CH₂—.

In some embodiments, LK¹ is —NH—. In some embodiments, LK¹ is a bond.

In some embodiments, LK² is C₁-C₆ alkylene, C₁-C₆ heteroalkylene, —(CH₂CH₂O)₁₋₃—, —(OCH₂CH₂)₁₋₃—, —O—, or —S—. In some embodiments, LK² is —(CH₂CH₂O)₁₋₃— or —(OCH₂CH₂)₁₋₃—. In some embodiments, LK² is —(CH₂CH₂O)₂—. In some embodiments, LK² is —CH₂CH₂O—. In some embodiments, LK² is —(OCH₂CH₂)₂—. In some embodiments, LK² is —OCH₂CH₂—.

In some embodiments, LK² is substituted or unsubstituted cycloalkylene, or substituted or unsubstituted heterocycloalkylene. In some embodiments, LK² is monocyclic. In some embodiments, LK² is 3-6 membered substituted or unsubstituted heterocycloalkylene. In some embodiments, LK² is

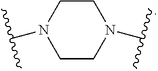 [SMILES: *N1CCN(*)CC1]

In some embodiments, LK² is O—, —S—, —S(═O)—, —S(═O)₂—, —S(═O)(═NR^{LK})—, C(O)—, —C(═N—OR^{LK})—, —C(═O)O—, —OC(═O)—, —C(═O)C(═O)—, —C(═O)NR^{LK}—, —NR^{LK}C(═O)—, —OC(═O)NR^{LK}—, —NR^{LK}C(═O)O—, —NR^{LK}C(═O)NR^{LK}—, —C(═O)NR^{LK}C(═O)—, —S(═O)₂NR^{LK}—, —NR^{LK}S(═O)₂—, or —NR^{LK}—.

In some embodiments, LK² is —O—. In some embodiments, LK² is —C(═O)NR^{LK}— or —NR^{LK}C(═O)—. In some embodiments, LK² is —C(═O)NH—. In some embodiments, LK² is —NHC(═O)—.

In some embodiments, LK² is a bond.

In some embodiments, LK³ is substituted or unsubstituted C₁-C₁₂ alkylene. In some embodiments, LK³ is substituted or unsubstituted C₁-C₃ alkylene. In some embodiments, LK³ is

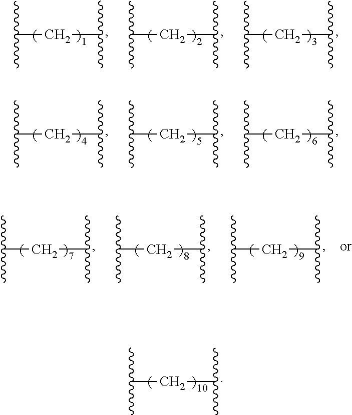

In some embodiments, LK³ is substituted or unsubstituted C₁-C₁₂ heteroalkylene. In some embodiments, LK³ is substituted or unsubstituted C₂-C₆ heteroalkylene. In some embodiments, LK³ is substituted or unsubstituted C₃-C₈ heteroalkylene.

In some embodiments, LK³ is C₁-C₆ alkylene, C₁-C₆ heteroalkylene, —(CH₂CH₂O)₁₋₃—, —(OCH₂CH₂)₁₋₃—, —O—, or —S—. In some embodiments, LK³ is substituted or unsubstituted C₁-C₁₂ heteroalkylene. In some embodiments, LK³ is substituted or unsubstituted C₂-C₆ heteroalkylene. In some embodiments, LK³ is substituted or unsubstituted C₃-C₈ heteroalkylene.

In some embodiments, LK³ is a bond. In some embodiments, LK³ is —NH—.

In some embodiments, the conjugate has a structure of TL-(CH₂)₂₋₈-s-NH—CHL. In some embodiments, the conjugate has a structure of TL-(CH₂)₂—NH-CHL. In some embodiments, the conjugate has a structure of TL-(CH₂)₃—NH-CHL. In some embodiments, the conjugate has a structure of TL-(CH₂)₄—NH-CHL. In some embodiments, the conjugate has a structure of TL-(CH₂)₅—NH-CHL. In some embodiments, the conjugate has a structure of TL-(CH₂)₆—NH-CHL. In some embodiments, the conjugate has a structure of TL-(CH₂)₇—NH-CHL. In some embodiments, the conjugate has a structure of TL-(CH₂)₈-NH-CHL.

In some embodiments, the linker has a structure of

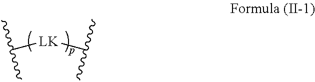

wherein each LK is independently —O—, —R^{LK}—, —N(R^{LK})₂⁺—, —OP(═O)(OR^{LK})O—, —S—, —S(═O)—, —S(═O)₂—, ═CH—, —C(═O)—, —C(═O)O—, —OC(═O)—, —OC(═O)O—, —C(═O)NR^{LK}—, —NR^{LK}C(═O)—, —OC(═O)NR^{LK}—, —NR^{LK}C(═O)O—, —NR^{LK}C(═O)NR^{LK}—, —NR^{LK}C(═S)NR^{LK}, CR^{LK}═N—, —N═CR^{LK}, —NR^{LK}S(═O)₂—, —S(═O)₂NR^{LK}—, —C(═O)NR^{LK}S(═O)₂—, —S(═O)₂NR^{LK}C(═O)—, substituted or unsubstituted C₁-C₃₀ alkylene, substituted or unsubstituted C₂-C₃₀ alkenylene, substituted or unsubstituted C₂-C₃₀ alkynylene, substituted or unsubstituted C₁-C₃₀ heteroalkylene, —(C₁-C₃₀ alkylene)-O—, —O—(C₁-C₃₀ alkylene)-, —(C₁-C₃₀ alkylene)-NR^{LK}—, —NR^{LK}—(C₁-C₃₀ alkylene)-, —(C₁-C₃₀ alkylene)-N(R^{LK})₂⁺—, —N(R^{LK})₂⁺—(C₁-C₃₀ alkylene)-, or a click chemistry residue; and
each R^{LK} is independently hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl; and
p is 1, 2, 3, 4, 5, 6, 7, 8, 9, 10, 11, 12, 13, 14, or 15.

In some embodiments, the linker has a structure of

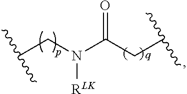

wherein each of q and p is independently 0, 1, 2, 3, 4, 5, 6, 7, 8, 9, 10, 11, 12, 13, 14, or 15. In some embodiments, the linker has a structure of

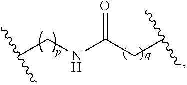

wherein each of q and p is independently 0, 1, 2, 3, 4, 5, 6, 7, 8, 9, 10, 11, 12, 13, 14, or 15. In some embodiments, the linker has a structure of

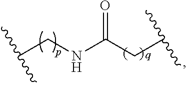

wherein each of q and p is independently 0, 1, 2, 3, 4, 5, 6, 7, 8, 9, 10, 11, 12, 13, 14, or 15, and each of methylene can be substituted or unsubstituted. In some embodiments, p is 0, 1, 2, 3, 4, or 5. In some embodiments, q is 0, 1, 2, 3, 4, or 5.

In some embodiments, the linker has a structure of

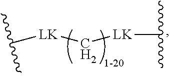

wherein each LK is independently —O—, —NR^{LK}—, —N(R^{LK})₂⁺—, —OP(═O)(OR^{LK})O—, —S—, —S(═O)—, —S(═O)₂—, —CH═CH—, ═CH—, —C≡C—, —C(═O)—, —C(═O)O—, —OC(═O)—, —OC(═O)O—, —C(═O)NR^{LK}—, —NR^{LK}C(═O)—, —OC(═O)NR^{LK}—, —NR^{LK}C(═O)O—, —NR^{LK}C(═O)NR^{LK}—, —NR^{LK}S(═O)₂—, —S(═O)₂NR^{LK}—, —C(═O)NR^{LK}S(═O)₂—, or —S(═O)₂NR^{LK}C(═O)—.

In some embodiments, the linker comprises substituted or unsubstituted C₁-C₃₀ alkylene, C₁-C₁₂ alkylene, C₁-C₈ alkylene, C₁-C₆ alkylene, or C₂-C₆ alkylene. In some embodiments, the linker comprises C₂-C₆ alkylene. In some embodiments, the linker comprises C₄-C₆ alkylene.

In some embodiments, the linker has a structure of

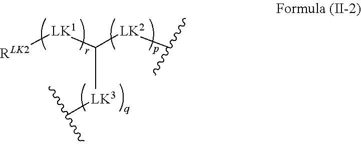

wherein each LK¹, LK², and LK³ is independently —O—, —NR^{LK}—, —N(R^{LK})₂⁺, —OP(═O)(OR^{LK})O—, —S—, —S(═O)—, —S(═O)₂—, ═CH—, —C(═O)—, —C(═O)O—, —OC(═O)—, —OC(═O)O—, —C(═O)NR^{LK}—, —NR^{LK}C(═O)—, —OC(═O)NR^{LK}—, —NR^{LK}C(═O)O—, —NR^{LK}C(═O)NR^{LK}—, —NR^{LK}C(═S)NR^{LK}—, —CR^{LK}═N—, —N═CR^{LK}, —NR^{LK}S(═O)₂—, —S(═O)₂NR^{LK}—, —C(═O)NR^{LK}S(═O)₂—, —S(═O)₂NR^{LK}C(═O)—, substituted or unsubstituted C₁-C₃₀ alkylene, substituted or unsubstituted C₂-C₃₀ alkenylene, substituted or unsubstituted C₂-C₃₀ alkynylene, substituted or unsubstituted C₁-C₃₀ heteroalkylene, substituted or unsubstituted C₁-C₁₅ arylene, —(C₁-C₃₀ alkylene)-O—, —O—(C₁-C₃₀ alkylene)-, —(C₁-C₃₀ alkylene)-NR^{LK}—, —NR^{LK}—(C₁-C₃₀ alkylene)-, —(C₁-C₃₀ alkylene)-N(R^{LK})₂⁺—, —N(R^{LK})₂⁺—(C₁-C₃₀ alkylene)-, or a click chemistry residue; and
R^{LK2} is hydrogen, azide, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted cycloalkynyl, substituted or unsubstituted C₃-C₃₀ cycloalkyl, substituted or unsubstituted C₂-C₃₀ heterocycloalkyl, substituted or unsubstituted aryl, or substituted or unsubstituted heteroaryl;
R^{LK} is hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₃₀ cycloalkyl, substituted or unsubstituted C₂-C₃₀ heterocycloalkyl, substituted or unsubstituted aryl, or substituted or unsubstituted heteroaryl;
X^{LK} is N or CR^{LK}; and
   each of r, p, and q is independently 0, 1, 2, 3, 4, 5, 6, 7, 8, 9, 10, 11, 12, 13, 14, or 15.

In some embodiments, the linker has a structure of

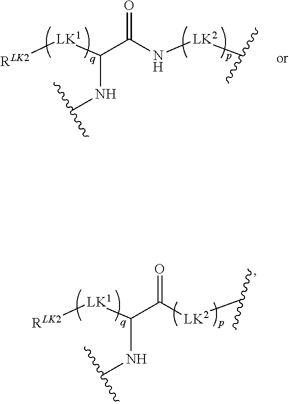

wherein
wherein each LK¹ and LK² is independently —O—, —NR^{LK}—, —N(R^{LK})₂⁺, —OP(═O)(OR^{LK})O—, —S—, —S(═O)—, —S(═O)₂—, —CH═CH—, ═CH—, —C≡C—, —C(═O)—, —C(═O)O—, —OC(═O)—, —OC(═O)O—, —C(═O)NR^{LK}—, —NR^{LK}C(═O)—, —OC(═O)NR^{LK}—, —NR^{LK}C(═O)O—, —NR^{LK}C(═O)NR^{LK}—, —NR^{LK}S(═O)₂—, —S(═O)₂NR^{LK}—, —C(═O)NR^{LK}S(═O)₂—, —S(═O)₂NR^{LK}C(═O)—, substituted or unsubstituted C₁-C₂₀ alkylene, or —(CHR^{LK}—CHR^{LK}—O)₁₋₁₀;
R^{LK} is hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, or substituted or unsubstituted C₂-C₇ heterocycloalkyl;
R^{LK2} is hydrogen, azide, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted cycloalkynyl, substituted or unsubstituted C₃-C₅ cycloalkyl, substituted or unsubstituted C₂-C₇ heterocycloalkyl, substituted or unsubstituted aryl, or substituted or unsubstituted heteroaryl;
q is 0-5; and p is 0-5.

In some embodiments, LK² is —O—, —NR^{LK}—, —N(R^{LK})₂⁺—, —OP(═O)(OR^{LK})O—, —S—, —S(═)—, —S(═O)₂—, —C(═O)—, —C(═O)O—, —OC(═O)—, —OC(═O)O—, —C(═O)NR^{LK}—, —NR^{LK}C(═O)—, —OC(═O)NR^{LK}—, —NR^{LK}C(═O)O—, —NR^{LK}C(═O)NR^{LK}—, —NR^{LK}S(═O)₂—, —S(═O)₂NR^{LK}—, —C(═O)NR^{LK}S(═O)₂—, —S(═O)₂NR^{LK}C(═O)—, substituted or unsubstituted C₁-C₆ alkylene, or —(CH₂—CH₂—O)₁₋₆—.

In some embodiments, LK¹ is —O—, —NR^{LK}—, —N(R^{LK})₂⁺—, —OP(═O)(OR^{LK})O—S—, —S(═)—, —S(═O)₂—, —CH═CH—, ═CH—, —C≡C—, —C(═O)—, —C(═O)O—, —OC(═O)—, —OC(═O)O—, —C(═O)NR^{LK}—, —NR^{LK}C(═O)—, —OC(═O)NR^{LK}—, —NR^{LK}C(═O)O—, —NR^{LK}C(═O)NR^{LK}—, —NR^{LK}S(═O)₂—, —S(═O)₂NR^{LK}—, —C(═O)NR^{LK}S(═O)₂—, —S(═O)₂NR^{LK}C(═O)—, substituted or unsubstituted C₁-C₂₀ alkylene, or —(CH₂—CH₂—O)₁₋₆—.

In some embodiments, R^{LK} is hydrogen or substituted or unsubstituted C₁-C₄ alkyl.

In some embodiments, R^{LK2} is hydrogen, substituted or unsubstituted C₁-C₄ alkyl, substituted or unsubstituted C₃-C₃₀ cycloalkyl, substituted or unsubstituted C₂-C₃₀ heterocycloalkyl, substituted or unsubstituted aryl, or substituted or unsubstituted heteroaryl.

In some embodiments, p is 1, 2, 3, 4, or 5. In some embodiments, q is 1, 2, 3, 4, or 5.

In some embodiments, R^{LK2} is hydrogen, substituted or unsubstituted C₆-C₁₀ aryl, substituted or unsubstituted C₅-C₉ heteroaryl, or a sterol.

In some embodiments, at least one L^{K} is unsubstituted C₃-C₂₀ alkylene.

In some embodiments, the linker comprises one or more of a substituted or unsubstituted C₆-C₁₀ aryl, substituted or unsubstituted C₅-C₉heteroaryl, a sterol, sulfonamide, phosphate ester, polyethylene glycol, or C₃-C₂₀ alkylene, or amino acid residues.

In some embodiments, the linker is configured to reversibly bind to a plasma protein such as albumin. In some embodiments, a dissociation constant (Kd) between the linker and human serum albumin is at most 15 μM, as determined at room temperature in human serum condition. In some embodiments, the Kd is from about 0.1 nM to about 10 μM. In some embodiments, the Kd is from about 10 nM to about 10 μM. In some embodiments, the Kd is from about 50 nM to about 1 μM. In some embodiments, the Kd is from about 100 nM to about 10 μM.

### Isomers Stereoisomers

In some embodiments, the compounds described herein exist as geometric isomers. In some embodiments, the compounds described herein possess one or more double bonds. The compounds presented herein include cis, trans, syn, anti, entgegen (E), and zusammen (Z) isomers as well as the corresponding mixtures thereof. In some situations, the compounds described herein possess one or more chiral centers and each center exists in the R configuration or S configuration. The compounds described herein include diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers, and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent.

### Tautomers

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

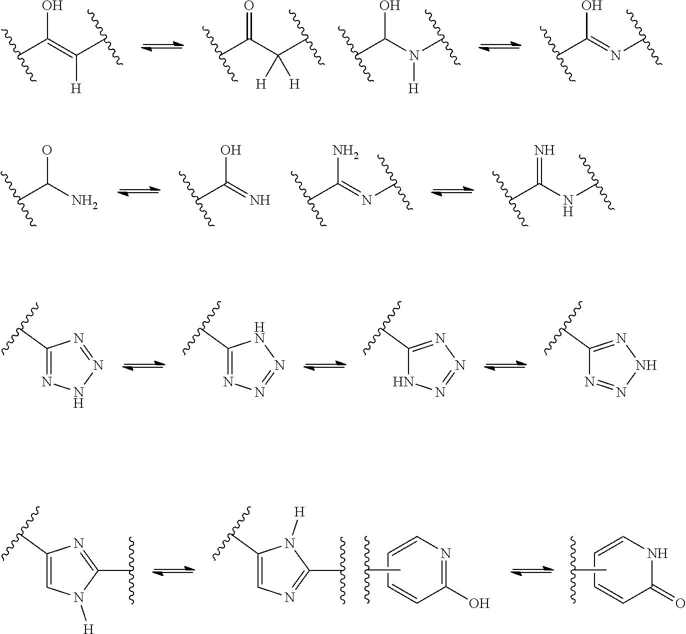

In some instances, the compounds disclosed herein exist in tautomeric forms. The structures of said compounds are illustrated in the one tautomeric form for clarity. The alternative tautomeric forms are expressly included in this disclosure.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds described herein, or a solvate, or stereoisomer thereof, include isotopes of hydrogen, carbon, nitrogen, oxygen, phosphorous, sulfur, fluorine, and chloride, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and the pharmaceutically acceptable salts, solvates, or stereoisomers thereof which contain the aforementioned isotopes and/or other isotopes of other atoms are within the scope of this disclosure. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are notable for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compound or a pharmaceutically acceptable salt, solvate, or stereoisomer thereof is prepared by any suitable method.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions. As used herein, a “pharmaceutically acceptable salt” refers to any salt of a compound that is useful for therapeutic purposes of a subject.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein, or by separately reacting a purified compound in its free form with a suitable acid or base, and isolating the salt thus formed.

Examples of pharmaceutically acceptable salts include those salts prepared by reaction of the compounds described herein with a mineral acid, organic acid, or inorganic base, such salts including acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, y-hydroxybutyrate, hydrochloride, hydrobromide, hydroiodide, 2-hydroxyethanesulfonate, iodide, isobutyrate, lactate, maleate, malonate, methanesulfonate, mandelate, metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undeconate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid, metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

In some embodiments, those compounds described herein which comprise a free acid group react with a suitable base, such as the hydroxide, carbonate, bicarbonate, or sulfate of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts, and the like. Illustrative examples of bases include sodium hydroxide, potassium hydroxide, choline hydroxide, sodium carbonate, N(C₁₋₄ alkyl)₄, and the like.

Representative organic amines useful for the formation of base addition salts include ethylamine, diethylamine, ethylenediamine, ethanolamine, diethanolamine, piperazine, and the like. It should be understood that the compounds described herein also include the quaternization of any basic nitrogen-containing groups they contain. In some embodiments, water or oil-soluble or dispersible products are obtained by such quaternization.

### Solvates.

In some embodiments, the compounds described herein exist as solvates. This disclosure provides for methods of treating diseases by administering such solvates. This disclosure further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein. Accordingly, one aspect of the present disclosure pertains to hydrates and solvates of compounds of the present disclosure and/or their pharmaceutical acceptable salts, as described herein, that can be isolated and characterized by methods known in the art, such as, thermogravimetric analysis (TGA), TGA-mass spectroscopy, TGA-Infrared spectroscopy, powder X-ray diffraction (PXRD), Karl Fisher titration, high resolution X-ray diffraction, and the like.

### Preparation of the Compounds.

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH, Inc. (Toronto, Canada), Bionet (Cornwall, U.K.), Chem Service Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Comwall, U.K.), Lancaster Synthesis (Windham, NH), Maybridge Chemical Co. Ltd. (Comwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatises that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modem Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatises that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modem Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as on-line. Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

### III. Pharmaceutical Compositions

The radiopharmaceutical conjugate described herein, including e.g., pharmaceutically acceptable salt or solvate thereof, can be administered per se as a pure chemical or as a component of a pharmaceutically acceptable formulation. In some embodiments, a conjugate described herein is combined with a pharmaceutically suitable or acceptable carrier selected on the basis of a chosen route of administration and standard pharmaceutical practice as described, for example, in Remington: The Science and Practice of Pharmacy (Gennaro, 21^{st} Ed. Mack Pub. Co., Easton, PA (2005)). Provided herein is a pharmaceutical composition comprising at least one conjugate described herein, or a stereoisomer, pharmaceutically acceptable salt, amide, ester, solvate, or N-oxide thereof, together with one or more pharmaceutically acceptable carriers. The carrier(s) (or excipient(s)) is acceptable or suitable if the carrier is compatible with the other ingredients of the composition and not deleterious to the recipient (i.e., the subject or patient) of the composition.

In one aspect, the disclosure provides a pharmaceutical composition comprising a conjugate of the present disclosure, or a pharmaceutically acceptable salt or solvate thereof, and a pharmaceutically acceptable excipient or carrier. In certain embodiments, the conjugate as described is substantially pure, in that it contains less than about 10%, less than about 5%, or less than about 1%, or less than about 0.1%, of other organic small molecules, such as unreacted intermediates or synthesis by-products that are created, for example, in one or more of the steps of a synthesis method.

Pharmaceutical compositions can include pharmaceutically acceptable carriers, diluents or excipients. Exemplary pharmaceutically acceptable carriers include solvents (aqueous or non-aqueous), solutions, emulsions, dispersion media, coatings, isotonic and absorption promoting or delaying agents, compatible with pharmaceutical administration. Such formulations can be contained in a liquid; emulsion, suspension, syrup or elixir, or solid form; tablet (coated or uncoated), capsule (hard or soft), powder, granule, crystal, or microbead. Supplementary components (e.g., preservatives, antibacterial, antiviral and antifungal agents) can also be incorporated into the compositions. Pharmaceutical compositions can be formulated to be compatible with a particular local or systemic route of administration. Thus, pharmaceutical compositions include carriers, diluents, or excipients suitable for administration by particular routes.

The compounds and pharmaceutical compositions of the current disclosure can be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. The term parenteral as used herein includes e.g., subcutaneous, intravenous, intramuscular, intrasternal, intraperitoneal, and infusion techniques. The term parenteral also includes injections, into the eye or ocular, intravitreal, intrabuccal, transdermal, intranasal, into the brain, including intracranial and intradural, into the joints, including ankles, knees, hips, shoulders, elbows, wrists, and the like, and in suppository form. In certain embodiments, the compounds and/or formulations are administered orally. In certain embodiments, the compounds and/or formulations are administered by systemic administration. In certain embodiments, the compounds and/or formulations are administered parenterally. In certain embodiments, the compounds and/or formulations are administered locally at a targeted site.

In some embodiments, conjugates, or pharmaceutically acceptable salts or solvates thereof, and pharmaceutical compositions described herein are administered via parenteral injection as liquid solution, which can include other chemical components, such as carriers, stabilizers, diluents, dispersing agents, suspending agents, thickening agents, preservatives, or excipients. Parenteral injections can be formulated for bolus injection or continuous infusion. The pharmaceutical compositions can be in a form suitable for parenteral injection as a sterile suspension, solution or emulsion in oily or aqueous vehicles, and can contain formulatory agents such as suspending, stabilizing or dispersing agents. Pharmaceutical formulations for parenteral administration include aqueous solutions of the active compounds in water soluble form. Solutions or suspensions used for parenteral, intradermal, or subcutaneous application can include: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid, gentisic acid, or sodium bisulfite; chelating agents such as ethylenediaminetetraacetic acid; buffers such as acetates, citrates or phosphates; surfactants such as polysorbate 80; and agents for the adjustment of tonicity such as sodium chloride or dextrose. pH can be adjusted with acids or bases, such as hydrochloric acid or sodium hydroxide. In some embodiments, the pharmaceutical composition comprises a reductant. The presence of a reductant can help minimize potential radiolysis. In some embodiments, the reductant is ascorbic acid, gentisic acid, sodium thiosulfate, citric acid, tartaric acid, or a combination thereof.

In some embodiments, conjugates, or pharmaceutically acceptable salts or solvates thereof, and pharmaceutical compositions described herein are administered via intravenous administration. In some embodiments, the pharmaceutical composition is formulated for intravenous administration.

Pharmaceutical compositions comprising the conjugates or pharmaceutically acceptable salts or solvates thereof described herein can be prepared according to standard techniques and further comprise a pharmaceutically acceptable carrier. In some embodiments, normal saline can be employed as the pharmaceutically acceptable carrier. Other suitable carriers include, e.g., water, buffered water, 0.9% isotonic saline, 0.4% saline, 0.3% glycine, and the like, including glycoproteins for enhanced stability, such as albumin, lipoprotein, globulin, etc. These compositions can be sterilized by conventional sterilization techniques. The resulting aqueous solutions may be packaged for use or filtered under aseptic conditions and lyophilized. In some embodiments, the lyophilized preparation is combined with a sterile aqueous solution prior to administration. The compositions can contain pharmaceutically acceptable auxiliary substances as appropriate to approximate physiological conditions, such as pH adjusting and buffering agents, tonicity adjusting agents and the like, for example, sodium acetate, sodium lactate, sodium chloride, potassium chloride, calcium chloride, sodium lactate, sorbitan monolaurate, triethanolamine oleate, etc. Pharmaceutical compositions can be selected according to their physical characteristic, including, but not limited to fluid volumes, viscosities and other parameters in accordance with the particular mode of administration selected. The amount of conjugates administered can depend upon the particular targeting moiety used, the disease state being treated, the therapeutic agent being delivered, and the judgment of the clinician.

The concentration of the conjugates or pharmaceutically acceptable salts or solvates thereof described herein in the pharmaceutical formulations can vary. In some embodiments, the conjugate is present in the pharmaceutical composition from about 0.05% to about 1% by weight, about 1% to about 2% by weight, about 2% to about 5% by weight, about 5% to about 10% by weight, about 10% to about 30% by weight, about 30% to about 50% by weight, about 50% to about 75% by weight, or about 75% to about 99% by weight.

Pharmaceutical compositions are administered in a manner appropriate to the disease to be treated. An appropriate dose and a suitable duration and frequency of administration will be determined by such factors as the condition of the subject, the type and severity of the subject's disease, the particular form of the active ingredient, and the method of administration. In some embodiments, an appropriate dose and treatment regimen provides the composition(s) in an amount sufficient to provide therapeutic and/or prophylactic benefit (e.g., an improved clinical outcome), or a lessening of symptom severity. Optimal doses are generally determined using experimental models and/or clinical trials. The optimal dose depends upon the body mass, weight, or blood volume of the subject.

The amount of conjugates or pharmaceutically acceptable salts or solvates thereof and/or pharmaceutical compositions administered can be sufficient to deliver a therapeutically effective dose of the particular subject. In some embodiments, conjugate dosages can be between about 0.1 pg and about 50 mg per kilogram of body weight, 1 μg and about 50 mg per kilogram of body weight, or between about 0.1 and about 10 mg/kg of body weight. Therapeutically effective dosages can also be determined at the discretion of a physician. By way of example only, the dose of the conjugate or a pharmaceutically acceptable salt or solvate thereof described herein for methods of treating a disease as described herein is about 0.001 mg/kg to about 1 mg/kg body weight of the subject per dose. In some embodiments, the dose of conjugate or a pharmaceutically acceptable salt or solvate thereof described herein for the described methods is about 0.001 mg to about 1000 mg per dose for the subject being treated. In some embodiments, a conjugate or a pharmaceutically acceptable salt or solvate thereof described herein is administered to a subject at a dosage of from about 0.01 mg to about 500 mg, from about 0.01 mg to about 100 mg, or from about 0.01 mg to about 50 mg. In some embodiments, a conjugate or a pharmaceutically acceptable salt or solvate thereof described herein is administered to a subject at a dosage of about 0.01 picomole to about 1 mole, about 0.1 picomole to about 0.1 mole, about 1 nanomole to about 0.1 mole, or about 0.01 micromole to about 0.1 millimole. In some embodiments, a conjugate or a pharmaceutically acceptable salt or solvate thereof described herein is administered to a subject at a dosage of about 0.0001 Gbq to about 1000 Gbq, 0.01 Gbq to about 1000 Gbq, about 0.5 Gbq to about 100 Gbq, or about 1 Gbq to about 50 Gbq. In some embodiments, a conjugate or a pharmaceutically acceptable salt or solvate thereof described herein is administered to a subject at a dosage of about 5kBq/kg to about 50,000kBq/kg body weight per dose. In some embodiments, a conjugate or a pharmaceutically acceptable salt or solvate thereof described herein is administered to a subject at a dosage of about 1kBq/kg to about 0.2GBq/kg body weight per dose. In some embodiments, a conjugate or a pharmaceutically acceptable salt or solvate thereof described herein is administered to a subject at a dosage of about 20 k Bq/kg to about 5,000kBq/kg body weight per dose. In some embodiments, a conjugate or a pharmaceutically acceptable salt or solvate thereof described herein is administered to a subject at a dosage of about 50 k Bq/kg to about 500 kBq/kg body weight per dose. In some embodiments, the dose is administered once a day, 1 to 3 times a week, 1 to 4 times a month, or 1 to 12 times a year.

The pharmaceutical formulations can be packaged in unit dosage form for ease of administration and uniformity of dosage. A unit dosage form can refer to physically discrete units suited as unitary dosages for the subject to be treated; each unit containing a predetermined quantity of active compound calculated to produce the desired therapeutic effect in association with the pharmaceutical carrier or excipient.

### IV. Method of Treatment

In one aspect, the disclosure provides methods of treating a disease or condition in a subject in need thereof. In some embodiments, the methods comprise administering a conjugate or a pharmaceutically acceptable salt or solvate thereof described herein, or a pharmaceutical composition comprising the same to the subject in need thereof. In some embodiments, provided herein is a method of providing a therapeutic and/or prophylactic benefit to a subject in need thereof comprising administering a compound or pharmaceutical composition described herein.

The methods can comprise administering to a subject a radiopharmaceutical composition that comprise a therapeutically effective amount of a conjugate or a pharmaceutically acceptable salt or solvate thereof. In some embodiments, the subject has cancer.

In some embodiments, the methods comprise administering to a subject a therapeutically effective amount of a conjugate or a pharmaceutically acceptable salt or solvate thereof. In some embodiments, the conjugate or pharmaceutically acceptable salt or solvate thereof is administered in a pharmaceutical composition. In some embodiments, the subject has cancer. In some embodiments, the cancer is a solid tumor or hematological cancer.

In one aspect, provided herein are methods for killing a cell comprising contacting the cell with a conjugate (or a pharmaceutically acceptable salt or solvate thereof) or a pharmaceutical composition comprising the same. In one aspect, provided herein are methods for delivering a radionuclide to a cell comprising administering a conjugate (or a pharmaceutically acceptable salt or solvate thereof) or a pharmaceutical composition comprising the same. After contacting a cell, the described conjugate can permeate into the cell. In some embodiments, the conjugate or pharmaceutically acceptable salt or solvate thereof binds to intracellular protein in a cell. In some embodiments, the cell comprises a mutated protein. In some embodiments, the cell comprises an overexpressed protein.

In one aspect, provided herein are methods for killing a cell harboring a mutated or overexpressed protein comprising contacting the cell with a conjugate (or a pharmaceutically acceptable salt or solvate thereof) or a pharmaceutical composition comprising the same, thereby delivering a dose of radiation to the cell. In some embodiments, the conjugate or pharmaceutically acceptable salt or solvate thereof releases a number of alpha particles by natural radioactive decay. In some embodiments, the conjugate or pharmaceutically acceptable salt or solvate thereof releases a number of beta particles, gamma rays, and/or Auger electrons by natural radioactive decay. The conjugate described herein can kill a cell by radiation. In some embodiments, the conjugate kills the cell directly by radiation. In some embodiments, the radiation creates, in the cell, oxidized bases, abasic sites, single-stranded breaks, double-stranded breaks, DNA crosslink, chromosomal rearrangement, or a combination thereof. In some embodiments, the conjugate kills the cell by inducing double-stranded DNA breaks. In some embodiments, the released alpha particles are sufficient to kill the cell. In some embodiments, the released alpha particles are sufficient to stop cell growth. In some embodiments, the conjugate kills the cell indirectly via the production of reactive oxygen species (ROS) such as free hydroxyl radicals. In some embodiments, the conjugate kills the cell indirectly by releasing tumor antigens from one or more different cells, which can have vaccine effect. In some embodiments, the conjugate kills the cell by abscopal effect. In some embodiments, the cell is a cancer cell. In some embodiments, the method comprises killing a cell with an alpha-particle emitting radionuclide. In one aspect, provided herein are methods for diagnosing cancer patients harboring a mutated or overexpressed protein comprising administering to a patient a conjugate described herein (or a pharmaceutically acceptable salt or solvate thereof) or a pharmaceutical composition comprising the same. In one aspect, provided herein are methods for imaging a cancer harboring a mutated protein comprising administering to a patient a conjugate described herein (or a pharmaceutically acceptable salt or solvate thereof) or a pharmaceutical composition comprising the same. In one aspect, provided herein are methods for imaging a cancer harboring an overexpressed protein comprising administering to a patient a conjugate described herein (or a pharmaceutically acceptable salt or solvate thereof) or a pharmaceutical composition comprising the same. In some embodiments, the method further comprises selecting or confirming that a tumor in the patient has a mutated protein. In some embodiments, the method further comprises selecting or confirming that a tumor in the patient has an overexpressed protein. In some embodiments, the method further comprises measuring the concentration of the conjugate accumulated in the patient. In some embodiments, the method further comprises measuring the amount of radiation emitted from the radionuclide. In some embodiments, the method further comprises analyzing the elimination or clearance profile of the conjugate in the patient. In some embodiments, the method further comprises measuring an elimination half-life of the conjugate in the patient. In some embodiments, the method further comprises analyzing the clearance profile of the conjugate in the patient. In some embodiments, the method of imaging or diagnosing cancer comprises administering a conjugate that comprises a radionuclide of Table 4B, such as ⁶⁸Ga. For example, conjugates of the present disclosure can be administered for patient selection purposes, such as to confirm the tumor has the appropriate expression of the intracellular target. As another example, conjugates of the present disclosure can be administered to a patient so that the patient's care team can make sure the conjugate is cleared from the body in a suitable timeframe so that undesired irradiation of other tissues is minimized.

In some embodiments, a method described herein comprises administering to a patient two conjugates of the present disclosure. In some embodiments, the two conjugates can have the same targeting ligand and/or linker. In some embodiments, a method described herein comprises administering (i) a conjugate of the present disclosure that comprises a radionuclide of Table 4B, and followed by (i) a conjugate of the present disclosure that comprises a radionuclide of Table 4A. In addition to the methods of treatment described above, the radiopharmaceutical compositions described herein can be used to image, and/or as part of a treatment for diseases. Conjugates for imaging applications, e.g., single-photon emission computed tomography (SPECT) and positron emission tomography (PET), can comprise a radionuclide suitable for use as imaging isotopes such as the isotopes in Table 4B. Accordingly, the conjugate can be administered as a companion diagnostic.

In one aspect, the disclosed conjugate or a pharmaceutically acceptable salt or solvate thereof is configured to treat cancer by ablating tumor cells. In some embodiments, the conjugate or a pharmaceutically acceptable salt or solvate thereof does not modulate the biology of the tumor cell and/or the surrounding stroma. In some embodiments, the conjugate or a pharmaceutically acceptable salt or solvate thereof does not modulate immune cells. In some embodiments, the ablating of tumor cells can lead to a downstream immunological cascade.

Non-limiting examples of cancers to be treated by the methods of the present disclosure can include melanoma (e.g., metastatic malignant melanoma), renal cancer (e.g., clear cell carcinoma), prostate cancer (e.g., hormone refractory prostate adenocarcinoma), pancreatic adenocarcinoma, breast cancer, colon cancer, lung cancer (e.g., non-small cell lung cancer), esophageal cancer, squamous cell carcinoma of the head and neck, liver cancer, ovarian cancer, cervical cancer, thyroid cancer, glioblastoma, glioma, leukemia, lymphoma, and other neoplastic malignancies. In some embodiments, a subject or population of subjects to be treated with a pharmaceutical composition of the present disclosure have a solid tumor. In some embodiments, a solid tumor is a melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, or Merkel cell carcinoma. In some embodiments, a subject or population of subjects to be treated with a pharmaceutical composition of the present disclosure have a hematological cancer. In some embodiments, the subject has a hematological cancer such as Diffuse large B cell lymphoma (“DLBCL”), Hodgkin's lymphoma (“HL”), Non-Hodgkin's lymphoma (“NHL”), Follicular lymphoma (“FL”), acute myeloid leukemia (“AML”), or Multiple myeloma (“MM”). In some embodiments, a subject or population of subjects to be treated having the cancer selected from the group consisting of ovarian cancer, lung cancer and melanoma.

In some embodiments, provided herein are methods and compositions for treating a disease or condition. Exemplary disease or condition includes refractory or recurrent malignancies whose growth may be inhibited using the methods of treatment of the present disclosure. In some embodiments, the disease or condition is a cancer. In some embodiments, the cancer is breast cancer, head and neck squamous cell carcinoma, non-small cell lung cancer, hepatocellular cancer, colorectal cancer, gastric adenocarcinoma, melanoma, or advanced cancer. In some embodiments, a cancer to be treated by the methods of treatment of the present disclosure is selected from the group consisting of carcinoma, squamous carcinoma, adenocarcinoma, sarcomata, endometrial cancer, breast cancer, ovarian cancer, cervical cancer, fallopian tube cancer, primary peritoneal cancer, colon cancer, colorectal cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, glioblastoma, glioma, squamous cell carcinoma of the head and neck, prostate cancer, pancreatic cancer, mesothelioma, sarcoma, hematological cancer, leukemia, lymphoma, neuroma, and combinations thereof. In some embodiments, a cancer to be treated by the methods of the present disclosure include, for example, carcinoma, squamous carcinoma (for example, cervical canal, eyelid, tunica conjunctiva, vagina, lung, oral cavity, skin, urinary bladder, tongue, larynx, and gullet), and adenocarcinoma (for example, prostate, small intestine, endometrium, cervical canal, large intestine, lung, pancreas, gullet, rectum, uterus, stomach, mammary gland, and ovary). In some embodiments, a cancer to be treated by the methods of the present disclosure further include sarcomata (for example, myogenic sarcoma), leukosis, neuroma, melanoma, and lymphoma. In some embodiments, a cancer to be treated by the methods of the present disclosure is breast cancer. In some embodiments, a cancer to be treated by the methods of treatment of the present disclosure is triple negative breast cancer (TNBC). In some embodiments, a cancer to be treated by the methods of treatment of the present disclosure is pancreatic cancer.

In some embodiments, the subject is 4 to 100 years old. In some embodiments, the subject is 5 to 10, 5 to 15, 5 to 18, 5 to 25, 5 to 35, 5 to 45, 5 to 55, 5 to 65, 5 to 75, 10 to 15, 10 to 18, 10 to 25, 10 to 35, 10 to 45, 10 to 55, 10 to 65, 10 to 75, 15 to 18, 15 to 25, 15 to 35, 15 to 45, 15 to 55, 15 to 65, 15 to 75, 18 to 25, 18 to 35, 18 to 45, 18 to 55, 18 to 65, 18 to 75, 25 to 35, 25 to 45, 25 to 55, 25 to 65, 25 to 75, 35 to 45, 35 to 55, 35 to 65, 35 to 75, 45 to 55, 45 to 65, 45 to 75, 55 to 65, 55 to 75, or 65 to 75 years old. In some embodiments, the subject is at least 5, 10, 15, 18, 25, 35, 45, 55, or 65 years old. In some embodiments, the subject is at most 10, 15, 18, 25, 35, 45, 55, 65, or 75 years old.

In addition to the methods of treatment described above, the compounds and compositions described herein can be used to image, and/or as part of a treatment for diseases. Conjugates for imaging applications, e.g., single-photon emission computed tomography (SPECT) and positron emission tomography (PET), can comprise a radionuclide suitable for use as imaging isotopes. Accordingly, the conjugate can be administered as a companion diagnostic.

### Combination Therapy

In some embodiments, a conjugate described herein can be administered alone or in combination with one or more additional therapeutic agents. For example, the combination therapy can include a composition comprising a conjugate described herein co-formulated with, and/or co-administered with, one or more additional therapeutic agents, e.g., one or more anti-cancer agents, e.g., cytotoxic or cytostatic agents, immune checkpoint inhibitors, hormone treatment, vaccines, and/or immunotherapies. In some embodiments, the conjugate is administered in combination with other therapeutic treatment modalities, including surgery, cryosurgery, and/or chemotherapy. Such combination therapies may advantageously utilize lower dosages of the administered therapeutic agents, thus avoiding possible toxicities or complications associated with the various monotherapies.

When administered in combination, two (or more) different treatments can be delivered to the subject during the course of the subject's affliction with the disorder, e.g., the two or more treatments are delivered after the subject has been diagnosed with the disorder and before the disorder has been cured or eliminated. In some embodiments, the delivery of one treatment is still occurring when the delivery of the second begins, so that there is overlap. This is sometimes referred to herein as “simultaneous” or “concurrent delivery.” In some embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In some embodiments, the herein-described conjugate is used in combination with a chemotherapeutic agent, e.g., a DNA damaging chemotherapeutic agent. Non-limiting examples of DNA damaging chemotherapeutic agents include topoisomerase I inhibitors, topoisomerase II inhibitors; alkylating agents; DNA intercalators; DNA intercalators and free radical generators such as bleomycin; and nucleoside mimetics. In some embodiments, the herein-described conjugate is used in combination with a radiation sensitizer, which makes tumor cells more sensitive to radiation therapy. In some embodiments, the herein-described conjugate is used in combination with a DNA damage repair inhibitor (or DNA damage response (DDR) inhibitor).

### EXAMPLES

### A: Synthesis of the Compounds

¹H NMR spectra were recorded on either a Brucker Avance III 400 (400 MHz), or Bruker Avance 300 (400 MHz) spectrometer. Chemical shifts are reported in ppm with solvent resonance as the internal standard (CDCl₃: 7.27 ppm, DMSO-d₆: 2.50 ppm, CD₃OD: 3.31 ppm). Data are reported as follows: chemical shift, integration, multiplicity (s=singlet, d=doublet, t=triplet, q=quartet, p=pentet, m=multiplet), and coupling constants (Hz).

Liquid chromatography was performed using forced flow (flash chromatography) on silica gel (SiO₂, 1000 mesh) or by column chromatography (silica gel, 1000 mesh). Thin layer chromatography (TLC) was performed on a 20-25 pm silica gel glass backed plates. Preparative TLC was performed on a 40-45 pm silica gel glass backed plates. Visualization was performed using ultraviolet light (254 nm), iodide, or KMnO4 in water.

Reaction solvents Tetrahydrofuran (THF), dichloromethane (DCM), toluene, N, N-dimethylformamide (DMF), methanol (MeOH) and 1,4-dioxane were supplied by WuXi EHS department which were purified with Pure-Solv MD-6 solvent purification system (Innovation technology Limited), by passing the solvents through 4A molecular sieve column. All reaction reagents were purchased from Alfa Aesar, Aldrich or domestic vendors which were used without further purification.

### Example A1. Conjugate Synthesis

Targeting ligands of Table 1 and metal chelators of FIGS. 1-15 can be synthesized according to methods known in the art. Modifications and derivatization of the targeting ligand and metal chelator can be made at suitable positions to allow the attachment of a linker that connects the targeting ligand and metal chelator. The resulting conjugate can then be labeled with a radionuclide.

### Example A2. Synthesis of Conjugates Compound 001 and Compound 001-Lu

Conjugates Compound 001 and Compound 001-Lu can be synthesized according to the following schemes.

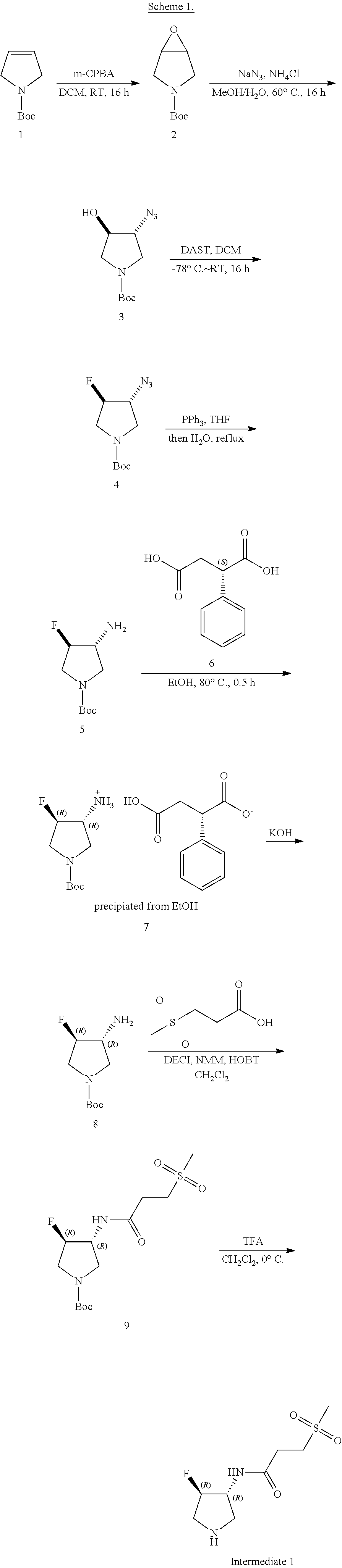

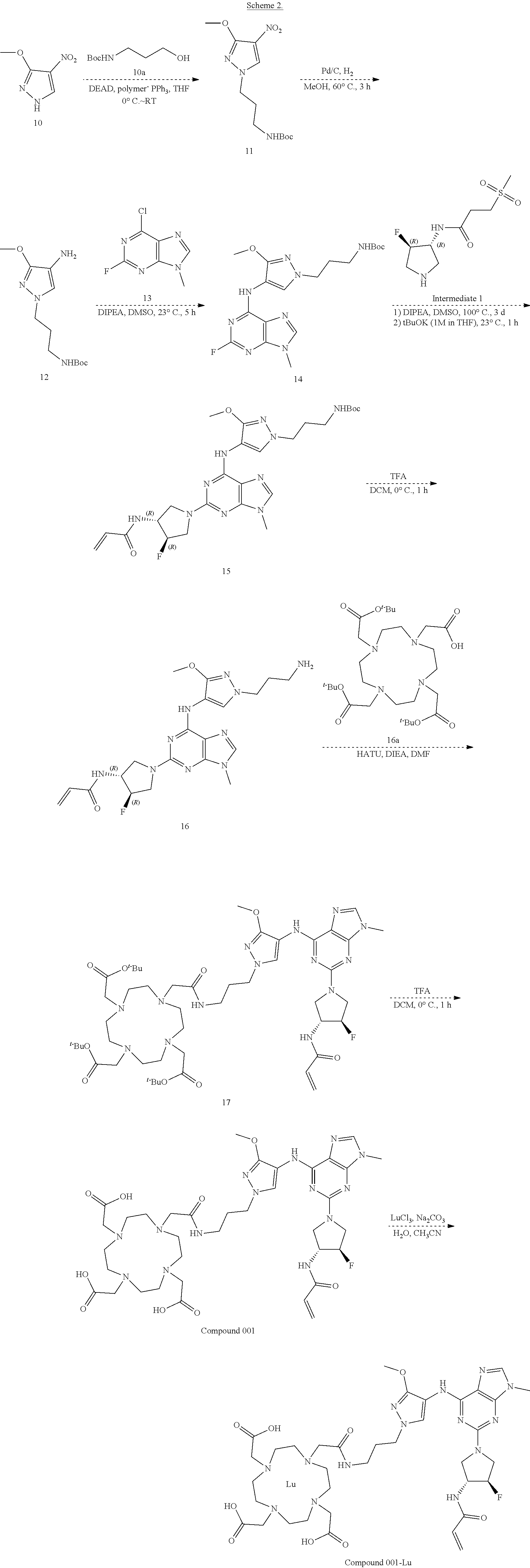

It is understood that the interaction between lutetium and the metal chelator is not shown in conjugate Compound 001-Lu.

### Example A3: General Synthesis Scheme for Compounds 001-006

Compounds 001 to 006 can be synthesized by the scheme below.

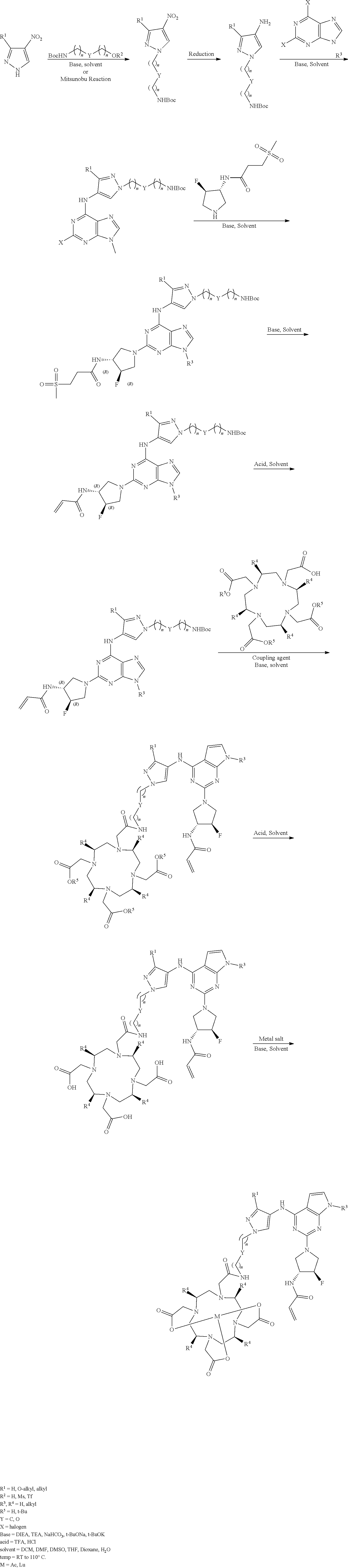

### Example A4: General Synthesis Scheme for Conjugate Compound 007

Compound 007 can be synthesized according to the scheme below.

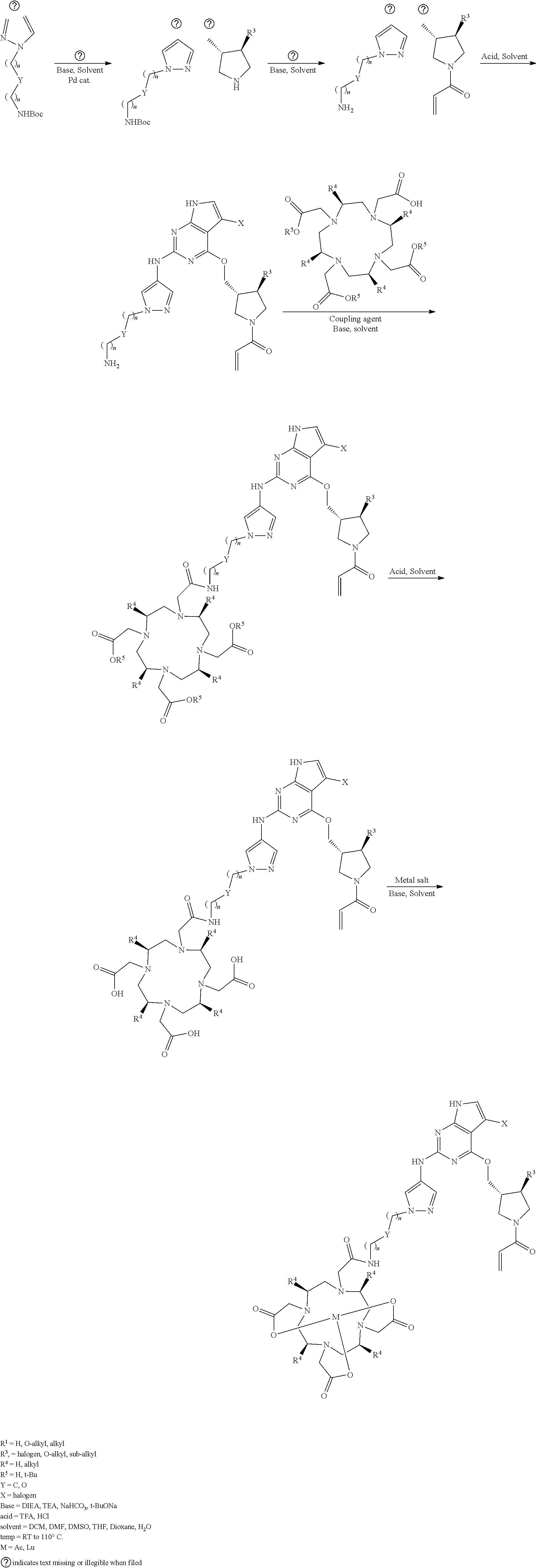

### Example A5: Synthesis of Compound 001 and Compound 001-Lu

### Preparation of Intermediate 1

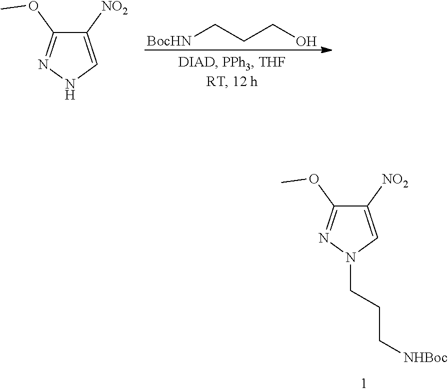

To a solution of 3-methoxy-4-nitro-1H-pyrazole (1 g, 7 mmol, 1 eq), PPh₃ (3.7 g, 14 mmol, 2 eq) and tert-butyl N-(3-hydroxypropyl)carbamate (1.5 g, 8.4 mmol, 1.4 mL, 1.2 eq) in THF (15 mL) was added DIAD (2.8 g, 14 mmol, 2.7 mL, 2 eq). The mixture was stirred at 25° C. for 12 h. The mixture was poured into water (30 mL) then extracted with ethyl acetate (30 mL×3). The combined organic phase was washed with brine (20 mL×2), dried over anhydrous Na₂SO₄, filtered, and concentrated to dryness. The residue was purified by column chromatography (SiO₂, petroleum ether/ethyl acetate=5/1). Intermediate 1, tert-butyl N-[3-(3-methoxy-4-nitro-pyrazol-1-yl)propyl]carbamate (1.3 g, 4.3 mmol, 62% yield), was obtained as a white solid. 1H NMR (400 MHz, DMSO-d6): δ (ppm) 1.38 (9H, s), 2.52-2.53 (2H, d, J=2.0 Hz), 2.91-2.96 (2H, m), 3.91-3.96 (3H, s), 4.00-4.04 (2H, t, J=6.8 Hz), 6.92 (1H, t, J=5.2 Hz), 8.71 (1H, s).

### Preparation of Intermediate 2

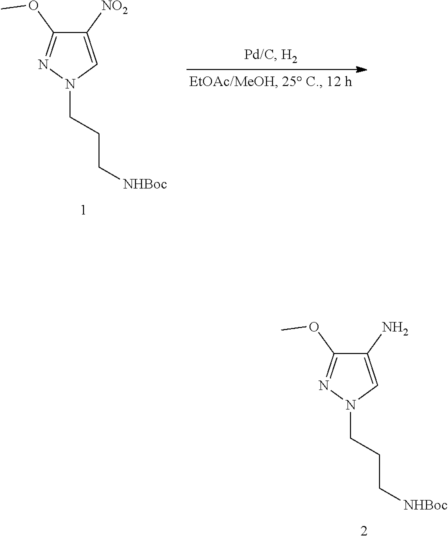 [SMILES: COc1nn(CCCNC(=O)OC(C)(C)C)cc1[N+](=O)[O-] ; COc1nn(CCCNC(=O)OC(C)(C)C)cc1N]

Pd/C (20 mg, 10% purity) was dissolved in ethyl acetate (10 mL) and the vessel was purged with N2 several times. Tert-butyl N-[3-(3-methoxy-4-nitro-pyrazol-1-yl)propyl]carbamate (200 mg, 666 umol, 1 eq) was added and the mixture was stirred at 25° C. for 12 h under H₂ (15 psi). LCMS showed the starting material was consumed and desired product was detected. The mixture was filtered and the filtrate was concentrated to dryness. Intermediate 2, tert-butyl N-[3-(4-amino-3-methoxy-pyrazol-1-yl)propyl]carbamate (220 mg, crude), was obtained as a green oil. 1H NMR (400 MHz, CDCl3): δ (ppm) 1.39 (9H, s), 1.70-1.75 (2H, m), 2.83-2.88 (2H, m), 3.73-3.78 (5H, m), 6.84-6.86 (1H, m), 6.94 (1H, s). MS (ESI) m/z: 270.3 (M+H)+, RT=0.887 min.

### Preparation of Intermediate 3

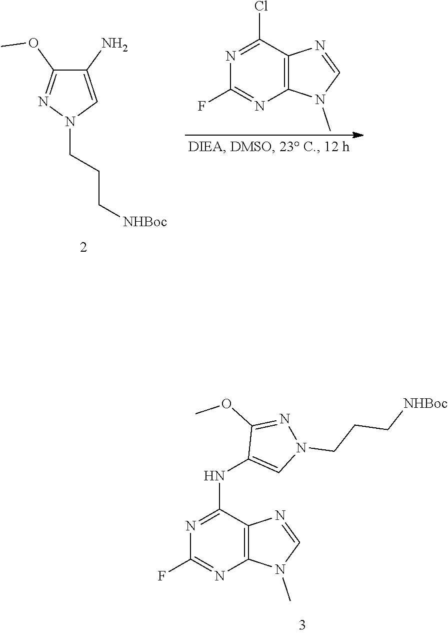 [SMILES: COc1nn(CCCNC(=O)OC(C)(C)C)cc1N ; Cn1cnc2c(Cl)nc(F)nc21 ; COc1nn(CCCNC(=O)OC(C)(C)C)cc1Nc1nc(F)nc2c1ncn2C]

To a solution of tert-butyl N-[3-(4-amino-3-methoxy-pyrazol-1-yl)propyl]carbamate (900 mg, 3.3 mmol, 1 eq) in DMSO (20 mL) was added DIEA (1.3 g, 10 mmol, 1.7 mL, 3 eq) and 6-chloro-2-fluoro-9-methyl-purine (850 mg, 3.4 mmol, 75% purity, 1 eq). The mixture was stirred at 23° C. for 5 h. LCMS analysis showed intermediate 2 was consumed and desired product was detected. Crude intermediate 3, tert-butyl N-[3-[4-[(2-fluoro-9-methyl-purin-6-yl)amino]-3-methoxy-pyrazol-1-yl]propyl]carbamate (1.2 g, crude), was obtained as a yellow solid and used for the next step without purification. MS (ESI) m/z: 421.3 (M+H)+, RT=0.771 min.

### Preparation of Intermediate 4

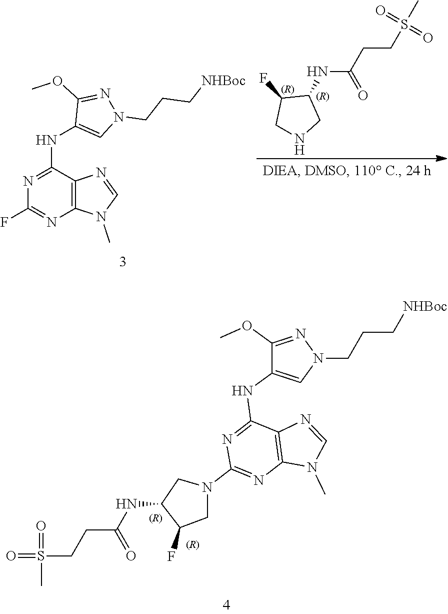

To a solution of tert-butyl N-[3-[4-[(2-fluoro-9-methyl-purin-6-yl)amino]-3-methoxy-pyrazol-1-yl]propyl]carbamate (1.3 g, 3.1 mmol, 1.3 eq) in DMSO (20 mL) was added DIEA (944 mg, 7.3 mmol, 1.3 mL, 3 eq) and N-[(3R,4R)-4-fluoropyrrolidin-3-yl]-3-methylsulfonyl-propanamide (669 mg, 2.4 mmol, 1 eq, HCl). The mixture was stirred at 110° C. for 72 h. LCMS analysis showed reactant 3 was consumed and desired product was detected. The mixture was poured into water (50 mL) then extracted with ethyl acetate (50 mL×2). The combined organic phase was washed with brine (50 mL×2), dried over anhydrous Na₂SO₄, filtered, and concentrated to dryness. The residue was purified by column chromatography (SiO₂, petroleum ether/ethyl acetate=0:1 to dichloromethane/methanol=10:1 gradient). Intermediate 4, tert-butyl N-[3-[4-[[2-[(3R,4R)-3-fluoro-4-(3-methylsulfonylpropanoylamino)pyrrolidin-1-yl]-9-methyl-purin-6-yl]amino]-3-methoxy-pyrazol-1-yl]propyl]carbamate (540 mg, 625.6 umol, 25.7% yield, 74% purity), was obtained as a brown-black oil. 1H NMR (400 MHz, DMSO-d₆): δ (ppm) 1.38 (9H, s), 1.84 (2H, t, J=6.8 Hz), 2.57 (2H, t, J=7.6 Hz), 2.94-3.00 (5H, m), 3.30-3.34 (2H, m), 3.63 (3H, s), 3.66-3.85 (7H, m), 3.92-4.00 (2H, m), 4.32-4.43 (1H, m), 5.00-5.16 (1H, m), 6.91 (1H, t, J=5.2 Hz), 7.80 (1H, s), 8.07 (1H, s), 7.84-7.94 (1H, m), 8.45 (1H, d, J=6.4 Hz). MS (ESI) m/z: 639.2 (M+H)⁺, RT=0.697 min.

### Preparation of Intermediate 5

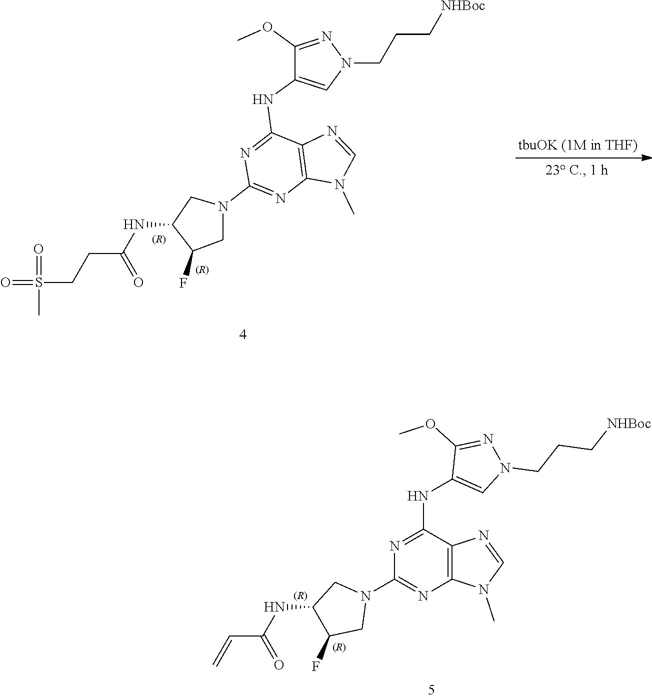

To a solution of tert-butyl N-[3-[4-[[2-[(3R,4R)-3-fluoro-4-(3-methylsulfonylpropanoylamino)pyrrolidin-1-yl]-9-methyl-purin-6-yl]amino]-3-methoxy-pyrazol-1-yl]propyl]carbamate (300 mg, 469.7 umol, 1 eq) in THF (1 mL) was added potassium 2-methylpropan-2-olate (527.1 mg, 4.7 mmol, 10 eq) at 25° C. for 1 h. The mixture was poured into water (100 mL) then extracted with ethyl acetate (40 mL×3). The combined organic phase was washed with brine (40 mL×2), dried over anhydrous Na₂SO₄, filtered, and concentrated to dryness. The residue was purified by prep-HPLC (column: Phenomenex Synergi C18 150*25 mm*10 um; mobile phase: [water(FA)-ACN]; B %: 26%-56%, 10 min). Intermediate 5, tert-butyl N-[3-[4-[[2-[(3R,4R)-3-fluoro-4-(prop-2-enoylamino)pyrrolidin-1-yl]-9-methyl-purin-6-yl]amino]-3-methoxy-pyrazol-1-yl]propyl]carbamate (200 mg, 333 umol, 71% yield, 93% purity), was obtained as a white solid. 1H NMR (400 MHz, CDCl3): δ (ppm) 1.41 (9H, s), 1.93-2.00 (2H, m), 3.08-3.18 (2H, m), 3.63 (3H, s), 3.87 (3H, m), 3.97 (4H, s), 4.67-4.76 (1H, m), 4.95-5.05 (1H, m), 5.28 (1H, s), 5.67 (1H, d, J=11.2 Hz), 6.15-6.27 (1H, m), 6.35-6.42 (1H, m), 6.75-6.89 (1H, m), 7.04-7.10 (1H, m), 7.46 (1H, s), 7.83 (1H, s). MS (ESI) m/z: 558.6 (M+H)+, RT=0.735 min.

### Preparation of Intermediate 6

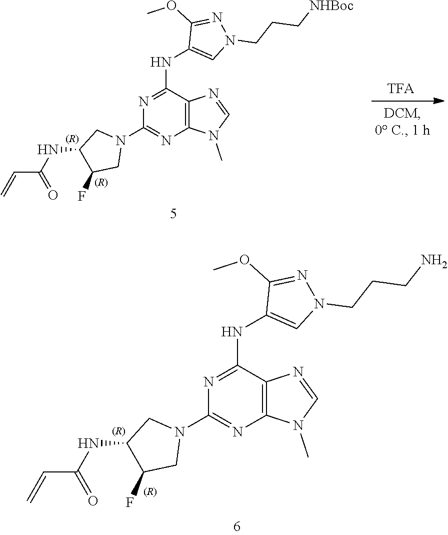

A mixture of tert-butyl N-[3-[4-[[2-[(3R,4R)-3-fluoro-4-(prop-2-enoylamino)pyrrolidin-1-yl]-9-methyl-purin-6-yl]amino]-3-methoxy-pyrazol-1-yl]propyl]carbamate (130 mg, 232.7 umol, 1 eq), TFA (79.6 mg, 698.2 umol, 51.7 uL, 3 eq) in DCM (3 mL) was degassed and purged with N2 for three times. The mixture was stirred at 25° C. for 2 h under N2 atmosphere. LCMS analysis showed reactant 5 was consumed and desired mass was detected. The reaction mixture was filtered and concentrated under reduced pressure to give Intermediate 6, N-[(3R,4R)-1-[6-[[1-(3-aminopropyl)-3-methoxy-pyrazol-4-yl]amino]-9-methyl-purin-2-yl]-4-fluoro-pyrrolidin-3-yl]prop-2-enamide (130 mg, crude, TFA), as a yellow solid. The crude material was used for the next step without further purification. MS (ESI) m/z:459.3 (M+H)+, RT=0.631 min.

### Preparation of Intermediate 7

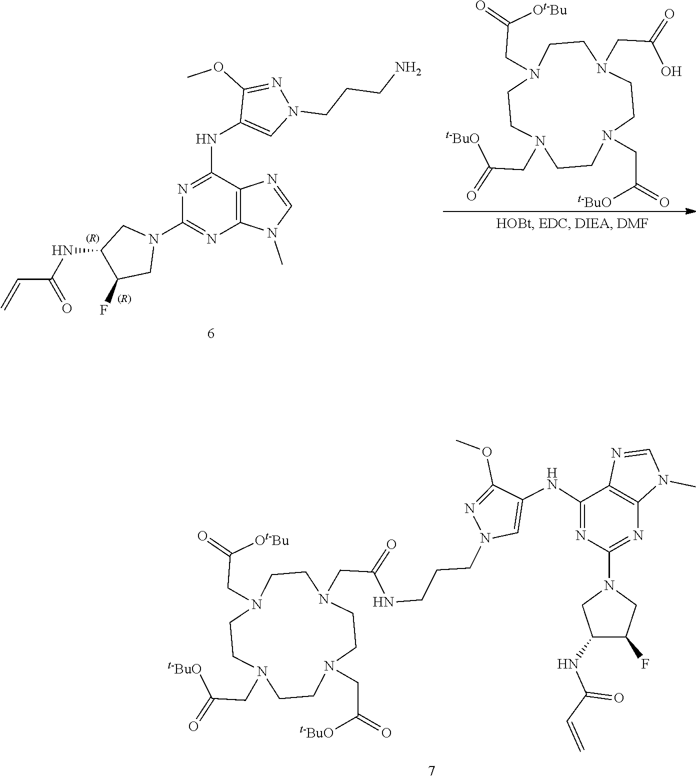

To a solution of N-[(3R,4R)-1-[6-[[1-(3-aminopropyl)-3-methoxy-pyrazol-4-yl]amino]-9-methyl-purin-2-yl]-4-fluoro-pyrrolidin-3-yl]prop-2-enamide (130 mg, 227.1 umol, 1 eq, TFA) in DMF (3 mL) was added 2-[4,7,10-tris(2-tert-butoxy-2-oxo-ethyl)-1,4,7,10-tetrazacyclododec-1-yl]acetic acid (156.1 mg, 272.5 umol, 1.2 eq), EDC (87.1 mg, 454.1 umol, 2 eq), DIEA (88 mg, 681.2 umol, 118.7 uL, 3 eq) and HOBt (61.4 mg, 454.1 umol, 2 eq). The mixture was stirred at 25° C. for 12 h. LCMS analysis showed intermediate 6 was consumed and desired mass was detected. The residue was purified by prep-HPLC (column: Phenomenex Synergi C18 150*25 mm* 10 um; mobile phase: [water(FA)-ACN]; B %: 13%-43%, 10 min). Intermediate 7, tert-butyl 2-[4,7-bis(2-tert-butoxy-2-oxo-ethyl)-10-[2-[3-[4-[[2-[(3R,4R)-3-fluoro-4-(prop-2-enoylamino)pyrrolidin-1-yl]-9-methyl-purin-6-yl]amino]-3-methoxy-pyrazol-1-yl]propylamino]-2-oxo-ethyl]-1,4,7,10-tetrazacyclododec-1-yl]acetate (200 mg, 185.6 umol, 81.7% yield, 94% purity), was obtained as a brown-black oil.

1H NMR (400 MHz, MeOH-d4): δ (ppm0 1.31-1.48 (27H, m), 2.62-3.24 (15H, m), 3.27 (3H, m), 3.39-3.53 (6H, m) 3.67 (3H, s), 3.74 (1H, s), 3.79-3.89 (3H, m), 3.94 (3H, s), 3.95-4.10 (6H, m) 4.56 (1H, dd, J=11.6, 5.6 Hz), 5.08-5.24 (1H, m) 5.65 (1H, dd, J=7.2, 4.8 Hz), 6.20-6.26 (2H, m), 7.73 (1H, s), 7.98-8.04 (1H, m). MS (ESI) m/z:1013.2 (M+H)+, RT=0.722 min.

### Preparation of Compound 001

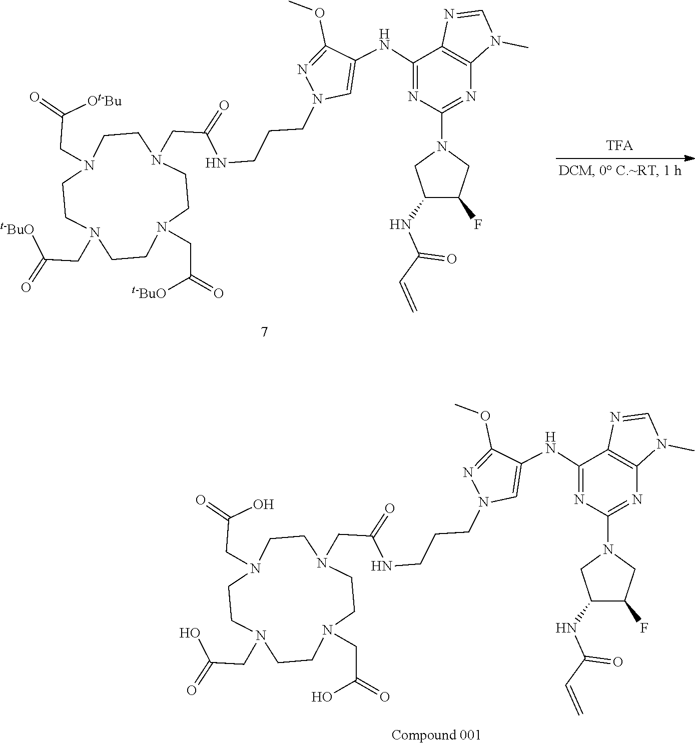 [SMILES: C=CC(=O)N[C@@H]1CN(c2nc(Nc3cn(CCCNC(=O)CN4CCN(CC(=O)OC(C)(C)C)CCN(CC(=O)OC(C)(C)C)CCN(CC(=O)OC(C)(C)C)CC4)nc3OC)c3ncn(C)c3n2)C[C@H]1F ; C=CC(=O)N[C@@H]1CN(c2nc(Nc3cn(CCCNC(=O)CN4CCN(CC(=O)O)CCN(CC(=O)O)CCN(CC(=O)O)CC4)nc3OC)c3ncn(C)c3n2)C[C@H]1F]

To a solution of tert-butyl 2-[4,7-bis(2-tert-butoxy-2-oxo-ethyl)-10-[2-[3-[4-[[2-[(3R,4R)-3-fluoro-4-(prop-2-enoylamino)pyrrolidin-1-yl]-9-methyl-purin-6-yl]amino]-3-methoxy-pyrazol-1-yl]propylamino]-2-oxo-ethyl]-1,4,7,10-tetrazacyclododec-1-yl]acetate (90 mg, 88.8 umol, 1 eq) in DCM (5 mL) was added TFA (50.6 mg, 444.1 umol, 32.9 uL, 5 eq) at 0° C. The mixture was stirred at 25° C. for 12 h. LCMS analysis showed intermediate 7 was consumed and desired product was detected. The reaction mixture was concentrated under reduced pressure to give a residue. The residue was purified by prep-HPLC (column: Phenomenex Synergi C18 150*25 mm* 10 um; mobile phase: [water(FA)-ACN]; B %: 4%-31%, 9 min). Compound 001, 2-[4,7-bis(carboxymethyl)-10-[2-[3-[4-[[2-[(3R,4R)-3-fluoro-4-(prop-2-enoylamino)pyrrolidin-1-yl]-9-methyl-purin-6-yl]amino]-3-methoxy-pyrazol-1-yl]propylamino]-2-oxo-ethyl]-1,4,7,10-tetrazacyclododec-1-yl]acetic acid (36.5 mg, 38.1 umol, 42.9% yield, 100% purity, TFA), was obtained as a white solid. 1H NMR (400 MHz, MeOH-d4): δ (ppm) 2.06 (2H, d, J=5.2 Hz), 3.07 (9H, brs), 3.18-3.29 (3H, m), 3.34-3.51 (1OH, m), 3.53-3.67 (3H, m), 3.74 (8H, d, J=14.4 Hz), 3.84-3.94 (2H, m), 3.98 (6H, s), 4.11 (3H, brs), 4.62 (1H, dd, J=11.2, 5.2 Hz), 5.13-5.29 (1H, m), 5.66-5.73 (1H, m), 6.29-6.33 (2H, m), 7.80 (1H, s), 8.06-8.18 (1H, in). MS (ESI) m/z:845.3 (M+H)⁺, RT=2.274 min.

### Preparation of Compound 001-Lu

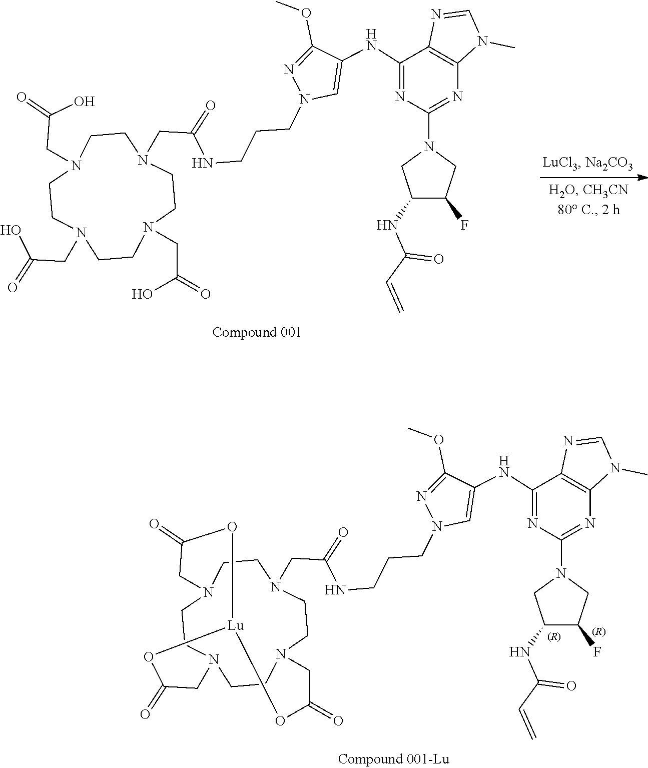

To a solution of 2-[4,7-bis(carboxymethyl)-10-[2-[3-[4-[[2-[(3R,4R)-3-fluoro-4-(prop-2-enoylamino)pyrrolidin-1-yl]-9-methyl-purin-6-yl]amino]-3-methoxy-pyrazol-1-yl]propylamino]-2-oxo-ethyl]-1,4,7,10-tetrazacyclododec-1-yl]acetic acid (40 mg, 41.7 umol, 1 eq, TFA) in CH₃CN (3 mL) was added NaCO₃ (3.5 mg, 41.7 umol, 1 eq) in H₂O (3 mL) and trichlorolutetium (11.7 mg, 41.7 umol, 1 eq). The mixture was stirred at 80° C. for 2 h. LCMS analysis showed Compound 001 was consumed and the desired product was detected. The mixture was poured into water (20 mL) then extracted with ethyl acetate (20 mL×2). The combined organic phase was washed with brine (10 mL×2), dried over anhydrous Na₂SO₄, filtered, and concentrated to dryness. The residue was purified by prep-HPLC (column: Waters Xbridge 150*25 mm* 5 um; mobile phase: [water (ammonia hydroxide v/v)-ACN]; B %: 4%-34%, 9 min). Compound 001-Lu, N-[(3R,4R)-4-fluoro-1-[6-[[3-methoxy-1-[3-[[2-(3,16,19-trioxo-2,17,18-trioxa-5,8,11,14-tetraza-1λ3-lutetatricyclo[9.6.3.25,14]docosan-8-yl)acetyl]amino]propyl]pyrazol-4-yl]amino]-9-methyl-purin-2-yl]pyrrolidin-3-yl]prop-2-enamide (18 mg, 17.7 umol, 42.4% yield, 100% purity), was obtained as a brown-black oil. ¹H NMR (400 MHz, MeOH-d₄): δ (ppm) 2.06-2.21 (2H, s), 2.28-2.60 (5H, m) 2.63-2.89 (7H, m), 3.12-3.30 (3H, m), 3.33 (4H, dt, J=3.2, 1.6 Hz), 3.37-3.54 (6H, m), 3.57-3.71 (2H, m), 3.84 (4H, s), 3.92 (1H, d, J=6.8 Hz), 3.95-4.05 (5H, m) 4.12 (2H, br s), 4.63 (1H, dd, J=12.0, 5.2 Hz), 5.15-5.31 (1H, m), 5.69-5.73 (1H, m), 6.26-6.34 (2H, m), 7.98-8.06 (1H, s), 8.45-8.55 (1H, s). MS (ESI) m/z:1016.3 (M+H)⁺, RT=2.032 min.

### Example A6: Synthesis of Compound 002 and Compound 002-Lu

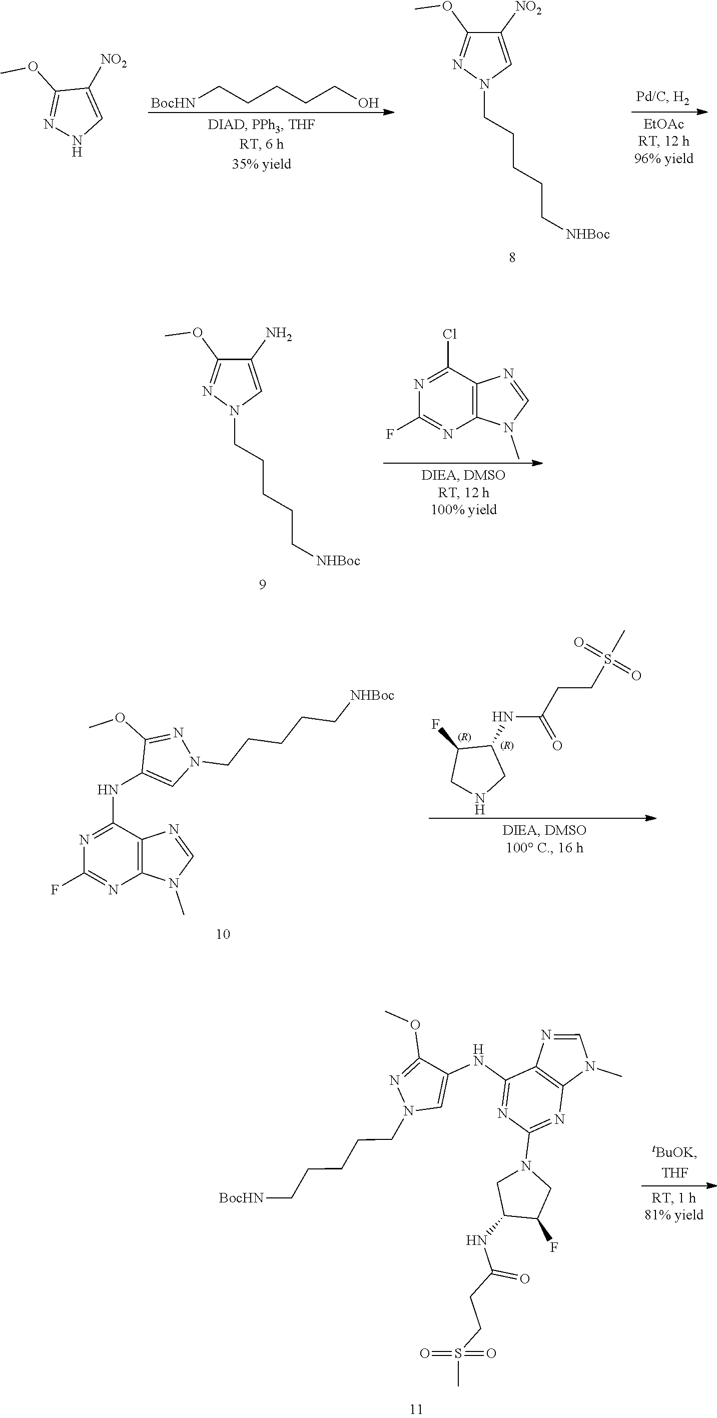

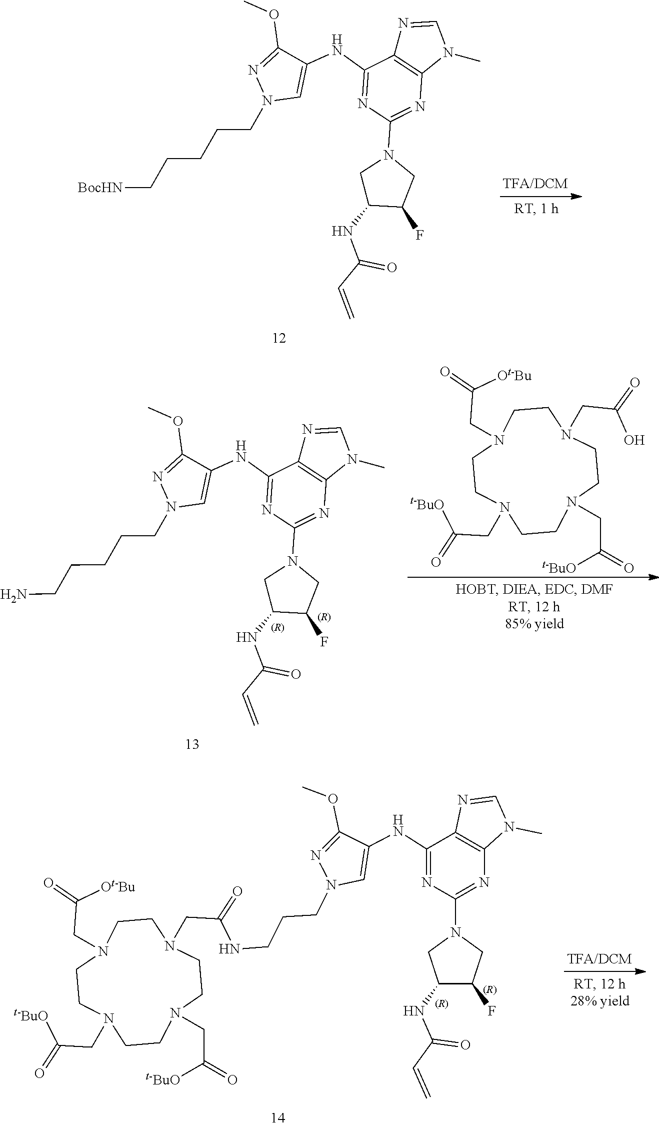

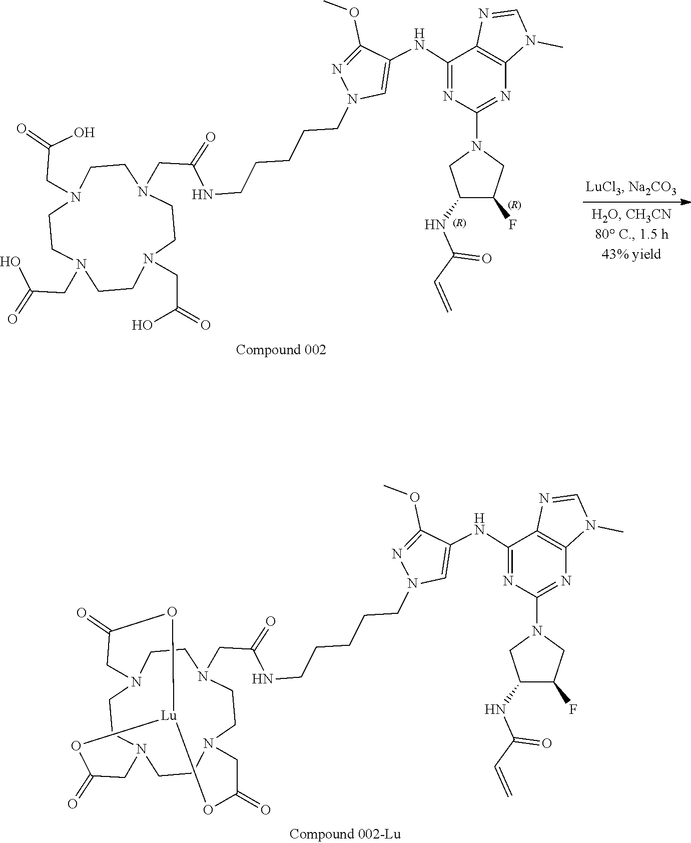

### Preparation of Intermediate 8

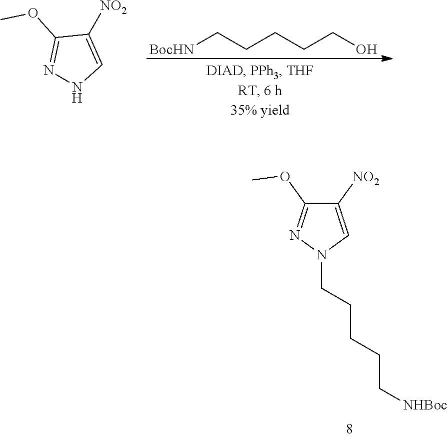

To a solution of 3-methoxy-4-nitro-1H-pyrazole (1 g, 7 mmol, 1 eq), tert-butyl N-(5-hydroxypentyl)carbamate (2.8 g, 14 mmol, 2.8 mL, 2 eq) and PPh₃ (3.7 g, 14 mmol, 2 eq) in THF (20 mL) was added DIAD (2.8 g, 14 mmol, 2.7 mL, 2 eq). The mixture was stirred at 25° C. for 6 h. TLC showed the starting material was consumed completely and new spots formed. The reaction mixture was concentrated under reduced pressure to remove solvent. The residue was diluted with H₂O (60 mL) and extracted with ethyl acetate (25 mL×3). The combined organic layers were washed with brine (30 mL), dried over Na₂SO₄, filtered, and concentrated under reduced pressure to give a residue. The crude product was purified by column chromatography (SiO₂, petroleum ether/ethyl acetate=20:1 to 5:1 gradient) to give Intermediate 8, tert-butyl N-[5-(3-methoxy-4-nitro-pyrazol-1-yl)pentyl]carbamate (800 mg, 2.4 mmol, 35% yield), as a yellow oil. 1H NMR (400 MHz, CDCl3) δ ppm 1.32-1.38 (2H, m), 1.44 (9H, s), 1.53 (2H, br d, J=7.2 Hz), 1.90 (2H, m), 3.11-3.16 (2H, m), 3.97 (2H, t, J=7.2 Hz), 4.04 (3H, s), 4.47-4.63 (1H, m), 7.98 (1H, s).

### Preparation of Intermediate 9

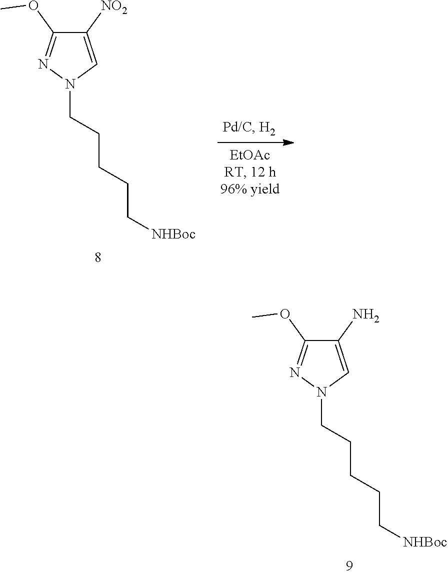 [SMILES: COc1nn(CCCCCNC(=O)OC(C)(C)C)cc1[N+](=O)[O-] ; COc1nn(CCCCCNC(=O)OC(C)(C)C)cc1N]

A suspension of Pd/C (50 mg, 10% purity) in ethyl acetate (20 mL) was purged with N₂ several times. Then, tert-butyl N-[5-(3-methoxy-4-nitro-pyrazol-1-yl)pentyl]carbamate (0.9 g, 2.7 mmol, 1 eq) was added and the mixture was stirred at 25° C. for 12 h under H₂ (15 psi). TLC showed intermediate 8 was consumed and a new more polar spot was observed. The mixture was filtered and the filtrate was concentrated. Intermediate 9, tert-butyl N-[5-(4-amino-3-methoxy-pyrazol-1-yl)pentyl]carbamate (785 mg, 2.6 mmol, 96% yield), was obtained as ayellow gel. 1H NMR (400 MHz, DMSO-d6) δ 6.91 (1H, s), 6.75 (1H, t, J=5.2 Hz), 3.75-3.68 (5H, m), 2.90-2.85 (2H, m), 1.66-1.57 (2H, m), 1.37-1.30 (11H, m), 1.20-1.16 (2H, m).

### Preparation of Intermediate 10

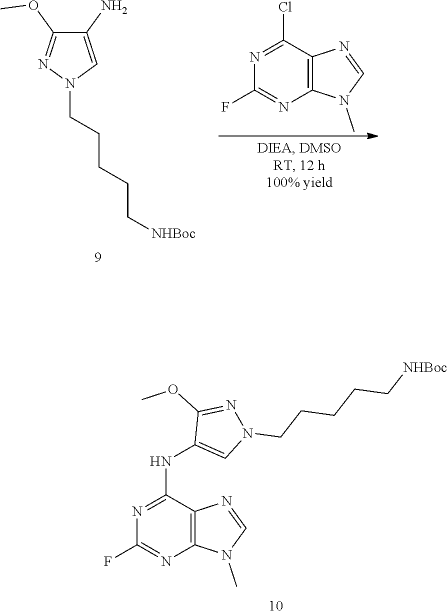 [SMILES: COc1nn(CCCCCNC(=O)OC(C)(C)C)cc1N ; Cn1cnc2c(Cl)nc(F)nc21 ; COc1nn(CCCCCNC(=O)OC(C)(C)C)cc1Nc1nc(F)nc2c1ncn2C]

To a solution of tert-butyl N-[5-(4-amino-3-methoxy-pyrazol-1-yl)pentyl]carbamate (780 mg, 2.6 mmol, 1 eq), 6-chloro-2-fluoro-9-methyl-purine (700 mg, 2.8 mmol, 75% purity, 1.1 eq) in DMSO (10 mL) was added DIEA (675.7 mg, 5.2 mmol, 910.7 uL, 2 eq). The mixture was stirred at 25° C. for 12 h under N₂ atmosphere. LCMS showed the starting material was consumed and one major peak with desired mass was detected. Intermediate 10, tert-butyl N-[5-[4-[(2-fluoro-9-methyl-purin-6-yl)amino]-3-methoxy-pyrazol-1-yl]pentyl]carbamate (1.17 g, 2.61 mmol, 100% yield), was obtained as a brown liquid in DMSO and used directly. MS (ESI) m/z: 449.3 (M+H)+, RT=0.898 min.

### Preparation of Intermediate 11

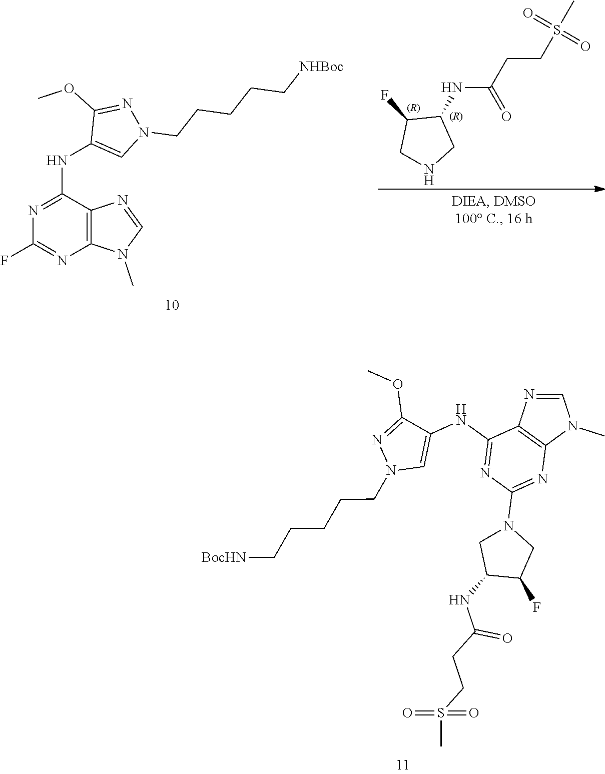

To a solution of tert-butyl N-[5-[4-[(2-fluoro-9-methyl-purin-6-yl)amino]-3-methoxy-pyrazol-1-yl]pentyl]carbamate (0.7 g, 1.5 mmol, 1 eq) and N-[(3R,4R)-4-fluoropyrrolidin-3-yl]-3-methylsulfonyl-propanamide (500 mg, 1.8 mmol, 1.2 eq, HCl) in DMSO (10 mL) was added DIEA (783.8 mg, 6.1 mmol, 1.1 mL, 4 eq). The mixture was stirred at 100° C. for 16 h. LCMS showed the starting material was consumed and one peak with desired mass was detected. The mixture was diluted with water (150 mL) then extracted with ethyl acetate (30 mL×3). The combined organic phase was washed with brine (30 mL), dried over anhydrous Na₂SO₄, filtered, and concentrated to dryness. The residue was purified by column chromatography (SiO₂, ethyl acetate:methanol=50:1 to 30:1 gradient) to give Intermediate 11, tert-butyl N-[5-[4-[[2-[(3R,4R)-3-fluoro-4-(3-methylsulfonylpropanoylamino)pyrrolidin-1-yl]-9-methyl-purin-6-yl]amino]-3-methoxy-pyrazol-1-yl]pentyl]carbamate (700 mg, 1.1 mmol, 69% yield), as a light-yellow solid. 1H NMR (400 MHz, CDCl3) δ 7.86 (1H, s), 7.53-7.42 (2H, m), 7.04 (1H, s), 5.21 (1H, d, J=50.8 Hz), 4.70-4.55 (2H, m), 4.02-3.90 (7H, m), 3.88-3.80 (2H, m), 3.64 (3H, s), 3.51-3.36 (2H, m), 3.16-3.04 (2H, m), 2.96 (3H, s), 2.80 (2H, t, J=6.8 Hz), 1.81-1.75 (2H, m), 1.53-1.43 (2H, m), 1.41-1.29 (11H, m). MS (ESI) m/z: 667.4 (M+H)+, RT=0.839 min.

### Preparation of Intermediate 12

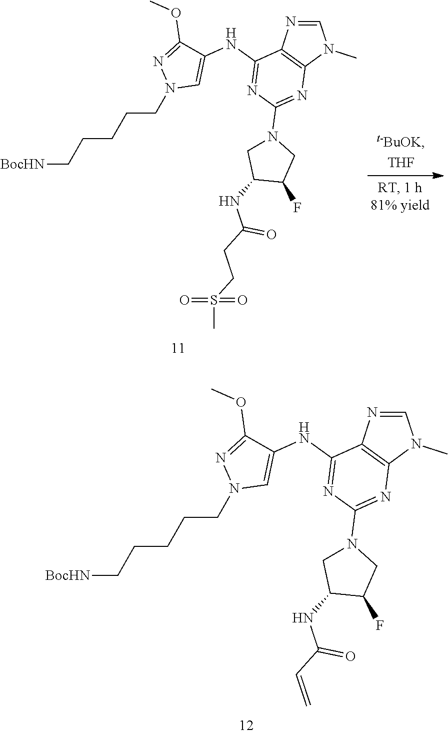 [SMILES: COc1nn(CCCCCNC(=O)OC(C)(C)C)cc1Nc1nc(N2C[C@@H](F)[C@H](NC(=O)CCS(C)(=O)=O)C2)nc2c1ncn2C ; C=CC(=O)N[C@@H]1CN(c2nc(Nc3cn(CCCCCNC(=O)OC(C)(C)C)nc3OC)c3ncn(C)c3n2)C[C@H]1F]

To a solution of tert-butyl N-[5-[4-[[2-[(3R,4R)-3-fluoro-4-(3-methylsulfonylpropanoylamino)pyrrolidin-1-yl]-9-methyl-purin-6-yl]amino]-3-methoxy-pyrazol-1-yl]pentyl]carbamate (700 mg, 1.1 mmol, 1 eq) in THF (30 mL) was added t-BuOK (1 M, 5.2 mL, 4.9 eq). The mixture was stirred at 25° C. for 1 h. LCMS showed reactant 11 was consumed completely and one major peak with desired mass was detected. The reaction mixture was quenched by addition H₂O (30 mL) and then extracted with ethyl acetate (15 mL×3). The combined organic layers were washed with brine (30 mL), dried over Na₂SO₄, filtered, and concentrated under reduced pressure to give intermediate 12, tert-butyl N-[5-[4-[[2-[(3R,4R)-3-fluoro-4-(prop-2-enoylamino)pyrrolidin-1-yl]-9-methyl-purin-6-yl]amino]-3-methoxy-pyrazol-1-yl]pentyl]carbamate (500 mg, 852.3 umol, 81% yield), as a purple solid. 1H NMR (400 MHz, CDCl3) δ ppm 1.33 (9H, br s), 1.37-1.49 (4H, m), 1.77-1.86 (2H, m), 3.01-3.19 (2H, m), 3.65 (3H, s), 3.86 (2H, m), 3.92-4.00 (7H, m), 4.52-4.62 (1H, m), 4.69-4.78 (1H, m), 5.19-5.35 (1H, m), 5.66 (1H, m), 6.15-6.24 (1H, m), 6.35-6.42 (1H, m), 6.98-7.20 (2H, m), 7.45 (1H, s), 7.86 (1H, s). MS (ESI) m/z: 587.3 (M+H)⁺, RT=0.668 min.

### Preparation of Intermediate 13

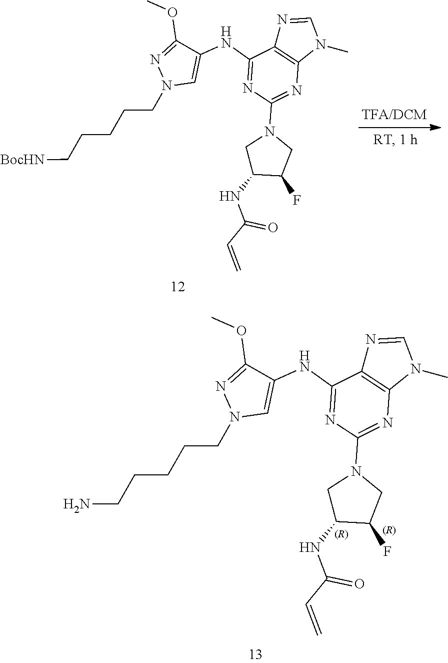

To a solution of tert-butyl N-[5-[4-[[2-[(3R,4R)-3-fluoro-4-(prop-2-enoylamino)pyrrolidin-1-yl]-9-methyl-purin-6-yl]amino]-3-methoxy-pyrazol-1-yl]pentyl]carbamate (250 mg, 426.1 umol, 1 eq) in DCM (10 mL) was added TFA (2 mL). The mixture was stirred at 25° C. for 1 h. LCMS showed compound 12 was consumed completely and one major peak with desired mass was detected. The reaction mixture was concentrated under reduced pressure to remove solvent. Intermediate 13, N-[(3R,4R)-1-[6-[[1-(5-aminopentyl)-3-methoxy-pyrazol-4-yl]amino]-9-methyl-purin-2-yl]-4-fluoro-pyrrolidin-3-yl]prop-2-enamide (260 mg, crude, TFA), was obtained as a brown oil. The crude product was used in the next step without further purification. 1H NMR (400 MHz, CD3OD) δ ppm 1.40-1.45 (2H, m) 1.70 (2H, m) 1.89 (2H, m) 2.92 (2H, brt, J=8.0 Hz) 3.78-3.86 (1H, m) 3.86-3.97 (5H, m) 3.98 (3H, s) 4.00-4.12 (4H, m) 4.60-4.66 (1H, m) 5.13-5.30 (1H, m) 5.70 (1H, m) 6.28 (1H, d, J=3.2 Hz) 8.05 (1H, s) 8.75. MS (ESI) m/z: 487.2 (M+H)+, RT=0.498 min.

### Preparation of Intermediate 14

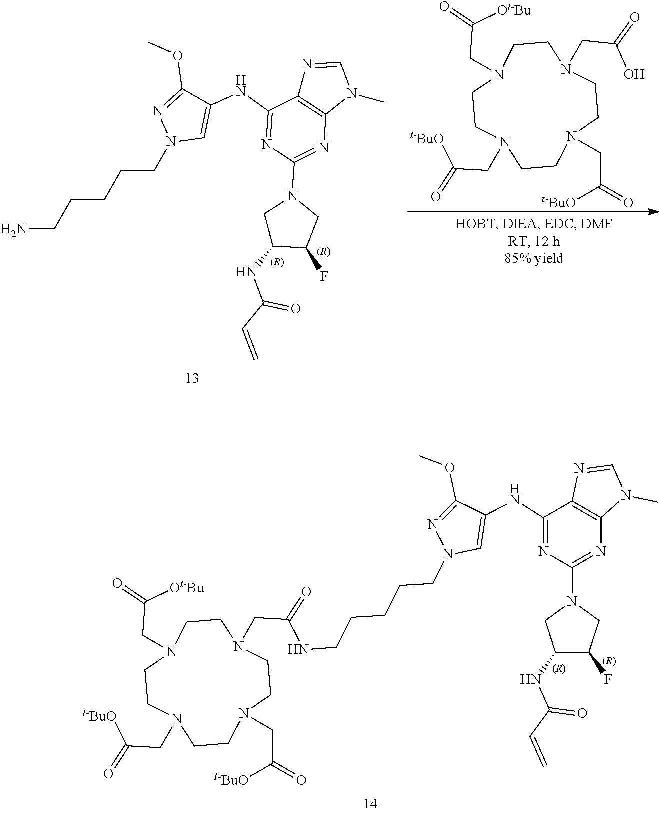

To a solution of N-[(3R,4R)-1-[6-[[1-(5-aminopentyl)-3-methoxy-pyrazol-4-yl]amino]-9-methyl-purin-2-yl]-4-fluoro-pyrrolidin-3-yl]prop-2-enamide (260 mg, 534.4 umol, 1 eq), 2-[4,7,10-tris(2-tert-butoxy-2-oxo-ethyl)-1,4,7,10-tetrazacyclododec-1-yl]acetic acid (367.3 mg, 641.3 umol, 1.2 eq), DIEA (345.3 mg, 2.7 mmol, 465.4 uL, 5 eq) and HOBt (86.7 mg, 641.3 umol, 1.2 eq) in DMF (10 mL) was added EDC (204.9 mg, 1.1 mmol, 2 eq). The mixture was stirred at 25° C. for 12 h. LCMS showed starting material was consumed completely and one major peak with the desired mass was detected. The mixture was filtered through a syringe filter and purified by prep-HPLC (column: Phenomenex luna C18 150*40 mm* 15 um; mobile phase: [water(TFA)-ACN]; B %: 20%-50%, 10 min). Intermediate 14, tert-butyl 2-[4,7-bis(2-tert-butoxy-2-oxo-ethyl)-10-[2-[5-[4-[[2-[(3R,4R)-3-fluoro-4-(prop-2-enoylamino)pyrrolidin-1-yl]-9-methyl-purin-6-yl]amino]-3-methoxy-pyrazol-1-yl]pentylamino]-2-oxo-ethyl]-1,4,7,10-tetrazacyclododec-1-yl]acetate (489 mg, 469.6 umol, 87.9% yield) was obtained as a colorless oil with 95% purity. 1H NMR (400 MHz, CD3OD) δ ppm 1.43 (31H, br s), 1.78-1.88 (2H, m), 2.56-3.30 (14H, m), 3.36-3.75 (12H, m), 3.82 (3H, s), 3.95-4.06 (9H, m), 4.62 (1H, m), 5.12-5.28 (1H, m), 5.70 (1H, m), 6.24-6.31 (2H, m), 8.07 (1H, s), 8.35 (1H, br s). MS (ESI) m/z: 1041.9 (M+H)+, RT=0.731 min.

### Preparation of Compound 002

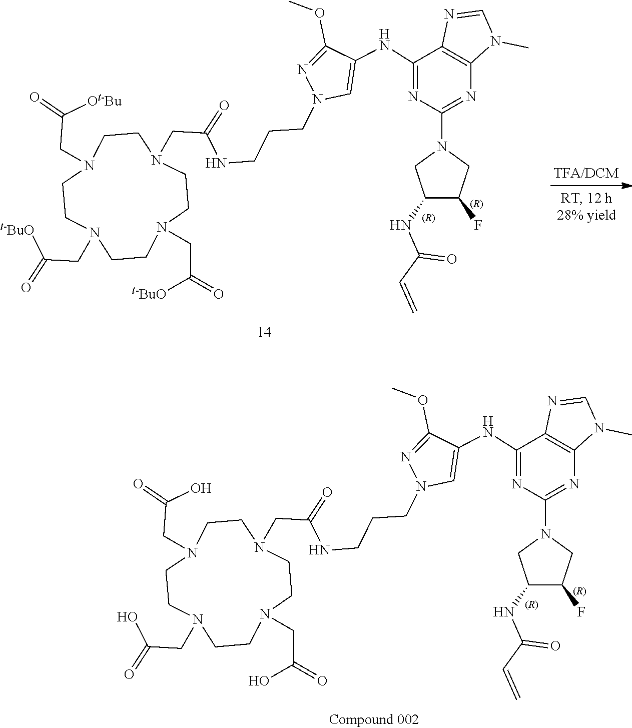

To a solution of tert-butyl 2-[4,7-bis(2-tert-butoxy-2-oxo-ethyl)-10-[2-[5-[4-[[2-[(3R,4R)-3-fluoro-4-(prop-2-enoylamino)pyrrolidin-1-yl]-9-methyl-purin-6-yl]amino]-3-methoxy-pyrazol-1-yl]pentylamino]-2-oxo-ethyl]-1,4,7,10-tetrazacyclododec-1-yl]acetate (250 mg, 240.1 umol, 1 eq) in DCM (9 mL) was added TFA (3 mL). The mixture was stirred at 25° C. for 12 h. LCMS showed compound 14 was consumed completely and one major peak with desired mass was detected. The mixture was filtered through a syringe filter and half of crude product was purified by prep-HPLC (column: 3_Phenomenex Luna C18 75*30 mm*3 um; mobile phase:[water(TFA)-ACN]; B %: 8%-38%, 7 min) to give Compound 002, 2-[4,7-bis(carboxymethyl)-10-[2-[5-[4-[[2-[(3R,4R)-3-fluoro-4-(prop-2-enoylamino)pyrrolidin-1-yl]-9-methyl-purin-6-yl]amino]-3-methoxy-pyrazol-1-yl]pentylamino]-2-oxo-ethyl]-1,4,7,10-tetrazacyclododec-1-yl]acetic acid (27.9 mg, 100% purity) as a white solid. The other half of crude material was used for the next step without further purification. 1H NMR (400 MHz, CD3OD) δ ppm 1.28-1.40 (2H, m), 1.50-1.61 (2H, m), 1.80-1.90 (2H, m), 1.92-3.29 (14H, m), 3.32-4.54 (24H, m), 4.62 (1H, m), 5.11-5.29 (1H, m), 5.65-5.75 (1H, m), 6.20-6.34 (2H, m), 8.05 (1H, br s), 8.17-8.44 (1H, m). MS (ESI) m/z: 873.5 (M+H)⁺, RT=0.748 min.

### Preparation of Compound 002-Lu

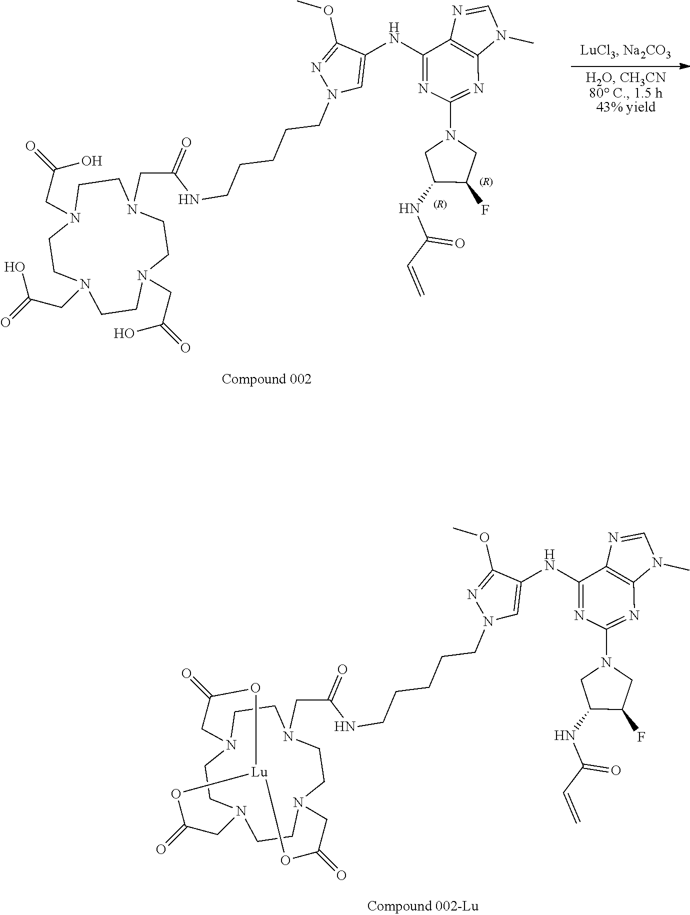

A solution of 2-[4,7-bis(carboxymethyl)-10-[2-[5-[4-[[2-[(3R,4R)-3-fluoro-4-(prop-2-enoylamino)pyrrolidin-1-yl]-9-methylpurin-6-yl]amino]-3-methoxy-pyrazol-1-yl]pentylamino]-2-oxo-ethyl]-1,4,7,10-tetrazacyclododec-1-yl]acetic acid (100 mg, 114.6 umol, 1 eq) in MeCN (5 mL) was adjusted to pH=6 with Na₂CO₃ (12.1 mg, 114.6 umol, 1 eq) in H₂O (2 mL). The trichlorolutetium (32 mg, 114.6 umol, 1 eq) was added to the mixture. The reaction was stirred at 80° C. for 1.5 h. LCMS showed the starting material was consumed completely and one main peak with desired mass was detected. The mixture was filtered through a syringe filter to give a clear solution. The crude material was purified by prep-HPLC (column: Waters Xbridge 150*25 mm*5 um; mobile phase: [water(NH₄HCO₃)— ACN]; B %: 4%-34%, 9 min). Compound 002-Lu, N-[(3R,4R)-4-fluoro-1-[6-[[3-methoxy-1-[5-[[2-(3,16,19-trioxo-2,17,18-trioxa-5,8,11,14-tetraza-1λ3-lutetatricyclo[9.6.3.25,14]docosan-8-yl)acetyl]amino]pentyl]pyrazol-4-yl]amino]-9-methyl-purin-2-yl]pyrrolidin-3-yl]prop-2-enamide (51.38 mg, 49.2 umol, 42.9% yield, 100% purity), was obtained as a white solid. 1H NMR (400 MHz, CD3OD) δ ppm 1.26-1.38 (2H, m), 1.50-1.67 (2H, m), 1.75-1.97 (2H, m), 2.18-3.26 (17H, m), 3.35-4.07 (17H, m), 4.56-4.67 (1H, m), 5.09-5.29 (1H, m), 5.61-5.72 (1H, m), 6.23-6.33 (2H, m), 7.75 (1H, s), 7.82-7.87 (1H, m), 8.08 (1H, s). MS (ESI) m/z: 523.3 (M+H)⁺, RT=0.557 min.

### Example A7: Synthesis of Compound 003 and Compound 003-Lu

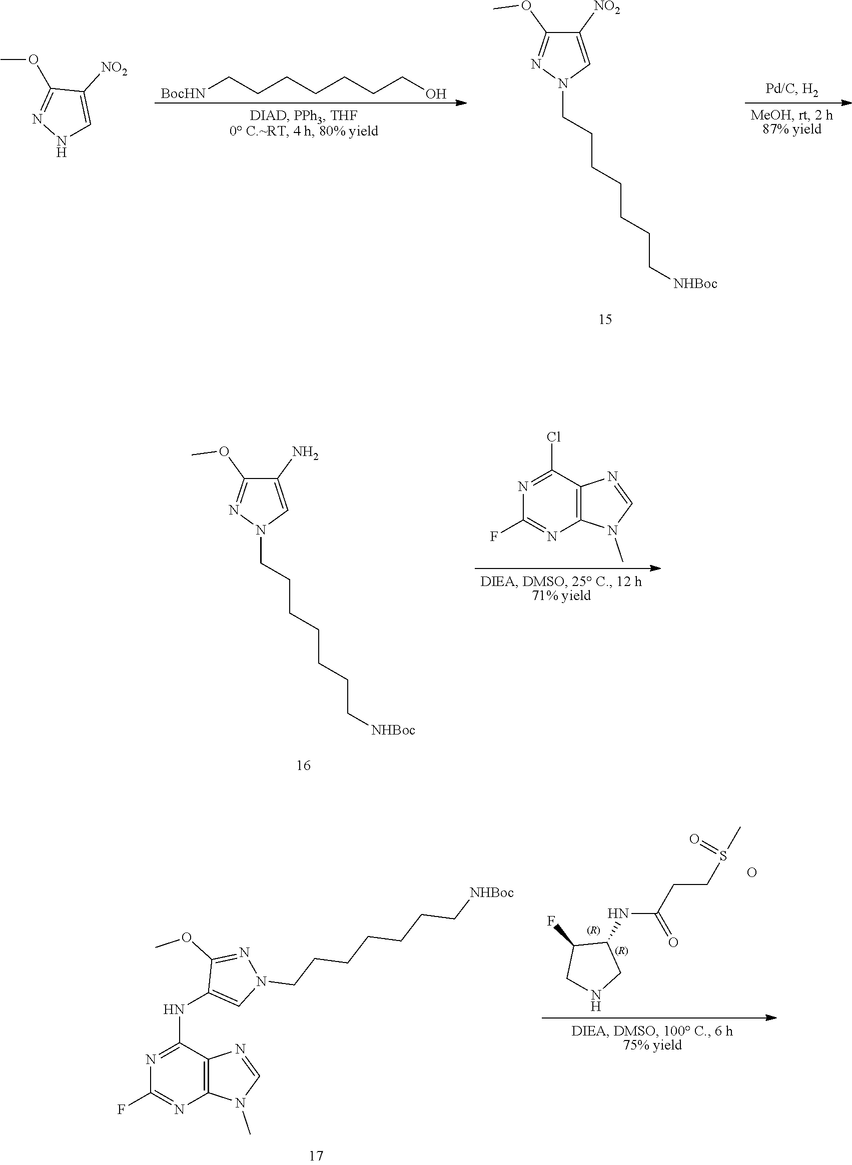

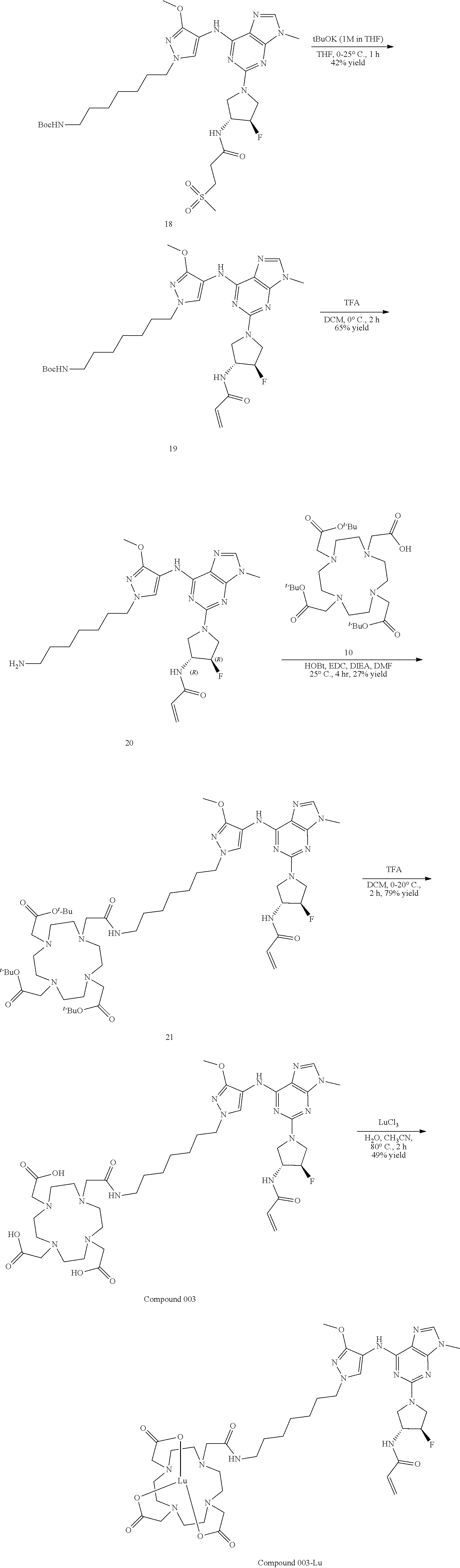

### Preparation of Intermediate 15

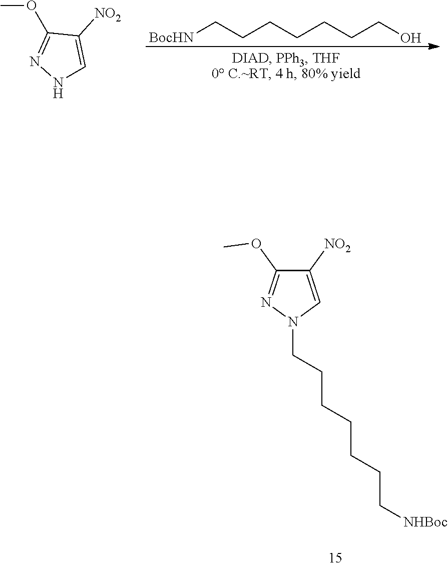

To a solution of 3-methoxy-4-nitro-1H-pyrazole (1 g, 7 mmol) and tert-butyl N-(7-hydroxyheptyl)carbamate (1.6 g, 7 mmol) and PPh₃ (2.2 g, 8.4 mmol) in THF (20 mL) was added DIAD (1.7 g, 8.4 mmol, 1.6 mL) at 0° C. The mixture was stirred at 25° C. for 4 h. TLC showed the starting material was consumed completely and one new spot formed. The reaction mixture was concentrated under reduced pressure to remove solvent. The residue was diluted with ethyl acetate (50 mL) and washed with water (30 mL). The organic layers were washed with brine (20 mL), dried over Na₂SO₄, filtered, and concentrated under reduced pressure to give a residue. The crude product was purified by column chromatography (SiO₂, petroleum ether/ethyl acetate=20:1 to 3:1 gradient). Intermediate 15, tert-butyl N-[7-(3-methoxy-4-nitro-pyrazol-1-yl)heptyl]carbamate (2 g, 80% yield), was obtained as a yellow oil and used for the next step without further purification.

### Preparation of Intermediate 16

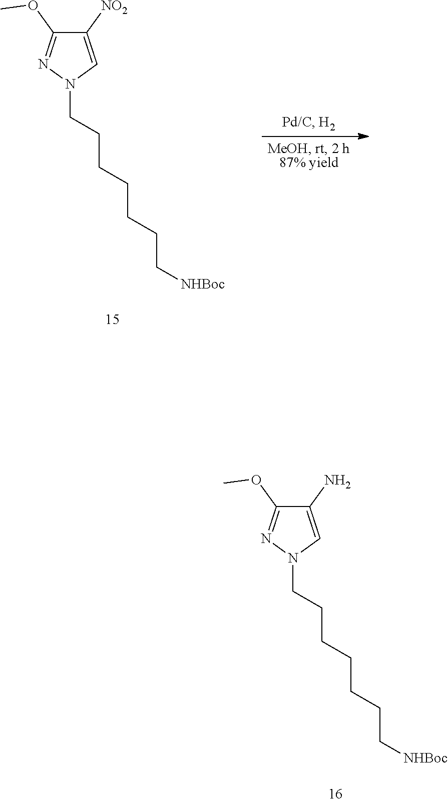 [SMILES: COc1nn(CCCCCCCNC(=O)OC(C)(C)C)cc1[N+](=O)[O-] ; "MeOH, rt, 2 h87% yield" ; "Pd/C, H2" ; COc1nn(CCCCCCCNC(=O)OC(C)(C)C)cc1N]

To a solution of tert-butyl N-[7-(3-methoxy-4-nitro-pyrazol-1-yl)heptyl]carbamate (2 g, 5.6 mmol) in MeOH (20 mL) was added Pd/C (50 mg, 5.1 mmol, 10% purity) under N₂. The suspension was degassed under vacuum and purged with H₂ several times. The mixture was stirred under H₂ (113.4 mg, 56.1 mmol) (15 psi) at 25° C. for 2 h. LCMS showed the desired mass was detected. The reaction mixture was filtered and the filtrate was concentrated in vacuo. The crude product was used into the next step without further purification. Intermediate 16, tert-butyl N-[7-(4-amino-3-methoxy-pyrazol-1-yl)heptyl]carbamate (1.6 g, 87% yield) was obtained as a brown-black oil. MS (ESI) m/z: 327.2 (M+H)+, RT=0.461 min.

### Preparation of Intermediate 17

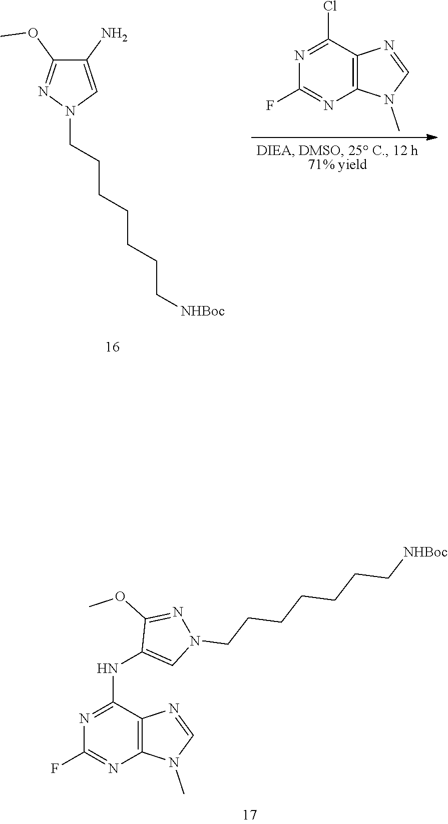 [SMILES: COc1nn(CCCCCCCNC(=O)OC(C)(C)C)cc1N ; Cn1cnc2c(Cl)nc(F)nc21 ; "DIEA, DMSO, 25° C., 12 h71% yield" ; COc1nn(CCCCCCCNC(=O)OC(C)(C)C)cc1Nc1nc(F)nc2c1ncn2C]

To a solution of tert-butyl N-[7-(4-amino-3-methoxy-pyrazol-1-yl)heptyl]carbamate (1.0 g, 3.1 mmol) in DMSO (15 mL) was added DIEA (989.81 mg, 7.7 mmol) and 6-chloro-2-fluoro-9-methyl-purine (628.7 mg, 3.4 mmol). The mixture was stirred at 25° C. for 12 h. LCMS showed the desired compound was detected. TLC showed the starting material was consumed completely and one new spot was formed. The reaction mixture was diluted with water (30 mL) then extracted with ethyl acetate (50 mL×2). The organic layers were washed with brine (30 mL×2), dried over Na₂SO₄, and then concentrated in vacuo. The crude product was purified by column chromatography (SiO₂, petroleum ether/ethyl acetate=10:1 to 1:2 gradient). Intermediate 17, tert-butyl N-[7-[4-[(2-fluoro-9-methyl-purin-6-yl)amino]-3-methoxy-pyrazol-1-yl]heptyl]carbamate (1.05 g, 72% yield) was obtained as a brown oil. 1H NMR (400 MHz, CDCl3): δ (ppm) 1.27 (6H, s), 1.36 (9H, s), 1.38-1.44 (2H, m), 1.70-1.83 (2H, m), 2.96-3.08 (2H, m), 3.73 (H, s), 3.85-3.91 (5H, m), 4.49 (1H, s), 7.54 (1H, s), 7.66 (1H, s), 7.94 (1H, s). MS (ESI) m/z: 477.3 (M+H)+, RT=0.675 min.

### Preparation of Intermediate 18

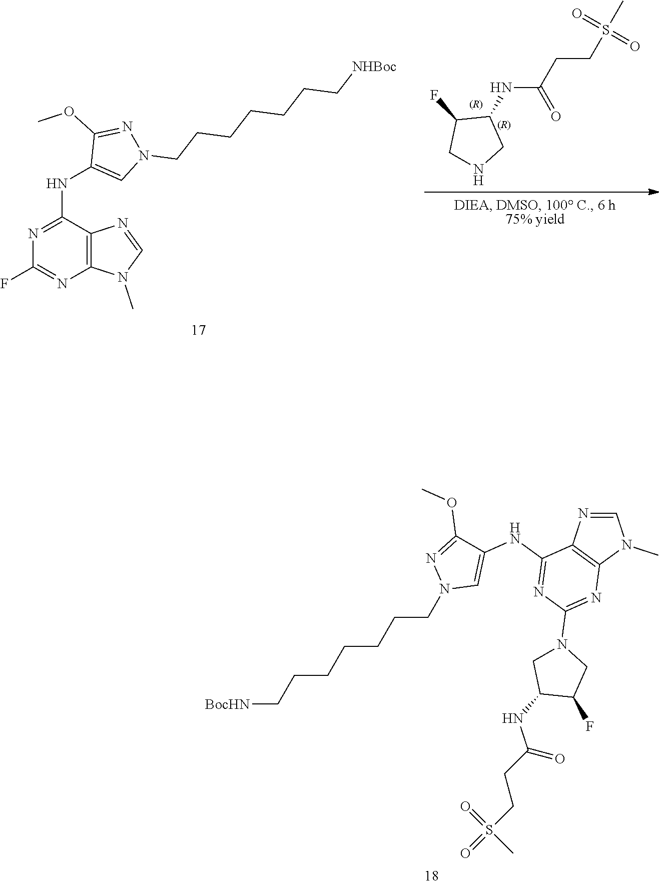

To a solution of tert-butyl N-[7-[4-[(2-fluoro-9-methyl-purin-6-yl)amino]-3-methoxy-pyrazol-1-yl]heptyl]carbamate (1 g, 2 mmol) and N-[(3R,4R)-4-fluoropyrrolidin-3-yl]-3-methylsulfonyl-propanamide (602 mg, 2.2 mmol) in DMSO (10 mL) was added DIEA (644.1 mg, 5 mmol). The mixture was stirred at 100° C. for 6 h. LCMS showed the desired compound was detected. The residue was diluted with water (50 mL) then extracted with ethyl acetate (30 mL×2). The organic layers were washed with brine (30 mL×2), dried over Na₂SO₄, and then concentrated in vacuo. The crude product was purified by column chromatography (SiO₂, petroleum ether/ethyl acetate=1:1 to 0:1 gradient). Intermediate 18, tert-butyl N-[7-[4-[[2-[(3R,4R)-3-fluoro-4-(3-methylsulfonylpropanoylamino)pyrrolidin-1-yl]-9-methyl-purin-6-yl]amino]-3-methoxy-pyrazol-1-yl]heptyl]carbamate (1.1 g, 75.4% yield) was obtained as a brown oil. 1H NMR (400 MHz, DMSO-d6) δ (ppm) 1.20-1.41 (19H, m), 1.66-1.79 (2H, m), 1.99 (1H, s), 2.88 (2H, q, J=6.8 Hz), 2.97 (3H, s), 3.32 (2H, s), 3.62 (3H, s), 3.66-3.81 (3H, m), 3.83 (3H, s), 3.94 (2H, t, J=6.8 Hz), 4.37 (1H, t, J=11.6), 4.98-5.24 (1H, m), 6.74 (1H, t, J=5.2 Hz), 7.76-7.89 (2H, m), 8.06 (1H, s), 8.45 (1H, d, J=6.8 Hz). MS (ESI) m/z: 695.4 (M+H)+, RT=0.598 min.

### Preparation of Intermediate 19

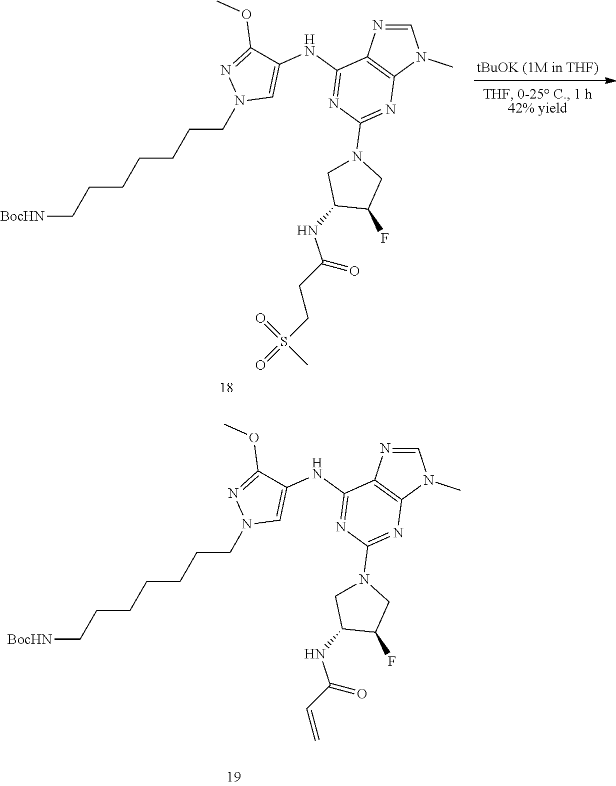 [SMILES: COc1nn(CCCCCCCNC(=O)OC(C)(C)C)cc1Nc1nc(N2C[C@@H](F)[C@H](NC(=O)CCS(C)(=O)=O)C2)nc2c1ncn2C ; "THF, 0-25° C., 1 h42% yield" ; "tBuOK (1M in THF)" ; C=CC(=O)N[C@@H]1CN(c2nc(Nc3cn(CCCCCCCNC(=O)OC(C)(C)C)nc3OC)c3ncn(C)c3n2)C[C@H]1F]

To a solution of tert-butyl N-[7-[4-[[2-[(3R,4R)-3-fluoro-4-(3-methylsulfonylpropanoylamino)pyrrolidin-1-yl]-9-methyl-purin-6-yl]amino]-3-methoxy-pyrazol-1-yl]heptyl]carbamate (0.8 g, 1.2 mmol) in THF (5 mL) was added t-BuOK (1 M, 3.45 mL) at 0° C. The mixture was stirred at 25° C. for 1 h. LCMS showed the desired compound was detected. TLC showed the starting material was consumed completely and one new spot was formed. The reaction mixture was diluted with H₂O (50 mL) and extracted with ethyl acetate (30 mL×2). The organic layers were washed with brine (30 ml×2), dried over Na₂SO₄, filtered, and concentrated under vacuum to give the crude product. The crude material was purified by column chromatography (SiO₂, petroleum ether/ethyl acetate=10:1 to 0:1 gradient). Intermediate 19, tert-butyl N-[7-[4-[[2-[(3R,4R)-3-fluoro-4-(prop-2-enoylamino)pyrrolidin-1-yl]-9-methyl-purin-6-yl]amino]-3-methoxy-pyrazol-1-yl]heptyl]carbamate (0.3 g, 42.39% yield), was obtained as a brown gum. 1H NMR (400 MHz, CDCl₃): δ (ppm) 1.18-1.48 (18H, m), 1.33-1.34 (1H, m), 1.77-1.83 (2H, m), 3.06 (2H, d, J=6.0 Hz), 3.59-3.73 (3H, m), 3.81-4.08 (9H, m), 4.46-4.85 (2H, m), 5.17-5.39 (1H, m), 5.68 (1H, d, J=10.0 Hz), 6.17 (1H, d, J=10.0 Hz), 6.38 (1H, d, J=17.2 Hz), 7.42-7.57 (1H, m), 7.85 (1H, s). MS (ESI) m/z: 615.4 (M+H)⁺, RT=0.617 min.

### Preparation of Intermediate 20

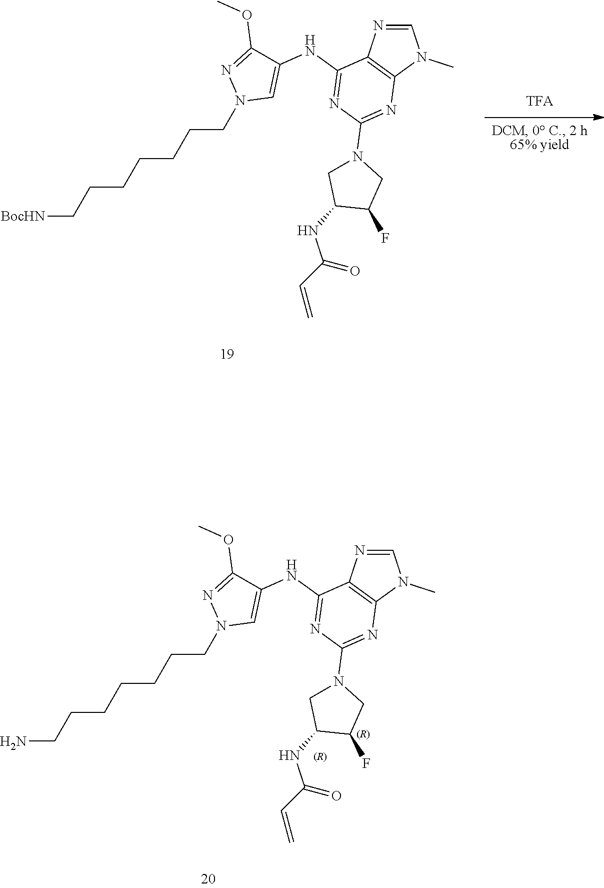

To a solution of tert-butyl N-[7-[4-[[2-[(3R,4R)-3-fluoro-4-(prop-2-enoylamino)pyrrolidin-1-yl]-9-methyl-purin-6-yl]amino]-3-methoxy-pyrazol-1-yl]heptyl]carbamate (300 mg, 488 umol) in DCM (10 mL) was added TFA (3.1 g, 27 mmol, 2 mL). The mixture was stirred at 0° C. for 2 h. LCMS showed the desired compound was detected. The reaction mixture was concentrated in vacuo. The crude product was used for the next step without further purification. Intermediate 20, N-[(3R,4R)-1-[6-[[1-(7-aminoheptyl)-3-methoxy-pyrazol-4-yl]amino]-9-methyl-purin-2-yl]-4-fluoro-pyrrolidin-3-yl]prop-2-enamide (200 mg, 65.2% yield), was obtained as a brown oil. MS (ESI) m/z: 515.4 (M+H)⁺, RT=0.447 min.

### Preparation of Intermediate 21

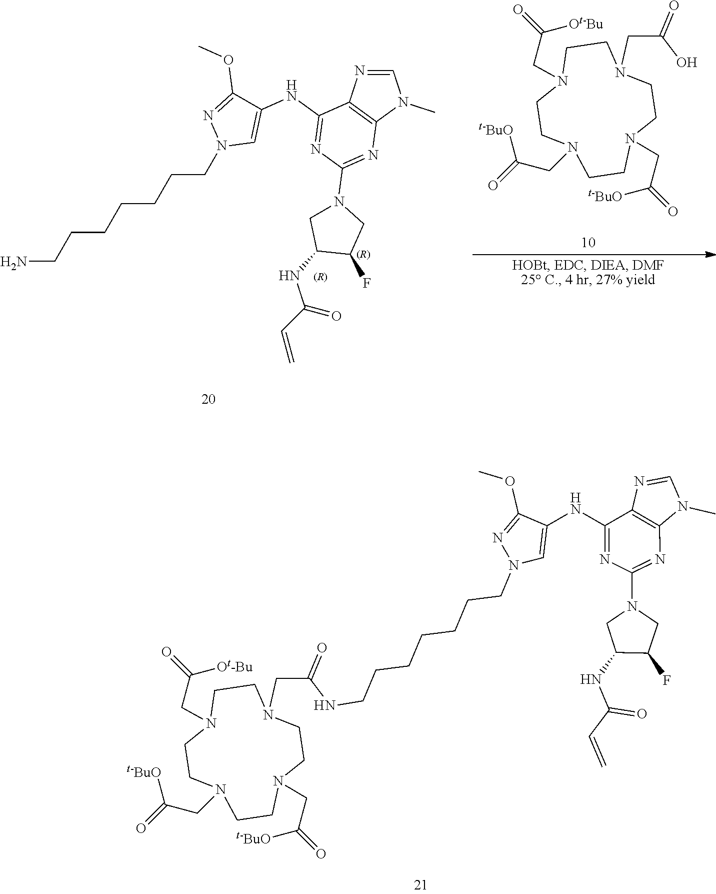

To a solution of N-[(3R,4R)-1-[6-[[1-(7-aminoheptyl)-3-methoxy-pyrazol-4-yl]amino]-9-methyl-purin-2-yl]-4-fluoro-pyrrolidin-3-yl]prop-2-enamide (200 mg, 388.7 umol) and HOBt (131.3 mg, 971.6 umol) and DIEA (125.6 mg, 971.6umol) in DMF (5 mL) was added 2-[4,7,10-tris(2-tert-butoxy-2-oxo-ethyl)-1,4,7,10-tetrazacyclododec-1-yl]acetic acid (222.6 mg, 388.7 umol) and EDCI (186.3 mg, 971.6 umol, 2.5 eq). The mixture was stirred at 25° C. for 4 h. LCMS showed the desired compound was detected. The reaction mixture was adjusted pH=5 using formic acid. The residue was purified by prep-HPLC (column: Phenomenex Synergi C18 150*25 mm* 10 um; mobile phase: [water(FA)-ACN]; B %: 18%-48%, 10 min). Intermediate 21, tert-butyl 2-[4,7-bis(2-tert-butoxy-2-oxo-ethyl)-10-[2-[7-[4-[[2-[(3R,4R)-3-fluoro-4-(prop-2-enoylamino)pyrrolidin-1-yl]-9-methyl-purin-6-yl]amino]-3-methoxy-pyrazol-1-yl]heptylamino]-2-oxo-ethyl]-1,4,7,10-tetrazacyclododec-1-yl]acetate (120 mg, 27.4% yield), was obtained as a white solid. 1H NMR (400 MHz, CDCl₃): δ (ppm) 1.23-1.55 (37H, m), 1.70-1.88 (4H, m), 2.52-3.05 (14H, m), 3.10-3.55 (10H, m), 3.61-3.75 (4H, m), 3.78-3.84 (1H, m), 3.90-4.12 (8H, m), 4.59-4.95 (1H, m), 5.29-5.78 (1H, m), 6.21-6.44 (1H, m), 7.14-7.21 (1H, m), 7.42-7.50 (1H, m), 7.92-8.11 (1H, m). MS (ESI) m/z: 1069.8 (M+H)⁺, RT=0.583 min.

### Preparation of Compound 003

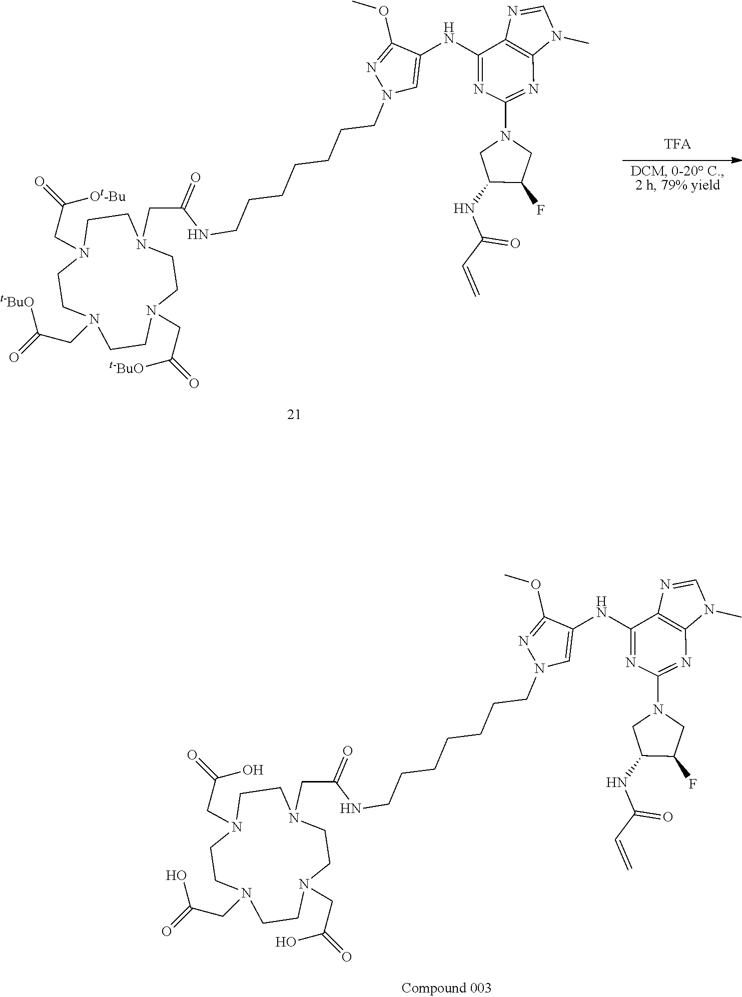 [SMILES: "TFA" ; C=CC(=O)N[C@@H]1CN(c2nc(Nc3cn(CCCCCCCNC(=O)CN4CCN(CC(=O)OC(C)(C)C)CCN(CC(=O)OC(C)(C)C)CCN(CC(=O)OC(C)(C)C)CC4)nc3OC)c3ncn(C)c3n2)C[C@H]1F ; "DCM, 0-20° C.,2 h, 79% yield" ; C=CC(=O)N[C@@H]1CN(c2nc(Nc3cn(CCCCCCCNC(=O)CN4CCN(CC(=O)O)CCN(CC(=O)O)CCN(CC(=O)O)CC4)nc3OC)c3ncn(C)c3n2)C[C@H]1F]

To a solution of tert-butyl 2-[4,7-bis(2-tert-butoxy-2-oxo-ethyl)-10-[2-[7-[4-[[2-[(3R,4R)-3-fluoro-4-(prop-2-enoylamino)pyrrolidin-1-yl]-9-methyl-purin-6yl]amino]-3-methoxy-pyrazol-1-yl]heptylamino]-2-oxo-ethyl]-1,4,7,10-tetrazacyclododec-1-yl]acetate (120 mg, 112.22 umol) in CH₂Cl₂ (2.5 mL) was added TFA (920 mg, 8.1 mmol) at 0° C. The mixture was stirred at 25° C. for 2 h. LCMS showed the desired mass was detected. The reaction mixture was concentrated in vacuo. The residue was purified by prep-HPLC (column: Phenomenex Synergi C18 150*25 mm* 10 um; mobile phase: [water(FA)-ACN]; B %: 13%-37%, 8 min). Compound 003, 2-[4,7-bis(carboxymethyl)-10-[2-[7-[4-[[2-[(3R,4R)-3-fluoro-4-(prop-2-enoylamino)pyrrolidin-1-yl]-9-methyl-purin-6-yl]amino]-3-methoxy-pyrazol-1-yl]heptylamino]-2-oxo-ethyl]-1,4,7,10-tetrazacyclododec-1-yl]acetic acid (80 mg, 79% yield), was obtained as a white solid. 1H NMR (400 MHz, CD₃OD): δ (ppm) 1.31 (6H, s), 1.48 (2H, s), 1.78-1.87 (2H, m), 2.10-2.33 (4H, m), 2.63-2.95 (6H, m), 2.99-3.18 (6H, m), 3.34-3.44 (3H, m), 3.44-3.60 (3H, m), 3.68-3.76 (5H, m), 3.78-3.93 (3H, m), 3.93-4.06 (8H, m), 4.59-4.66 (1H, m), 5.11-5.32 (1H, m), 5.64-5.73 (1H, m), 6.25-6.34 (2H, m), 7.77-7.89 (1H, m), 8.06-8.14 (1H, m). MS (ESI) m/z: 901.6 (M+H)⁺, RT=0.458 min.

### Preparation of Compound 003-Lu

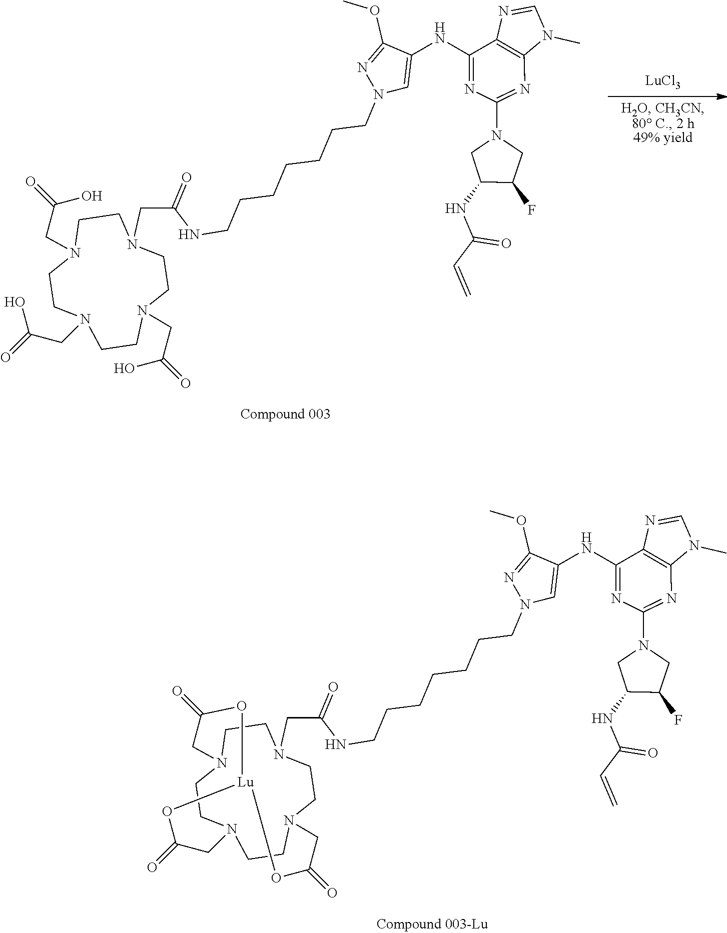

To a solution of 2-[4,7-bis(carboxymethyl)-10-[2-[7-[4-[[2-[(3R,4R)-3-fluoro-4-(prop-2-enoylamino)pyrrolidin-1-yl]-9-methylpurin-6-yl]amino]-3-methoxy-pyrazol-1-yl]heptylamino]-2-oxo-ethyl]-1,4,7,10-tetrazacyclododec-1-yl]acetic acid (50 mg, 55.5 umol) in CH₃CN (2 mL) and H₂O (2 mL) was added LuCl₃ (15.5 mg, 55.5umol). The mixture was stirred at 80° C. for 12 h. LCMS showed the desired compound was detected. The reaction mixture was concentrated in vacuo. The residue was purified by prep-HPLC (column: Phenomenex Synergi C18 150*25 mm*10 um; mobile phase: [water(FA)-ACN]; B %: 5%-35%, 10 min). Compound 003-Lu, N-[(3R,4R)-4-fluoro-1-[6-[[3-methoxy-1-[7-[[2-(3,16,19-trioxo-2,17,18-trioxa-5,8,11,14-tetraza-1λ3-lutetatricyclo[9.6.3.25,14]docosan-8-yl)acetyl]amino]heptyl]pyrazol-4-yl]amino]-9-methyl-purin-2-yl]pyrrolidin-3-yl]prop-2-enamide (30 mg, 49.4% yield), was obtained as a white solid. 1H NMR (400 MHz, CD₃OD): δ (ppm) 1.35 (6H, s), 1.54 (2H, s), 1.76-1.87 (2H, m), 2.20-2.90 (14H, m), 3.12-3.22 (4H, m), 3.37 (4H, d, J=8.8 Hz), 3.56-3.67 (2H, m), 3.72 (3H, s), 3.74-3.95 (4H, m), 3.97 (3H, s), 3.98-4.05 (4H, m), 4.62 (1H, d, J=5.2 Hz), 5.09-5.31 (1H, m), 5.69 (1H, d, J=4.8 Hz), 6.25-6.34 (2H, m), 7.75 (1H, s), 8.06-8.12 (1H, m). MS (ESI) m/z: 537.4 (M/2+H)⁺, RT=0.472 min.

### Example A8: Synthesis of Compound 004 and Compound 004-Lu

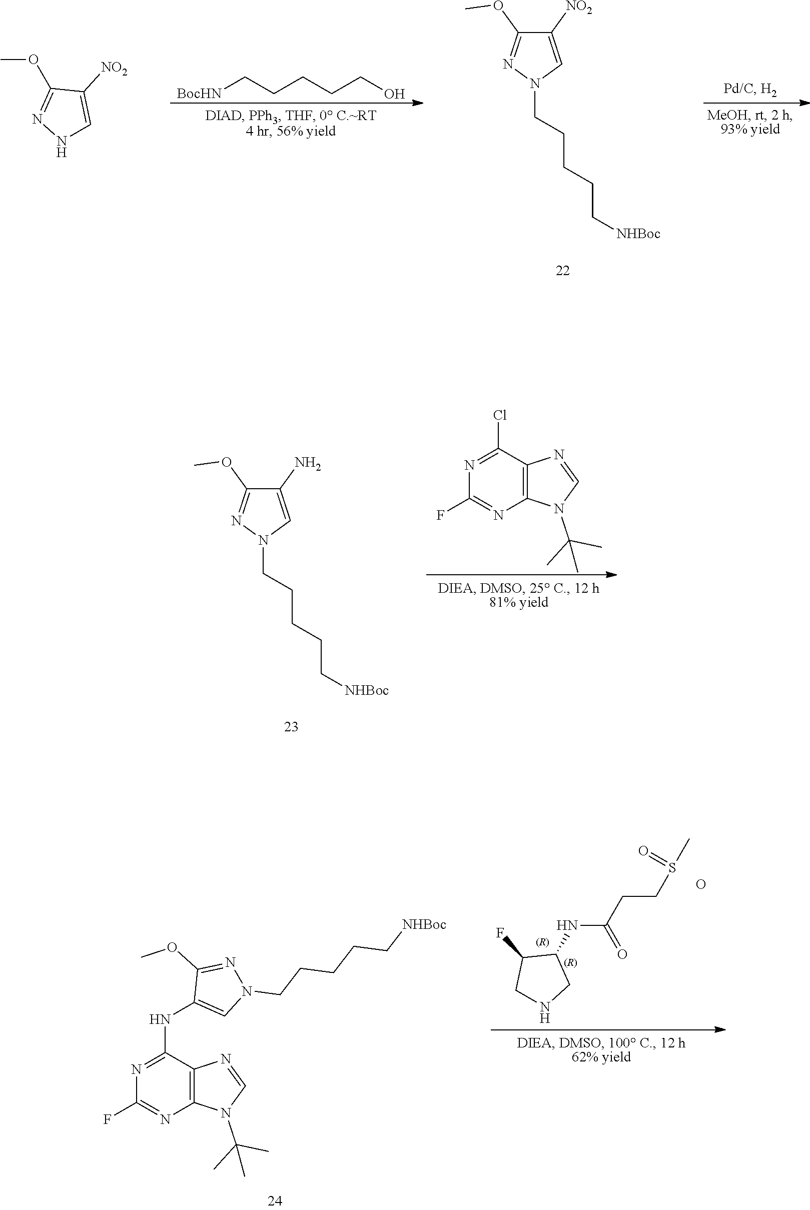

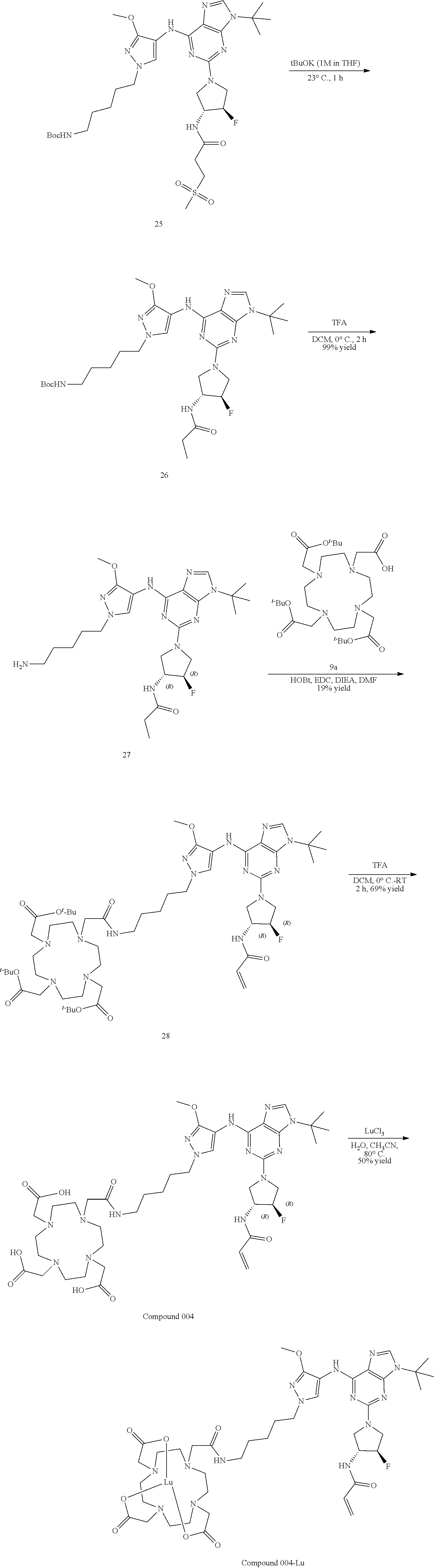

### Preparation of Intermediate 22

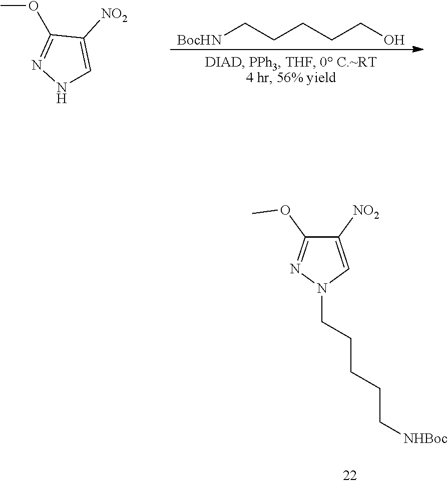

To a solution of 3-methoxy-4-nitro-1H-pyrazole (1 g, 7 mmol) and tert-butyl N-(5-hydroxypentyl)carbamate (1.4 g, 7 mmol, 1.4 mL) and PPh₃ (2.2 g, 8.4 mmol) in THF (20 mL) was added DIAD (1.7 g, 8.4 mmol, 1.6 mL) at 0° C. The mixture was stirred at 25° C. for 4 h. TLC showed the starting material was consumed completely and one new spot was formed. The reaction mixture was concentrated under reduced pressure to remove solvent. The residue was diluted with ethyl acetate (50 mL) and washed with water (30 mL). The organic layers were washed with brine (20 mL) dried over Na₂SO₄, filtered, and concentrated under reduced pressure to give a residue. The crude product was purified by column chromatography (SiO₂, petroleum ether/ethyl acetate=20:1 to 3:1 gradient). Intermediate 22, tert-butyl N-[5-(3-methoxy-4-nitro-pyrazol-1-yl)pentyl]carbamate (1.3 g, 57% yield) was obtained as a brown oil. 1H NMR (400 MHz, CDCl3): δ (ppm) 1.16-1.49 (2H, m), 1.30-1.38 (2H, m), 1.44 (9H, s), 1.90 (2H, q, J=7.4 Hz), 3.97 (2H, t, J=7.2 Hz), 4.04 (3H, s), 4.36-4.67 (1H, m), 7.98 (1H, s).

### Preparation of Intermediate 23

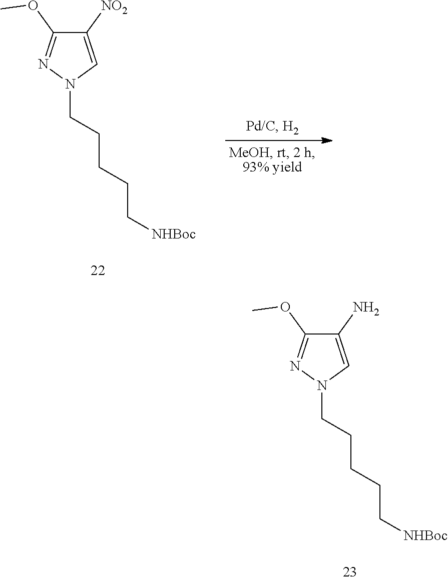 [SMILES: COc1nn(CCCCCNC(=O)OC(C)(C)C)cc1[N+](=O)[O-] ; "MeOH, rt, 2 h,93% yield" ; "Pd/C, H2" ; COc1nn(CCCCCNC(=O)OC(C)(C)C)cc1N]

To a solution of tert-butyl N-[5-(3-methoxy-4-nitro-pyrazol-1-yl)pentyl]carbamate (1.3 g, 4 mmol) in MeOH (20 mL) was added Pd/C (50 mg, 4 mmol) under N₂. The suspension was degassed under vacuum and purged with H₂ several times. The mixture was stirred under H₂ (79.97 mg, 39.56 mmol) (15 psi) at 25° C. for 2 h. LCMS showed the desired compound was detected. The reaction mixture was filtered and the filtrate was concentrated in vacuo. The crude product was used for the next step without further purification. Intermediate 23, tert-butyl N-[5-(4-amino-3-methoxy-pyrazol-1-yl)pentyl]carbamate (1.1 g, 93% yield), was obtained as a brown oil. MS (ESI) m/z: 299.1 (M+H)⁺, RT=0.417 min.

### Preparation of Intermediate 24

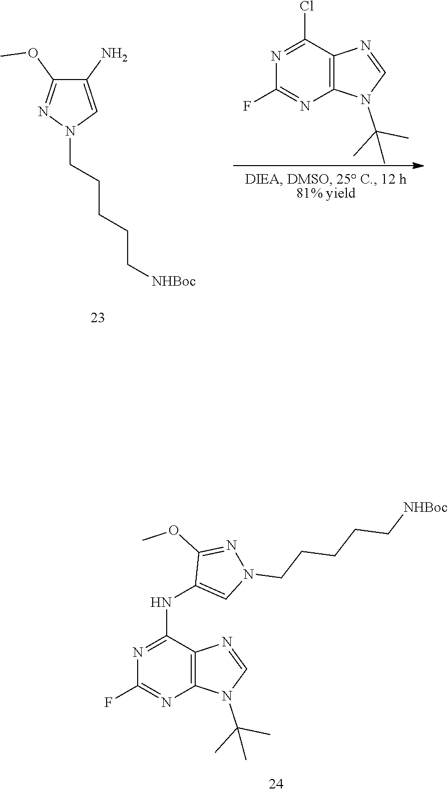

To a solution of tert-butyl N-[5-(4-amino-3-methoxy-pyrazol-1-yl)pentyl]carbamate (150 mg, 502.7 umol) in DMSO (5 mL) was added DIEA (162.4 mg, 1.3 mmol) and 9-tert-butyl-6-chloro-2-fluoro-purine (115 mg, 502.7 umol). The mixture was stirred at 25° C. for 12 h. LCMS showed the desired compound was detected. TLC showed the starting material was consumed completely and one new spot was formed. The reaction mixture was diluted with water (30 mL) then extracted with ethyl acetate (30 mL×2). The organic layers were washed with brine (30 mL×2), dried over Na₂SO₄, and then concentrated in vacuo. The crude product was purified by column chromatography (SiO₂, petroleum ether/ethyl acetate=20:1 to 1:1 gradient). Intermediate 24, tert-butyl N-[5-[4-[(9-tert-butyl-2-fluoro-purin-6-yl)amino]-3-methoxy-pyrazol-1-yl]pentyl]carbamate (200 mg, 81% yield) was obtained as a brown-black oil. 1H NMR (400 MHz, DMSO-d₆) δ (ppm) 1.30-1.38 (2H, m), 1.43 (9H, s), 1.48-1.56 (2H, m), 1.79 (9H, s), 1.86 (H, q, J=7.6 Hz), 3.03-3.19 (2H, m), 3.93-4.01 (5H, m), 4.57 (1H, s), 7.73 (1H, s), 7.89-7.94 (1H, m), 8.00 (1H, s). MS (ESI) m/z: 491.3 (M+H)⁺, RT=0.878 min.

### Preparation of Intermediate 25

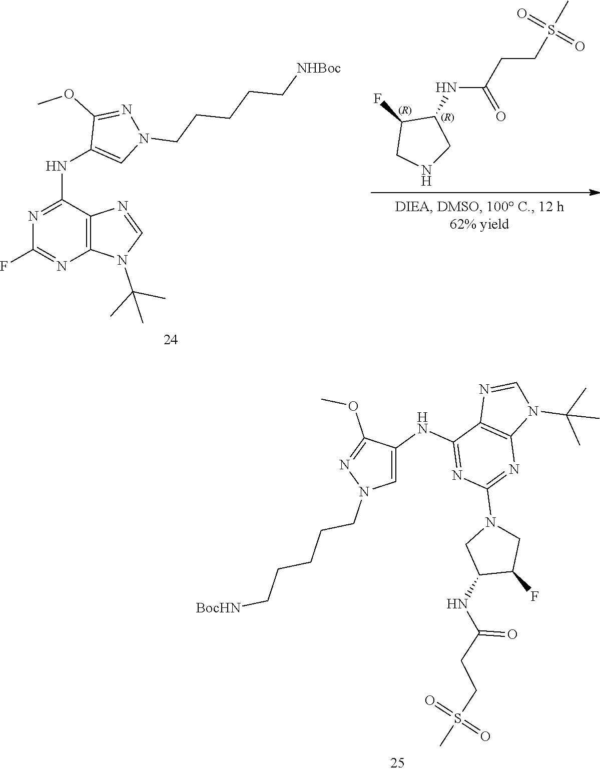

To a solution of tert-butyl N-[5-[4-[(9-tert-butyl-2-fluoro-purin-6-yl)amino]-3-methoxy-pyrazol-1-yl]pentyl]carbamate (200 mg, 407.7 umol) in DMSO (5 mL) was added DIEA (158.1 mg, 1.2 mmol, 213 uL) and N-[(3R,4R)-4-fluoropyrrolidin-3-yl]-3-methylsulfonyl-propanamide (146 mg, 530.4 umol). The mixture was stirred at 100° C. for 12 h. TLC showed the starting material remained and one new spot was formed. The reaction mixture was diluted with water (30 mL) then extracted with ethyl acetate (30 mL×2). The organic layers were washed with brine (30 mL×2), dried over Na₂SO₄, and then concentrated in vacuo. The crude product was purified by column chromatography (SiO₂, petroleum ether/ethyl acetate=10:1 to 0:1 gradient). Intermediate 25, tert-butyl N-[5-[4-[[9-tert-butyl-2-[(3R,4R)-3-fluoro-4-(3-methylsulfonylpropanoylamino)pyrrolidin-1-yl]purin-6-yl]amino]-3-methoxy-pyrazol-1-yl]pentyl]carbamate (180 mg, 62% yield), was obtained as a brown oil. 1H NMR (400 MHz, CDCl₃): δ (ppm) 1.28 (9H, d, J=2.4 Hz), 1.43 (6H, s), 1.74 (9H, s), 1.78-1.79 (1H, m), 2.95 (3H, s), 2.99 (1H, s), 3.02-3.22 (4H, m), 3.36-3.46 (2H, m), 3.96-3.99 (7H, m), 4.57-4.71 (2H, m), 5.11-5.30 (1H, m), 7.53 (1H, s), 7.66 (1H, s), 7.81-8.06 (2H, m). MS (ESI) m/z: 709.5 (M+H)⁺, RT=0.601 min.

### Preparation of Intermediate 26

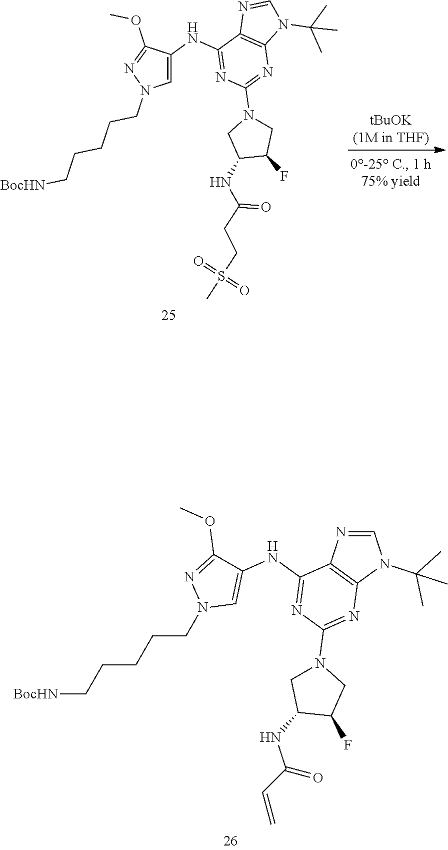 [SMILES: COc1nn(CCCCCNC(=O)OC(C)(C)C)cc1Nc1nc(N2C[C@@H](F)[C@H](NC(=O)CCS(C)(=O)=O)C2)nc2c1ncn2C(C)(C)C ; C=CC(=O)N[C@@H]1CN(c2nc(Nc3cn(CCCCCNC(=O)OC(C)(C)C)nc3OC)c3ncn(C(C)(C)C)c3n2)C[C@H]1F]

To a solution of tert-butyl N-[5-[4-[[9-tert-butyl-2-[(3R,4R)-3-fluoro-4-(3-methylsulfonylpropanoylamino)pyrrolidin-1-yl]purin-6-yl]amino]-3-methoxy-pyrazol-1-yl]pentyl]carbamate (180 mg, 253.9 umol) in THF (5 mL) was added t-BuOK (1 M, 761.8 uL) at 0° C. The mixture was stirred at 25° C. for 1 h. TLC showed the starting material was consumed completely and one new spot formed. The residue was diluted with water (20 mL) then extracted with ethyl acetate (20 mL×2). The organic layers were washed with brine (15 mL), dried over Na₂SO₄, filtered, and concentrated in vacuo. The residue was purified by prep-TLC (SiO₂, petroleum ether:ethyl acetate=0:1). Intermediate 26, tert-butyl N-[5-[4-[[9-tert-butyl-2-[(3R,4R)-3-fluoro-4-(prop-2-enoylamino)pyrrolidin-1-yl]purin-6-yl]amino]-3-methoxy-pyrazol-1-yl]pentyl] carbamate (120 mg, 75% yield) was obtained as a brown gum. 1H NMR (400 MHz, CDCl3): δ (ppm) 1.25-1.30 (9H, m), 1.38-1.49 (6H, m), 1.76 (9H, s), 3.02-3.17 (2H, m), 3.84-4.04 (9H, m), 4.52-4.86 (2H, m), 5.66 (1H, d, J=9.6 Hz), 6.11-6.22 (1H, m), 6.37 (1H, d, J=1.2 Hz), 7.56-7.68 (1H, m), 7.93-8.05 (1H, m). MS (ESI) m/z: 629.4 (M+H)⁺, RT=0.624 min.

### Preparation of Intermediate 27

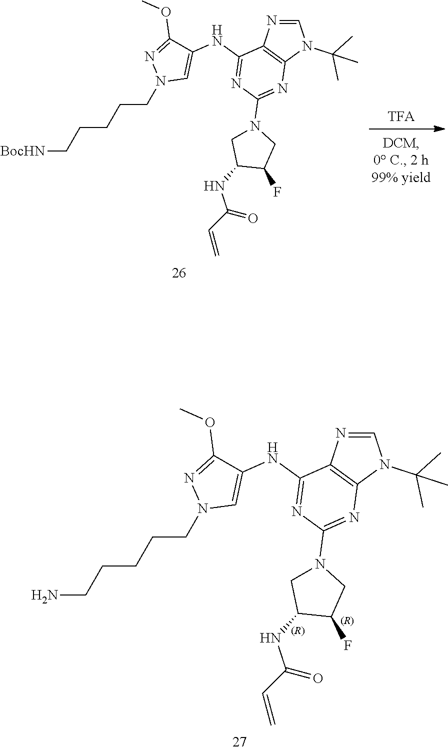

To a solution of tert-butyl N-[5-[4-[[9-tert-butyl-2-[(3R,4R)-3-fluoro-4-(prop-2-enoylamino)pyrrolidin-1-yl]purin-6-yl]amino]-3-methoxy-pyrazol-1-yl]pentyl]carbamate (120 mg, 190.9 umol) in DCM (10 mL) was added TFA (3.1 g, 27 mmol, 2 mL). The mixture was stirred at 0° C. for 2 h. LCMS showed the desired compound was detected. The reaction mixture was concentrated in vacuo. The crude product was used for the next step without further purification. Intermediate 27, N-[(3R,4R)-1-[6-[[1-(5-aminopentyl)-3-methoxy-pyrazol-4-yl]amino]-9-tert-butyl-purin-2-yl]-4-fluoro-pyrrolidin-3-yl]prop-2-enamide (0.1 g, 99% yield) was obtained as a brown oil. MS (ESI) m/z: 529.4 (M+H)⁺, RT=0.492 min.

### Preparation of Intermediate 28

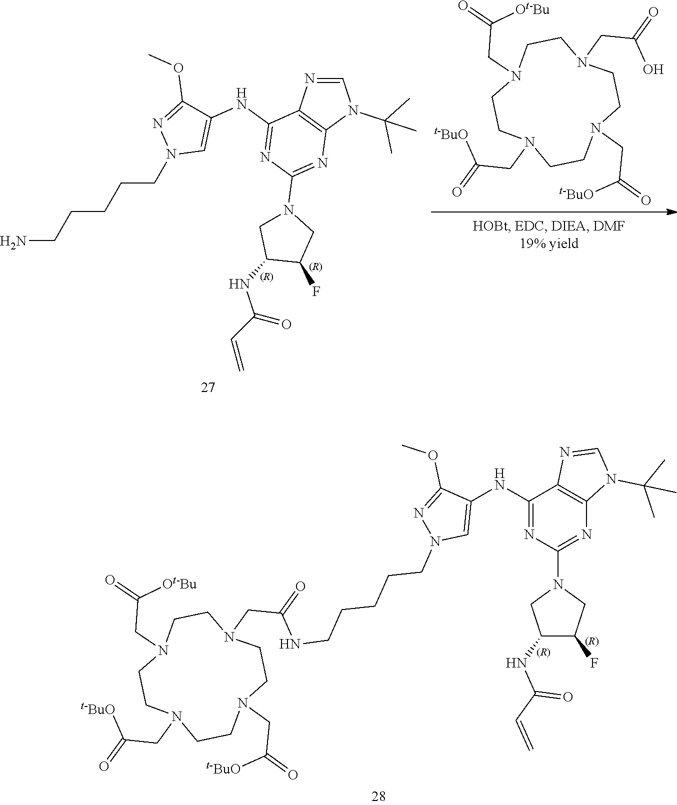

To a solution of N-[(3R,4R)-1-[6-[[1-(5-aminopentyl)-3-methoxy-pyrazol-4-yl]amino]-9-tert-butyl-purin-2-yl]-4-fluoropyrrolidin-3-yl]prop-2-enamide (100 mg, 189.2 umol) and HOBt (63.9 mg, 472.9 umol) and DIEA (61.1 mg, 472.9 umol) in DMF (5 mL) was added 2-[4,7,10-tris(2-tert-butoxy-2-oxo-ethyl)-1,4,7,10-tetrazacyclododec-1-yl]acetic acid (130 mg, 227 umol and EDC (90.7 mg, 472.9 umol). The mixture was stirred at 25° C. for 4 h. LCMS showed the desired mass was detected. The reaction mixture was adjusted pH=5 by formic acid. The residue was purified by prep-HPLC (column: Phenomenex Synergi C18 150*25 mm* 10 um; mobile phase: [water(FA)-ACN]; B %: 18%-48%, 10 min). Intermediate 28, tert-butyl 2-[4,7-bis(2-tert-butoxy-2-oxo-ethyl)-10-[2-[5-[4-[[9-tert-butyl-2-[(3R,4R)-3-fluoro-4-(prop-2-enoylamino)pyrrolidin-1-yl]purin-6-yl]amino]-3-methoxy-pyrazol-1-yl]pentylamino]-2-oxo-ethyl]-1,4,7,10-tetrazacyclododec-1-yl]acetate (40 mg, 19.5% yield), was obtained as a white solid. 1H NMR (400 MHz, CD₃OD): δ (ppm) 1.25-1.35 (6H, m), 1.43-1.46 (27H, m), 1.75 (16H, s), 2.68 (4H, s), 2.87 (6H, d, J=4.4 Hz), 3.11-3.30 (8H, m), 3.56 (2H, s), 3.89-4.05 (10H, m), 4.68-4.82 (1H, m), 5.54-5.63 (1H, m), 6.31-6.41 (2H, m), 7.30 (1H, s), 7.55-7.60 (1H, m), 8.06 (1H, s), 9.23 (1H, d, J=2.0 Hz). MS (ESI) m/z: 542.6 (M/2+H)⁺, RT=0.581 min.

### Preparation of Compound 004

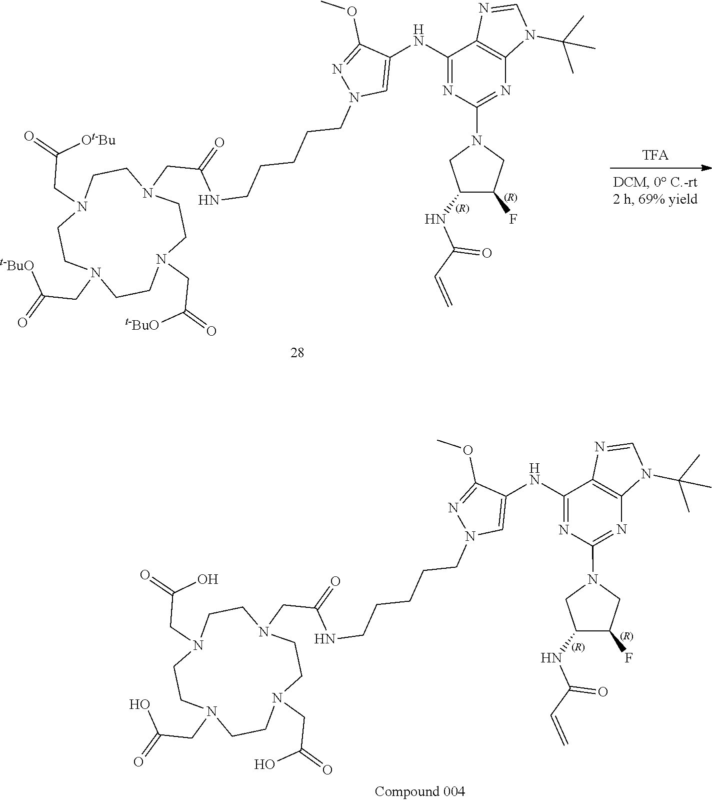

To a solution of tert-butyl 2-[4,7-bis(2-tert-butoxy-2-oxo-ethyl)-10-[2-[5-[4-[[9-tert-butyl-2-[(3R,4R)-3-fluoro-4-(prop-2-enoylamino)pyrrolidin-1-yl]purin-6-yl]amino]-3-methoxy-pyrazol-1-yl]pentylamino]-2-oxo-ethyl]-1,4,7,10-tetrazacyclododec-1-yl]acetate (30 mg, 27.7 umol) in DCM (3 mL) was added TFA (1.54 g, 13.5 mmol) at 0° C. The mixture was stirred at 25° C. for 1 h. LCMS showed 94% of the desired mass was detected. The reaction mixture was concentrated in vacuo and the residue was purified by prep-HPLC (column: Phenomenex Synergi C18 150*25 mm* 10 um; mobile phase: [water(FA)-ACN]; B %: 11%-41%, 10 min). Compound 004, 2-[4-[2-[5-[4-[[9-tert-butyl-2-[(3R,4R)-3-fluoro-4-(prop-2-enoylamino)pyrrolidin-1-yl]purin-6-yl]amino]-3-methoxy-pyrazol-1-yl]pentylamino]-2-oxo-ethyl]-7,10-bis(carboxymethyl)-1,4,7,10-tetrazacyclododec-1-yl]acetic acid (18 mg, 67.5% yield), was obtained as a white solid. 1H NMR (400 MHz, CD3OD): δ (ppm) 1.26-1.34 (2H, m), 1.49-1.59 (2H, m), 1.79 (8H, s), 1.81-1.89 (2H, m), 3.00 (8H, d, J=15.6 Hz), 3.17 (2H, t, J=6.8 Hz), 3.31-3.49 (11H, m), 3.56 (2H, s), 3.69 (H, s), 3.78-4.09 (8H, m), 4.63 (1H, d, J=5.2 Hz), 5.11-5.35 (1H, m), 5.68 (H, d, J=3.6 Hz), 6.22-6.35 (2H, m), 7.89 (1H, s), 8.10 (1H, s). MS (ESI) m/z: 915.8 (M/2+H)⁺, RT=0.791 min.

### Preparation of Compound 004-Lu

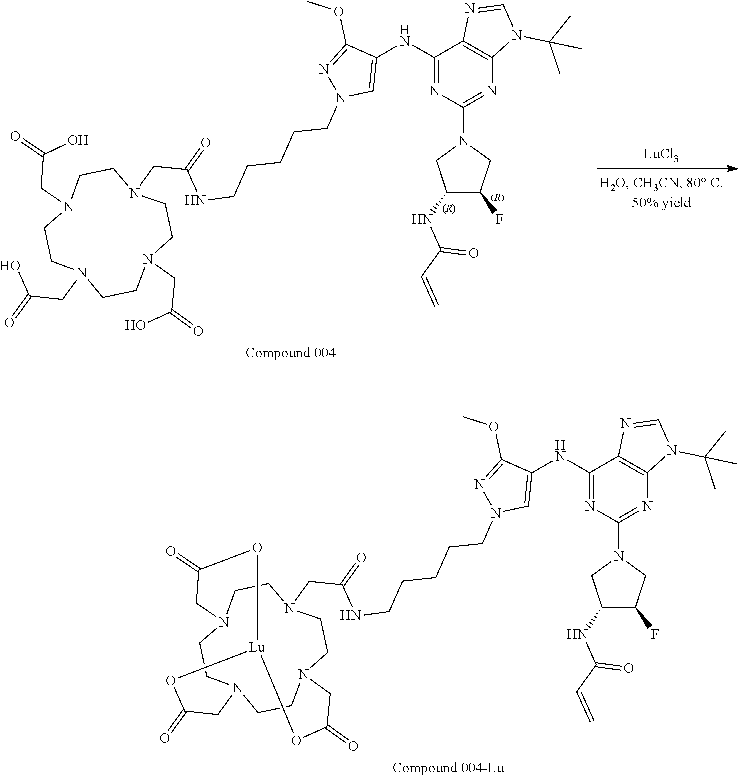

To a solution of 2-[4-[2-[5-[4-[[9-tert-butyl-2-[(3R,4R)-3-fluoro-4-(prop-2-enoylamino)pyrrolidin-1-yl]purin-6-yl]amino]-3-methoxy-pyrazol-1-yl]pentylamino]-2-oxo-ethyl]-7,10-bis(carboxymethyl)-1,4,7,10-tetrazacyclododec-1-yl]acetic acid (0.1 g, 97.2 umol, TFA) in MeCN (1 mL) and H₂O (1 mL) was added LuCl₃ (27.4 mg, 97.2 umol). The mixture was stirred at 80° C. for 2 h. LCMS showed the desired mass was detected. The residue was purified by prep-HPLC (column: Phenomenex C18 75*30 mm*3 um; mobile phase: [water(FA)-ACN]; B %: 18%-48%, 7 min). Compound 004-Lu, N-[(3R,4R)-1-[9-tert-butyl-6-[[3-methoxy-1-[5-[[2-(3,16,19-trioxo-2,17,18-trioxa-5,8,11,14-tetraza-1λ3-lutetatricyclo[9.6.3.25,14]docosan-8-yl)acetyl]amino]pentyl]pyrazol-4yl]amino]purin-2-yl]-4-fluoro-pyrrolidin-3-yl]prop-2-enamide (53 mg, 48.8 umol, 50% yield), was obtained as a white solid. 1H NMR (400 MHz, CD3OD): δ (ppm) 1.32 (2H, s), 1.50-1.66 (2H, m), 1.78 (9H, s), 1.83-1.96 (2H, m), 2.25-2.90 (12H, m), 3.01-3.29 (6H, m), 3.38 (4H, d, J=12.0 Hz), 3.54-3.69 (2H, m), 3.70-4.13 (11H, m), 4.59-4.70 (1H, m), 5.09-5.30 (1H, m), 5.68 (1H, d, J=2.4 Hz), 6.24-6.40 (2H, m), 7.87 (1H, s), 8.11 (1H, s). MS (ESI) m/z: 544.4 (M/2+H)+, RT=0.504 min.

### Example A9: Synthesis of Compound 005 and Compound 005-Lu

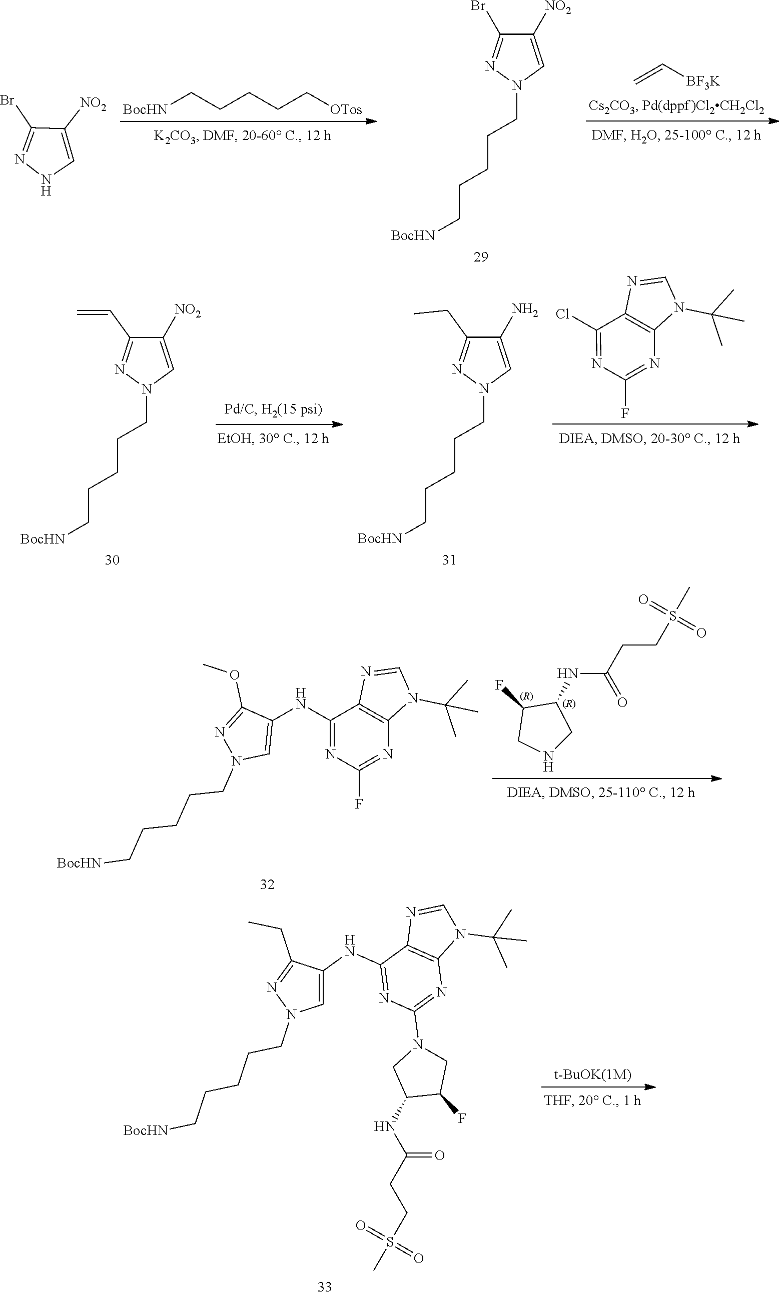

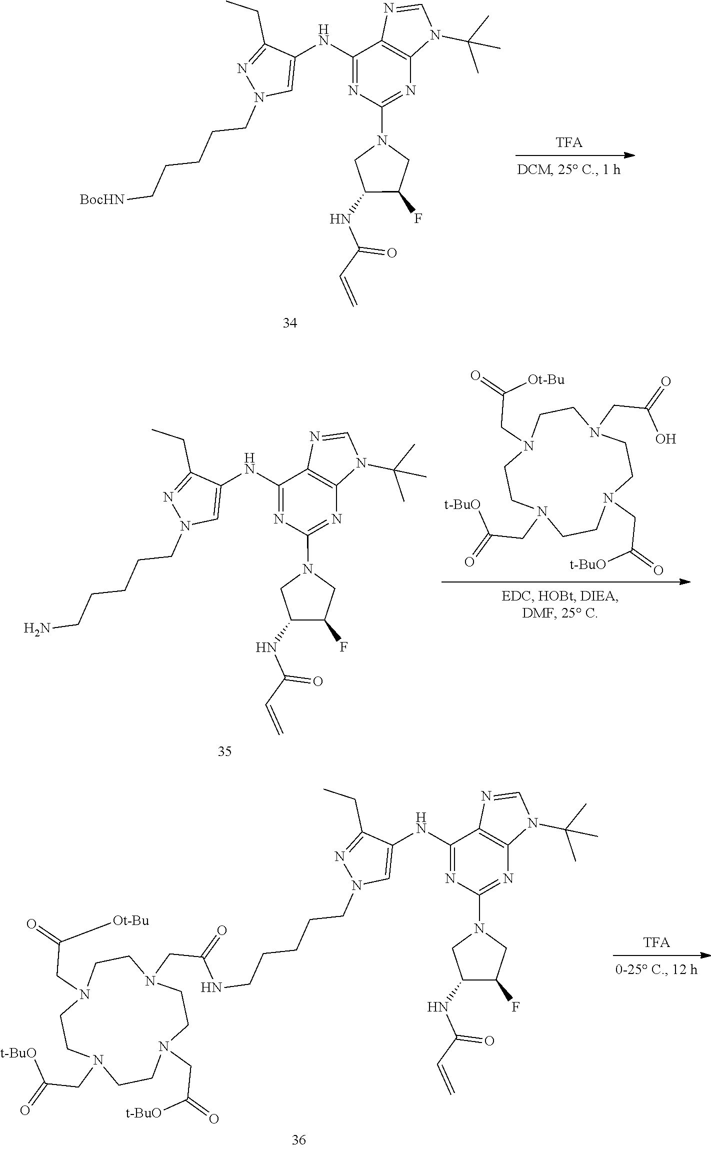 [SMILES: C=CC(=O)N[C@@H]1CN(c2nc(Nc3cn(CCCCCNC(=O)OC(C)(C)C)nc3CC)c3ncn(C(C)(C)C)c3n2)C[C@H]1F ; C=CC(=O)N[C@@H]1CN(c2nc(Nc3cn(CCCCCN)nc3CC)c3ncn(C(C)(C)C)c3n2)C[C@H]1F ; CC(C)(C)OC(=O)CN1CCN(CC(=O)O)CCN(CC(=O)OC(C)(C)C)CCN(CC(=O)OC(C)(C)C)CC1 ; C=CC(=O)N[C@@H]1CN(c2nc(Nc3cn(CCCCCNC(=O)CN4CCN(CC(=O)OC(C)(C)C)CCN(CC(=O)OC(C)(C)C)CCN(CC(=O)OC(C)(C)C)CC4)nc3CC)c3ncn(C(C)(C)C)c3n2)C[C@H]1F]

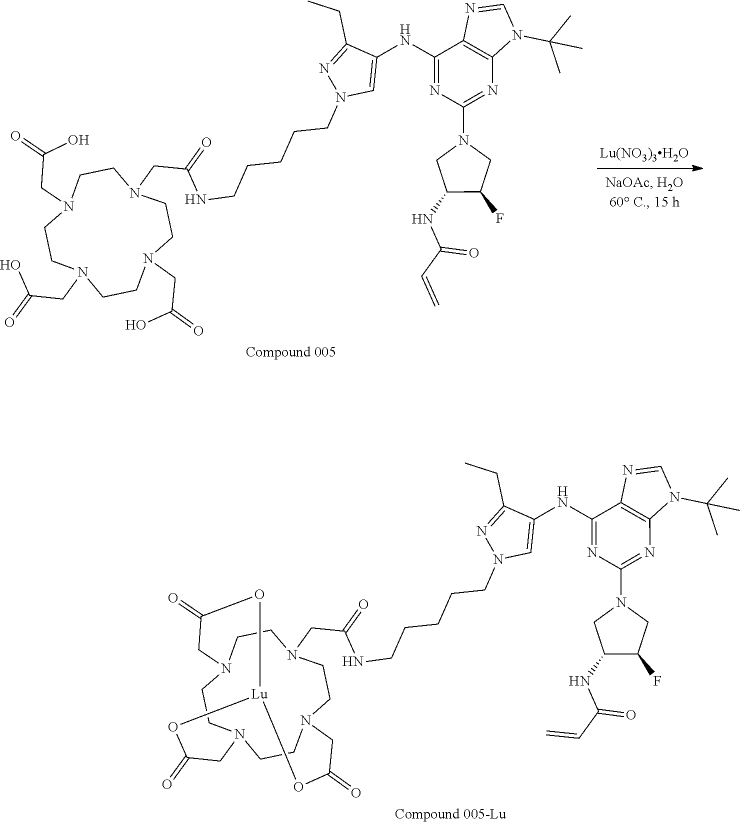

### Preparation of Intermediate 29

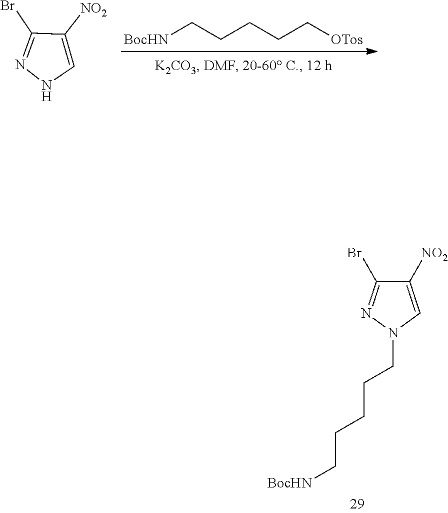

To a solution of 5-((tert-butoxycarbonyl)amino)pentyl 4-methylbenzenesulfonate (7.9 g, 21.1 mmol, 96% purity, 1 eq) in DMF (80 mL) was added 3-bromo-4-nitro-1H-pyrazole (4.1 g, 21.1 mmol, 1 eq) and K₂CO₃ (8.7 g, 63.2 mmol, 3 eq) at 25° C. The mixture was stirred at 60° C. for 12 h. TLC showed the starting material was consumed completely. The mixture was partitioned between ethyl acetate (200 mL)/water (300 mL) and layers were separated. The aqueous phase was extracted with ethyl acetate (200 mL×3). The combined organic layers were washed with brine (200 mL), dried over Na₂SO₄, filtered, and concentrated under vacuum. The residue was purified by prep-HPLC: column: Welch Xtimate C18 250×70 mm #10 um; mobile phase: [water(NH4HCO3)-CH3CN]; B %: 35%-65%, 20 min and lyophilization to provide intermediate 29, tert-butyl (5-(3-bromo-4-nitro-1H-pyrazol-1-yl)pentyl)carbamate (4.4 g, 8.3 mmol, 39.5% yield, 71.2% purity), as yellow oil. MS (ESI) m/z: 277.0 (M−100+H)+, RT=0.830 min.

### Preparation of Intermediate 30

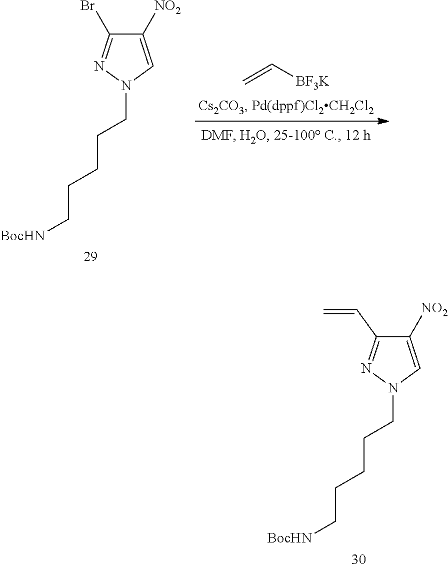

The solution of tert-butyl (5-(3-bromo-4-nitro-1H-pyrazol-1-yl)pentyl)carbamate (3.4 g, 9.1 mmol, 1 eq) in DMF (68 mL) and H₂O (14 mL) was added potassium vinyltrifluoroborate (3.6 g, 27 mmol, 3 eq), Cs₂CO₃ (8.8 g, 27 mmol, 3 eq) and Pd(dppf)Cl₂·CH₂Cl₂ (2.2 g, 2.7 mmol, 0.3 eq) under N₂ at 25° C. Then the mixture was stirred at 100° C. for 12 h. LCMS showed starting material was consumed. The mixture was partitioned between ethyl acetate (500 mL)/water (500 mL) and the layers were separated. The aqueous phase was extracted with ethyl acetate (500 mL×3). The combined organic layers were washed with brine (500 mL×2), dried over Na₂SO₄, filtered, and concentrated under vacuum. The residue was purified by column chromatography (SiO₂, petroleum ether:ethyl acetate=1:0 to 0:1 gradient) to provide intermediate 30, tert-butyl (5-(4-nitro-3-vinyl-1H-pyrazol-1-yl)pentyl)carbamate (3.35 g, crude), as yellow oil. 1H NMR (400 MHz, CDCl3) δ ppm 8.12 (s, 1H), 7.21 (dd, J=17.61, 11.13 Hz, 1H), 6.19 (dd, J=17.61, 1.59 Hz, 1H), 5.53 (dd, J=11.19, 1.53 Hz, 1H), 4.54 (br s, 1H), 4.22-4.11 (m, 2H), 3.13 (q, J=6.15 Hz, 2H), 1.94 (quin, J=7.43 Hz, 2H), 1.58-1.48 (m, 2H), 1.44 (s, 9H), 1.40-1.33 (m, 2H). MS (ESI) m/z: 225.2 (M−100+H)+, R.T.=0.837 min.

### Preparation of Intermediate 31

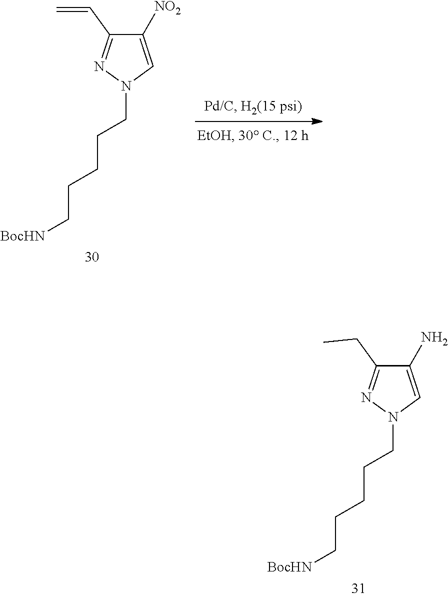 [SMILES: C=Cc1nn(CCCCCNC(=O)OC(C)(C)C)cc1[N+](=O)[O-] ; CCc1nn(CCCCCNC(=O)OC(C)(C)C)cc1N]

To a solution of tert-butyl N-[5-(4-nitro-3-vinyl-pyrazol-1-yl)pentyl]carbamate (4 g, 12.3 mmol, 1 eq) in EtOH (80 mL) was added Pd/C (4 g, 10% purity) under Ar. Then the suspension was degassed under vacuum and purged with H₂ several times. The mixture was stirred under H₂ (15 psi) at 30° C. for 12 h. TLC showed the starting material was consumed completely. The mixture was filtered and the filtrate was concentrated under reduced pressure to provide intermediate 31, tert-butyl N-[5-(4-amino-3-ethyl-pyrazol-1-yl)pentyl]carbamate (2.98 g, crude), as brown oil. The crude material was used for the next step without further purification. 1H NMR (400 MHz, CDCl3) δ ppm 6.93 (s, 1H), 4.55 (br s, 1H), 3.90-4.01 (m, 2H), 3.09 (m, 2H), 2.74 (br s, 2H), 2.52-2.62 (m, 2H), 1.73-1.85 (m, 2H), 1.43 (s, 11H), 1.20-1.34 (m, 5H). MS (ESI) m/z: 297.3 (M+H)⁺, R.T.=0.583 min.

### Preparation of Intermediate 32

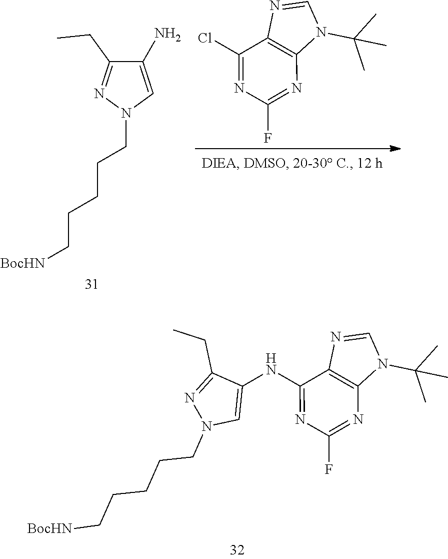 [SMILES: CCc1nn(CCCCCNC(=O)OC(C)(C)C)cc1N ; CC(C)(C)n1cnc2c(Cl)nc(F)nc21 ; CCc1nn(CCCCCNC(=O)OC(C)(C)C)cc1Nc1nc(F)nc2c1ncn2C(C)(C)C]

To a solution of tert-butyl (5-(4-amino-3-ethyl-1H-pyrazol-1-yl)pentyl)carbamate (1.6 g, 5.4 mmol, 1 eq) in DMSO (40 mL) was added DIEA (2.1 g, 16.2 mmol, 2.8 mL, 3 eq) and 9-(tert-butyl)-6-chloro-2-fluoro-9H-purine (1.5 g, 6.5 mmol, 1.2 eq) at 25° C. The mixture was stirred at 30° C. for 12 h. TLC showed the starting material was consumed completely. The residue was partitioned between ethyl acetate (80 mL) and H₂O (150 mL). The organic phase was separated, washed with brine (30 mL×3), dried over Na₂SO₄, filtered, and concentrated under reduced pressure to provide intermediate 32, tert-butyl(5-(4-((9-(tert-butyl)-2-fluoro-9H-purin-6-yl)amino)-3-ethyl-1H-pyrazol-1-yl)pentyl)carbamate (2.6 g, crude), as brown oil. The crude material was used for the next step without further purification.

### Preparation of Intermediate 33

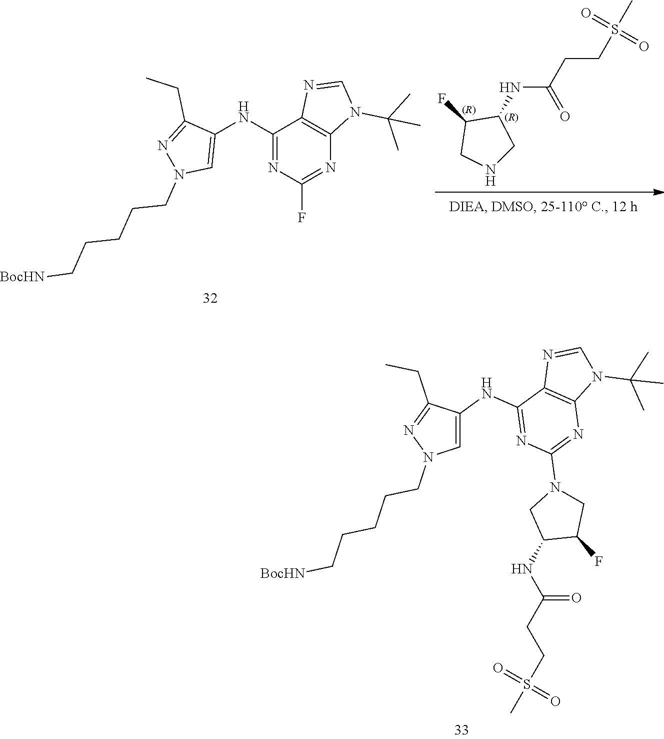

To a solution of tert-butyl (5-(4-((9-(tert-butyl)-2-fluoro-9H-purin-6-yl)amino)-3-ethyl-1H-pyrazol-1-yl)pentyl)carbamate (2.5 g, 5.1 mmol, 1 eq) in DMSO (36 mL) was added DIEA (2 g, 15.4 mmol, 2.7 mL, 3 eq) and N-((3R,4R)-4-fluoropyrrolidin-3-yl)-3-(methylsulfonyl)propanamide (1.7 g, 6.1 mmol, 1.2 eq, HCl) at 25° C. The mixture was stirred at 110° C. for 12 h under N₂. TLC showed a new spot was generated. The residue was partitioned between H₂O (200 mL) and ethyl acetate (80 mL). The organic phase was separated, washed with brine (50 mL×3), dried over Na₂SO₄, filtered, and concentrated under reduced pressure. The residue was purified by column chromatography (SiO₂, ethyl acetate:methanol=1:0 to 10:1 gradient) to provide intermediate 33, tert-butyl (5-(4-((9-(tert-butyl)-2-((3R,4R)-3-fluoro-4-(3-(methylsulfonyl)propanamido)pyrrolidin-1-yl)-9H-purin-6-yl)amino)-3-ethyl-1H-pyrazol-1-yl)pentyl)carbamate (2.6 g, 3.4 mmol, 65.9% yield, 91.6% purity), as a brown oil. 1H NMR (400 MHz, CDCl3) δ ppm 8.14-8.02 (m, 1H), 7.71-7.56 (m, 1H), 5.38-4.96 (m, 1H), 4.80-4.43 (m, 2H), 4.22-4.07 (m, 2H), 4.04-3.84 (m, 3H), 3.76 (t, J=6.4 Hz, 5H), 3.51-3.32 (m, 2H), 3.29-3.03 (m, 2H), 3.00-2.92 (m, 3H), 2.84-2.63 (m, 4H), 1.86 (t, J=3.3 Hz, 3H), 1.80-1.72 (m, 9H), 1.34-1.24 (m, 12H) MS (ESI) m/z: 707.4 (M+H)+, RT=0.695 min.

### Preparation of Intermediate 34

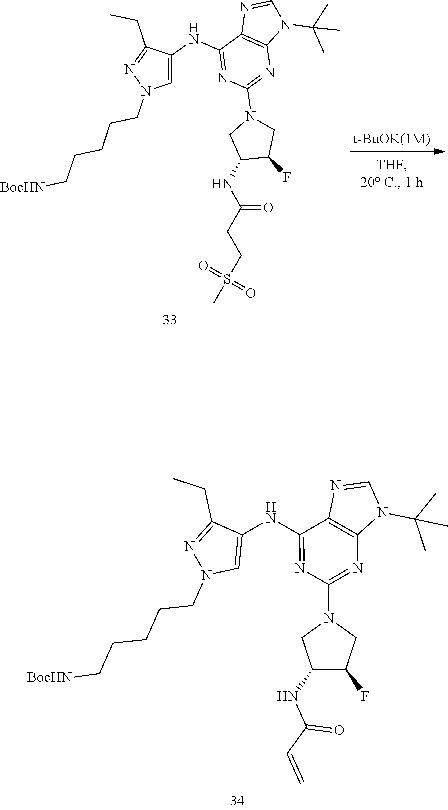 [SMILES: CCc1nn(CCCCCNC(=O)OC(C)(C)C)cc1Nc1nc(N2C[C@@H](F)[C@H](NC(=O)CCS(C)(=O)=O)C2)nc2c1ncn2C(C)(C)C ; C=CC(=O)N[C@@H]1CN(c2nc(Nc3cn(CCCCCNC(=O)OC(C)(C)C)nc3CC)c3ncn(C(C)(C)C)c3n2)C[C@H]1F]

To a solution of tert-butyl (5-(4-((9-(tert-butyl)-2-((3R,4R)-3-fluoro-4-(3-(methylsulfonyl)propanamido)pyrrolidin-1-yl)-9H-purin-6-yl)amino)-3-ethyl-1H-pyrazol-1-yl)pentyl)carbamate (1 g, 1.4 mmol, 1 eq) in THF (27 mL) was added t-BuOK (1 M in THF, 4.2 mL, 3 eq) at 0° C. The mixture was stirred at 20° C. for 1 h under N₂. TLC showed a new spot was generated. The residue was partitioned between H₂O (10 mL) and ethyl acetate (10 mL). The organic phase was separated, washed with brine (5 mL×3), dried over Na₂SO₄, filtered, and concentrated under reduced pressure. The residue was purified by prep-TLC (SiO₂, ethyl acetate:methanol=20:1) to provide intermediate 34, tert-butyl (5-(4-((2-((3R,4R)-3-acrylamido-4-fluoropyrrolidin-1-yl)-9-(tert-butyl)-9H-purin-6-yl)amino)-3-ethyl-TH-pyrazol-1-yl)pentyl)carbamate (610 mg, 973.3 umol, 68.8% yield), as yellow oil. ¹H NMR (400 MHz, CDCl₃) δ ppm 8.08 (s, 1H), 7.61 (s, 1H), 7.20 (s, 1H), 6.36 (dd, J=1.5, 17.0 Hz, 1H), 6.13 (dd, J=10.3, 17.0 Hz, 1H), 5.66 (dd, J=1.3, 10.3 Hz, 1H), 5.31 (s, 1H), 5.19 (s, 1H), 4.79-4.68 (m, 1H), 4.59-4.47 (m, 1H), 4.17-4.05 (m, 2H), 4.04-3.90 (m, 2H), 3.89-3.78 (m, 1H), 3.50 (s, 4H), 3.23-2.98 (m, 2H), 2.73 (q, J=7.6 Hz, 2H), 1.95-1.82 (m, 2H), 1.73 (s, 5H), 1.57-1.40 (m, 3H), 1.39-1.15 (m, 15H).

### Preparation of Intermediate 35

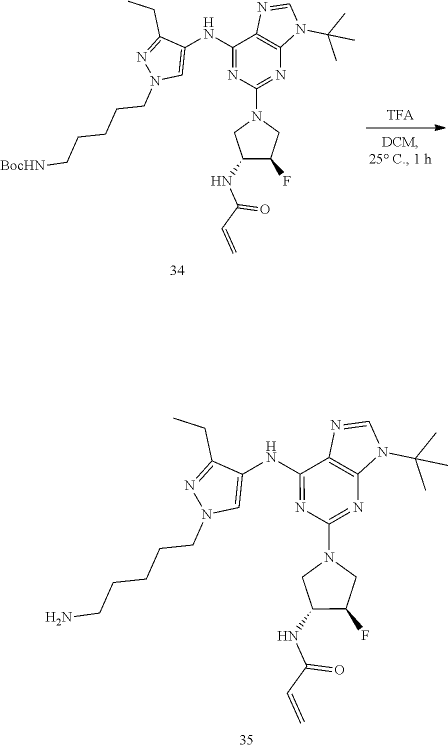 [SMILES: C=CC(=O)N[C@@H]1CN(c2nc(Nc3cn(CCCCCNC(=O)OC(C)(C)C)nc3CC)c3ncn(C(C)(C)C)c3n2)C[C@H]1F ; C=CC(=O)N[C@@H]1CN(c2nc(Nc3cn(CCCCCN)nc3CC)c3ncn(C(C)(C)C)c3n2)C[C@H]1F]

To a solution of tert-butyl (5-(4-((2-((3R,4R)-3-acrylamido-4-fluoropyrrolidin-1-yl)-9-(tert-butyl)-9H-purin-6-yl)amino)-3-ethyl-TH-pyrazol-1-yl)pentyl)carbamate (560 mg, 893.5 umol, 1 eq) in DCM (7 mL) was added TFA (2 g, 17.9 mmol, 1.3 mL, 20 eq) at 25° C. The mixture was stirred at 25° C. for 1 h. TLC showed the starting material was consumed completely. The reaction mixture was concentrated to provide Intermediate 35, N-((3R,4R)-1-(6-((1-(5-aminopentyl)-3-ethyl-1H-pyrazol-4-yl)amino)-9-(tert-butyl)-9H-purin-2-yl)-4-fluoropyrrolidin-3-yl)acrylamide (1.33 g, crude, TFA), as yellow oil. The crude material was used for the next step without further purification.

### Preparation of Intermediate 36

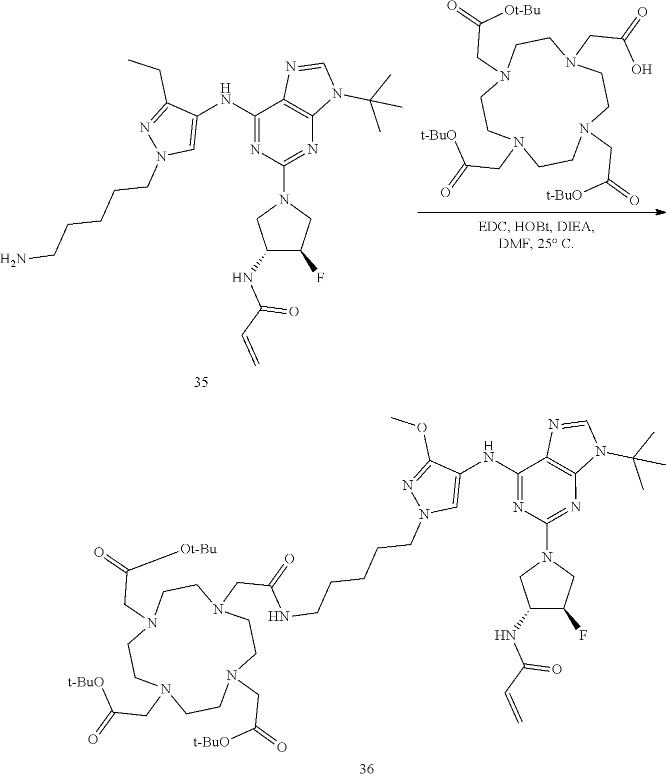 [SMILES: C=CC(=O)N[C@@H]1CN(c2nc(Nc3cn(CCCCCN)nc3CC)c3ncn(C(C)(C)C)c3n2)C[C@H]1F ; CC(C)(C)OC(=O)CN1CCN(CC(=O)O)CCN(CC(=O)OC(C)(C)C)CCN(CC(=O)OC(C)(C)C)CC1 ; C=CC(=O)N[C@@H]1CN(c2nc(Nc3cn(CCCCCNC(=O)CN4CCN(CC(=O)OC(C)(C)C)CCN(CC(=O)OC(C)(C)C)CCN(CC(=O)OC(C)(C)C)CC4)nc3OC)c3ncn(C(C)(C)C)c3n2)C[C@H]1F]

To a solution of 2-[4,7,10-tris(2-tert-butoxy-2-oxo-ethyl)-1,4,7,10-tetrazacyclododec-1-yl]acetic acid (665.1 mg, 1.2 mmol, 1.2 eq) in DMF (15 mL) was added HOBt (261.5 mg, 1.9 mmol, 2 eq) and EDC (371 mg, 1.9 mmol, 2 eq) and the mixture was stirred at 25° C. for 0.2 h. Then to the mixture was added N-[(3R,4R)-1-[6-[[1-(5-aminopentyl)-3-ethyl-pyrazol-4-yl]amino]-9-tert-butyl-purin-2-yl]-4-fluoro-pyrrolidin-3-yl]prop-2-enamide (0.6 g, 967.7 umol, 1 eq, TFA) and DIEA (375.2 mg, 2.9 mmol, 505.7 uL, 3 eq). The mixture was stirred at 25° C. for 12 h. LCMS showed the desired mass was detected. The mixture was filtered and the filtrate was concentrated under vacuum. The residue was purified by Prep-HPLC (column: Phenomenex luna C18 250*50 mm*10 um; mobile phase: [water (TFA)-ACN]; B %: 25%-55%, 10 min) to give Intermediate 36, tert-butyl 2-[4,7-bis(2-tert-butoxy-2-oxo-ethyl)-10-[2-[5-[4-[[9-tert-butyl-2-[(3R,4R)-3-fluoro-4-(prop-2-enoylamino)pyrrolidin-1-yl]purin-6-yl]amino]-3-ethyl-pyrazol-1-yl]pentylamino]-2-oxo-ethyl]-1,4,7,10-tetrazacyclododec-1-yl]acetate (0.8 g, 739.8 umol, 76.5% yield) as white solid. MS (ESI) m/z: 1081.9 (M+H)⁺, RT=0.711 min.

### Preparation of Compound 005

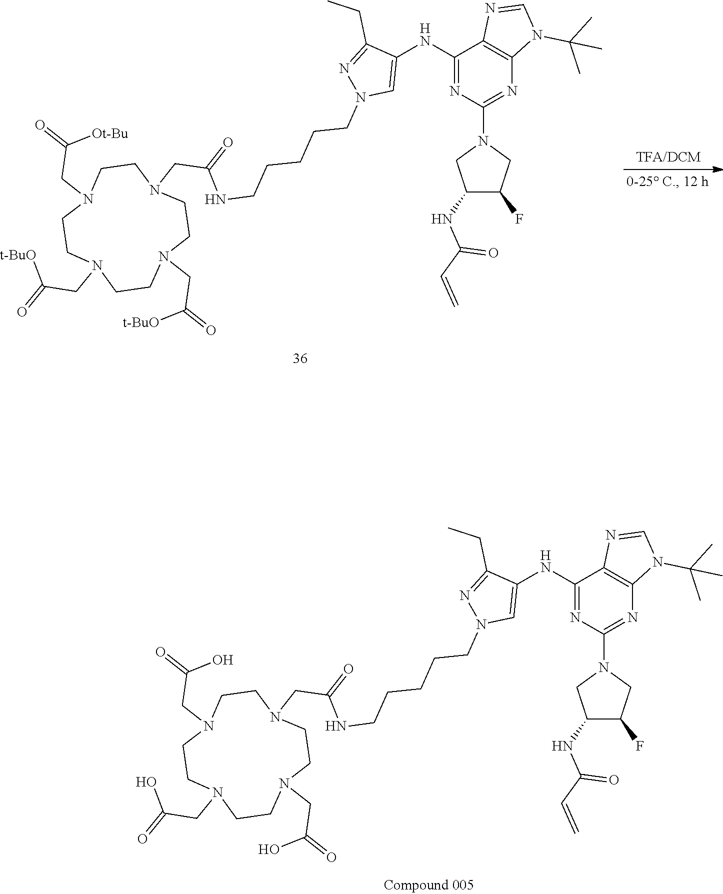 [SMILES: C=CC(=O)N[C@@H]1CN(c2nc(Nc3cn(CCCCCNC(=O)CN4CCN(CC(=O)OC(C)(C)C)CCN(CC(=O)OC(C)(C)C)CCN(CC(=O)OC(C)(C)C)CC4)nc3CC)c3ncn(C(C)(C)C)c3n2)C[C@H]1F ; C=CC(=O)N[C@@H]1CN(c2nc(Nc3cn(CCCCCNC(=O)CN4CCN(CC(=O)O)CCN(CC(=O)O)CCN(CC(=O)O)CC4)nc3CC)c3ncn(C(C)(C)C)c3n2)C[C@H]1F]

To a mixture of tert-butyl 2-[4,7-bis(2-tert-butoxy-2-oxo-ethyl)-10-[2-[5-[4-[[9-tert-butyl-2-[(3R,4R)-3-fluoro-4-(prop-2-enoylamino)pyrrolidin-1-yl]purin-6-yl]amino]-3-ethyl-pyrazol-1-yl]pentylamino]-2-oxo-ethyl]-1,4,7,10-tetrazacyclododec-1-yl]acetate (330 mg, 305.2 umol, 1 eq) in DCM (3 mL) was added TFA (1 mL) at 0° C. The mixture was stirred at 25° C. for 12 h. LCMS showed that the reaction was complete. The mixture was concentrated under vacuum. The residue was purified by prep-HPLC (column: Phenomenex Luna C18 75*30 mm*3 um; mobile phase: [water (FA)-ACN]; B %: 20%-60%, 8 min) to give Compound 005, 2-[4-[2-[5-[4-[[9-tert-butyl-2-[(3R,4R)-3-fluoro-4-(prop-2-enoylamino)pyrrolidin-1-yl]purin-6-yl]amino]-3-ethyl-pyrazol-1-yl]pentylamino]-2-oxo-ethyl]-7,10-bis(carboxymethyl)-1,4,7,10-tetrazacyclododec-1-yl]acetic acid (70 mg, 74.9 umol, 24.5% yield, 97.7% purity), as a white solid. 1H NMR (400 MHz, CD3OD): δ (ppm) 8.61-8.62 (2H, m), 8.14-8.18 (1H, m), 7.91 (1H, s), 7.83 (1H, s), 6.22-6.29 (1H, m), 6.10-6.15 (1H, m), 5.59-5.62 (1H, m), 5.07-5.20 (1H, d), 4.05-4.07 (1H, m), 4.00-4.02 (2H, m), 3.97-3.99 (2H, m), 3.81 (1H, s), 3.57-3.60 (2H, m), 3.47 (6H, s), 3.06 (2H, s), 3.03-3.05 (3H, m), 2.90-2.96 (11H, m), 2.70 (4H, m), 2.60-2.62 (2H, m), 1.74-1.76 (2H, m), 1.70 (9H, s), 1.43-1.45 (2H, m), 1.23-1.24 (2H, m), 1.10-1.13 (3H, m). MS (ESI) m/z: 913.4 (M+H)⁺, RT=1.916 min.

### Preparation of Compound 005-Lu

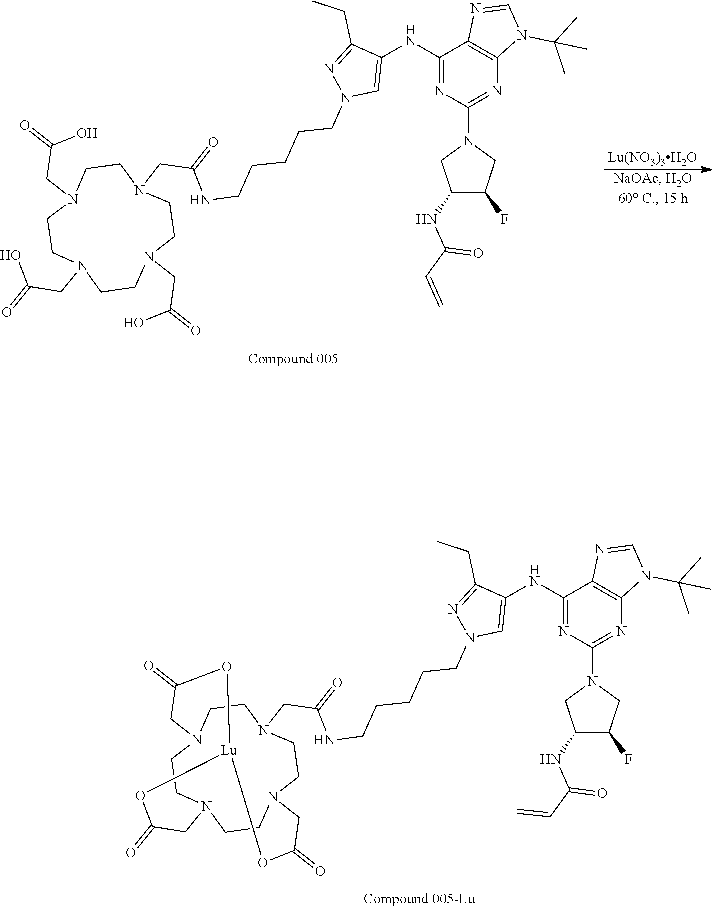

To a mixture of 2-[4-[2-[5-[4-[[9-tert-butyl-2-[(3R,4R)-3-fluoro-4-(prop-2-enoylamino)pyrrolidin-1-yl]purin-6-yl]amino]-3-ethyl-pyrazol-1-yl]pentylamino]-2-oxo-ethyl]-7,10-bis(carboxymethyl)-1,4,7,10-tetrazacyclododec-1-yl]acetic acid (0.3 g, 292.1 umol, 1 eq, FA) in aqueous NaOAc solution (0.2 M, 12.6 mL, 8.6 eq) was added lutetium(III);trinitrate;hydrate (110.7 mg, 292.1 umol, 1 eq) under N₂. The mixture was stirred at 60° C. for 15 h. LCMS showed the desired product was detected. The mixture was concentrated under vacuum to give a residue. The residue was purified by prep-HPLC (column: Phenomenex luna C18 100*40 mm*3 um; mobile phase: [water (FA)-ACN]; B %: 10%-50%, 8 min) to give Compound 005-Lu, N-[(3R,4R)-1-[9-tert-butyl-6-[[3-ethyl-1-[5-[[2-(3,16,19-trioxo-2,17,18-trioxa-5,8,11,14-tetraza-1λ3-lutetatricyclo[9.6.3.25,14]docosan-8-yl)acetyl]amino]pentyl]pyrazol-4-yl]amino]purin-2-yl]-4-fluoro-pyrrolidin-3-yl]prop-2-enamide D-020-Lu (60 mg, 55.2 umol, 18.9% yield, 99% purity), as white solid. 1H NMR (400 MHz, CD₃OD) δ (ppm) 8.25 (s, 1H), 7.90 (s, 1H), 6.40-6.23 (m, 2H), 5.77-5.57 (m, 1H), 5.31-5.07 (m, 1H), 4.70-4.60 (m, 1H), 4.14 (t, J=6.7 Hz, 2H), 4.08-3.86 (m, 3H), 3.86-3.71 (m, 2H), 3.67-3.55 (m, 2H), 3.51-3.32 (m, 6H), 3.30-3.03 (m, 6H), 2.86-2.49 (m, 9H), 2.47-2.23 (m, 4H), 1.97-1.85 (m, 2H), 1.79 (s, 9H), 1.67-1.47 (m, 2H), 1.35-1.24 (m, 5H). MS (ESI) m/z: 1085.5 (M+H)+, RT=2.424 min.

### Example A10: Synthesis of Compound 006 and Compound 006-Lu

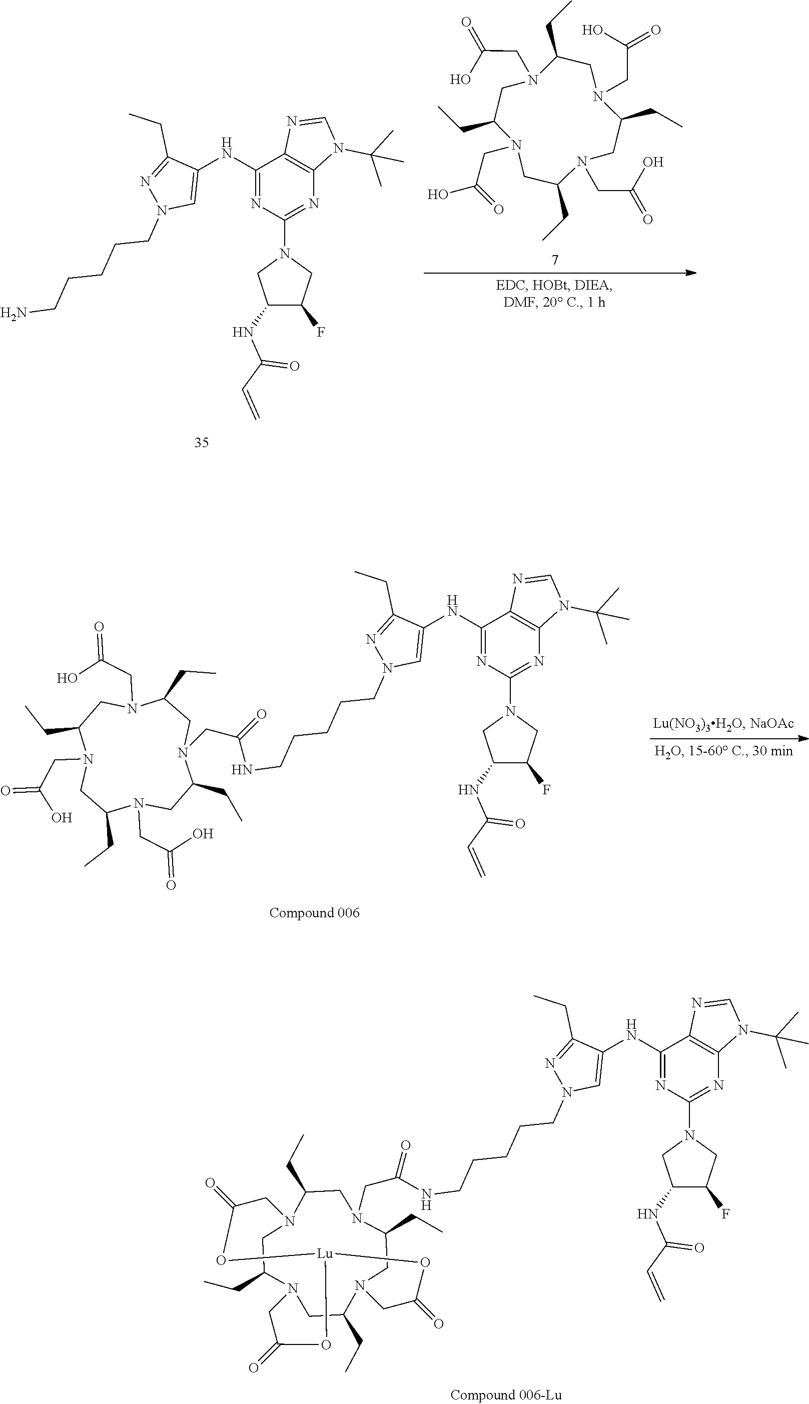

### Preparation of Compound 006

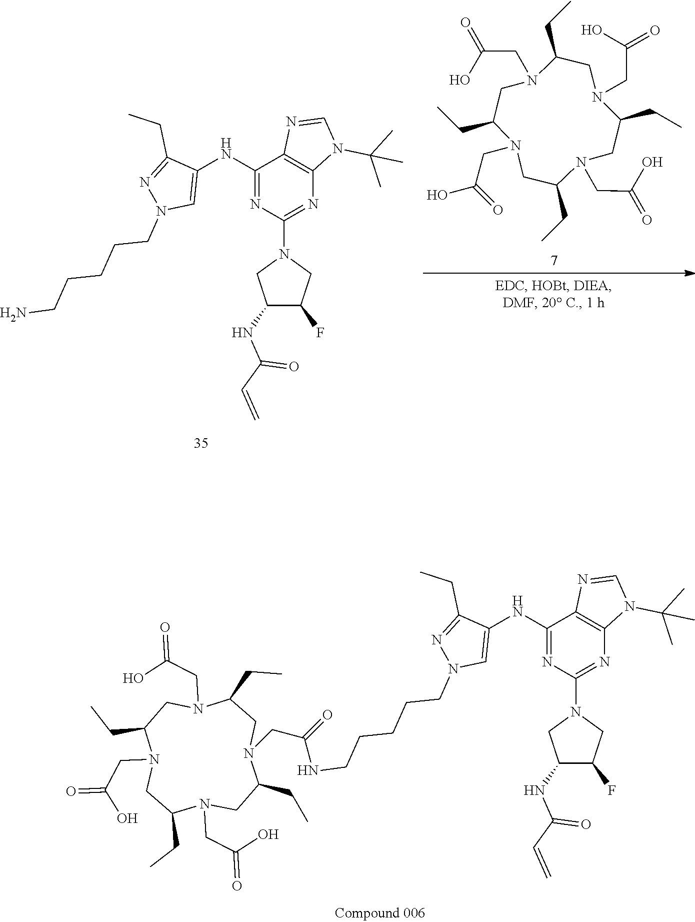

To a solution of 2,2′,2″,2′″-((2S,5S,8S,11S)-2,5,8,11-tetraethyl-1,4,7,10-tetraazacyclododecane-1,4,7,10-tetrayl)tetraacetic acid (798.5 mg, 1.6 mmol, 2 eq) in DMF (12 mL) was added DIEA (2 g, 15.5 mmol, 2.7 mL, 20 eq), HOBt (250.6 mg, 1.9 mmol, 2.4 eq), EDC (651.8 mg, 3.4 mmol, 4.4 eq) and N-((3R,4R)-1-(6-((1-(5-aminopentyl)-3-ethyl-1H-pyrazol-4-yl)amino)-9-(tert-butyl)-9H-purin-2-yl)-4-fluoropyrrolidin-3-yl)acrylamide (1.2 g, 772.8 umol, 1 eq, TFA) at 0° C. The mixture was stirred at 20° C. for 1 h. LCMS showed that starting material was consumed and desired mass was detected. The reaction mixture was directly purified by prep-HPLC: column: Phenomenex luna C18 (250×70 mm, 15 um); mobile phase: [water(TFA)-ACN]; B %: 20%-50%, 20 min and the eluent was lyophilization to provide Compound 006, 2,2′,2″-((2S,5S,8S,11S)-10-(2-((5-(4-((2-((3R,4R)-3-acrylamido-4-fluoropyrrolidin-1-yl)-9-(tert-butyl)-9H-purin-6-yl)amino)-3-ethyl-1H-pyrazol-1-yl)pentyl)amino)-2-oxoethyl)-2,5,8,11-tetraethyl-1,4,7,10-tetraazacyclododecane-1,4,7-triyl)triacetic acid (475 mg, 412.3 umol, 53.4% yield, 99% purity, TFA), as white solid. 1H NMR (400 MHz, CD₃OD) δ ppm 8.52 (d, J=11.01 Hz, 1H), 8.15 (s, 1H), 6.33-6.19 (m, 2H), 5.73-5.65 (m, 1H), 5.30-5.10 (m, 1H), 4.61 (dd, J=11.38, 5.25 Hz, 1H), 4.29-3.69 (m, 14H), 3.64-3.39 (m, 4H), 3.26-2.80 (m, 10H), 2.73 (q, J=7.59 Hz, 2H), 2.13-1.90 (m, 5H), 1.84 (s, 10H), 1.62-1.29 (m, 8H), 1.25 (t, J=7.63 Hz, 3H), 1.20-0.93 (m, 12H). MS (ESI) m/z: 1025.6 (M+H)⁺, R.T.=2.171 min.

### Preparation of Compound 006-Lu

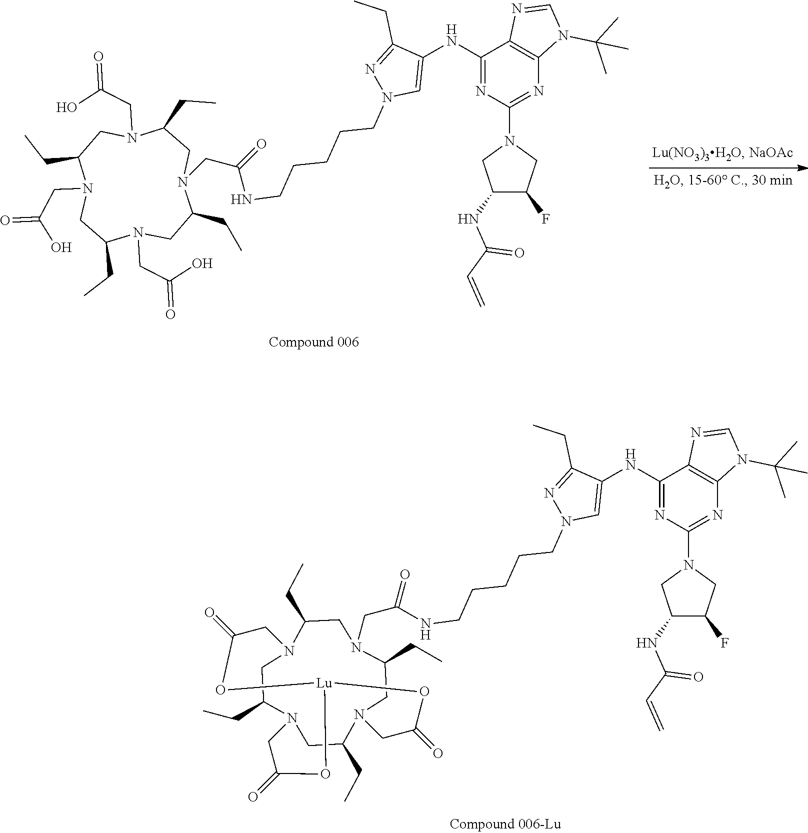

To a solution of 2-[(2S,5S,8S,11S)-4-[2-[5-[4-[[9-tert-butyl-2-[(3R,4R)-3-fluoro-4-(prop-2-enoylamino)pyrrolidin-1-yl]purin-6-yl]amino]-3-ethyl-pyrazol-1-yl]pentylamino]-2-oxo-ethyl]-7,10-bis(carboxymethyl)-2,5,8,11-tetraethyl-1,4,7,10-tetrazacyclododec-1-yl]acetic acid (180 mg, 175.6 umol, 1 eq) and NaOAc (144 mg, 1.8 mmol, 10 eq) in H₂O (2 mL) was added Lu(NO₃)₃·H₂O (119.8 mg, 316 umol, 1.8 eq) at 15° C. The mixture was stirred at 60° C. for 30 min. LCMS showed the starting material was consumed and desired mass was detected. The reaction mixture was concentrated to dryness. The residue was purified by prep-HPLC: column: Phenomenex luna C18 (250*70 mm, 15 um); mobile phase: [water(TFA)-MeCN]; B %: 20%-50%, 20 min and the eluent was directly lyophilized to provide Compound 006-Lu, N-[(3R,4R)-1-[9-tert-butyl-6-[[3-ethyl-1-[5-[[2-[(6S,9S,12S,21S)-6,9,12,21-tetraethyl-3,16,19-trioxo-2,17,18-trioxa-5,8,11,14-tetraza-1λ3-lutetatricyclo[9.6.3.25,14]docosan-8-yl]acetyl]amino]pentyl]pyrazol-4-yl]amino]purin-2-yl]-4-fluoro-pyrrolidin-3-yl]prop-2-enamide (25.8 mg, 21.3 umol, 12% yield, 99% purity), as white solid. 1H NMR (400 MHz, CD3OD) δ ppm 8.82 (s, 1H), 8.07 (s, 1H), 6.32-6.20 (m, 2H), 5.74-5.63 (m, 1H), 5.26-5.08 (m, 1H), 4.59 (dd, J=11.57, 5.94 Hz, 1H), 4.15 (t, J=6.32 Hz, 2H), 4.01-3.88 (m, 2H), 3.87-3.80 (m, 1H), 3.80-3.68 (m, 2H), 3.68-3.51 (m, 3H), 3.50-3.34 (m, 5H), 3.25-3.01 (m, 5H), 2.78 (t, J=10.63 Hz, 3H), 2.69 (q, J=7.63 Hz, 2H), 2.54-2.37 (m, 5H), 1.86 (s, 10H), 1.83-1.68 (m, 4H), 1.63-1.51 (m, 2H), 1.38-1.19 (m, 9H), 1.06-0.92 (m, 12H)

MS (ESI) m/z: 1197.6 (M+H)+, R.T.=2.169 min.

### Example A11: Synthesis of Compound 007 and Compound 007-Lu

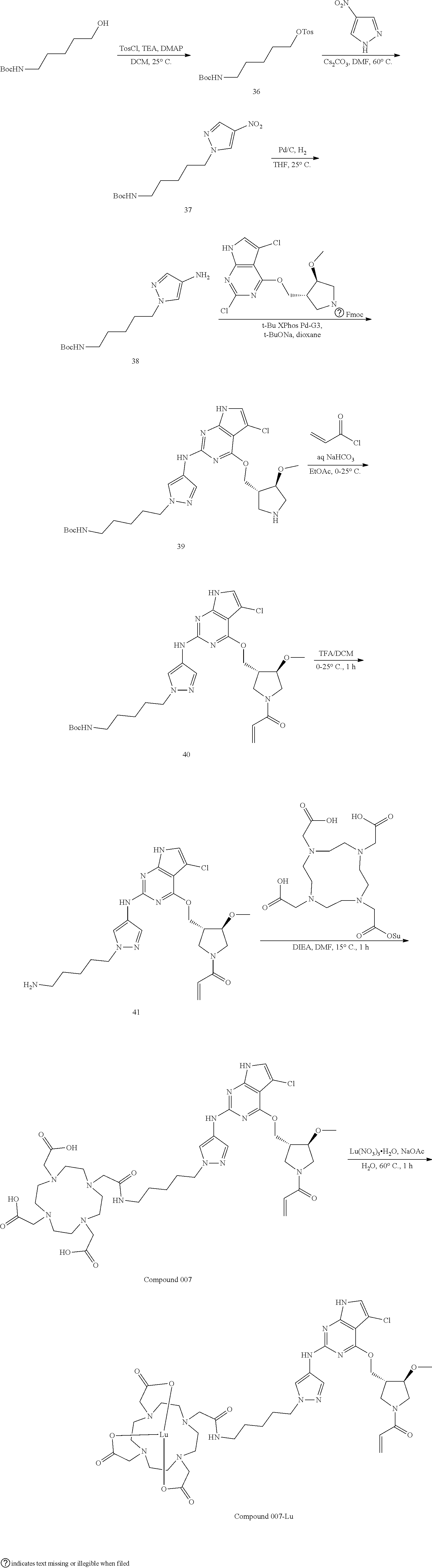

### Preparation of Intermediate 36

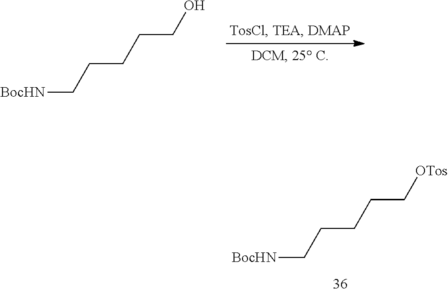

To a solution of tert-butyl (5-hydroxypentyl)carbamate (13 g, 64 mmol, 13 mL, 1 eq) and TosCl (24.4 g, 128 mmol, 2 eq) in DCM (130 mL) was added TEA (19.4 g, 192 mmol, 26.7 mL, 3 eq) and DMAP (781.3 mg, 6.4 mmol, 0.1 eq) at 0° C. The mixture was warmed to 25° C. and stirred for 12 h. LCMS showed the material was consumed completely and one major peak of desired compound was detected. The residue was poured into ice-water (200 mL). The aqueous phase was extracted with DCM (200 mL×3). The combined organic phase was washed with brine (200 mL), dried with anhydrous Na₂SO₄, filtered, and concentrated under vacuum. The residue was purified by column chromatography (SiO₂, petroleum ether:ethyl acetate=1:0 to 0:1 gradient) to give Intermediate 7, 5-((tert-butoxycarbonyl)amino)pentyl 4-methylbenzenesulfonate (18.8 g, 52.59 mmol, 82.24% yield), as white solid. MS (ESI) m/z: 358.2 (M+H)+, RT=0.842 min

### Preparation of Intermediate 37

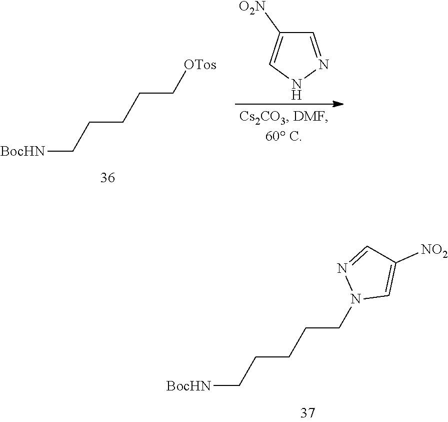

The solution of 5-((tert-butoxycarbonyl)amino)pentyl 4-methylbenzenesulfonate (8 g, 22.4 mmol, 1 eq) in DMF (110 mL) was added 4-nitro-1H-pyrazole 2A (2.8 g, 24.6 mmol, 1.1 eq) and Cs₂CO₃ (21.9 g, 67.1 mmol, 3 eq). The mixture was stirred at 60° C. for 12 h. LCMS showed the material was consumed completely and one major peak of desired compound was detected. The residue was poured into ice-water (200 mL). The aqueous phase was extracted with ethyl acetate (100 mL). The combined organic phase was washed with brine (100 mL), dried with anhydrous Na₂SO₄, filtered, and concentrated under vacuum to give Intermediate 37, tert-butyl (5-(4-nitro-1H-pyrazol-1-yl)pentyl)carbamate (7.3 g, crude), as yellow solid. The crude material was used for the next step without further purification. 1H NMR (400 MHz, CDCl3) δ ppm 8.12 (1H, s) 8.07 (1H, s) 4.15 (2H, t, J=7.09 Hz) 3.12 (2H, d, J=4.16 Hz) 1.94 (2H, q, J=7.43 Hz) 1.52 (2H, m) 1.44 (9H, s) 1.34 (2H, m).

### Preparation of Intermediate 38

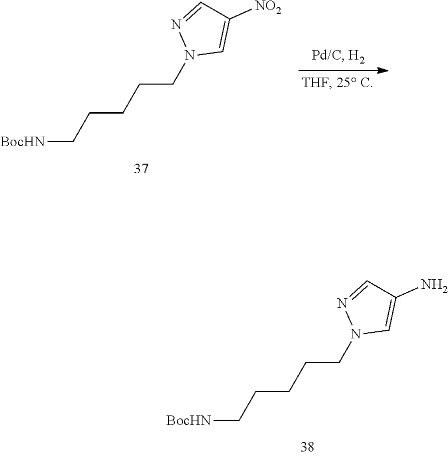 [SMILES: CC(C)(C)OC(=O)NCCCCCn1cc([N+](=O)[O-])cn1 ; CC(C)(C)OC(=O)NCCCCCn1cc(N)cn1]

To a solution of tert-butyl (5-(4-nitro-1H-pyrazol-1-yl)pentyl)carbamate (7.3 g, 24.5 mmol, 1 eq) in THF (73 mL) was added Pd/C (3.5 g, 10% purity) under N2. The suspension was degassed under vacuum and purged with H₂ several times. The mixture was stirred under H₂ (15 psi) at 25° C. for 2 h. LCMS showed the material was consumed completely and one major peak of desired compound was detected. The reaction mixture was filtered and the filtrate was concentrated under vacuum to give Intermediate 38, tert-butyl (5-(4-amino-1H-pyrazol-1-yl)pentyl)carbamate (6 g, 21.7 mmol, 88.5% yield, 97% purity), as white oil. The crude material was used for the next step without further purification. 1H NMR (400 MHz, CD3OD) δ ppm 7.20 (1H, m) 7.12 (1H, s) 4.00 (2H, t, J=6.97 Hz) 3.00 (2H, m) 1.79 (2H, quin, J=7.31 Hz) 1.43 (11H, m) 1.27 (2H, m). MS (ESI) m/z: 269.2 (M+H)+, RT=0.613 min.

### Preparation of Intermediate 39

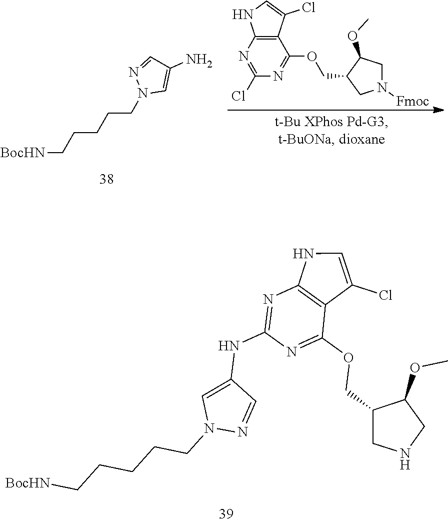

The reaction was run under multiple batches (7×).

To a solution of (9H-fluoren-9-yl)methyl (3R,4R)-3-(((2,5-dichloro-7H-pyrrolo[2,3-d]pyrimidin-4-yl)oxy)methyl)-4-methoxypyrrolidine-1-carboxylate (300 mg, 55 6.2 umol, 1 eq) and tert-butyl (5-(4-amino-1H-pyrazol-1-yl)pentyl)carbamate (149.3 mg, 556.2 umol, 1 eq) in dioxane (3 mL) was added [2-(2-aminophenyl) phenyl]-methylsulfonyloxy-palladium;ditert-butyl-[2-(2,4,6-triisopropylphenyl) phenyl]phosphane (44.12 mg, 55.6 umol, 0.1 eq) and t-BuONa (106.9 mg, 1.1 mmol, 2 eq), then the mixture was stirred at 90° C. for 12 h. LCMS showed the starting material was consumed and one major peak of desired compound was detected. All batches were combined and poured into ice-water (30 mL). The aqueous phase was extracted with ethyl acetate (30 mL×3). The combined organic phase was washed with brine (30 mL), dried with anhydrous Na₂SO₄, filtered, and concentrated under vacuum. The residue was purified by prep-HPLC(column: Welch Xtimate C18 250*70 mm #10 um; mobile phase: [water (NH3H2O)-ACN]; B %: 1%-60%, 20 min) to give Intermediate 39, (9H-fluoren-9-yl)methyl (3R,4R)-3-(((2-((1-(5-((tert-butoxycarbonyl)amino)pentyl)-1H-pyrazol-4-yl)amino)-5-chloro-7H-pyrrolo[2,3-d]pyrimidin-4-yl)oxy)methyl)-4-methoxypyrrolidine-1-carboxylate 13 (260 mg, 351.7 umol, 9% yield, 74% purity), as yellow solid. MS (ESI) m/z: 549.3 (M+H)⁺, RT=1.539 min.

### Preparation of Intermediate 40

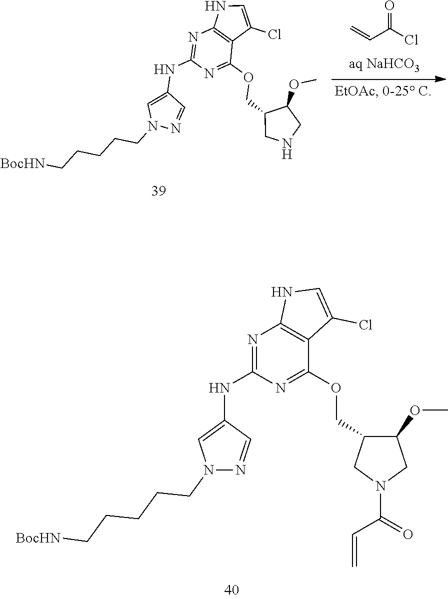

To a solution of tert-butyl (5-(4-((5-chloro-4-(((3R,4R)-4-methoxypyrrolidin-3-yl)methoxy)-7H-pyrrolo[2,3-d]pyrimidin-2-yl)amino)-1H-pyrazol-1 yl)pentyl) carbamate (270 mg, 491.8 umol, 1 eq) in ethyl acetate (3.2 mL) was added saturated NaHCO₃solution (1.6 mL) at 0° C. and stirred for 10 min. To the reaction, acryloyl chloride (49 mg, 540.9 umol, 44.1 uL, 1.1 eq) was added dropwise and the resulting mixture was stirred at 25° C. for 0.5 h. LCMS showed the starting material was consumed and one major peak of desired compound was detected. The reaction mixture was poured into water (20 mL). The aqueous phase was extracted with ethyl acetate (20 mL×3). The combined organic phase was washed with brine (20 mL), dried with anhydrous Na₂SO₄, filtered, and concentrated under vacuum. The residue was purified by prep-HPLC(column: Waters Xbridge Prep OBD C18 150*40 mm*10 um; mobile phase: [water(NH4HCO3)-ACN]; B %: 30%-60%, 8 min) to give Intermediate 40, tert-butyl (5-(4-((4-(((3R,4R)-1-acryloyl-4-methoxypyrrolidin-3-yl)methoxy)-5-chloro-7H-pyrrolo[2,3-d]pyrimidin-2-yl)amino)-1H-pyrazol-1-yl)pentyl)carbamate 14 (210 mg, 348.2 umol, 71% yield), as yellow solid. MS (ESI) m/z: 603.3 (M+H)+, RT=1.749 min.

### Preparation of Intermediate 41

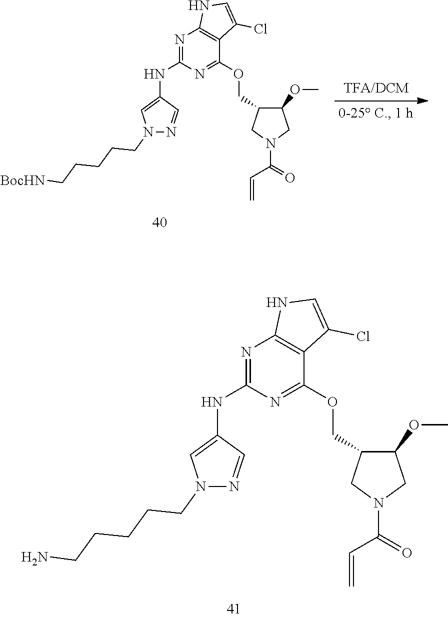 [SMILES: C=CC(=O)N1C[C@H](COc2nc(Nc3cnn(CCCCCNC(=O)OC(C)(C)C)c3)nc3[nH]cc(Cl)c23)[C@@H](OC)C1 ; C=CC(=O)N1C[C@H](COc2nc(Nc3cnn(CCCCCN)c3)nc3[nH]cc(Cl)c23)[C@@H](OC)C1]

To a solution of tert-butyl (5-(4-((4-(((3R,4R)-1-acryloyl-4-methoxypyrrolidin-3-yl)methoxy)-5-chloro-7H-pyrrolo[2,3-d]pyrimidin-2-yl)amino)-1H-pyrazol-1-yl)pentyl)carbamate 14 (150 mg, 248.7 umol, 1 eq) in DCM (1.5 mL) was added TFA (1.1 g, 10 mmol, 736.6 uL, 40 eq) at 0° C., then the mixture was stirred at 25° C. for 1 h. LCMS showed the starting material was consumed and one major peak of desired compound was detected. The reaction mixture was concentrated under vacuum to give Intermediate 41, 1-((3R,4R)-3-(((2-((1-(5-aminopentyl)-1H-pyrazol-4-yl)amino)-5-chloro-7H-pyrrolo[2,3-d]pyrimidin-4-yl)oxy)methyl)-4-methoxypyrrolidin-1-yl)prop-2-en-1-one 15 (153 mg, crude, TFA), as yellow oil. The crude material was used for the next step without further purification. MS (ESI) m/z: 503.2 (M+H)+, RT=0.601 min.

### Preparation of Compound 007

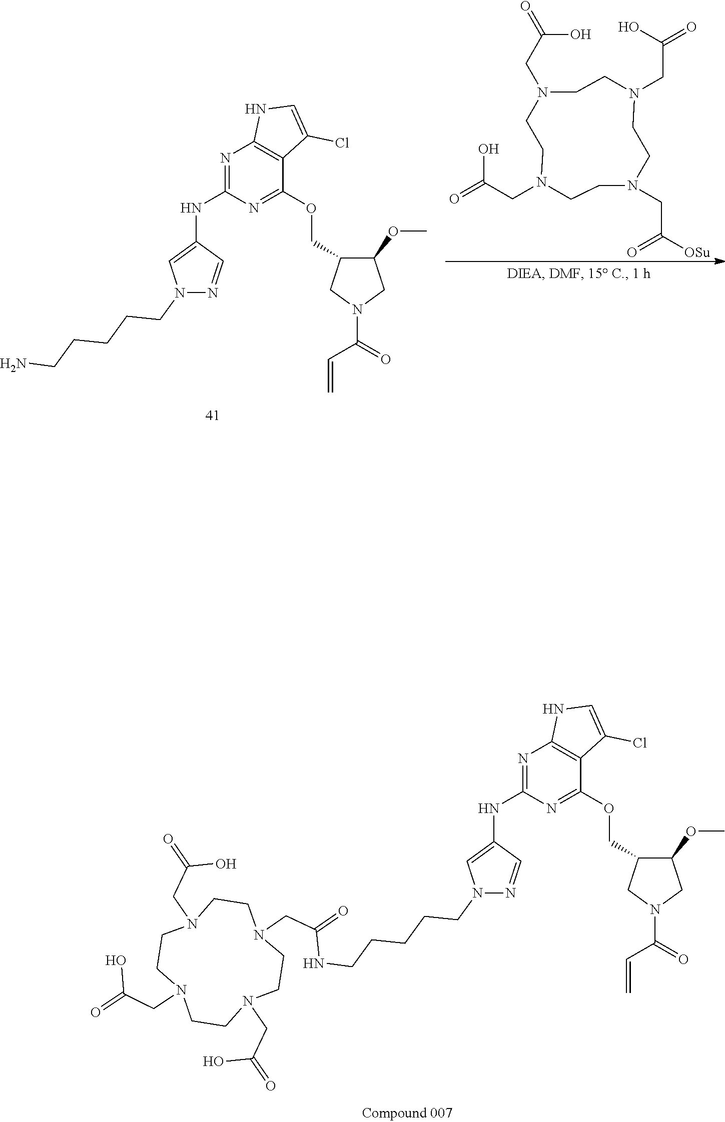

To a solution of 1-((3R,4R)-3-(((2-((1-(5-aminopentyl)-1H-pyrazol-4-yl)amino)-5-chloro-7H-pyrrolo[2,3-d]pyrimidin-4-yl)oxy)methyl)-4-methoxypyrrolidin-1-yl)prop-2-en-1-one (143 mg, 231.8 umol, 1 eq, TFA) and DIEA (898.6 mg, 7 mmol, 1.2 mL, 30 eq) in DMF (1.4 mL) was added 2,2′,2″-(10-(2-((2,5-dioxopyrrolidin-1-yl)oxy)-2-oxoethyl)-1,4,7,10-tetraazacyclododecane-1,4,7-triyl) 2,2′,2″-(10-(2-((2,5-dioxopyrrolidin-1-yl)oxy)-2-oxoethyl)-1,4,7,10-tetraazacyclododecane-1,4,7-triyl)triacetic acid (285.3 mg, 463.5 umol, 2 eq, TFA). The mixture was stirred at 15° C. for 1 h. LCMS showed the material was consumed and one major peak of desired compound was detected. The reaction mixture was filtered and the filtrate was directly purified by prep-HPLC(column: C18-4 150*30 mm*5 um; mobile phase: [water(TFA)-ACN]; B %: 10%-40%, 20 min) to give Compound 007, 2,2′,2″-(10-(2-((5-(4-((4-(((3R,4R)-1-acryloyl-4-methoxypyrrolidin-3yl)methoxy)-5-chloro-7H-pyrrolo[2,3-d]pyrimidin-2-yl)amino)-1H-pyrazol-1-yl)pentyl)amino)-2-oxoethyl)-1,4,7,10-tetraazacyclododecane-1,4,7-triyl)triacetic acid (160.7 mg, 180.7 umol, 78% yield, 100% purity), as white solid. ¹H NMR (400 MHz, CD3OD) δ ppm 7.97 (1H, s) 7.62 (1H, s) 6.85 (1H, s) 6.60 (1H, m) 6.26 (1H, dt, J=16.88, 1.81 Hz) 5.74 (1H, dt, J=10.35, 2.39 Hz) 4.54 (2H, m) 4.14 (3H, m) 4.06 (1H, m) 3.78 (11H, m) 3.38 (7H, m) 3.25 (14H, m) 2.91 (1H, m) 1.87 (2H, q, J=7.22 Hz) 1.54 (2H, q, J=7.16 Hz) 1.33 (2H, m). MS (ESI) m/z: 889.3 (M+H)⁺, RT=1.956 min.

### Preparation of Compound 007-Lu

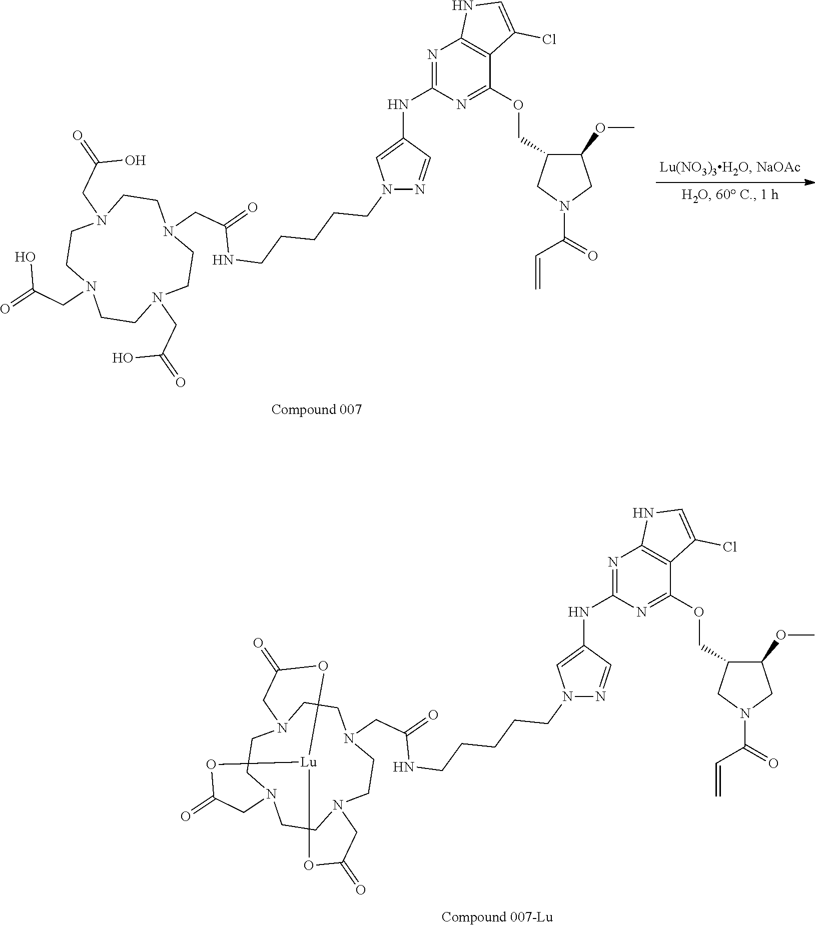

To a solution of 2-[4,7-bis(carboxymethyl)-10-[2-[5-[4-[[5-chloro-4-[[(3R,4R)-4-methoxy-1-prop-2-enoyl-pyrrolidin-3-yl]methoxy]-7H-pyrrolo[2,3-d]pyrimidin-2-yl]amino]pyrazol-1-yl]pentylamino]-2-oxo-ethyl]-1,4,7,10-tetrazacyclododec-1-yl]acetic acid (98 mg, 110.2 umol, 1 eq) and NaOAc (63.3 mg, 771.5 umol, 7 eq) in H₂O (1 mL) was added lutetium(III);trinitrate;hydrate (54.3 mg, 143.3 umol, 1.3 eq). The mixture was stirred at 60° C. for 1 h. LCMS showed the starting material was consumed completely and one major peak of desired compound was detected. The reaction mixture was filtered and the filtrate was purified by prep-HPLC(column: Waters Xbridge BEH C18 100*30 mm*10 um; mobile phase: [water(NH4HCO3)-ACN]; B %: 15%-35%, 8 min) to give crude product (46.5 mg, 42.2 umol, 96% purity). The crude product was further purified by prep-HPLC (column: Waters Xbridge BEH C18 100*30 mm*10 um; mobile phase: [water(NH4HCO3)-ACN]; B %: 25%-45%, 8 min) to give Compound 007-Lu, N-[5-[4-[[5-chloro-4-[[(3R,4R)-4-methoxy-1-prop-2-enoyl-pyrrolidin-3-yl]methoxy]-7H-pyrrolo [2,3-d]pyrimidin-2-yl]amino]pyrazol-1-yl]pentyl]-2-(3,16,19-trioxo-2,17,18-trioxa-5,8,11,14-tetraza-1λ3-lutetatricyclo[9.6.3.25,14]docosan-8-yl)acetamide D-021-Lu (25.5 mg, 24 umol, 54.8% yield, 99% purity), as white solid. 1H NMR (400 MHz, CD3OD) δ ppm 8.04 (1H, d, J=5.00 Hz) 7.62 (1H, d, J=3.50 Hz) 6.86 (1H, s) 6.61 (1H, ddd, J=16.76, 10.44, 3.69 Hz) 6.26 (1H, dd, J=16.76, 1.75 Hz) 5.75 (1H, m) 4.54 (2H, m) 4.13 (2H, t, J=6.38 Hz) 4.06 (1H, m) 3.72 (7H, m) 3.38 (10H, m) 3.19 (4H, m) 2.78 (8H, m) 2.38 (4H, d, J=13.26 Hz) 2.21 (1H, m) 1.89 (2H, q, J=7.00 Hz) 1.58 (2H, m) 1.29 (2H, m). MS (ESI) m/z: 1060.9 (M+H)+, RT=2.752 min.

### Example A12: Synthesis of 177-Lutetium Chelated Radiopharmaceutical Conjugates of Table 2B

Conjugates of Table 2A are synthesized according to the same methods described in Example A1-A11.

### General Procedure for ¹⁷⁷Lu-Labeling

[¹⁷⁷Lu]LuCl₃ in HCl (50 MBq) is added to a mixture of a conjugate of Table 2A (1 nmol) in NaOAc buffer (5% EtOH, 0.25 M, pH 5.0-5.5, total volume 120 μL) in a 1.8 mL Eppendorf tube. The resulting mixture is heated at 80° C. in a thermal mixer at a shaking speed of 600 rpm for 15-30 min. If necessary, the mixture is purified using a C8 column. Radiochemical purity is determined by radio-RP-HPLC and iTLC.

Accordingly, conjugates of Table 2B are synthesized according to Example A12.

### Example A13: Synthesis of 225-Actinium Chelated Conjugates of Table 2C

Conjugates of Table 2A are synthesized according to the same methods described in Example A1-A11.

### General Procedure for ²²⁵Ac-Labeling

[²⁵Ac]Ac(NO₃)₃ in 1 mM HCl (50 kBq) is added to a mixture of a conjugate of Table 2A (1 nmol) in NaOAc buffer (100 μL, 0.4 M, pH 5.5-6.5) in a 1.8 mL Eppendorf tube. The resulting mixture is heated at 80-100° C. in a thermal mixer at a shaking speed of 500 rpm for 15-30 min. Radiochemical purity is determined by iTLC.

Accordingly, conjugates of Table 2C are synthesized according to Example A13.

### Example A14: Exemplary Procedure for Synthesizing Covalently Modified Protein

A compound selected from Table 2A, Table 2B, and Table 2C is administered to a subject having an intracellular protein comprising a cysteine. The intracellular protein can be selected from KRAS, FGFR1, FGFR2, FGFR3, FGFR4, FGFRL1, TP53, IDH1, GNAS, FBXW7, CTNNB1, DNMT3A, EGFR, BTK, ERBB2, ERBB3, and JAK3. The intracellular protein can have one or more mutations. The intracellular protein can be non-mutated. The compound covalently binds with the protein in vivo via a cysteine residue, thereby generating a covalently modified protein.

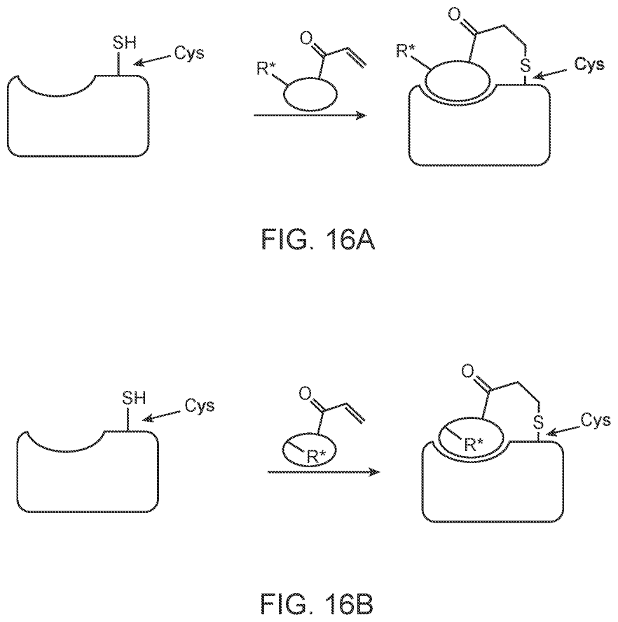

A covalently modified protein is illustrated in FIG. 16A and FIG. 16B, as an example. The compound in FIG. 16A or FIG. 16B can be any a radiopharmaceutical compound described herein that contains an electrophilic functional group

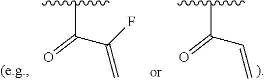

FIG. 16A or FIG. 16B also illustrates that the electrophilic functional group reacts with a cysteine residue of the protein.

### B: Biological Assays

### Example B1. KRAS G13C Nucleotide Exchange Assay

A KRAS (G13C) Nucleotide Exchange Assay kit is used to evaluate compound antagonistic binding. The assay kit (BPS Bioscience. Catalog #79859) utilizes KRAS (G13C) labeled with BODIPY-GDP to determine if compounds can affect the nucleotide exchange (GDP to GTP) in KRAS signaling. Compounds are typically characterized using both protocols: fixed GTP concentrations and fixed inhibitor concentrations.

### Fixed GTP Concentration Characterization

All reagents are prepared following manufacturer's protocol. 5 μL BODIPY-GDP and 10 μL of KRAS buffer are added to each well. A 3-fold serial dilution of compound in 5% DMSO is prepared. Typically, compounds are tested in the range of 0 μM to 50 μM, but the range could be modified depending on the characterization needs. 5 μL of diluted compound is added to the wells and the plate is centrifuged briefly to ensure all components are mixed before incubation at ambient temperature. After incubation for 2 hours, 10 μM GTP and 25 mM EDTA are mixed at a 1:1 ratio and 5 μL is added to the wells. The plate is then incubated for 1 hour at ambient temperature. After incubation, fluorescence is measured at Ex_{470 nm}/Em_{525 nm}.

Data is analyzed by plotting fluorescence vs GTP concentration for control wells and compound wells.

### Fixed Inhibitor Concentration Characterization

All reagents are prepared following manufacturer's protocol. 5 μL of compound is added to each test well. Typically, 10 μM of compound is used, but the concentration could be modified depending on characterization needs. For both test wells and control wells, 10 μL of KRAS buffer and 5 μL BODIFY-GDP are added. 5 μL of compound buffer (e.g. 5% DMSO) is added to control wells. The plate is briefly centrifuged to ensure all reagents are mixed before incubation at ambient temperature. GTP reagent is serially diluted 0 mM to 1 mM in water. After 2 hours, 2.5 μL of the prepared GTP is add to all wells along with 2.5 μL EDTA at 25 mM. The plate is briefly centrifuged to mix all reagents and incubated at ambient temperature for 1 hour. After incubation, fluorescence is measured at Ex_{470 nm}/Em_{525 nm}.

Data is analyzed by plotting fluorescence vs compound concentration. EC50 is calculated as the compound concentration that elicited 50% fluorescence.

### Example B2. Phosphorylated ERK1/ERK2 ELISA

An ERK phosphorylation ELISA kit is used to characterize compounds for KRAS-based cell activity. The sandwich ELISA (RnD Systems. Catalog #DYC1018B) measures human ERK1 that is dually phosphorylated at T202/Y204 and ERK2. Cell lysates from cells before and after treatment with compounds are prepared using kit manufacturer protocols.

All reagents are prepared following the kit's protocol. The wells of a 96-well microtiter plate are coated with 100 μL of 8.0 pg/mL of capture antibody in PBS. The plate is sealed and left to incubate overnight at ambient temperature. After incubation, the wells are washed with 400 μL of Wash Buffer for a total of 3 washes. Wells are then blocked by adding 300 μL of Block Buffer to each well and incubating at ambient temperature for 1-2 hours. After removal of the Block Buffer by aspirating and washing, 100 μL of standards and samples are added to the plate in duplicates and incubated for 2 hours at ambient temperature. The aspiration and wash steps are repeated to remove unbound standard and samples and 100 μL of 400 ng/mL detection antibody is applied to each well. After 2 hours at ambient temperature, detection antibody is removed by washing and 100 μL of Streptavidin-HRP is added to each well. Incubation of Streptavidin-HRP is at ambient temperature for 20 minutes, after which Streptavidin-HRP is removed by washing. 100 μL of Substrate Solution is added to each well and incubated for 20 minutes before 50 μL of Stop Solution is added to each well.

The optical density of each well is immediately analyzed at 450 nm with a wavelength correction at 540 nm. The standards are plotted by averaging the optical density for each replicate and fitted with a four-parameter logistic curve fit. Concentrations of phosphorylated ERK in cell lysate samples are determined by extrapolation from the standard curve and then adjusted for any dilution factors.

### Example B3. Inhibition of KRas G13C-Dependent Cell Growth

The cellular inhibition of KRas G13C by specific compounds are measured by the inhibition of growth of cells dependent on the KRas G13C mutation.

MiaPaca-2 (ATCC, CRL-1420), NCI-H358 (ATCC CRL-5807), A549 (ATCC CCL-185), and NCI-H1975 (CRL-5908) cell lines are cultured according to ATCC cell culture recommendations. Cells are plated in sterile 96-well plates at a concentration of 60,000 cells/well and allowed to attach for 12-18 hours. Diluted compounds are added to the cells with a final concentration of 0.5% DMSO, in 200 uL volume of media. The compounds and remaining media are left to culture for 72 hours. At the end of the 72 hour incubation time, the plates are removed from the incubator and left to equilibrate to room temperature for use in the Cell Titer Glo 2.0 Cell Viability Assay (Promega Catalog #G9241). All reagents are thawed and allowed to equilibrate to room temperature before use in the assay. Reagent preparation followed the manufacturer's protocol. After reagent dilution, 25 uL of the CTG reagent is added to each well of the 96 well plate and set to shake for 20 minutes at room temperature. The plate is read on a SpectraMax iD5 using the Softmax Pro 7 Software from Molecular Devices. The Cell Titer Glo Luminescence protocol is used, reading the plate at a wavelength of 595 nM.

Resulting OD values are normalized by subtracting the background values of wells that did not contain cells, and then normalized to 100% by using the DMSO-only treated well. Subsequent cell growth inhibition is calculated for the compound dilution curve and graphed in Graphpad Prism.

### Example B4. In Vitro Kinase Assay of Ligand Targeting Radionuclide Conjugates

### Kinase Assay Protocol Description

For most assays, kinase-tagged T7 phage strains were prepared in an E. coli host derived from the BL21 strain. For the EGFR assays, wild-type EGFR, or mutant EGFR (T790M, L858R) were utilized. The EGFR constructs span amino acids R669 to V1011 of the EGFR protein. E. coli were grown to log-phase and infected with T7 phage and incubated with shaking at 32° C. until lysis. The lysates were centrifuged and filtered to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific binding. Binding reactions were assembled by combining kinases, liganded affinity beads, and test compounds in 1× binding buffer (20% SeaBlock, 0.17×PBS, 0.05% Tween 20, 6 mM DTT). Test compounds were prepared as 111× stocks in 100% DMSO. Kds were determined using an 11-point 3-fold compound dilution series with three DMSO control points. All compounds for Kd measurements are distributed by acoustic transfer (non-contact dispensing) in 100% DMSO. The compounds were then diluted directly into the assays such that the final concentration of DMSO was 0.9%. All reactions performed in polypropylene 384-well plate. Each was a final volume of 0.02 ml. The assay plates were incubated at room temperature with shaking for 1 hour and the affinity beads were washed with wash buffer (lx PBS, 0.05% Tween 20). The beads were then re-suspended in elution buffer (lx PBS, 0.05% Tween 20, 0.5 μM nonbiotinylated affinity ligand) and incubated at room temperature with shaking for 30 minutes. The kinase concentration in the eluates was measured by qPCR.

TABLE 5
Binding Affinity of Compounds 001-007 (Kd, nM)

| | | EGFR | Binding |
|---|---|---|---|
| | EGFR | (L858R, | Affinity |
| | (WT) | T790M) | ratio of |
| Compound ID | Kd (nM) | Kd (nM) | WT/DM |
| Compound 001 | C | A | A |
| Compound 001-Lu | C | A | A |
| Compound 002 | C | A | A |
| Compound 002-Lu | C | A | A |
| Compound 003 | C | A | A |
| Compound 003-Lu | C | A | A |
| Compound 004 | B | B | B |
| Compound 004-Lu | C | A | B |
| Compound 005 | A | B | C |
| Compound 005-Lu | A | B | C |
| Compound 006 | A | B | B |
| Compound 007 | B | B | C |
| Compound 007-Lu | B | A | C |

EGFR (WT) Kd (nM): 2 < C ≤ 100 nM; 100 < B ≤ 1000 nM; 1000 < A ≤ 2500 nM;
EGFR (L858R, T790M) Kd (nM): 0 < A ≤ 1 nM; 1 < B ≤ 25 nM
Binding Affinity ratio of WT/DM: 4 < A ≤ 25; 25 < B ≤ 100; 100 < C ≤ 1000

1. A conjugate comprising:
a) a targeting ligand, wherein the targeting ligand covalently binds to an intracellular protein, wherein the intracellular protein is mutated;
b) a linker; and
c) a metal chelator.

2. The conjugate of claim 1, further comprising a radionuclide, wherein the radionuclide is bound to the metal chelator.

3. (canceled)

4. The conjugate of claim 1, wherein the intracellular mutated protein comprises one or more mutant-specific cysteine residues that are absent in a corresponding wild-type sequence of the intracellular mutated protein.

5. (canceled)

6. (canceled)

7. (canceled)

8. The conjugate of claim 1, wherein the targeting ligand selectively binds to at least one of the endogenous cysteine residues in the intracellular mutated protein over a same cysteine residue in a corresponding wild-type protein.

9. A conjugate comprising:
a) a targeting ligand, wherein the targeting ligand covalently binds to an intracellular protein, wherein the intracellular protein is overexpressed compared to a corresponding wild-type protein, and wherein the intracellular protein comprises one or more endogenous cysteine residues;
b) a linker; and
c) a metal chelator.

10. The conjugate of claim 9, further comprising a radionuclide, wherein the radionuclide is bound to the metal chelator.

11. The conjugate of claim 1, wherein the targeting ligand comprises an electrophilic functional group.

12. The conjugate of claim 11, wherein the electrophilic functional group covalently binds to the intracellular protein at a cysteine residue.

13. (canceled)

14. (canceled)

15. (canceled)

16. (canceled)

17. (canceled)

18. (canceled)

19. The conjugate of claim 11, wherein the electrophilic functional group comprises a structure of Formula (Ia): 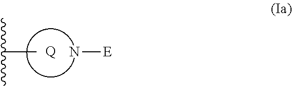
wherein,
ring Q is an optionally substituted 3 to 10 membered heterocycloalkylene; and
E comprises a structure of Formula (Ic), 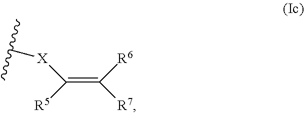
wherein,
X is C(═O), OC(═O), P(═O)OR², C(═S), S(═O)ₙ, or OS(O)ₙ;
n is 1 or 2;
R² is H or substituted or unsubstituted C₁-C₃ alkyl;
R⁵ and R⁷ are each independently selected from H, —CN, halogen, substituted or unsubstituted C₁-C₆ alkyl, substituted or unsubstituted C₁-C₆ heteroalkyl, substituted or unsubstituted C₃-C₇ cycloalkyl, or substituted or unsubstituted C₂-C₇ heterocycloalkyl; or R⁵ and R⁷ taken together form a bond; and
R⁶ is H, halogen, —CN, C₁-C₆ alkyl, C₁-C₆ heteroalkyl, heteroaryl, aryl, alkylaminylalkyl, dialkylaminylalkyl, cycloalkyl or heterocycloalkyl, each of which is optionally substituted.

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. The conjugate of claim 19, wherein the electrophilic functional group comprises a structure selected from 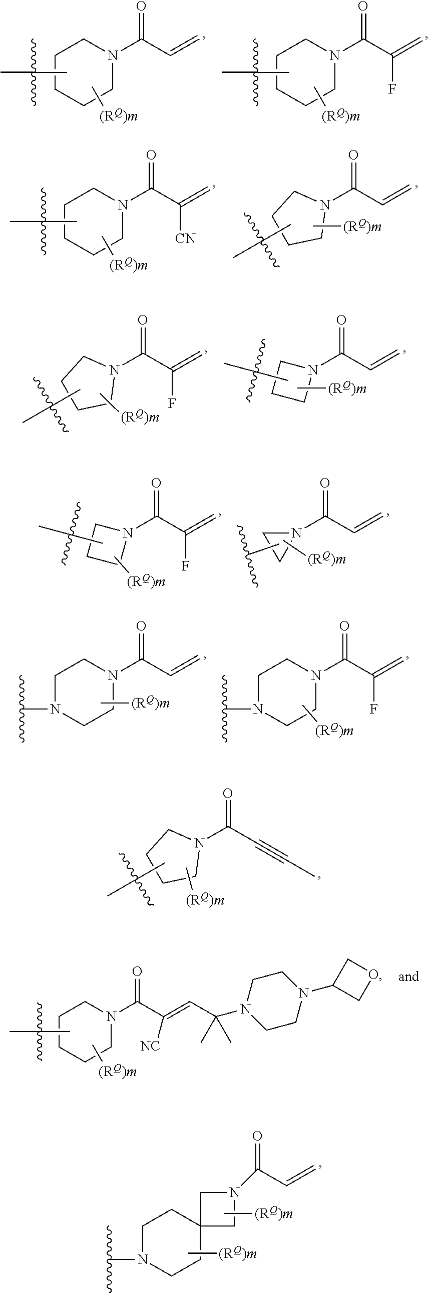
wherein
m is 0, 1, 2, 3, 4, or 5; and
wherein each R^{Q} is independently D, halogen, —CN, —NH₂, —NH(alkyl), —N(alkyl)₂, —OH, oxo, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, or arylsulfone, wherein each of the alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, or arylsulfone is optionally substituted.

36. (canceled)

37. (canceled)

38. The conjugate of claim 11, wherein the electrophilic functional group comprises a structure selected from

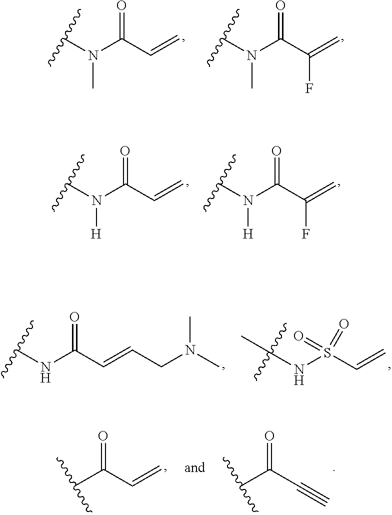

39. (canceled)

40. The conjugate of claim 1, wherein the intracellular protein is a tumor associated protein.

41. (canceled)

42. The conjugate of any one of claim 1, wherein the intracellular mutated protein is a GTPase KRas (KRAS protein), Tumor Protein P53 (TP53 protein), Isocitrate Dehydrogenase (NADP(+)) 1 (IDH1 protein), Guanine Nucleotide binding protein (GNAS protein), F-Box And WD Repeat Domain Containing 7 (FBXW7 protein), Catenin beta-1 (CTNNB1 protein), DNA (cytosine-5)-methyltransferase 3A (DNMT3A protein), Epidermal growth factor receptor (EGFR protein), Bruton's tyrosine kinase (BTK protein), Janus kinase 3 (JAK3 protein), Fibroblast growth factor receptor (FGFR protein), Human epidermal growth factor receptor 2 (HER2), or Human epidermal growth factor receptor 3 (HER3).

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. The conjugate of any one of claim 1, wherein the targeting ligand form a covalent bond with a lysine residue or a serine residue of the intracellular mutated protein.

70. (canceled)

71. (canceled)

72. (canceled)

73. (canceled)

74. (canceled)

75. The conjugate of claim 1, wherein the targeting ligand is a compound selected from Table 1.

76. (canceled)

77. (canceled)

78. (canceled)

79. (canceled)

80. (canceled)

81. The conjugate of claim 1, wherein the linker is configured to covalently attach the targeting ligand to the metal chelator.

82. (canceled)

83. (canceled)

84. (canceled)

85. (canceled)

86. (canceled)

87. (canceled)

88. The conjugate of claim 2, wherein the radionuclide is actinium-225.

89. (canceled)

90. (canceled)

91. (canceled)

92. (canceled)

93. (canceled)

94. A pharmaceutical composition comprising a conjugate of claim 1 and a pharmaceutically acceptable excipient or carrier.

95. (canceled)

96. A method of treating cancer in a subject in need thereof, comprising administering to the subject a conjugate of claim 1.

97. (canceled)

98. (canceled)

99. (canceled)

100. (canceled)

101. (canceled)

102. (canceled)

103. (canceled)

104. (canceled)

105. (canceled)

106. (canceled)

107. (canceled)

108. A covalently modified protein, comprising: (a) an intracellular protein (e.g., mutated protein) comprising a cysteine residue; and (b) a conjugate of claim 1.

109. (canceled)


SEQUENCE LISTING (3 sequences)

SEQ ID NO: 1 - protein, 189 aa, unidentified [sequence omitted]

SEQ ID NO: 2 - protein, 188 aa, unidentified [sequence omitted]

SEQ ID NO: 3 - protein, 5 aa, synthetic construct
EAAAK